SEQUENCE LISTING (236 sequences)

SEQ ID NO: 1 - protein, 123 aa, Homo sapiens
MDLLHKNMKHLWFFLLLVAAPRWVLSQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAR

SEQ ID NO: 2 - protein, 26 aa, Homo sapiens
MDLLHKNMKHLWFFLLLVAAPRWVLS

SEQ ID NO: 3 - protein, 97 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAR

SEQ ID NO: 4 - protein, 25 aa, synthetic construct [sequence omitted]

SEQ ID NO: 5 - protein, 122 aa, synthetic construct
EVQLQQSGPELVKPGASMKISCKASGYSFTGSTMNWVKQSPGKNLEWIGLINPYNGGTSYNQKFEGKATLTVDKSSSTAYMELLSLTSVDSAVYYCASRGKNGYDGRDFDCWGQGTSVTVSS

SEQ ID NO: 6 - protein, 8 aa, synthetic construct
GYSFTGST

SEQ ID NO: 7 - protein, 8 aa, synthetic construct
INPYNGGT

SEQ ID NO: 8 - protein, 15 aa, synthetic construct
ASRGKNGYDGRDFDC

SEQ ID NO: 9 - protein, 25 aa, synthetic construct
EVQLQQSGPELVKPGASMKISCKAS

SEQ ID NO: 10 - protein, 17 aa, synthetic construct
MNWVKQSPGKNLEWIGL

SEQ ID NO: 11 - protein, 38 aa, synthetic construct
SYNQKFEGKATLTVDKSSSTAYMELLSLTSVDSAVYYC

SEQ ID NO: 12 - protein, 11 aa, synthetic construct
WGQGTSVTVSS

SEQ ID NO: 13 - protein, 112 aa, synthetic construct
DIVLTQSPLSLPVNIGDQASISCKSTKSLLNSGGFTYLDWYLQKPGQSPQLLIYLVSNRFSGVPDRFSGSGSGTDFTLKISRVEAEDLGVYYCFQSNYFPFTFGSGTKLEIK

SEQ ID NO: 14 - protein, 11 aa, synthetic construct
KSLLNSGGFTY

SEQ ID NO: 15 - skipped

SEQ ID NO: 16 - protein, 9 aa, synthetic construct
FQSNYFPFT

SEQ ID NO: 17 - protein, 26 aa, synthetic construct
DIVLTQSPLSLPVNIGDQASISCKST

SEQ ID NO: 18 - protein, 17 aa, synthetic construct
LDWYLQKPGQSPQLLIY

SEQ ID NO: 19 - protein, 36 aa, synthetic construct
NRFSGVPDRFSGSGSGTDFTLKISRVEAEDLGVYYC

SEQ ID NO: 20 - protein, 10 aa, synthetic construct
FGSGTKLEIK

SEQ ID NO: 21 - protein, 114 aa, synthetic construct
EVMLVESGGGLVQPGGSLKLSCAASGFTFSSYGMSWVRQTPDKRLELVATINSNGVNTYYPDSVKGRFTISRDNAKNTLYLQMSSLKSEDTAMYYCARVPFDYWGQGTSLTVSS

SEQ ID NO: 22 - protein, 8 aa, synthetic construct
GFTFSSYG

SEQ ID NO: 23 - protein, 8 aa, synthetic construct
INSNGVNT

SEQ ID NO: 24 - protein, 7 aa, synthetic construct
ARVPFDY

SEQ ID NO: 25 - protein, 25 aa, synthetic construct
EVMLVESGGGLVQPGGSLKLSCAAS

SEQ ID NO: 26 - protein, 17 aa, synthetic construct
MSWVRQTPDKRLELVAT

SEQ ID NO: 27 - protein, 38 aa, synthetic construct
YYPDSVKGRFTISRDNAKNTLYLQMSSLKSEDTAMYYC

SEQ ID NO: 28 - protein, 11 aa, synthetic construct
WGQGTSLTVSS

SEQ ID NO: 29 - protein, 106 aa, synthetic construct
DIVMTQSPAIMSASLGEKVTMSCRASSSVDYIYWYQQKSDASPKLWIYYTSTLAPGVPARFSGSGSGNSYSLTISSMEGEDAATYYCQQFTSSPWTFGGGTRLEIK

SEQ ID NO: 30 - protein, 5 aa, synthetic construct
SSVDY

SEQ ID NO: 31 - skipped

SEQ ID NO: 32 - protein, 9 aa, synthetic construct
QQFTSSPWT

SEQ ID NO: 33 - protein, 26 aa, synthetic construct
DIVMTQSPAIMSASLGEKVTMSCRAS

SEQ ID NO: 34 - protein, 17 aa, synthetic construct
IYWYQQKSDASPKLWIY

SEQ ID NO: 35 - protein, 36 aa, synthetic construct
TLAPGVPARFSGSGSGNSYSLTISSMEGEDAATYYC

SEQ ID NO: 36 - protein, 10 aa, synthetic construct
FGGGTRLEIK

SEQ ID NO: 37 - protein, 121 aa, synthetic construct
EVQLQQSGAEFVRSGASVKLSCTASGFNIKDYYMHWVKQRPEQGLDWIGWIDPENGDADYDPKFQGKATMTADTSSNTAYLHLSSLTSEDAAVYYCNACHYYGYEDAMDYWGQGTSVTVSS

SEQ ID NO: 38 - protein, 8 aa, synthetic construct
GFNIKDYY

SEQ ID NO: 39 - protein, 8 aa, synthetic construct
IDPENGDA

SEQ ID NO: 40 - protein, 14 aa, synthetic construct
NACHYYGYEDAMDY

SEQ ID NO: 41 - protein, 25 aa, synthetic construct
EVQLQQSGAEFVRSGASVKLSCTAS

SEQ ID NO: 42 - protein, 17 aa, synthetic construct
MHWVKQRPEQGLDWIGW

SEQ ID NO: 43 - protein, 38 aa, synthetic construct
DYDPKFQGKATMTADTSSNTAYLHLSSLTSEDAAVYYC

SEQ ID NO: 44 - protein, 11 aa, synthetic construct
WGQGTSVTVSS

SEQ ID NO: 45 - protein, 107 aa, synthetic construct
DIQMTQSPSSLSASLGERVSLTCRASQEISGYLSWLQQKPDGTIKRLIYAASTLDSGVPKRFSGSRSGSDYSLTISSLESEDFADYYCLQYAGYPRTFGGGTRLEIK

SEQ ID NO: 46 - protein, 6 aa, synthetic construct
QEISGY

SEQ ID NO: 47 - skipped

SEQ ID NO: 48 - protein, 9 aa, synthetic construct
LQYAGYPRT

SEQ ID NO: 49 - protein, 26 aa, synthetic construct
DIQMTQSPSSLSASLGERVSLTCRAS

SEQ ID NO: 50 - protein, 17 aa, synthetic construct
LSWLQQKPDGTIKRLIY

SEQ ID NO: 51 - protein, 36 aa, synthetic construct
TLDSGVPKRFSGSRSGSDYSLTISSLESEDFADYYC

SEQ ID NO: 52 - protein, 10 aa, synthetic construct
FGGGTRLEIK

SEQ ID NO: 53 - protein, 113 aa, synthetic construct
EVQLVESGPSLVKPSQTLSLICSVTGDSITSGYWNWVRKFPGNKLEYMGYISYSGSAHYNPSLKSRISITRDTSKNQYYLQLNSMTTEDTATYYCASYSFAYWGQGTLVTVSA

SEQ ID NO: 54 - protein, 8 aa, synthetic construct
GDSITSGY

SEQ ID NO: 55 - protein, 7 aa, synthetic construct
ISYSGSA

SEQ ID NO: 56 - protein, 7 aa, synthetic construct
ASYSFAY

SEQ ID NO: 57 - protein, 25 aa, synthetic construct
EVQLVESGPSLVKPSQTLSLICSVT

SEQ ID NO: 58 - protein, 17 aa, synthetic construct
WNWVRKFPGNKLEYMGY

SEQ ID NO: 59 - protein, 38 aa, synthetic construct
HYNPSLKSRISITRDTSKNQYYLQLNSMTTEDTATYYC

SEQ ID NO: 60 - protein, 11 aa, synthetic construct
WGQGTLVTVSA

SEQ ID NO: 61 - protein, 107 aa, synthetic construct
DIVMTQSPATLSVTPGDSVSLSCRANQSIDNNLHWYQQRSHESPRLLINYASQSISGIPSRFSGSGSGTDFTLSISSVETEDFGMYFCQQSYGWPYTFGGGTKLEIK

SEQ ID NO: 62 - protein, 6 aa, synthetic construct
QSIDNN

SEQ ID NO: 63 - skipped

SEQ ID NO: 64 - protein, 9 aa, synthetic construct
QQSYGWPYT

SEQ ID NO: 65 - protein, 26 aa, synthetic construct
DIVMTQSPATLSVTPGDSVSLSCRAN

SEQ ID NO: 66 - protein, 17 aa, synthetic construct
LHWYQQRSHESPRLLIN

SEQ ID NO: 67 - protein, 36 aa, synthetic construct
QSISGIPSRFSGSGSGTDFTLSISSVETEDFGMYFC

SEQ ID NO: 68 - protein, 10 aa, synthetic construct
FGGGTKLEIK

SEQ ID NO: 69 - protein, 122 aa, synthetic construct
  VARIANT 9: note = P or A
  VARIANT 11: note = L or F
  VARIANT 13: note = K or R
  VARIANT 14: note = P or S
  VARIANT 18: note = M or V
  VARIANT 20: note = L or I
  VARIANT 23: note = K or T
  VARIANT 27: note = Y or F
  VARIANT 28: note = S or N
  VARIANT 29: note = F or I
  VARIANT 30: note = T or K
  VARIANT 31: note = G or D
  VARIANT 32: note = S or Y
  VARIANT 33: note = T or Y
  VARIANT 35: note = N or H
  VARIANT 40: note = S or R
  VARIANT 42: note = G or E
  VARIANT 43: note = K or Q
  VARIANT 44: note = N or G
  VARIANT 46: note = E or D
  VARIANT 50: note = L or W
  VARIANT 52: note = N or D
  VARIANT 54: note = Y or E
  VARIANT 57: note = G or D
  VARIANT 58: note = T or A
  VARIANT 59: note = S or D
  VARIANT 61: note = N or D
  VARIANT 62: note = Q or P
  VARIANT 65: note = E or Q
  VARIANT 70: note = L or M
  VARIANT 72: note = V or A
  VARIANT 74: note = K or T
  VARIANT 77: note = S or N
  VARIANT 81: note = M or L
  VARIANT 82: note = E or H
  VARIANT 84: note = L or S
  VARIANT 89: note = V or E
  VARIANT 91: note = S or A
  VARIANT 97: note = A or N
  VARIANT 98: note = S or A
  VARIANT 99: note = R or C
  VARIANT 100: note = G or H
  VARIANT 101: note = K or Y
  VARIANT 102: note = N or Y
  VARIANT 105: note = D or E
  VARIANT 106: note = G or D
  VARIANT 107: note = R or absent
  VARIANT 108: note = D or A
  VARIANT 109: note = F or M
  VARIANT 111: note = C or Y
EVQLQQSGXEXVXXGASXKXSCXASGXXXXXXXMXWVKQXPXXXLXWIGXIXPXNGXXXYXXKFXGKATXTXDXSSXTAYXXLXSLTSXDXAVYYCXXXXXXGYXXXXXDXWGQGTSVTVSS

SEQ ID NO: 70 - protein, 114 aa, synthetic construct
  VARIANT 3: note = M or Q
  VARIANT 9: note = G or P
  VARIANT 10: note = G or S
  VARIANT 13: note = Q or K
  VARIANT 15: note = G or S
  VARIANT 16: note = G or Q
  VARIANT 17: note = S or T
  VARIANT 19: note = K or S
  VARIANT 21: note = S or I
  VARIANT 23: note = A or S
  VARIANT 24: note = A or V
  VARIANT 25: note = S or T
  VARIANT 27: note = F or D
  VARIANT 28: note = T or S
  VARIANT 29: note = F or I
  VARIANT 30: note = S or T
  VARIANT 32: note = Y or G
  VARIANT 33: note = G or Y
  VARIANT 34: note = M or W
  VARIANT 35: note = S or N
  VARIANT 39: note = Q or K
  VARIANT 40: note = T or F
  VARIANT 42: note = D or G
  VARIANT 43: note = K or N
  VARIANT 44: note = R or K
  VARIANT 47: note = L or Y
  VARIANT 48: note = V or M
  VARIANT 49: note = A or G
  VARIANT 50: note = T or Y
  VARIANT 52: note = N or S
  VARIANT 53: note = S or Y
  VARIANT 54: note = N or S
  VARIANT 56: note = V or S
  VARIANT 57: note = N or A
  VARIANT 58: note = T or H
  VARIANT 60: note = Y or N
  VARIANT 61: note = P or absent
  VARIANT 62: note = D or P
  VARIANT 64: note = V or L
  VARIANT 66: note = G or S
  VARIANT 68: note = F or I
  VARIANT 69: note = T or S
  VARIANT 71: note = S or T
  VARIANT 74: note = N or T
  VARIANT 75: note = A or S
  VARIANT 78: note = T or Q
  VARIANT 79: note = L or Y
  VARIANT 83: note = M or L
  VARIANT 84: note = S or N
  VARIANT 86: note = L or M
  VARIANT 87: note = K or T
  VARIANT 88: note = S or T
  VARIANT 93: note = M or T
  VARIANT 98: note = R or S
  VARIANT 99: note = V or Y
  VARIANT 100: note = P or S
  VARIANT 102: note = D or A
  VARIANT 109: note = S or L
  VARIANT 110: note = L or V
  VARIANT 114: note = S or A
EVXLVESGXXLVXPXXXLXLXCXXXGXXXXSXXXXWVRXXPXXXLEXXXXIXXXGXXXYXXXSXKXRXXIXRDXXKNXXYLQXXSXXXEDTAXYYCAXXXFXYWGQGTXXTVSX

SEQ ID NO: 71 - protein, 330 aa, Homo sapiens [sequence omitted]

SEQ ID NO: 72 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 73 - protein, 12 aa, Homo sapiens
EPKSCDKTHTCP

SEQ ID NO: 74 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 75 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 78 - protein, 107 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 107 aa, Homo sapiens [sequence omitted]

SEQ ID NO: 80 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 81 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 82 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 83 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 84 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 85 - protein, 12 aa, synthetic construct
EPKSSDKTHTCP

SEQ ID NO: 86 - protein, 452 aa, synthetic construct
EVQLQQSGPELVKPGASMKISCKASGYSFTGSTMNWVKQSPGKNLEWIGLINPYNGGTSYNQKFEGKATLTVDKSSSTAYMELLSLTSVDSAVYYCASRGKNGYDGRDFDCWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 444 aa, synthetic construct
EVMLVESGGGLVQPGGSLKLSCAASGFTFSSYGMSWVRQTPDKRLELVATINSNGVNTYYPDSVKGRFTISRDNAKNTLYLQMSSLKSEDTAMYYCARVPFDYWGQGTSLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 451 aa, synthetic construct
EVQLQQSGAEFVRSGASVKLSCTASGFNIKDYYMHWVKQRPEQGLDWIGWIDPENGDADYDPKFQGKATMTADTSSNTAYLHLSSLTSEDAAVYYCNACHYYGYEDAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 443 aa, synthetic construct
EVQLVESGPSLVKPSQTLSLICSVTGDSITSGYWNWVRKFPGNKLEYMGYISYSGSAHYNPSLKSRISITRDTSKNQYYLQLNSMTTEDTATYYCASYSFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 219 aa, synthetic construct
DIVLTQSPLSLPVNIGDQASISCKSTKSLLNSGGFTYLDWYLQKPGQSPQLLIYLVSNRFSGVPDRFSGSGSGTDFTLKISRVEAEDLGVYYCFQSNYFPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 213 aa, synthetic construct
DIVMTQSPAIMSASLGEKVTMSCRASSSVDYIYWYQQKSDASPKLWIYYTSTLAPGVPARFSGSGSGNSYSLTISSMEGEDAATYYCQQFTSSPWTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 214 aa, synthetic construct
DIQMTQSPSSLSASLGERVSLTCRASQEISGYLSWLQQKPDGTIKRLIYAASTLDSGVPKRFSGSRSGSDYSLTISSLESEDFADYYCLQYAGYPRTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 214 aa, synthetic construct
DIVMTQSPATLSVTPGDSVSLSCRANQSIDNNLHWYQQRSHESPRLLINYASQSISGIPSRFSGSGSGTDFTLSISSVETEDFGMYFCQQSYGWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 330 aa, Mus musculus
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 95 - protein, 452 aa, synthetic construct
EVQLQQSGPELVKPGASMKISCKASGYSFTGSTMNWVKQSPGKNLEWIGLINPYNGGTSYNQKFEGKATLTVDKSSSTAYMELLSLTSVDSAVYYCASRGKNGYDGRDFDCWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 96 - protein, 444 aa, synthetic construct
EVMLVESGGGLVQPGGSLKLSCAASGFTFSSYGMSWVRQTPDKRLELVATINSNGVNTYYPDSVKGRFTISRDNAKNTLYLQMSSLKSEDTAMYYCARVPFDYWGQGTSLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 97 - protein, 451 aa, synthetic construct
EVQLQQSGAEFVRSGASVKLSCTASGFNIKDYYMHWVKQRPEQGLDWIGWIDPENGDADYDPKFQGKATMTADTSSNTAYLHLSSLTSEDAAVYYCNACHYYGYEDAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 98 - protein, 443 aa, synthetic construct
EVQLVESGPSLVKPSQTLSLICSVTGDSITSGYWNWVRKFPGNKLEYMGYISYSGSAHYNPSLKSRISITRDTSKNQYYLQLNSMTTEDTATYYCASYSFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 99 - protein, 452 aa, synthetic construct
QVQLVQSGAEVKKPGASMKVSCKASGYSFTGSTMNWVRQAPGQGLEWMGWINPYNGGTNYAQKFQGRVTMTRDTSISTAYMELSRLRSDDTAVYYCASRGKNGYDGRDFDCWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 100 - protein, 219 aa, Mus musculus
ELVMTQSPLSLSVSLGDQASISCRSSQSLVHTNGNTYLHWYLQKPGLSPKLLIYIVSNRFSGVPDRFSGSGSGTDFTLKISRVEAEDLGVYFCSQVTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 101 - protein, 331 aa, synthetic construct
DIVLTQSPLSLPVNIGDQASISCKSTKSLLNSGGFTYLDWYLQKPGQSPQLLIYLVSNRFSGVPDRFSGSGSGTDFTLKISRVEAEDLGVYYCFQSNYFPFTFGSGTKLEIKELVMTQSPLSLSVSLGDQASISCRSSQSLVHTNGNTYLHWYLQKPGLSPKLLIYIVSNRFSGVPDRFSGSGSGTDFTLKISRVEAEDLGVYFCSQVTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 102 - protein, 325 aa, synthetic construct
DIVMTQSPAIMSASLGEKVTMSCRASSSVDYIYWYQQKSDASPKLWIYYTSTLAPGVPARFSGSGSGNSYSLTISSMEGEDAATYYCQQFTSSPWTFGGGTRLEIKELVMTQSPLSLSVSLGDQASISCRSSQSLVHTNGNTYLHWYLQKPGLSPKLLIYIVSNRFSGVPDRFSGSGSGTDFTLKISRVEAEDLGVYFCSQVTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 103 - protein, 326 aa, synthetic construct
DIQMTQSPSSLSASLGERVSLTCRASQEISGYLSWLQQKPDGTIKRLIYAASTLDSGVPKRFSGSRSGSDYSLTISSLESEDFADYYCLQYAGYPRTFGGGTRLEIKELVMTQSPLSLSVSLGDQASISCRSSQSLVHTNGNTYLHWYLQKPGLSPKLLIYIVSNRFSGVPDRFSGSGSGTDFTLKISRVEAEDLGVYFCSQVTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 104 - protein, 326 aa, synthetic construct
DIVMTQSPATLSVTPGDSVSLSCRANQSIDNNLHWYQQRSHESPRLLINYASQSISGIPSRFSGSGSGTDFTLSISSVETEDFGMYFCQQSYGWPYTFGGGTKLEIKELVMTQSPLSLSVSLGDQASISCRSSQSLVHTNGNTYLHWYLQKPGLSPKLLIYIVSNRFSGVPDRFSGSGSGTDFTLKISRVEAEDLGVYFCSQVTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 105 - protein, 331 aa, synthetic construct
EIVLTQSPATLSLSPGERATLSCKSTKSLLNSGGFSYLDWYQQKPGQAPRLLIYLVSNRFTGIPARFSGSGSGTDFTLTISSLEPEDFAVYYCFQSNYFPFTFGQGTKLEIKELVMTQSPLSLSVSLGDQASISCRSSQSLVHTNGNTYLHWYLQKPGLSPKLLIYIVSNRFSGVPDRFSGSGSGTDFTLKISRVEAEDLGVYFCSQVTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 106 - protein, 445 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 447 aa, synthetic construct
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 450 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGYYDILTGYYYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 214 aa, synthetic construct
DIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 220 aa, synthetic construct
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 214 aa, synthetic construct
EIVLTQSPATLSLSPGERATLSCRASQSVSRYLAWYQQKPGQAPRLLIYDASNRATGIPARFSGSGSGTDSTLTISSLEPEDFAVYYCQQRSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 115 aa, synthetic construct
QVQLQGAGAGLLKPSETLSLTCGASGGSFSGYYWSWIRQPPGKGLEWIGEINHSGSTNYNPSLKSRVTISVDTSKNQFSLKLSSVTAADTAVYYCARVINWFDPWGQGTLVTVSS

SEQ ID NO: 113 - protein, 445 aa, synthetic construct
QVQLQGAGAGLLKPSETLSLTCGASGGSFSGYYWSWIRQPPGKGLEWIGEINHSGSTNYNPSLKSRVTISVDTSKNQFSLKLSSVTAADTAVYYCARVINWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 450 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGLGYSGSYYAPNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 219 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 120 aa, synthetic construct
QVQLQGAGAGLLKPSETLSLTCGASGGSFSGYYWSWIRQPPGKGLEWIGEINHSGSTNYNPSLKSRVTISVDTSKNQFSLKLSSVTAADTAVYYCARGLGYSGSYYAPNWGQGTLVTVSS

SEQ ID NO: 117 - protein, 450 aa, synthetic construct
QVQLQGAGAGLLKPSETLSLTCGASGGSFSGYYWSWIRQPPGKGLEWIGEINHSGSTNYNPSLKSRVTISVDTSKNQFSLKLSSVTAADTAVYYCARGLGYSGSYYAPNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 123 aa, synthetic construct
QVQLQGAGAGLLKPSETLSLTCGASGGSFSGYYWSWIRQPPGKGLEWIGEINHSGSTNYNPSLKSRVTISVDTSKNQFSLKLSSVTAADTAVYYCARGYYDILTGYYYYFDYWGQGTLVTVSS

SEQ ID NO: 119 - protein, 453 aa, synthetic construct
QVQLQGAGAGLLKPSETLSLTCGASGGSFSGYYWSWIRQPPGKGLEWIGEINHSGSTNYNPSLKSRVTISVDTSKNQFSLKLSSVTAADTAVYYCARGYYDILTGYYYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 453 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGYYDILTGYYYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 6 aa, Homo sapiens
PCPAPE

SEQ ID NO: 122 - protein, 107 aa, Mus musculus
LLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPK

SEQ ID NO: 123 - protein, 107 aa, Mus musculus
GSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 124 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 125 - protein, 122 aa, synthetic construct
QVQLVQSGAEVKKPGASMKVSCKASGYSFTGSTMNWVRQAPGQGLEWMGWINPYNGGTNYAQKFQGRVTMTRDTSISTAYMELSRLRSDDTAVYYCASRGKNGYDGRDFDCWGQGTLVTVSS

SEQ ID NO: 126 - protein, 25 aa, synthetic construct
QVQLVQSGAEVKKPGASMKVSCKAS

SEQ ID NO: 127 - protein, 17 aa, synthetic construct
MNWVRQAPGQGLEWMGW

SEQ ID NO: 128 - protein, 38 aa, synthetic construct
NYAQKFQGRVTMTRDTSISTAYMELSRLRSDDTAVYYC

SEQ ID NO: 129 - protein, 11 aa, synthetic construct
WGQGTLVTVSS

SEQ ID NO: 130 - protein, 112 aa, synthetic construct
EIVLTQSPATLSLSPGERATLSCKSTKSLLNSGGFSYLDWYQQKPGQAPRLLIYLVSNRFTGIPARFSGSGSGTDFTLTISSLEPEDFAVYYCFQSNYFPFTFGQGTKLEIK

SEQ ID NO: 131 - protein, 11 aa, synthetic construct
KSLLNSGGFSY

SEQ ID NO: 132 - protein, 26 aa, synthetic construct
EIVLTQSPATLSLSPGERATLSCKST

SEQ ID NO: 133 - protein, 17 aa, synthetic construct
LDWYQQKPGQAPRLLIY

SEQ ID NO: 134 - protein, 36 aa, synthetic construct
NRFTGIPARFSGSGSGTDFTLTISSLEPEDFAVYYC

SEQ ID NO: 135 - protein, 10 aa, synthetic construct
FGQGTKLEIK

SEQ ID NO: 136 - protein, 452 aa, synthetic construct
QVQLVQSGAEVKKPGASMKVSCKASGYSFTGSTMNWVRQAPGQGLEWMGWINPYNGGTNYAQKFQGRVTMTRDTSISTAYMELSRLRSDDTAVYYCASRGKNGYDGRDFDCWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 219 aa, synthetic construct
EIVLTQSPATLSLSPGERATLSCKSTKSLLNSGGFSYLDWYQQKPGQAPRLLIYLVSNRFTGIPARFSGSGSGTDFTLTISSLEPEDFAVYYCFQSNYFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 443 aa, synthetic construct
EVQLVESGPSLVKPSQTLSLICSVTGDAITSGYWNWVRKFPGNKLEYMGYISYSGSAHYNPSLKSRISITRDTSKNQYYLQLNSMTTEDTATYYCASYSFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 139 - protein, 443 aa, synthetic construct
EVQLVESGPSLVKPSQTLSLICSVTGDAITSGYWNWVRKFPGNKLEYMGYISYSGSAHYNPSLKSRISITRDTSKNQYYLQLNSMTTEDTATYYCASYSFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 214 aa, synthetic construct
DIVMTQSPATLSVTPGDSVSLSCRAQQSIDNQLHWYQQRSHESPRLLINYASQSISGIPSRFSGSGSGTDFTLSISSVETEDFGMYFCQQSYGWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 113 aa, synthetic construct
EVQLVESGPSLVKPSQTLSLICSVTGDAITSGYWNWVRKFPGNKLEYMGYISYSGSAHYNPSLKSRISITRDTSKNQYYLQLNSMTTEDTATYYCASYSFAYWGQGTLVTVSA

SEQ ID NO: 142 - protein, 8 aa, synthetic construct
GDAITSGY

SEQ ID NO: 143 - protein, 107 aa, synthetic construct
DIVMTQSPATLSVTPGDSVSLSCRAQQSIDNQLHWYQQRSHESPRLLINYASQSISGIPSRFSGSGSGTDFTLSISSVETEDFGMYFCQQSYGWPYTFGGGTKLEIK

SEQ ID NO: 144 - protein, 6 aa, synthetic construct
QSIDNQ

SEQ ID NO: 145 - protein, 26 aa, synthetic construct
DIVMTQSPATLSVTPGDSVSLSCRAQ

SEQ ID NO: 146 - protein, 214 aa, synthetic construct
DIVMTQSPATLSVTPGDSVSLSCRASQSIDNQLHWYQQRSHESPRLLINYASQSISGIPSRFSGSGSGTDFTLSISSVETEDFGMYFCQQSYGWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 107 aa, synthetic construct
DIVMTQSPATLSVTPGDSVSLSCRASQSIDNQLHWYQQRSHESPRLLINYASQSISGIPSRFSGSGSGTDFTLSISSVETEDFGMYFCQQSYGWPYTFGGGTKLEIK

SEQ ID NO: 148 - protein, 26 aa, synthetic construct
DIVMTQSPATLSVTPGDSVSLSCRAS

SEQ ID NO: 149 - protein, 326 aa, synthetic construct
DIVMTQSPATLSVTPGDSVSLSCRAQQSIDNQLHWYQQRSHESPRLLINYASQSISGIPSRFSGSGSGTDFTLSISSVETEDFGMYFCQQSYGWPYTFGGGTKLEIKELVMTQSPLSLSVSLGDQASISCRSSQSLVHTNGNTYLHWYLQKPGLSPKLLIYIVSNRFSGVPDRFSGSGSGTDFTLKISRVEAEDLGVYFCSQVTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 150 - protein, 326 aa, synthetic construct
DIVMTQSPATLSVTPGDSVSLSCRASQSIDNQLHWYQQRSHESPRLLINYASQSISGIPSRFSGSGSGTDFTLSISSVETEDFGMYFCQQSYGWPYTFGGGTKLEIKELVMTQSPLSLSVSLGDQASISCRSSQSLVHTNGNTYLHWYLQKPGLSPKLLIYIVSNRFSGVPDRFSGSGSGTDFTLKISRVEAEDLGVYFCSQVTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 151 - protein, 113 aa, synthetic construct
  VARIANT 28: note = S or A
EVQLVESGPSLVKPSQTLSLICSVTGDXITSGYWNWVRKFPGNKLEYMGYISYSGSAHYNPSLKSRISITRDTSKNQYYLQLNSMTTEDTATYYCASYSFAYWGQGTLVTVSA

SEQ ID NO: 152 - protein, 8 aa, synthetic construct
  VARIANT 3: note = S or A
GDXITSGY

SEQ ID NO: 153 - protein, 107 aa, synthetic construct
  VARIANT 26: note = N or Q or S
  VARIANT 32: note = N or Q
DIVMTQSPATLSVTPGDSVSLSCRAXQSIDNXLHWYQQRSHESPRLLINYASQSISGIPSRFSGSGSGTDFTLSISSVETEDFGMYFCQQSYGWPYTFGGGTKLEIK

SEQ ID NO: 154 - protein, 6 aa, synthetic construct
  VARIANT 6: note = N or Q
QSIDNX

SEQ ID NO: 155 - protein, 26 aa, synthetic construct
  VARIANT 26: note = N or Q or S
DIVMTQSPATLSVTPGDSVSLSCRAX

SEQ ID NO: 156 - protein, 122 aa, synthetic construct
  VARIANT 1: note = E or Q
  VARIANT 5: note = Q or V
  VARIANT 9: note = P or A
  VARIANT 11: note = L or V
  VARIANT 12: note = V or K
  VARIANT 20: note = I or V
  VARIANT 38: note = K or R
  VARIANT 40: note = S or A
  VARIANT 43: note = K or Q
  VARIANT 44: note = N or G
  VARIANT 48: note = I or M
  VARIANT 50: note = L or W
  VARIANT 59: note = S or N
  VARIANT 61: note = N or A
  VARIANT 65: note = E or Q
  VARIANT 67: note = K or R
  VARIANT 68: note = A or V
  VARIANT 70: note = L or M
  VARIANT 72: note = V or R
  VARIANT 74: note = K or T
  VARIANT 76: note = S or I
  VARIANT 84: note = L or S
  VARIANT 85: note = S or R
  VARIANT 87: note = T or R
  VARIANT 89: note = V or D
  VARIANT 91: note = S or T
  VARIANT 117: note = S or L
XVQLXQSGXEXXKPGASMKXSCKASGYSFTGSTMNWVXQXPGXXLEWXGXINPYNGGTXYXQKFXGXXTXTXDXSXSTAYMELXXLXSXDXAVYYCASRGKNGYDGRDFDCWGQGTXVTVSS

SEQ ID NO: 157 - protein, 25 aa, synthetic construct
  VARIANT 1: note = E or Q
  VARIANT 5: note = Q or V
  VARIANT 9: note = P or A
  VARIANT 11: note = L or V
  VARIANT 12: note = V or K
  VARIANT 20: note = I or V
XVQLXQSGXEXXKPGASMKXSCKAS

SEQ ID NO: 158 - protein, 17 aa, synthetic construct
  VARIANT 5: note = K or R
  VARIANT 7: note = S or A
  VARIANT 10: note = K or Q
  VARIANT 11: note = N or G
  VARIANT 15: note = I or M
  VARIANT 17: note = L or W
MNWVXQXPGXXLEWXGX

SEQ ID NO: 159 - protein, 38 aa, synthetic construct
  VARIANT 1: note = S or N
  VARIANT 3: note = N or A
  VARIANT 7: note = E or Q
  VARIANT 9: note = K or R
  VARIANT 10: note = A or V
  VARIANT 12: note = L or M
  VARIANT 14: note = V or R
  VARIANT 16: note = K or T
  VARIANT 18: note = S or I
  VARIANT 26: note = L or S
  VARIANT 27: note = S or R
  VARIANT 29: note = T or R
  VARIANT 31: note = V or D
  VARIANT 33: note = S or A or T
XYXQKFXGXXTXTXDXSXSTAYMELXXLXSXDXAVYYC

SEQ ID NO: 160 - protein, 11 aa, synthetic construct
  VARIANT 6: note = S or L
WGQGTXVTVSS

SEQ ID NO: 161 - protein, 112 aa, synthetic construct
  VARIANT 1: note = D or E
  VARIANT 9: note = L or A
  VARIANT 10: note = S or T
  VARIANT 12: note = P or S
  VARIANT 13: note = V or L
  VARIANT 14: note = N or S
  VARIANT 15: note = I or P
  VARIANT 17: note = D or E
  VARIANT 18: note = Q or R
  VARIANT 20: note = S or T
  VARIANT 21: note = I or L
  VARIANT 36: note = T or S
  VARIANT 42: note = L or Q
  VARIANT 48: note = S or A
  VARIANT 50: note = Q or R
  VARIANT 61: note = S or T
  VARIANT 63: note = V or I
  VARIANT 65: note = D or A
  VARIANT 79: note = K or T
  VARIANT 82: note = R or S
  VARIANT 83: note = V or L
  VARIANT 85: note = A or P
  VARIANT 88: note = L or F
  VARIANT 89: note = G or A
  VARIANT 105: note = S or Q
XIVLTQSPXXLXXXXGXXAXXSCKSTKSLLNSGGFXYLDWYXQKPGQXPXLLIYLVSNRFXGXPXRFSGSGSGTDFTLXISXXEXEDXXVYYCFQSNYFPFTFGXGTKLEIK

SEQ ID NO: 162 - protein, 11 aa, synthetic construct
  VARIANT 10: note = T or S
KSLLNSGGFXY

SEQ ID NO: 163 - protein, 26 aa, synthetic construct
  VARIANT 1: note = D or E
  VARIANT 9: note = L or A
  VARIANT 10: note = S or T
  VARIANT 12: note = P or S
  VARIANT 13: note = V or L
  VARIANT 14: note = N or S
  VARIANT 15: note = I or P
  VARIANT 17: note = D or E
  VARIANT 18: note = Q or R
  VARIANT 20: note = S or T
  VARIANT 21: note = I or L
XIVLTQSPXXLXXXXGXXAXXSCKST

SEQ ID NO: 164 - protein, 17 aa, synthetic construct
  VARIANT 5: note = L or Q
  VARIANT 11: note = S or A
  VARIANT 13: note = Q or R
LDWYXQKPGQXPXLLIY

SEQ ID NO: 165 - protein, 36 aa, synthetic construct
  VARIANT 4: note = S or T
  VARIANT 6: note = V or I
  VARIANT 8: note = D or A
  VARIANT 22: note = K or T
  VARIANT 25: note = R or S
  VARIANT 26: note = V or L
  VARIANT 28: note = A or P
  VARIANT 31: note = L or F
  VARIANT 32: note = G or A
NRFXGXPXRFSGSGSGTDFTLXISXXEXEDXXVYYC

SEQ ID NO: 166 - protein, 10 aa, synthetic construct
  VARIANT 3: note = S or Q
FGXGTKLEIK

SEQ ID NO: 167 - protein, 113 aa, synthetic construct
QVQLQESGPGLVKPSETLSLTCTVSGDAITSGYWNWIRQPPGKGLEYMGYISYSGSAHYNPSLKSRITISRDTSKNQYSLKLSSVTAADTAVYYCASYSFAYWGQGTLVTVSS

SEQ ID NO: 168 - protein, 25 aa, synthetic construct
QVQLQESGPGLVKPSETLSLTCTVS

SEQ ID NO: 169 - protein, 17 aa, synthetic construct
WNWIRQPPGKGLEYMGY

SEQ ID NO: 170 - protein, 38 aa, synthetic construct
HYNPSLKSRITISRDTSKNQYSLKLSSVTAADTAVYYC

SEQ ID NO: 171 - protein, 113 aa, synthetic construct
QVQLQESGPGLVKPSQTLSLTCTVSWDAITSGYWNWVRQHPGKGLEYMGYISYSGSAHYNPSLKSRITISRDTSKNQYSLKLSSVTAADTATYYCASYSFAYWGQGTLVTVSS

SEQ ID NO: 172 - protein, 8 aa, synthetic construct
WDAITSGY

SEQ ID NO: 173 - protein, 25 aa, synthetic construct
QVQLQESGPGLVKPSQTLSLTCTVS

SEQ ID NO: 174 - protein, 17 aa, synthetic construct
WNWVRQHPGKGLEYMGY

SEQ ID NO: 175 - protein, 38 aa, synthetic construct
HYNPSLKSRITISRDTSKNQYSLKLSSVTAADTATYYC

SEQ ID NO: 176 - protein, 113 aa, synthetic construct
QVQLQESGPGLVKPSETLSLTCSVSWDAITSGYWNWVRQPPGNKLEYMGYISYSGSAHYNPSLKSRITISRDTSKNQYSLTLTSMTAADTALYYCASYSFAYWGQGTLVTVSS

SEQ ID NO: 177 - skipped

SEQ ID NO: 178 - protein, 25 aa, synthetic construct
QVQLQESGPGLVKPSETLSLTCSVS

SEQ ID NO: 179 - protein, 17 aa, synthetic construct
WNWVRQPPGNKLEYMGY

SEQ ID NO: 180 - protein, 38 aa, synthetic construct
HYNPSLKSRITISRDTSKNQYSLTLTSMTAADTALYYC

SEQ ID NO: 181 - protein, 113 aa, synthetic construct
QVQLQESGPGLVKPSETLSLTCTVSWDAITSGYWNWVRQPPGNKLEYMGYISYSGSAHYNPSLKSRITISRDTSKNQYSLKLSSVTAADTAVYYCASYSFAYWGQGTLVTVSS

SEQ ID NO: 182 - protein, 113 aa, synthetic construct
QVQLQESGPGLVKPSQTLSLTCTVSWDAITSGYWNWVRQHPGNKLEYMGYISYSGSAHYNPSLKSRITISRDTSKNQYSLKLSSVTAADTAVYYCASYSFAYWGQGTLVTVSS

SEQ ID NO: 183 - protein, 17 aa, synthetic construct
WNWVRQHPGNKLEYMGY

SEQ ID NO: 184 - protein, 113 aa, synthetic construct
  VARIANT 16: note = E or Q
  VARIANT 23: note = T or S
  VARIANT 26: note = W or G
  VARIANT 37: note = V or I
  VARIANT 40: note = P or H
  VARIANT 43: note = K or N
  VARIANT 44: note = G or K
  VARIANT 92: note = V or T
QVQLQESGPGLVKPSXTLSLTCXVSXDAITSGYWNWXRQXPGXXLEYMGYISYSGSAHYNPSLKSRITISRDTSKNQYSLKLSSVTAADTAXYYCASYSFAYWGQGTLVTVSS

SEQ ID NO: 185 - protein, 8 aa, synthetic construct
  VARIANT 1: note = W or G
XDAITSGY

SEQ ID NO: 186 - protein, 25 aa, synthetic construct
  VARIANT 16: note = E or Q
  VARIANT 23: note = T or S
QVQLQESGPGLVKPSXTLSLTCXVS

SEQ ID NO: 187 - protein, 17 aa, synthetic construct
  VARIANT 4: note = V or I
  VARIANT 7: note = P or H
  VARIANT 10: note = K or N
  VARIANT 11: note = G or K
WNWXRQXPGXXLEYMGY

SEQ ID NO: 188 - protein, 38 aa, synthetic construct
  VARIANT 35: note = V or T
HYNPSLKSRITISRDTSKNQYSLKLSSVTAADTAXYYC

SEQ ID NO: 189 - protein, 107 aa, synthetic construct
EIVMTQSPDFQSVTPKEKVTITCRASQSIDNQLHWYQQKPDQSPKLLINYASQSISGVPSRFSGSGSGTDFTLTISSLEAEDAATYYCQQSYGWPYTFGQGTKLEIK

SEQ ID NO: 190 - protein, 26 aa, synthetic construct
EIVMTQSPDFQSVTPKEKVTITCRAS

SEQ ID NO: 191 - protein, 17 aa, synthetic construct
LHWYQQKPDQSPKLLIN

SEQ ID NO: 192 - protein, 36 aa, synthetic construct
QSISGVPSRFSGSGSGTDFTLTISSLEAEDAATYYC

SEQ ID NO: 193 - protein, 107 aa, synthetic construct
DIVMTQSPPSLSVTPGEPASLSCRASQSIDNQLHWYLQKPGQSPQLLINYASQSISGVTDRFSGSGSGTDFTLKISRVEAEDVGVYFCQQSYGWPYTFGGGTKVEIK

SEQ ID NO: 194 - protein, 26 aa, synthetic construct
DIVMTQSPPSLSVTPGEPASLSCRAS

SEQ ID NO: 195 - protein, 17 aa, synthetic construct
LHWYLQKPGQSPQLLIN

SEQ ID NO: 196 - protein, 36 aa, synthetic construct
QSISGVTDRFSGSGSGTDFTLKISRVEAEDVGVYFC

SEQ ID NO: 197 - protein, 10 aa, synthetic construct
FGGGTKVEIK

SEQ ID NO: 198 - protein, 107 aa, synthetic construct
DIVMTQSPPSLSVTPGESASISCRASQSIDNQLHWYLQKPGQSPQLLINYASQSISGVTDRFSGSGSGTDFTLKISRVEAEDVGMYFCQQSYGWPYTFGGGTKVEIK

SEQ ID NO: 199 - protein, 26 aa, synthetic construct
DIVMTQSPPSLSVTPGESASISCRAS

SEQ ID NO: 200 - protein, 36 aa, synthetic construct
QSISGVTDRFSGSGSGTDFTLKISRVEAEDVGMYFC

SEQ ID NO: 201 - protein, 107 aa, synthetic construct
DIQMTQSPSSLSASVGDRVTITCRASQSIDNQLHWYQQKPGKSPNLLINYASQSISGVPSRFSGSGSGTDFTLTISSLQPEDFATYFCQQSYGWPYTFGGGTKVEIK

SEQ ID NO: 202 - protein, 26 aa, synthetic construct [sequence omitted]

SEQ ID NO: 203 - protein, 17 aa, synthetic construct
LHWYQQKPGKSPNLLIN

SEQ ID NO: 204 - protein, 36 aa, synthetic construct
QSISGVPSRFSGSGSGTDFTLTISSLQPEDFATYFC

SEQ ID NO: 205 - protein, 107 aa, synthetic construct
EIVLTQSPDFQSVTPKEKVTITCRASQSIDNQLHWYQQKPDQSPKLLIKYASQSISGVPSRFSGSGSGTDFTLTISSLEAEDAATYFCQQSYGWPYTFGGGTKLEIK

SEQ ID NO: 206 - protein, 26 aa, synthetic construct
EIVLTQSPDFQSVTPKEKVTITCRAS

SEQ ID NO: 207 - protein, 17 aa, synthetic construct
LHWYQQKPDQSPKLLIK

SEQ ID NO: 208 - protein, 36 aa, synthetic construct
QSISGVPSRFSGSGSGTDFTLTISSLEAEDAATYFC

SEQ ID NO: 209 - protein, 107 aa, synthetic construct
  VARIANT 1: note = D or E
  VARIANT 3: note = V or Q
  VARIANT 4: note = M or L
  VARIANT 9: note = P or D or S
  VARIANT 10: note = S or F
  VARIANT 11: note = L or Q
  VARIANT 13: note = V or A
  VARIANT 14: note = T or S
  VARIANT 15: note = P or V
  VARIANT 16: note = G or K
  VARIANT 17: note = E or D
  VARIANT 18: note = K or R or S or P
  VARIANT 19: note = V or A
  VARIANT 20: note = T or S
  VARIANT 21: note = L or I
  VARIANT 22: note = T or S
  VARIANT 37: note = Q or L
  VARIANT 41: note = G or D
  VARIANT 42: note = Q or K
  VARIANT 45: note = K or N or Q
  VARIANT 49: note = N or K
  VARIANT 59: note = P or T
  VARIANT 60: note = S or D
  VARIANT 74: note = T or K
  VARIANT 77: note = R or S
  VARIANT 78: note = L or V
  VARIANT 79: note = E or Q
  VARIANT 80: note = A or P
  VARIANT 83: note = V or F or A
  VARIANT 84: note = A or G
  VARIANT 85: note = T or V or M
  VARIANT 87: note = F or Y
  VARIANT 100: note = G or Q
  VARIANT 104: note = L or V
XIXXTQSPXXXSXXXXXXXXXXCRASQSIDNQLHWYXQKPXXSPXLLIXYASQSISGVXXRFSGSGSGTDFTLXISXXXXEDXXXYXCQQSYGWPYTFGXGTKXEIK

SEQ ID NO: 210 - protein, 26 aa, synthetic construct
  VARIANT 1: note = D or E
  VARIANT 3: note = V or Q
  VARIANT 4: note = M or L
  VARIANT 9: note = P or D or S
  VARIANT 10: note = S or F
  VARIANT 11: note = L or Q
  VARIANT 13: note = V or A
  VARIANT 14: note = T or S
  VARIANT 15: note = P or V
  VARIANT 16: note = G or K
  VARIANT 17: note = E or D
  VARIANT 18: note = K or R or S or P
  VARIANT 19: note = V or A
  VARIANT 20: note = T or S
  VARIANT 21: note = L or I
  VARIANT 22: note = T or S
XIXXTQSPXXXSXXXXXXXXXXCRAS

SEQ ID NO: 211 - protein, 17 aa, synthetic construct
  VARIANT 5: note = Q or L
  VARIANT 9: note = G or D
  VARIANT 10: note = Q or K
  VARIANT 13: note = K or N or Q
  VARIANT 17: note = N or K
LHWYXQKPXXSPXLLIX

SEQ ID NO: 212 - protein, 36 aa, synthetic construct
  VARIANT 7: note = P or T
  VARIANT 8: note = S or D
  VARIANT 22: note = T or K
  VARIANT 25: note = R or S
  VARIANT 26: note = L or V
  VARIANT 27: note = E or Q
  VARIANT 28: note = A or P
  VARIANT 31: note = V or F or A
  VARIANT 32: note = A or G
  VARIANT 33: note = T or V or M
  VARIANT 35: note = F or Y
QSISGVXXRFSGSGSGTDFTLXISXXXXEDXXXYXC

SEQ ID NO: 213 - protein, 10 aa, synthetic construct
  VARIANT 3: note = G or Q
  VARIANT 7: note = L or V
FGXGTKXEIK

SEQ ID NO: 214 - protein, 443 aa, synthetic construct
  VARIANT 16: note = E or Q
  VARIANT 23: note = T or S
  VARIANT 26: note = W or G
  VARIANT 37: note = V or I
  VARIANT 40: note = P or H
  VARIANT 43: note = K or N
  VARIANT 44: note = G or K
  VARIANT 92: note = V or T
QVQLQESGPGLVKPSXTLSLTCXVSXDAITSGYWNWXRQXPGXXLEYMGYISYSGSAHYNPSLKSRITISRDTSKNQYSLKLSSVTAADTAXYYCASYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 443 aa, synthetic construct
QVQLQESGPGLVKPSETLSLTCTVSGDAITSGYWNWIRQPPGKGLEYMGYISYSGSAHYNPSLKSRITISRDTSKNQYSLKLSSVTAADTAVYYCASYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 443 aa, synthetic construct
QVQLQESGPGLVKPSQTLSLTCTVSWDAITSGYWNWVRQHPGKGLEYMGYISYSGSAHYNPSLKSRITISRDTSKNQYSLKLSSVTAADTATYYCASYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 443 aa, synthetic construct
QVQLQESGPGLVKPSETLSLTCSVSWDAITSGYWNWVRQPPGNKLEYMGYISYSGSAHYNPSLKSRITISRDTSKNQYSLTLTSMTAADTALYYCASYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 443 aa, synthetic construct
QVQLQESGPGLVKPSETLSLTCTVSWDAITSGYWNWVRQPPGNKLEYMGYISYSGSAHYNPSLKSRITISRDTSKNQYSLKLSSVTAADTAVYYCASYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 443 aa, synthetic construct
QVQLQESGPGLVKPSQTLSLTCTVSWDAITSGYWNWVRQHPGNKLEYMGYISYSGSAHYNPSLKSRITISRDTSKNQYSLKLSSVTAADTAVYYCASYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 214 aa, synthetic construct
  VARIANT 1: note = D or E
  VARIANT 3: note = V or Q
  VARIANT 4: note = M or L
  VARIANT 9: note = P or D or S
  VARIANT 10: note = S or F
  VARIANT 11: note = L or Q
  VARIANT 13: note = V or A
  VARIANT 14: note = T or S
  VARIANT 15: note = P or V
  VARIANT 16: note = G or K
  VARIANT 17: note = E or D
  VARIANT 18: note = K or R or S or P
  VARIANT 19: note = V or A
  VARIANT 20: note = T or S
  VARIANT 21: note = L or I
  VARIANT 22: note = T or S
  VARIANT 37: note = Q or L
  VARIANT 41: note = G or D
  VARIANT 42: note = Q or K
  VARIANT 45: note = K or N or Q
  VARIANT 49: note = N or K
  VARIANT 59: note = P or T
  VARIANT 60: note = S or D
  VARIANT 74: note = T or K
  VARIANT 77: note = R or S
  VARIANT 78: note = L or V
  VARIANT 79: note = E or Q
  VARIANT 80: note = A or P
  VARIANT 83: note = V or F or A
  VARIANT 84: note = A or G
  VARIANT 85: note = T or V or M
  VARIANT 87: note = F or Y
  VARIANT 100: note = G or Q
  VARIANT 104: note = L or V
XIXXTQSPXXXSXXXXXXXXXXCRASQSIDNQLHWYXQKPXXSPXLLIXYASQSISGVXXRFSGSGSGTDFTLXISXXXXEDXXXYXCQQSYGWPYTFGXGTKXEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 221 - protein, 214 aa, synthetic construct
EIVMTQSPDFQSVTPKEKVTITCRASQSIDNQLHWYQQKPDQSPKLLINYASQSISGVPSRFSGSGSGTDFTLTISSLEAEDAATYYCQQSYGWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 214 aa, synthetic construct
DIVMTQSPPSLSVTPGEPASLSCRASQSIDNQLHWYLQKPGQSPQLLINYASQSISGVTDRFSGSGSGTDFTLKISRVEAEDVGVYFCQQSYGWPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 214 aa, synthetic construct
DIVMTQSPPSLSVTPGESASISCRASQSIDNQLHWYLQKPGQSPQLLINYASQSISGVTDRFSGSGSGTDFTLKISRVEAEDVGMYFCQQSYGWPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 224 - protein, 214 aa, synthetic construct
DIQMTQSPSSLSASVGDRVTITCRASQSIDNQLHWYQQKPGKSPNLLINYASQSISGVPSRFSGSGSGTDFTLTISSLQPEDFATYFCQQSYGWPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 225 - protein, 214 aa, synthetic construct
EIVLTQSPDFQSVTPKEKVTITCRASQSIDNQLHWYQQKPDQSPKLLIKYASQSISGVPSRFSGSGSGTDFTLTISSLEAEDAATYFCQQSYGWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 226 - protein, 443 aa, synthetic construct
  VARIANT 28: note = S or A
EVQLVESGPSLVKPSQTLSLICSVTGDXITSGYWNWVRKFPGNKLEYMGYISYSGSAHYNPSLKSRISITRDTSKNQYYLQLNSMTTEDTATYYCASYSFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 214 aa, synthetic construct
  VARIANT 26: note = N or Q or S
  VARIANT 32: note = N or Q
DIVMTQSPATLSVTPGDSVSLSCRAXQSIDNXLHWYQQRSHESPRLLINYASQSISGIPSRFSGSGSGTDFTLSISSVETEDFGMYFCQQSYGWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 452 aa, synthetic construct
  VARIANT 1: note = E or Q
  VARIANT 5: note = Q or V
  VARIANT 9: note = P or A
  VARIANT 11: note = L or V
  VARIANT 12: note = V or K
  VARIANT 20: note = I or V
  VARIANT 38: note = K or R
  VARIANT 40: note = S or A
  VARIANT 43: note = K or Q
  VARIANT 44: note = N or G
  VARIANT 48: note = I or M
  VARIANT 50: note = L or W
  VARIANT 59: note = S or N
  VARIANT 61: note = N or A
  VARIANT 65: note = E or Q
  VARIANT 67: note = K or R
  VARIANT 68: note = A or V
  VARIANT 70: note = L or M
  VARIANT 72: note = V or R
  VARIANT 74: note = K or T
  VARIANT 76: note = S or I
  VARIANT 84: note = L or S
  VARIANT 85: note = S or R
  VARIANT 87: note = T or R
  VARIANT 89: note = V or D
  VARIANT 91: note = S or T
  VARIANT 117: note = S or L
XVQLXQSGXEXXKPGASMKXSCKASGYSFTGSTMNWVXQXPGXXLEWXGXINPYNGGTXYXQKFXGXXTXTXDXSXSTAYMELXXLXSXDXAVYYCASRGKNGYDGRDFDCWGQGTXVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 219 aa, synthetic construct
  VARIANT 1: note = D or E
  VARIANT 9: note = L or A
  VARIANT 10: note = S or T
  VARIANT 12: note = P or S
  VARIANT 13: note = V or L
  VARIANT 14: note = N or S
  VARIANT 15: note = I or P
  VARIANT 17: note = D or E
  VARIANT 18: note = Q or R
  VARIANT 20: note = S or T
  VARIANT 21: note = I or L
  VARIANT 36: note = T or S
  VARIANT 42: note = L or Q
  VARIANT 48: note = S or A
  VARIANT 50: note = Q or R
  VARIANT 61: note = S or T
  VARIANT 63: note = V or I
  VARIANT 65: note = D or A
  VARIANT 79: note = K or T
  VARIANT 82: note = R or S
  VARIANT 83: note = V or L
  VARIANT 85: note = A or P
  VARIANT 88: note = L or F
  VARIANT 89: note = G or A
  VARIANT 105: note = S or Q
XIVLTQSPXXLXXXXGXXAXXSCKSTKSLLNSGGFXYLDWYXQKPGQXPXLLIYLVSNRFXGXPXRFSGSGSGTDFTLXISXXEXEDXXVYYCFQSNYFPFTFGXGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 443 aa, synthetic construct
QVQLQESGPGLVKPSETLSLTCTVSGDAITSGYWNWIRQPPGKGLEYMGYISYSGSAHYNPSLKSRITISRDTSKNQYSLKLSSVTAADTAVYYCASYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 231 - protein, 443 aa, synthetic construct
QVQLQESGPGLVKPSQTLSLTCTVSWDAITSGYWNWVRQHPGKGLEYMGYISYSGSAHYNPSLKSRITISRDTSKNQYSLKLSSVTAADTATYYCASYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 232 - protein, 443 aa, synthetic construct
QVQLQESGPGLVKPSETLSLTCSVSWDAITSGYWNWVRQPPGNKLEYMGYISYSGSAHYNPSLKSRITISRDTSKNQYSLTLTSMTAADTALYYCASYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 233 - protein, 443 aa, synthetic construct
QVQLQESGPGLVKPSETLSLTCTVSWDAITSGYWNWVRQPPGNKLEYMGYISYSGSAHYNPSLKSRITISRDTSKNQYSLKLSSVTAADTAVYYCASYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 234 - protein, 443 aa, synthetic construct
QVQLQESGPGLVKPSQTLSLTCTVSWDAITSGYWNWVRQHPGNKLEYMGYISYSGSAHYNPSLKSRITISRDTSKNQYSLKLSSVTAADTAVYYCASYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 235 - protein, 443 aa, synthetic construct
  VARIANT 16: note = E or Q
  VARIANT 23: note = T or S
  VARIANT 26: note = W or G
  VARIANT 37: note = V or I
  VARIANT 40: note = P or H
  VARIANT 43: note = K or N
  VARIANT 44: note = G or K
  VARIANT 92: note = V or T
QVQLQESGPGLVKPSXTLSLTCXVSXDAITSGYWNWXRQXPGXXLEYMGYISYSGSAHYNPSLKSRITISRDTSKNQYSLKLSSVTAADTAXYYCASYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 236 - protein, 6 aa, synthetic construct
HHHHHH

(19) United States
(12) Patent Application Publication
(10) Pub. No.: US 2026/0274993 A1
(43) Pub. Date: Sep. 17, 2026

(54) VH4-34 ANTIGEN-BINDING MOLECULES

(71) Applicant: Hummingbird Bioscience Pte. Ltd., Singapore (SG)

(72) Inventors: Shalini Paliwal, Singapore (SG); Dipti Thakkar, Singapore (SG); Wen Jie Chin, Singapore (SG); Warren Lee, Singapore (SG); Jacklyn Jie Ying Neo, Singapore (SG); Brendon Hanson, Singapore (SG); Konrad Paszkiewicz, Singapore (SG); Piers Ingram, Singapore (SG); Jerome Boyd-Kirkup, Singapore (SG); Adelene Sim, Singapore (SG)

(21) Appl. No.: 19/163,241

(22) PCT Filed: Mar. 8, 2024
(86) PCT No.: PCT/IB2024/000118

§ 371 Date: Sep. 8, 2025

Related U.S. Application Data
(60) Provisional application No. 63/451,053, filed on Mar. 9, 2023.

(51) Int. Cl. C07K 16/42; A61K 39/00; A61K 40/31; A61K 40/41; A61P 37/06; G01N 33/68

(52) U.S. Cl. CPC C07K 16/4208; A61K 40/31; A61K 40/416; A61P 37/06; G01N 33/686; A61K 2039/505; A61K 2239/13; C07K 2317/14; C07K 2317/24; C07K 2317/31; C07K 2317/52; C07K 2317/565; C07K 2317/732; C07K 2317/76; G01N 2333/70503; G01N 2800/52

(57) ABSTRACT

Provided herein are VH4-34 antigen binding molecules (e.g., VH4-34 antibodies). compositions comprising the same, and methods of use.

### CROSS-REFERENCE

This application claims the benefit of and priority to U.S. Provisional Patent Application No. 63/451,053, filed Mar. 9, 2023, which is incorporated by reference in its entirety herein.

### TECHNICAL FIELD

The present disclosure relates to the fields of molecular biology, more specifically antibody technology. The present disclosure also relates to methods of medical treatment and prophylaxis.

### BACKGROUND

The production of B cells expressing BCRs specific for self-antigen is inherent in the process of B cell development. In healthy individuals, approximately 75% of newly-formed B cells are autoreactive. However, through negative selection, autoreactive B cells are either eliminated or made anergic, and so autoreactive B cells are ordinarily prevented from maturing into memory or plasma B cells. Failure in B cell tolerance mechanisms can lead to the presence in the periphery of mature B cells expressing autoreactive antibodies.

VH4-34 is an intrinsically autoreactive antibody heavy chain variable region (VH) subregion comprising a hydrophobic patch in the first framework region (FR1) that binds to a carbohydrate moiety expressed on erythrocytes and some glycoproteins (Young et al. PNAS USA (2015) 112(44):13447-54). Antibodies comprising VH4-34 are not ordinarily present in the serum of healthy individuals. However, in diseases such as systemic lupus erythematosus (SLE). VH4-34-expressing autoreactive B cells are overrepresented in the memory B cell and plasma B cell compartments, and are implicated in disease pathogenesis. A monoclonal rat anti-idiotypic antibody 9G4 binds to antibodies comprising VH4-34 (Stevenson, et al. Blood (1986) 68: 430, Richardson et al. J Immunol (2013) 191(10):4926-4939).

### SUMMARY OF THE DISCLOSURE

Described herein, in certain embodiments, are VH4-34 antigen-binding molecules comprising:
(a)
   (i) a heavy chain variable (VH) region comprising:
      a HC-CDR1 having the amino acid sequence of SEQ ID NO: 172:
      a HC-CDR2 having the amino acid sequence of SEQ ID NO: 55;
      a HC-CDR3 having the amino acid sequence of SEQ ID NO: 56; and
   (ii) a light chain variable (VL) region comprising:
      a LC-CDR1 having the amino acid sequence of SEQ ID NO: 144;
      a LC-CDR2 having the amino acid sequence YAS:
      a LC-CDR3 having the amino acid sequence of SEQ ID NO: 64; or
(b)
   (i) a heavy chain variable (VH) region comprising:
      a HC-CDR1 having the amino acid sequence of SEQ ID NO: 142;
      a HC-CDR2 having the amino acid sequence of SEQ ID NO: 55;
      a HC-CDR3 having the amino acid sequence of SEQ ID NO: 56, and
   (ii) a light chain variable (VL) region comprising:
      a LC-CDR1 having the amino acid sequence of SEQ ID NO: 144:
      a LC-CDR2 having the amino acid sequence YAS;
      a LC-CDR3 having the amino acid sequence of SEQ ID NO: 64; or
(c)
   (i) a heavy chain variable (VH) region comprising:
      a HC-CDR1 having the amino acid sequence of SEQ ID NO: 38;
      a HC-CDR2 having the amino acid sequence of SEQ ID NO: 39;
      a HC-CDR3 having the amino acid sequence of SEQ ID NO: 40, and
   (ii) a light chain variable (VL) region comprising:
      a LC-CDR1 having the amino acid sequence of SEQ ID NO: 46;
      a LC-CDR2 having the amino acid sequence AAS;
      a LC-CDR3 having the amino acid sequence of SEQ ID NO: 48; or
(d)
   (i) a heavy chain variable (VH) region comprising:
      a HC-CDR1 having the amino acid sequence of SEQ ID NO: 54;
      a HC-CDR2 having the amino acid sequence of SEQ ID NO: 55;
      a HC-CDR3 having the amino acid sequence of SEQ ID NO: 56, and
   (ii) a light chain variable (VL) region comprising:
      a LC-CDR1 having the amino acid sequence of SEQ ID NO: 62;
      a LC-CDR2 having the amino acid sequence YAS;
      a LC-CDR3 having the amino acid sequence of SEQ ID NO: 64; or
(e)
   (i) a heavy chain variable (VH) region comprising:
      a HC-CDR1 having the amino acid sequence of SEQ ID NO: 6;
      a HC-CDR2 having the amino acid sequence of SEQ ID NO: 7;
      a HC-CDR3 having the amino acid sequence of SEQ ID NO: 8; and
   (ii) a light chain variable (VL) region comprising:
      a LC-CDR1 having the amino acid sequence of SEQ ID NO: 131;
      a LC-CDR2 having the amino acid sequence LVS;
      a LC-CDR3 having the amino acid sequence of SEQ ID NO: 16; or
(f)
   (i) a heavy chain variable (VH) region comprising:
      a HC-CDR1 having the amino acid sequence of SEQ ID NO: 22;
      a HC-CDR2 having the amino acid sequence of SEQ ID NO: 23;
      a HC-CDR3 having the amino acid sequence of SEQ ID NO: 24; and
   (ii) a light chain variable (VL) region comprising:
      a LC-CDR1 having the amino acid sequence of SEQ ID NO: 30;
      a LC-CDR2 having the amino acid sequence YTS;
      a LC-CDR3 having the amino acid sequence of SEQ ID NO: 32; or
(g)
   (i) a heavy chain variable (VH) region comprising:
      a HC-CDR1 having the amino acid sequence of SEQ ID NO: 6;
      a HC-CDR2 having the amino acid sequence of SEQ ID NO: 7;
      a HC-CDR3 having the amino acid sequence of SEQ ID NO: 8; and
   (ii) a light chain variable (VL) region comprising:
      a LC-CDR1 having the amino acid sequence of SEQ ID NO: 162;
      a LC-CDR2 having the amino acid sequence LVS;
      a LC-CDR3 having the amino acid sequence of SEQ ID NO: 16; or
(h)
   (i) a heavy chain variable (VH) region comprising:
      a HC-CDR1 having the amino acid sequence of SEQ ID NO: 142;
      a HC-CDR2 having the amino acid sequence of SEQ ID NO: 55;
      a HC-CDR3 having the amino acid sequence of SEQ ID NO: 56; and
   (ii) a light chain variable (VL) region comprising:
      a LC-CDR1 having the amino acid sequence of SEQ ID NO: 144;
      a LC-CDR2 having the amino acid sequence YAS;
      a LC-CDR3 having the amino acid sequence of SEQ ID NO: 64; or
(i)
   (i) a heavy chain variable (VH) region comprising:
      a HC-CDR1 having the amino acid sequence of SEQ ID NO: 185;
      a HC-CDR2 having the amino acid sequence of SEQ ID NO: 55;
      a HC-CDR3 having the amino acid sequence of SEQ ID NO: 56; and
   (ii) a light chain variable (VL) region comprising:
      a LC-CDR1 having the amino acid sequence of SEQ ID NO: 144;
      a LC-CDR2 having the amino acid sequence YAS;
      a LC-CDR3 having the amino acid sequence of SEQ ID NO: 64; or
(j)
   (i) a heavy chain variable (VH) region comprising:
      a HC-CDR1 having the amino acid sequence of SEQ ID NO: 6;
      a HC-CDR2 having the amino acid sequence of SEQ ID NO: 7;
      a HC-CDR3 having the amino acid sequence of SEQ ID NO: 8; and
   (ii) a light chain variable (VL) region comprising:
      a LC-CDR1 having the amino acid sequence of SEQ ID NO: 14;
      a LC-CDR2 having the amino acid sequence LVS;
      a LC-CDR3 having the amino acid sequence of SEQ ID NO: 16.

In some embodiments, the VH4-34 antigen-binding molecule comprises: a VH region comprising an amino acid sequence having at least 80% sequence identity to the amino acid sequence of SEQ ID NO: 5, 21, 37, 53, 125, 141, 151, 156, 167, 171, 176, 181, 182, or 184; and a VL region comprising an amino acid sequence having at least 80% sequence identity to the amino acid sequence of SEQ ID NO: 13, 29, 45, 61, 130, 143, 147, 153, 161, 189, 193, 198, 201, 205 or 209.

In some embodiments, the VH4-34 antigen-binding molecule comprises: (i) a VH region comprising an amino acid sequence having at least 80% sequence identity to the amino acid sequence of SEQ ID NO: 182; and a VL region comprising an amino acid sequence having at least 80% sequence identity to the amino acid sequence of SEQ ID NO: 198; or (ii) a VH region comprising an amino acid sequence having at least 80% sequence identity to the amino acid sequence of SEQ ID NO: 182; and a VL region comprising an amino acid sequence having at least 80% sequence identity to the amino acid sequence of SEQ ID NO: 193; or (iii) a VH region comprising an amino acid sequence having at least 80% sequence identity to the amino acid sequence of SEQ ID NO: 167; and a VL region comprising an amino acid sequence having at least 80% sequence identity to the amino acid sequence of SEQ ID NO: 189; or (iv) a VH region comprising an amino acid sequence having at least 80% sequence identity to the amino acid sequence of SEQ ID NO: 21; and a VL region comprising an amino acid sequence having at least 80% sequence identity to the amino acid sequence of SEQ ID NO: 29; or (v) a VH region comprising an amino acid sequence having at least 80% sequence identity to the amino acid sequence of SEQ ID NO: 37; and a VL region comprising an amino acid sequence having at least 80% sequence identity to the amino acid sequence of SEQ ID NO: 45; or (vi) a VH region comprising an amino acid sequence having at least 80% sequence identity to the amino acid sequence of SEQ ID NO: 53; and a VL region comprising an amino acid sequence having at least 80% sequence identity to the amino acid sequence of SEQ ID NO: 61; or (vii) a VH region comprising an amino acid sequence having at least 80% sequence identity to the amino acid sequence of SEQ ID NO: 125; and a VL region comprising an amino acid sequence having at least 80% sequence identity to the amino acid sequence of SEQ ID NO: 130; or (viii) a VH region comprising an amino acid sequence having at least 80% sequence identity to the amino acid sequence of SEQ ID NO: 141; and a VL region comprising an amino acid sequence having at least 80% sequence identity to the amino acid sequence of SEQ ID NO: 143; or (ix) a VH region comprising an amino acid sequence having at least 80% sequence identity to the amino acid sequence of SEQ ID NO: 141; and a VL region comprising an amino acid sequence having at least 80% sequence identity to the amino acid sequence of SEQ ID NO: 147; or (x) a VH region comprising an amino acid sequence having at least 80% sequence identity to the amino acid sequence of SEQ ID NO: 156; and a VL region comprising an amino acid sequence having at least 80% sequence identity to the amino acid sequence of SEQ ID NO: 161; or (xi) a VH region comprising an amino acid sequence having at least 80% sequence identity to the amino acid sequence of SEQ ID NO: 151; and a VL region comprising an amino acid sequence having at least 80% sequence identity to the amino acid sequence of SEQ ID NO: 153; or (xii) a VH region comprising an amino acid sequence having at least 80% sequence identity to the amino acid sequence of SEQ ID NO: 184; and a VL region comprising an amino acid sequence having at least 80% sequence identity to the amino acid sequence of SEQ ID NO: 189, 193, 198, 201, 205 or 209; or (xiii) a VH region comprising an amino acid sequence having at least 80% sequence identity to the amino acid sequence of SEQ ID NO: 167; and a VL region comprising an amino acid sequence having at least 80% sequence identity to the amino acid sequence of SEQ ID NO: 189, 193, 198, 201, 205 or 209; or (xiv) a VH region comprising an amino acid sequence having at least 80% sequence identity to the amino acid sequence of SEQ ID NO: 171; and a VL region comprising an amino acid sequence having at least 80% sequence identity to the amino acid sequence of SEQ ID NO: 189, 193, 198, 201, 205 or 209; or (xv) a VH region comprising an amino acid sequence having at least 80% sequence identity to the amino acid sequence of SEQ ID NO: 176; and a VL region comprising an amino acid sequence having at least 80% sequence identity to the amino acid sequence of SEQ ID NO: 189, 193, 198, 201, 205 or 209; or (xvi) a VH region comprising an amino acid sequence having at least 80% sequence identity to the amino acid sequence of SEQ ID NO: 181; and a VL region comprising an amino acid sequence having at least 80% sequence identity to the amino acid sequence of SEQ ID NO: 189, 193, 198, 201, 205 or 209; or (xvii) a VH region comprising an amino acid sequence having at least 80% sequence identity to the amino acid sequence of SEQ ID NO: 182; and a VL region comprising an amino acid sequence having at least 80% sequence identity to the amino acid sequence of SEQ ID NO: 189, 193, 198, 201, 205 or 209; or (xviii) a VH region comprising an amino acid sequence having at least 80% sequence identity to the amino acid sequence of SEQ ID NO: 5; and a VL region comprising an amino acid sequence having at least 80% sequence identity to the amino acid sequence of SEQ ID NO: 13.

In some embodiments, the VH4-34 antigen-binding molecule inhibits interaction between VH4-34 and a molecule comprising an N-acetyl lactosamine moiety, optionally wherein the molecule comprising an N-acetyl lactosamine moiety is selected from: an I/I carbohydrate and a CD45 isoform B220. In some embodiments, the VH4-34 antigen-binding molecule further comprises an Fc region. In some embodiments, the VH4-34 antigen-binding molecule is a multispecific antigen-binding molecule, and wherein the VH4-34 antigen-binding molecule further comprises an antigen-binding domain which binds to an antigen other than VH4-34.

Described herein, in certain embodiments, are chimeric antigen receptors (CAR) comprising the VH4-34 antigen-binding molecule described herein.

Described herein, in certain embodiments, are nucleic acids, or a plurality of nucleic acids, optionally isolated, encoding the VH4-34 antigen-binding molecule described herein, or a CAR described herein.

Described herein, in certain embodiments, are expression vectors, or a plurality of expression vectors, comprising a nucleic acid or a plurality of nucleic acids described herein.

Described herein, in certain embodiments, are cells comprising the VH4-34 antigen-binding molecule described herein, a CAR according described herein, a nucleic acid or a plurality of nucleic acids described herein, or an expression vector or a plurality of expression vectors described herein.

Described herein, in certain embodiments, are methods comprising culturing a cell described herein under conditions suitable for expression of an antigen-binding molecule or CAR by the cell.

Described herein, in certain embodiments, are compositions comprising the VH4-34 antigen-binding molecule described herein, a CAR described herein, a nucleic acid or a plurality of nucleic acids described herein, an expression vector or a plurality of expression vectors described herein, or a cell described herein, and a pharmaceutically acceptable carrier, diluent, excipient or adjuvant.

Described herein, in certain embodiments, are VH4-34 antigen-binding molecule described herein, a CAR described herein, a nucleic acid or a plurality of nucleic acids described herein, an expression vector or a plurality of expression vectors described herein, or a cell described herein for use in a method of treatment or prevention of a disease or condition characterised by expression of VH4-34.

Described herein, in certain embodiments, are VH4-34 antigen-binding molecule described herein, a CAR described herein, a nucleic acid or a plurality of nucleic acids described herein, an expression vector or a plurality of expression vectors described herein, or a cell described herein for use in a method of medical treatment or prophylaxis.

Described herein, in certain embodiments, are VH4-34 antigen-binding molecule described herein, a CAR described herein, a nucleic acid or a plurality of nucleic acids described herein, an expression vector or a plurality of expression vectors described herein, or a cell described herein for use in a method of treatment or prevention of a disease or condition characterised by expression of VH4-34. Further described herein, in certain embodiments, are VH4-34 antigen-binding molecule described herein, a CAR described herein, a nucleic acid or a plurality of nucleic acids described herein, an expression vector or a plurality of expression vectors described herein, or a cell described herein for use in the manufacture of a medicament for treating or preventing a disease or condition characterised by expression of VH4-34. In some embodiments, the disease or condition characterised by expression of VH4-34 is an autoimmune disease. In some embodiments, the autoimmune disease is selected from the group consisting of: autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD), and Wiskott-Aldrich syndrome. In some embodiments, the autoimmune disease is cold agglutinin disease (CAD). In some embodiments, the autoimmune disease is systemic lupus erythematosus (SLE). In some embodiments, the disease or condition characterised by expression of VH4-34 is cancer. In some embodiments, the cancer is a B cell malignancy. In some embodiments, the B cell malignancy is selected from the group consisting of: a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, and mantle cell lymphoma. In some embodiments, the B cell malignancy is chronic lymphocytic leukemia (CLL). In some embodiments, the disease or condition characterised by expression of VH4-34 is selected from: a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, mantle cell lymphoma, autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD), and Wiskott-Aldrich syndrome.

Described herein, in certain embodiments, are methods of treating or preventing a disease or condition characterised by expression of VH4-34 in a subject, the method comprising administering to the subject the VH4-34 antigen-binding molecule described herein, a CAR described herein, a nucleic acid or a plurality of nucleic acids described herein, an expression vector or a plurality of expression vectors described herein, or a cell described herein. In some embodiments, the disease or condition characterised by expression of VH4-34 is an autoimmune disease. In some embodiments, the autoimmune disease is selected from the group consisting of autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD), and Wiskott-Aldrich syndrome. In some embodiments, the autoimmune disease is cold agglutinin disease (CAD). In some embodiments, the autoimmune disease is systemic lupus erythematosus (SLE). In some embodiments, the disease or condition characterised by expression of VH4-34 is cancer. In some embodiments, the cancer is a B cell malignancy. In some embodiments, the B cell malignancy is selected from the group consisting of a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, and mantle cell lymphoma. In some embodiments, the B cell malignancy is chronic lymphocytic leukemia (CLL). In some embodiments, the disease or condition characterised by expression of VH4-34 is selected from: a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, mantle cell lymphoma, autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD), and Wiskott-Aldrich syndrome.

Described herein, in certain embodiments, are uses of the VH4-34 antigen-binding molecule described herein to potentiate killing of cells expressing VH4-34.

Described herein, in certain embodiments, are in vitro complexes, optionally isolated, comprising the VH4-34 antigen-binding molecule described herein bound to VH4-34.

Described herein, in certain embodiments, are methods for detecting VH4-34 in a sample, comprising contacting a sample containing, or suspected to contain, VH4-34 with the VH4-34 antigen-binding molecule described herein, and detecting the formation of a complex of the VH4-34 antigen-binding molecule with VH4-34.

Described herein, in certain embodiments, are methods of selecting or stratifying a subject for treatment with a VH4-34-targeted agent, the method comprising contacting, in vitro, a sample from the subject with the VH4-34 antigen-binding molecule described herein, and detecting the formation of a complex of the VH4-34 antigen-binding molecule with VH4-34.

Described herein, in certain embodiments, are uses of the VH4-34 antigen-binding molecule described herein as an in vitro or in vivo diagnostic or prognostic agent.

Described herein, in certain embodiments, are uses of the VH4-34 antigen-binding molecule described herein in a method for detecting, localizing or imaging a disease or condition characterised by expression of VH4-34, optionally wherein the disease or condition characterised by expression of VH4-34 is selected from: a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, mantle cell lymphoma, autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD) and Wiskott-Aldrich syndrome.

### BRIEF DESCRIPTION OF THE DRAWINGS

Embodiments and experiments illustrating the principles of the present disclosure will now be discussed with reference to the accompanying figures.

FIGS. 1A and 1B depict a graph and table showing dose-response binding of anti-VH4-34 antibody D011-1E11p to the hydrophobic patch of VH4-34 on CHO cell lines overexpressing surface VH4-34 antibodies (Taba, Zano. 3A6), compared with negative control CHO cells expressing antibodies Zano FR1 and VSTB112, and untransfected CHO (WT). (FIG. 1A) shows binding by D011-1E11p, with EC₅₀ values tabulated in (FIG. 1B).

FIGS. 2A and 2B depict graphs and a table showing dose-response binding of three anti-VH4-34 antibodies D011-7016, D011-6039 and D011-6040 to the hydrophobic patch of VH4-34 on CHO cell lines overexpressing surface VH4-34 antibodies (Taba. Zano, 3A6), compared with negative control CHO cells expressing antibodies Zano FR1, Taba FR1, 3A6 FR1, VH4-34 negative cell lines 10D1F and/or VSTB112, and untransfected CHO (WT). (FIG. 2A) shows binding by the antibodies, with EC₅₀ values tabulated in (FIG. 2B).

FIGS. 3A and 3B depict graphs and a table showing dose-response binding of anti-VH4-34 antibodies D011-7016, D011-7016-LD1 and D011-7016-LD2 to the hydrophobic patch of VH4-34 on CHO cell lines overexpressing surface VH4-34 antibodies (Taba, Zano, 3A6), compared with negative control CHO cells expressing antibodies Zano FR1, Taba FR1, 3A6 FR1 and VSTB112, and untransfected CHO (WT). (FIG. 3A) shows binding by the antibodies, with EC₅₀ values tabulated in (FIG. 3B).

FIGS. 4A and 4B depict graphs and a table showing dose-response binding of anti-VH4-34 antibodies D011-7016, D011-6039 and D011-6040 to VH4-34-expressing cell line, OCI-Ly3. Pfeiffer cell line is used as a negative control. (FIG. 4A) shows binding by the antibodies, with EC₅₀ values tabulated in (FIG. 4B).

FIGS. 5A and 5B depict graphs and a table showing dose-response binding of humanised clone D011-1E11-5T2 to cell lines that endogenously express VH4-34-HBL-1 and GA-10, and negative-control cell line MM.1S. (FIG. 5A) shows binding by the antibody, with EC₅₀ values tabulated in (FIG. 5B).

FIGS. 6A-6D depict graphs and tables showing ADCC of (FIG. 6A, FIG. 6B) tabalumab- or (FIG. 6C, FIG. 6D) zanolimumab-expressing CHO cells induced by D011-7016.hIgG1, hIgG1 isotype control antibody was used as negative control. (FIG. 6A, FIG. 6C) show binding of the antibody as a function of luminescence output of the reporter cell, with associated EC₅₀ values tabulated in (FIG. 6B) and (FIG. 6D).

FIGS. 7A and 7B depict a graph and table showing ADCP induced by D011-7016.hIgG1. Rituximab.IgG1 was used as a positive control and hIgG1 isotype control antibody as negative control. (FIG. 7A) shows binding of the antibody as a function of % CFSE of CD14⁺ cells, with EC₅₀ values tabulated in (FIG. 7B).

FIGS. 8A-8C depict graphs and images showing that anti-VH4-34 antibody D011-7016.hIgG1 blocks cold agglutination. (FIG. 8A) ELISA binding of D011-7016.mIgG2a to IGM-55.5, a VH4-34 antibody. (FIG. 8B) Antibody IGM-55.5 induces cold agglutination of red blood cells at 4° C. (FIG. 8C) D011-7016.hIgG1 inhibits cold agglutination caused by antibody IGM-55.5 at 4° C.

FIGS. 9A-9C depict graphs showing the clearance of VH4-34 antibody Zanolimumab by clone D011-7016.mIgG2a from mouse circulation. (FIG. 9A) summarises the sera Zanolimumab concentration across different timepoints shown in (FIG. 9B). FIG. 9C depict a graph of relative VH-34+B cells detected in mice at the time points indicated after treatment with control antibody or D011-7016.

FIGS. 10A-10C depict graphs showing binding affinity of humanized anti-VH4-34 antibodies to (FIG. 10A) 3A6 antibody (containing VH4-34 antigen). (FIG. 10B) 3A6-FR1 (VH4-34 FR1 mutant) and (FIG. 10C) VSTB112 negative control.

FIGS. 11A-11C depict graphs and a table showing (FIG. 11A) percentage binding and (FIG. 11B) mean fluorescence intensity (MFI) of humanized anti-VH4-34 antibodies to VH4-34-expressing cell line OCI-Ly3 and negative control Pfeiffer cell line. EC₅₀ values are tabulated in (FIG. 11C).

FIG. 12 depict images showing that addition of humanized anti-VH4-34 antibodies (H0L0, H5L1, and H5L2) but not control antibodies reverses cold agglutination caused by antibody IGM-55.5 at 4° C. and 10° C.

FIG. 13 depicts a volcano plot of IGHV4-34 differentially expressed genes. Studies where human IGHV4-34 is differentially expressed in disease samples compared to normal controls were analysed. The log 2 fold change and Benjamini-Hochberg adjusted p-values were determined and plotted in a volcano plot.

### DETAILED DESCRIPTION

The present disclosure provides VH4-34 antigen-binding molecules having combinations of desirable biophysical and/or functional properties. The VH4-34-binding antibodies of the present disclosure can be used in the treatment and prevention of disease characterised by expression of VH4-34. For example, the VH4-34 antigen-binding molecules described herein are useful for depleting the blood of immunoglobulin (e.g. IgG, IgM) comprising VH4-34, and depleting subjects of cells (e.g. plasmablasts, plasma cells, memory B cells) expressing VH4-34. The antibodies are highly selective, and do not deplete immunoglobulins/B cells expressing immunoglobulins lacking VH4-34.

### Definitions

To facilitate an understanding of the present disclosure, a number of terms and phrases are defined below.

An “antigen-binding molecule” refers to a molecule which binds to a target antigen, and encompasses monoclonal antibodies, polyclonal antibodies, monospecific and multispecific antibodies (e.g., bispecific antibodies, trispecific antibodies etc.), and antibody fragments (e.g. Fv, scFv, Fab, scFab, F(ab′)₂, Fab₂, diabodies, triabodies, scFv-Fc, minibodies, single domain antibodies (e.g. VhH), etc.), as long as they display binding to the relevant target molecule(s).

As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. In general, antibodies are multimeric proteins that contain four polypeptide chains. Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (VL) and one constant region (CL). The heavy chain consists of one variable region (VH) and at least three constant regions (CH1, CH2 and CH3). The variable regions determine the binding specificity of the antibody. Each variable region contains three hypervariable regions known as complementarity determining regions (CDRs) flanked by four relatively conserved regions known as framework regions (FRs). The extent of the FRs and CDRs has been defined (Kabat, E. A., et al. (1991) Sequences of Proteins of Immunological Interest, Fifth Edition, U.S. Department of Health and Human Services, NIH Publication No. 91-3242; and Chothia. C. et al. (1987) J. Mol. Biol. 196:901-917). The three CDRs, referred to as CDR1, CDR2, and CDR3, contribute to the antibody binding specificity. Naturally occurring antibodies have been used as starting material for engineered antibodies, such as chimeric antibodies and humanized antibodies. Examples of antibody-based antigen-binding fragments include Fab, Fab′, (Fab′)2, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

The terms “variable domain” and “variable region” are used interchangeably and refer to the portions of the antibody or immunoglobulin domains that exhibit variability in their sequence and that are involved in determining the specificity and binding affinity of a particular antibody. Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three-dimensional space to form an antigen-binding surface.

The terms “recipient”, “individual”, “subject”, “host”, and “patient”, are used interchangeably herein and in some embodiments, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human. None of these terms require the supervision of medical personnel.

As used herein, the term “effective amount” refers to the amount of a compound (e.g., a compound of the present disclosure) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA (1975).

The terms “a” and “an” as used herein mean “one or more” and include the plural unless the context is inappropriate.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5.000-fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

“About” a number, as used herein, refers to range including the number and ranging from 10% below that number to 10% above that number. “About” a range refers to 10% below the lower limit of the range, spanning to 10% above the upper limit of the range.

“Percent (%) identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

Where a nucleic acid sequence is disclosed herein, the reverse complement thereof is also expressly contemplated.

Methods described herein may preferably be performed in vitro. The term “in vitro” is intended to encompass procedures performed with cells in culture whereas the term “in vivo” is intended to encompass procedures with/on intact multi-cellular organisms.

### VH4-34

Immunoglobulin heavy variable 4-34 (VH4-34) is the protein encoded in humans by IGHV4-34 gene, having the amino acid sequence of SEQ ID NO: 1 (UniProt KB: P06331-1, v1). The N-terminal 26 amino acids form a signal peptide (SEQ ID NO: 2), which is cleaved from the mature form of VH4-34 shown in SEQ ID NO: 3.

VH4-34 biology is described e.g. in Schickel et al., J Exp Med. (2017) 214(7): 1991-2003 and Pugh-Bemard et al., J Clin Invest. (2001) 108(7):1061-70 (both of which are hereby incorporated by reference in their entirety). VH4-34 is an intrinsically autoreactive antibody heavy chain variable region (VH) subregion, specifically, the VH subregion formed by heavy chain framework region 1 (HC-FR1), heavy chain complementarity-determining region 1 (HC-CDR1). FR2. HC-CDR2 and FR-3 (i.e. [HC-FR1]-[HC-CDR1]-[HC-FR2]-[HC-CDR2]-[HC-FR3]). VH4-34 comprises a hydrophobic patch in the first framework region (FR1) that recognises I/i carbohydrates expressed by erythrocytes. The unique autoreactive hydrophobic patch of VH4-34 can be detected with the monoclonal rat anti-idiotypic antibody 9G4 (see e.g. Stevenson, et al. Blood (1986) 68: 430, Potter et al., J Exp Med. (1993) 178:1419-1428 and Richardson et al. J Immunol (2013) 191(10):4926-4939).

FR1 of VH4-34 is shown in SEQ ID NO: 4. Autoreactivity requires two motifs within FR1: Q32-W33 and A49-V50-Y51 (amino acid residues numbered relative to SEQ ID NO: 1), which together form the hydrophobic patch. In other VH4 family members these motifs are instead E32-S33 and T49-V50-S51. The hydrophobic patch of VH4-34 FR1 recognises the linear lactosamine moiety of cell surface glycoproteins bearing N-acetyl lactosamine (see e.g. Young et al. PNAS USA (2015) 112(44):13447-54).

B cells expressing VH4-34 are common in the naïve B cell repertoire, but B cell tolerance mechanisms normally prevent VH4-34-expressing B cells from entering germinal centres and switching to memory B cells or plasma cells, and so they are not typically found amongst memory and plasma B cells of healthy individuals. However, in diseases such as systemic lupus erythematosus (SLE) and cold agglutinin disease, autoreactive VH4-34-expressing B cells are overrepresented in the memory and plasma cell compartments, and implicated in disease pathogenesis.

Bhat et al., J Rheumatol (2002) 29:2114-21 found that 54% of SLE patients had elevated levels of VH4-34 IgG, and that among those patients, 30% of patients also had elevated levels of VH4-34 IgM. Patients with VH4-34 IgM were observed to have significantly less severe disease than patients with circulating VH4-34 only of the IgG isotype, indicating that isotype switching of VH4-34 encoded immunoglobulins from IgM to IgG might correlate with disease progression in SLE.

In this specification, reference to “VH4-34” encompasses: human VH4-34 and variants, isoforms or fragments thereof. In some embodiments, VH4-34 comprises or consists of an amino acid sequence having at 70% or greater amino acid sequence identity, preferably one of 75%, 80%, 85%, 90%, 91%, 92%, 93%, 94%, 95%, 96%, 97%, 98%, 99% or 100% amino acid sequence identity to the amino acid sequence of SEQ ID NO: 1. In some embodiments. VH4-34 comprises or consists of an amino acid sequence having at 70% or greater amino acid sequence identity, preferably one of 75%, 80%, 85%, 90%, 91%, 92%, 93%, 94%, 95%, 96%, 97%, 98%, 99% or 100% amino acid sequence identity to the amino acid sequence of SEQ ID NO: 3. In some embodiments, VH4-34 comprises or consists the amino acid sequence of SEQ ID NO: 3.

Isoforms, fragments and variants of human VH4-34 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature VH4-34 from a given species, e.g. human. Variants of human VH4-34 may optionally be characterised as having 70% or greater amino acid sequence identity, preferably one of 75%, 80%, 85%, 90%, 91%, 92%, 93%, 94%, 95%, 96%, 97%, 98%, 99% or greater amino acid sequence identity to the amino acid sequence of SEQ ID NO: 1 or 3.

Isoforms, fragments and variants of VH4-34 may optionally possess a functional property/activity of human VH4-34, which may e.g. determined by analysis using a suitable assay for the functional property/activity. For example, an isoform, fragment or variant of human VH4-34 may form antibody:antigen complexes with anti-idiotypic antibody 9G4.

In some embodiments, the VH4-34 comprises an amino acid sequence having at 70% or greater amino acid sequence identity, preferably one of 75%, 80%, 85%, 90%, 91%, 92%, 93%, 94%, 95%, 96%, 97%, 98%, 99% or 100% amino acid sequence identity to the amino acid sequence of SEQ ID NO: 4. In some embodiments, the VH4-34 comprises the amino acid sequence of SEQ ID NO: 4.

In some embodiments, the VH4-34 comprises Q at the position corresponding to position 32 of SEQ ID NO: 1. In some embodiments, the VH4-34 comprises W at the position corresponding to position 33 of SEQ ID NO: 1. In some embodiments, the VH4-34 comprises A at the position corresponding to position 49 of SEQ ID NO: 1. In some embodiments, the VH4-34 comprises V at the position corresponding to position 50 of SEQ ID NO: 1. In some embodiments, the VH4-34 comprises Y at the position corresponding to position 51 of SEQ ID NO: 1. In some embodiments, the VH4-34 comprises one or more of (e.g. 1, 2, 3, 4 or all of); Q at the position corresponding to position 32. W at the position corresponding to position 33, A at the position corresponding to position 49, V at the position corresponding to position 50 and Y at the position corresponding to position 51 of SEQ ID NO: 1. In some embodiments, the VH4-34 comprises Q at the position corresponding to position 32, W at the position corresponding to position 33, A at the position corresponding to position 49, V at the position corresponding to position 50 and Y at the position corresponding to position 51 of SEQ ID NO: 1.

Herein, a position or region of an amino acid sequence which ‘corresponds’ to a specified position/region of a reference amino acid sequence can be identified by sequence alignment of the subject sequence to the reference sequence, e.g. using sequence alignment software such as ClustalOmega (Soding, J. 2005, Bioinformatics 21, 951-960).

In some embodiments, VH4-34 is provided as an isolated peptide/polypeptide. In some embodiments, VH4-34 is provided in a molecule comprising VH4-34. In some embodiments, VH4-34 is provided in a polypeptide complex.

In some embodiments. VH4-34 are provided in an antigen-binding molecule comprising VH4-34. In some embodiments, VH4-34 are provided in a polypeptide complex formed between two or more polypeptides (i.e. wherein one or more polypeptides of the polypeptide complex are or comprise VH4-34). For example, VH4-34 is provided in an antigen-binding molecule formed of a polypeptide complex comprising one or more immunoglobulin light chains and one or more immunoglobulin heavy chains, wherein an immunoglobulin heavy chain comprises a heavy chain variable region (VH) comprising VH4-34. By way of illustration, the monoclonal antibodies zanolimumab, patritumab and tabalumab described herein are each formed of a polypeptide complex comprising two immunoglobulin light chains, and two immunoglobulin heavy chains comprising a VH region comprising VH4-34.

A cell encoding/comprising/expressing VH4-34 and/or a peptide/polypeptide/polypeptide complex (e.g. an antigen-binding molecule) comprising VH4-34 may comprise endogenous nucleic acid (e.g. genomic DNA) encoding VH4-34 and/or a peptide/polypeptide/polypeptide complex (e.g. an antigen-binding molecule) comprising VH4-34. Such cells may be referred to as VH4-34 lineage cells.

In some embodiments, a cell encoding/comprising/expressing VH4-34 and/or a peptide/polypeptide/polypeptide complex (e.g. an antigen-binding molecule) comprising VH4-34 is a B cell or a precursor thereof. In some embodiments, a cell encoding/comprising/expressing VH4-34 and/or a peptide/polypeptide/polypeptide complex (e.g. an antigen-binding molecule) comprising VH4-34 is a B cell, a naïve B cell, a plasmablast, a plasma B cell, a memory B cell or a B cell precursor. In some embodiments, a cell is selected from a plasmablast, a plasma B cell and a memory B cell.

As used herein, A “naïve” B cell refers to a mature B cell which has not encountered the antigen for which the BCR of the B cell is specific. A naïve B cell may also be referred to as a mature naïve B cell, or a mature B cell. A naïve B cell may be characterised by expression of one or more of the following (e.g. at the cell surface): CD19, CD20, CD24, CD40, CD38, CD45. CD21, MHC Class II, IgM and IgD. A naïve B cell may be characterised by lack of expression (e.g. at the cell surface) of CD27.

As used herein, a “plasma” B cell refers to a B cell which expresses large amounts of soluble antibody. A plasma B cell may be characterised by expression of one or more of the following (e.g. at the cell surface): CD27, CD38, CD138, CD78, CD126, CXCR4 and BCMA. A plasma B cell may be characterised by lack of expression (e.g. at the cell surface): of CD20 and/or CD24. A “plasmablast” refers to cells of a short-lived differentiation stage between a post-germinal centre B-cell and a mature plasma cell. Plasmablasts have a high proliferative capacity, and an almost fully mature plasma cell phenotype.

As used herein, a “memory” B cell refers to a B cell formed in a germinal centre following a primary immune response. A memory B cell may be characterised by expression of one or more of the following (e.g. at the cell surface): CD19, CD20, CD21, CD24, CD27, CD95, CD148, MHC Class II and TACI.

As used herein, a “B cell precursor” refers to a cell type upstream of a naïve B cell in the course of B cell development. B cell development is described e.g. in Pieper et al., J Allergy Clin Immunol (2013) 131(4):959-71, which is hereby incorporated by reference in its entirety. In some embodiments, a precursor cell to a naïve B cell may be selected from: a stem cell, a pro-B cell, an early pro-B cell, a late pro-B cell, a pre-B cell, a large pre-B cell, a small pre-B cell or an immature B cell. A stem cell may be a hematopoietic stem cell, and may e.g. be characterised by expression (e.g. surface expression) of CD34, and/or lack of expression (e.g. surface expression) of CD10. An early pro-B cell may be characterised by expression (e.g. surface expression) of CD10, CD43, CD45 and/or MHC class II. A late pro-B cell may be characterised by expression (e.g. surface expression) of CD19, CD40, CD43, CD45 and/or MHC class II. A large pre-B cell may be characterised by expression (e.g. surface expression) of pre-BCR, CD19, CD40, CD43, CD45 and/or MHC class II. A small pre-B cell may be characterised by expression (e.g. surface expression) of pre-BCR, CD19, CD40, CD45 and/or MHC class II. An immature B cell may be characterised by expression (e.g. surface expression) of CD10, CD19, CD20, CD24, CD38, CD40, CD45, IgM and/or MHC class II, and/or lack of expression (e.g. surface expression) of CD27.

In some embodiments, a B cell is further characterised by reference to the class of immunoglobulin expressed by the cell. In some embodiments, a B cell is an IgM-, IgD-IgG-, IgE-, or IgA-expressing B cell.

In some embodiments, a B cell is an IgM- and/or IgD-expressing B cell. In some embodiments, a B cell is an IgG-, IgE-, or IgA-expressing B cell. In some embodiments, a B cell is an IgM-expressing B cell. In some embodiments, a B cell is an IgG-expressing B cell.

In some embodiments, the VH4-34 binding molecule comprises an immunoglobulin of isotype IgG and/or IgM. In some embodiments, the VH4-34 binding molecule comprises an immunoglobulin of isotype IgG. In some embodiments, the VH4-34 binding molecule comprises an immunoglobulin of isotype IgM.

In some embodiments, a peptide/polypeptide/polypeptide complex (e.g. an antigen-binding molecule) comprising VH4-34 is an immunoglobulin of isotype IgG and/or IgM. In some embodiments, a peptide/polypeptide/polypeptide complex (e.g. an antigen-binding molecule) comprising VH4-34 is an immunoglobulin of isotype IgG. In some embodiments, a peptide/polypeptide/polypeptide complex (e.g. an antigen-binding molecule) comprising VH4-34 is an immunoglobulin of isotype IgM.

In some embodiments, a cell encoding/comprising/expressing VH4-34 and/or a peptide/polypeptide/polypeptide complex (e.g. an antigen-binding molecule) comprising VH4-34 is a B cell encoding/comprising/expressing IgG and/or IgM comprising VH4-34. In some embodiments, a cell encoding/comprising/expressing VH4-34 and/or a peptide/polypeptide/polypeptide complex (e.g. an antigen-binding molecule) comprising VH4-34 is a B cell encoding/comprising/expressing IgG comprising VH4-34. In some embodiments, a cell encoding/comprising/expressing VH4-34 and/or a peptide/polypeptide/polypeptide complex (e.g. an antigen-binding molecule) comprising VH4-34 is a B cell encoding/comprising/expressing IgM comprising VH4-34.

Polypeptide complexes (e.g. antigen-binding molecules) comprising VH4-34 include antibodies to which the anti-idiotypic antibody 9G4 binds. Such antibodies may be referred to as “9G4-reactive” antibodies. Polypeptide complexes (e.g. antigen-binding molecules) comprising VH4-34 include the 9G4-reactive antibodies e.g. zanolimumab (also known as HuMAX-CD4; CAS No. 652153-01-0), patritumab (also known as AMG-888; CAS No. 1262787-83-6) and tabalumab (also known as LY-2127399; CAS No. 1143503-67-6).

### VH4-34 Antigen-Binding Molecules

The present disclosure provides antigen-binding molecules capable of binding to (i.e. which bind to) VH4-34 and/or antigen-binding molecules comprising VH4-34.

The VH4-34 antigen-binding molecule of the present disclosure comprises a moiety or moieties capable of binding to VH4-34. In some embodiments, the moiety capable of binding to VH4-34 comprises an antibody heavy chain variable region (VH) and an antibody light chain variable region (VL) of an antibody capable of specific binding to VH4-34. Moieties capable of binding to VH4-34 may comprise or consist of an aptamer capable of binding to the target antigen, e.g. a nucleic acid aptamer (reviewed, for example, in Zhou and Rossi Nat Rev Drug Discov. 2017 16(3):181-202). In some embodiments, a moiety may comprise or consist of an antigen-binding peptide/polypeptide, e.g. a peptide aptamer, thioredoxin, monobody, anticalin. Kunitz domain, avimer, knottin, fynomer, atrimer, DARPin, affibody, nanobody (i.e. a single-domain antibody (sdAb)), affilin, armadillo repeat protein (ArmRP), OBody or fibronectin—reviewed e.g. in Reverdatto et al., Curr Top Med Chem. 2015; 15(12): 1082-1101, which is hereby incorporated by reference in its entirety (see also e.g. Boersma et al., J Biol Chem (2011) 286:41273-85 and Emanuel et al., Mabs (2011) 3:38-48).

As used herein, a “peptide” refers to a chain of two or more amino acid monomers linked by peptide bonds. A peptide typically has a length in the region of about 2 to 50 amino acids. A “polypeptide” is a polymer chain of two or more peptides. Polypeptides typically have a length greater than about 50 amino acids.

The VH4-34 antigen-binding molecules of the present disclosure generally comprise an antigen-binding domain comprising a VH and a VL of an antibody capable of specific binding to VH4-34. The antigen-binding domain formed by a VH and a VL may also be referred to herein as an Fv region.

An antigen-binding molecule may be, or may comprise, an antigen-binding polypeptide, or an antigen-binding polypeptide complex. An antigen-binding molecule may comprise more than one polypeptide which together form an antigen-binding domain. The polypeptides may associate covalently or non-covalently. In some embodiments the polypeptides form part of a larger polypeptide comprising the polypeptides (e.g. in the case of scFv comprising VH and VL, or in the case of scFab comprising VH-CHT and VL-CL).

An antigen-binding molecule may refer to a non-covalent or covalent complex of more than one polypeptide (e.g. 2, 3, 4, 6, or 8 polypeptides), e.g. an IgG-like antigen-binding molecule comprising two heavy chain polypeptides and two light chain polypeptides.

The VH4-34 antigen-binding molecules of the present disclosure may be designed and prepared using the sequences of monoclonal antibodies (mAbs) capable of binding to VH4-34. Antigen-binding regions of antibodies, such as single chain variable fragment (scFv). Fab and F(ab′)₂ fragments may also be used/provided. An “antigen-binding region” is any fragment of an antibody which binds to the target for which the given antibody is specific.

Antibodies generally comprise six complementarity-determining regions CDRs; three in the heavy chain variable (VH) region: HC-CDRT, HC-CDR2 and HC-CDR3, and three in the light chain variable (VL) region: LC-CDRI. LC-CDR2, and LC-CDR3. The six CDRs together define the paratope of the antibody, which is the part of the antibody which binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5th Ed. Public Health Service, National Institutes of Health, Bethesda, MD (1991). Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibody clones described herein were defined according to the international IMGT (ImMunoGeneTics) information system (LeFranc et al., Nucleic Acids Res. (2015) 43 (Database issue):D413-22), which uses the IMGT V-DOMAIN numbering rules as described in Lefranc et al., Dev. Comp. Immunol. (2003) 27:55-77. In some embodiments, the CDRs and FRs of antigen-binding molecules referred to herein are defined according to the IMGT information system, the Kabat system or the Chothia system. In some embodiments, the CDRs and FRs of antigen-binding molecules referred to herein are defined according to the IMGT information system.

In some embodiments, the VH4-34 antigen-binding molecule comprises the CDRs of an antigen-binding molecule which binds to VH4-34. In some embodiments, the VH4-34 antigen-binding molecule comprises the FRs of an antigen-binding molecule which binds to VH4-34. In some embodiments, the VH4-34 antigen-binding molecule comprises the CDRs and the FRs of an antigen-binding molecule which binds to VH4-34. That is, in some embodiments the VH4-34 antigen-binding molecule comprises the VH region and/or the VL region of an antigen-binding molecule which binds to VH4-34.

In some embodiments the VH4-34 antigen-binding molecule comprises the CDRs, FRs and/or the VH and/or VL regions of a VH4-34-binding antibody clone described herein, or CDRs, FRs and/or VH and/or VL regions which are derived from those of a VH4-34-binding antibody clone described herein. In some embodiments, a VH4-34-binding antibody clone is selected from: D011-1E11p, D011-6039, D011-6040, D011-7016, D011-1E11-5T2, D011-7016-LD1, D011-7016-LD2, D011-7016-LD2_H0L0, D011-7016-LD2_H1L1, D011-7016-LD2_H1L2, D011-7016-LD2_H1L3, D011-7016-LD2_H1L4, D011-7016-LD2_H3L1, D011-7016-LD2_H3L2, D011-7016-LD2_H3L3, D011-7016-LD2_H3L4, D011-7016-LD2_H4L1, D011-7016-LD2_H4L2, D011-7016-LD2_H4L3, D011-7016-LD2_H4L4, D011-7016-LD2_H5L1, D011-7016-LD2_H5L2, D011-7016-LD2_H5L3, and D011-7016-LD2_H5L4.

In some embodiments the VH4-34 antigen-binding molecule comprises a VH region according to any one of (1) to (8) below:
(1) (D011-1E11p; D011-1E11-5T2) a VH region comprising:
   a HC-CDR1 having the amino acid sequence of SEQ ID NO: 6
   a HC-CDR2 having the amino acid sequence of SEQ ID NO: 7
   a HC-CDR3 having the amino acid sequence of SEQ ID NO: 8,
   or a variant thereof in which one or two or three amino acids in one or more of the HC-CDR1, HC-CDR2, or HC-CDR3 are substituted with another amino acid.
(2) (D011-6039) a VH region comprising:
   a HC-CDR1 having the amino acid sequence of SEQ ID NO: 22
   a HC-CDR2 having the amino acid sequence of SEQ ID NO: 23
   a HC-CDR3 having the amino acid sequence of SEQ ID NO: 24,
   or a variant thereof in which one or two or three amino acids in one or more of the HC-CDR1, HC-CDR2, or HC-CDR3 are substituted with another amino acid.
(3) (D011-6040) a VH region comprising:
   a HC-CDR1 having the amino acid sequence of SEQ ID NO: 38
   a HC-CDR2 having the amino acid sequence of SEQ ID NO: 39
   a HC-CDR3 having the amino acid sequence of SEQ ID NO: 40,
   or a variant thereof in which one or two or three amino acids in one or more of the HC-CDR1, HC-CDR2, or HC-CDR3 are substituted with another amino acid.
(4) (D011-7016) a VH region comprising:
   a HC-CDR1 having the amino acid sequence of SEQ ID NO: 54
   a HC-CDR2 having the amino acid sequence of SEQ ID NO: 55
   a HC-CDR3 having the amino acid sequence of SEQ ID NO: 56,
   or a variant thereof in which one or two or three amino acids in one or more of the HC-CDR1, HC-CDR2, or HC-CDR3 are substituted with another amino acid.
(5) (D011-7016-LD1; D011-7016-LD2; D011-7016-LD2_H0) a VH region comprising:
   a HC-CDR1 having the amino acid sequence of SEQ ID NO: 142
   a HC-CDR2 having the amino acid sequence of SEQ ID NO: 55
   a HC-CDR3 having the amino acid sequence of SEQ ID NO: 56,
   or a variant thereof in which one or two or three amino acids in one or more of the HC-CDR1, HC-CDR2, or HC-CDR3 are substituted with another amino acid.
(6) (D011-7016; D011-7016-LD1; D011-7016-LD2 consensus) a VH region comprising:
   a HC-CDR1 having the amino acid sequence of SEQ ID NO: 152
   a HC-CDR2 having the amino acid sequence of SEQ ID NO: 55
   a HC-CDR3 having the amino acid sequence of SEQ ID NO: 56,
   or a variant thereof in which one or two or three amino acids in one or more of the HC-CDR1, HC-CDR2, or HC-CDR3 are substituted with another amino acid.
(7) (D011-7016-LD2 HCON) a VH region comprising:
   a HC-CDR1 having the amino acid sequence of SEQ ID NO: 185
   a HC-CDR2 having the amino acid sequence of SEQ ID NO: 55
   a HC-CDR3 having the amino acid sequence of SEQ ID NO: 56,
   or a variant thereof in which one or two or three amino acids in one or more of the HC-CDR1, HC-CDR2, or HC-CDR3 are substituted with another amino acid.
(8) (D011-7016-LD2_H1; D011-7016-LD2_H3; D011-7016-LD2_H4; D011-7016-LD2_H5) a VH region comprising:
   a HC-CDR1 having the amino acid sequence of SEQ ID NO: 172
   a HC-CDR2 having the amino acid sequence of SEQ ID NO: 55
   a HC-CDR3 having the amino acid sequence of SEQ ID NO: 56,
   or a variant thereof in which one or two or three amino acids in one or more of the HC-CDR1, HC-CDR2, or HC-CDR3 are substituted with another amino acid.

In some embodiments the VH4-34 antigen-binding molecule comprises a VH region according to one of (9) to (21) below:
(9) (D011-1E11p) a VH region comprising:
   a HC-FR1 having the amino acid sequence of SEQ ID NO: 9
   a HC-FR2 having the amino acid sequence of SEQ ID NO: 10
   a HC-FR3 having the amino acid sequence of SEQ ID NO: 11
   a HC-FR4 having the amino acid sequence of SEQ ID NO: 12,
   or a variant thereof in which one or two or three amino acids in one or more of the HC-FR1, HC-FR2, HC-FR3, or HC-FR4 are substituted with another amino acid.
(10) (D011-6039) a VH region comprising:
   a HC-FR1 having the amino acid sequence of SEQ ID NO: 25
   a HC-FR2 having the amino acid sequence of SEQ ID NO: 26
   a HC-FR3 having the amino acid sequence of SEQ ID NO: 27
   a HC-FR4 having the amino acid sequence of SEQ ID NO: 28,
   or a variant thereof in which one or two or three amino acids in one or more of the HC-FR1, HC-FR2, HC-FR3, or HC-FR4 are substituted with another amino acid.
(11) (D011-6040) a VH region comprising:
   a HC-FR1 having the amino acid sequence of SEQ ID NO: 41
   a HC-FR2 having the amino acid sequence of SEQ ID NO: 42
   a HC-FR3 having the amino acid sequence of SEQ ID NO: 43
   a HC-FR4 having the amino acid sequence of SEQ ID NO: 44,
   or a variant thereof in which one or two or three amino acids in one or more of the HC-FR1. HC-FR2, HC-FR3, or HC-FR4 are substituted with another amino acid.
(12) (D011-7016; D011-7016-LD1; D011-7016-LD2) a VH region comprising:
   a HC-FR1 having the amino acid sequence of SEQ ID NO: 57
   a HC-FR2 having the amino acid sequence of SEQ ID NO: 58
   a HC-FR3 having the amino acid sequence of SEQ ID NO: 59
   a HC-FR4 having the amino acid sequence of SEQ ID NO: 60,
   or a variant thereof in which one or two or three amino acids in one or more of the HC-FR1, HC-FR2, HC-FR3, or HC-FR4 are substituted with another amino acid.
(13) (D011-1E11-5T2) a VH region comprising:
   a HC-FR1 having the amino acid sequence of SEQ ID NO: 126
   a HC-FR2 having the amino acid sequence of SEQ ID NO: 127
   a HC-FR3 having the amino acid sequence of SEQ ID NO: 128
   a HC-FR4 having the amino acid sequence of SEQ ID NO: 129.
   or a variant thereof in which one or two or three amino acids in one or more of the HC-FR1, HC-FR2, HC-FR3, or HC-FR4 are substituted with another amino acid.
(14) (D011-1E11p; D011-1E11-5T2 consensus) a VH region comprising:
   a HC-FR1 having the amino acid sequence of SEQ ID NO: 157
   a HC-FR2 having the amino acid sequence of SEQ ID NO: 158
   a HC-FR3 having the amino acid sequence of SEQ ID NO: 159
   a HC-FR4 having the amino acid sequence of SEQ ID NO: 160,
   or a variant thereof in which one or two or three amino acids in one or more of the HC-FR1, HC-FR2, HC-FR3, or HC-FR4 are substituted with another amino acid.
(15) (D011-1E11p; D011-1E11-5T2) a VH region comprising:
   a HC-FR1 having the amino acid sequence of SEQ ID NO: 9, 126 or 157
   a HC-FR2 having the amino acid sequence of SEQ ID NO: 10, 127 or 158
   a HC-FR3 having the amino acid sequence of SEQ ID NO: 11, 128 or 159
   a HC-FR4 having the amino acid sequence of SEQ ID NO: 12, 129 or 160,
   or a variant thereof in which one or two or three amino acids in one or more of the HC-FR1, HC-FR2, HC-FR3, or HC-FR4 are substituted with another amino acid.
(16) (D011-7016-LD2 HCON) a VH region comprising:
   a HC-FR1 having the amino acid sequence of SEQ ID NO: 186
   a HC-FR2 having the amino acid sequence of SEQ ID NO: 187
   a HC-FR3 having the amino acid sequence of SEQ ID NO: 188
   a HC-FR4 having the amino acid sequence of SEQ ID NO: 129.
   or a variant thereof in which one or two or three amino acids in one or more of the HC-FR1. HC-FR2, HC-FR3, or HC-FR4 are substituted with another amino acid.
(17) (D011-7016-LD2_H0) a VH region comprising:
   a HC-FR1 having the amino acid sequence of SEQ ID NO: 168
   a HC-FR2 having the amino acid sequence of SEQ ID NO: 169
   a HC-FR3 having the amino acid sequence of SEQ ID NO: 170
   a HC-FR4 having the amino acid sequence of SEQ ID NO: 129,
   or a variant thereof in which one or two or three amino acids in one or more of the HC-FR1, HC-FR2, HC-FR3, or HC-FR4 are substituted with another amino acid.
(18) (D011-7016-LD2_HI) a VH region comprising:
   a HC-FR1 having the amino acid sequence of SEQ ID NO: 173
   a HC-FR2 having the amino acid sequence of SEQ ID NO: 174
   a HC-FR3 having the amino acid sequence of SEQ ID NO: 175
   a HC-FR4 having the amino acid sequence of SEQ ID NO: 129.
   or a variant thereof in which one or two or three amino acids in one or more of the HC-FR1, HC-FR2, HC-FR3, or HC-FR4 are substituted with another amino acid.
(19) (D011-7016-LD2_H3) a VH region comprising:
   a HC-FR1 having the amino acid sequence of SEQ ID NO: 178
   a HC-FR2 having the amino acid sequence of SEQ ID NO: 179
   a HC-FR3 having the amino acid sequence of SEQ ID NO: 180
   a HC-FR4 having the amino acid sequence of SEQ ID NO: 129,
   or a variant thereof in which one or two or three amino acids in one or more of the HC-FR1, HC-FR2, HC-FR3, or HC-FR4 are substituted with another amino acid.
(20) (D011-7016-LD2_H4) a VH region comprising:
   a HC-FR1 having the amino acid sequence of SEQ ID NO: 168
   a HC-FR2 having the amino acid sequence of SEQ ID NO: 179
   a HC-FR3 having the amino acid sequence of SEQ ID NO: 170
   a HC-FR4 having the amino acid sequence of SEQ ID NO: 129,
   or a variant thereof in which one or two or three amino acids in one or more of the HC-FR1, HC-FR2, HC-FR3, or HC-FR4 are substituted with another amino acid.
(21) (D011-7016-LD2_H5) a VH region comprising:
   a HC-FR1 having the amino acid sequence of SEQ ID NO: 173
   a HC-FR2 having the amino acid sequence of SEQ ID NO: 183
   a HC-FR3 having the amino acid sequence of SEQ ID NO: 170
   a HC-FR4 having the amino acid sequence of SEQ ID NO: 129.
   or a variant thereof in which one or two or three amino acids in one or more of the HC-FR1. HC-FR2, HC-FR3, or HC-FR4 are substituted with another amino acid.

In some embodiments the VH4-34 antigen-binding molecule comprises a VH region comprising the CDRs according to one of (1) to (8) above, and the FRs according to one of (9) to (21) above.

In some embodiments the VH4-34 antigen-binding molecule comprises a VH region according to one of (22) to (30) below:
(22) (D011-1E11p) a VH region comprising the CDRs according to (1) and the FRs according to (9) or (11).
(23) (D011-6039) a VH region comprising the CDRs according to (2) and the FRs according to (10).
(24) (D011-6040) a VH region comprising the CDRs according to (3) and the FRs according to (11).
(25) (D011-7016) a VH region comprising the CDRs according to (4) and the FRs according to (12).
(26) (D011-1E11-5T2) a VH region comprising the CDRs according to (1) and the FRs according to (13), (14) or (15).
(27) (D011-7016-LD1; D011-7016-LD2) a VH region comprising the CDRs according to (5) and the FRs according to (12).
(28) (D011-7016-LD2 HCON) a VH region comprising the CDRs according to (7) and the FRs according to (16).
(29) (D011-7016-LD2_H0) a VH region comprising the CDRs according to (5) and the FRs according to (17).
(30) (D011-7016-LD2_H1; D011-7016-LD2_H3; D011-7016-LD2_H4; D011-7016-LD2_H5) a VH region comprising the CDRs according to (8) and the FRs according to any one of (18), (19), (20), or (21).

In some embodiments the VH4-34 antigen-binding molecule comprises a VH region according to one of (31) to (46) below:
(31) (D011-1E11P; D011-6040 consensus) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 69.
(32) (D011-6093; D011-7016 consensus) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 70.
(33) (D011-1E11p; D011-1E11-5T2 consensus) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 156.
(34) (D011-7016; D011-7016-LD1; D011-7016-LD2 consensus) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 151.
(35) (D011-7016-LD2 HCON) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 184.
(36) (D011-1E11p) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 5.
(37) (D011-6039) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 21.
(38) (D011-6040) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 37.
(39) (D011-7016) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 53.
(40) (D011-1E11-5T2) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 125.
(41) (D011-7016-LD1; D011-7016-LD2) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 141.
(42) (D011-7016-LD2_H0) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 167.
(43) (D011-7016-LD2_H1) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 171.
(44) (D011-7016-LD2_H3) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 176.
(45) (D011-7016-LD2_H4) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 181.
(46) (D011-7016-LD2_H5) a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 182.

In some embodiments the VH4-34 antigen-binding molecule comprises a VL region according to one of (47) to (56) below:
(47) (D011-1E11p) a VL region comprising:
   a LC-CDR1 having the amino acid sequence of SEQ ID NO: 14
   a LC-CDR2 having the amino acid sequence LVS
   a LC-CDR3 having the amino acid sequence of SEQ ID NO: 16,
   or a variant thereof in which one or two or three amino acids in one or more of the LC-CDR1, LC-CDR2, or LC-CDR3 are substituted with another amino acid.
(48) (D011-6039) a VL region comprising:
   a LC-CDR1 having the amino acid sequence of SEQ ID NO: 30
   a LC-CDR2 having the amino acid sequence YTS
   a LC-CDR3 having the amino acid sequence of SEQ ID NO: 32,
   or a variant thereof in which one or two or three amino acids in one or more of the LC-CDR1, LC-CDR2, or LC-CDR3 are substituted with another amino acid.
(49) (D011-6040) a VL region comprising:
   a LC-CDR1 having the amino acid sequence of SEQ ID NO: 46
   a LC-CDR2 having the amino acid sequence AAS
   a LC-CDR3 having the amino acid sequence of SEQ ID NO: 48,
or a variant thereof in which one or two or three amino acids in one or more of the LC-CDR1, LC-CDR2, or LC-CDR3 are substituted with another amino acid.
(50) (D011-7016) a VL region comprising:
   a LC-CDR1 having the amino acid sequence of SEQ ID NO: 62
   a LC-CDR2 having the amino acid sequence YAS
   a LC-CDR3 having the amino acid sequence of SEQ ID NO: 64,
   or a variant thereof in which one or two or three amino acids in one or more of the LC-CDR1, LC-CDR2, or LC-CDR3 are substituted with another amino acid.
(51) (D011-1E11-5T2) a VL region comprising:
   a LC-CDR1 having the amino acid sequence of SEQ ID NO: 131
   a LC-CDR2 having the amino acid sequence LVS
   a LC-CDR3 having the amino acid sequence of SEQ ID NO: 16,
   or a variant thereof in which one or two or three amino acids in one or more of the LC-CDR1, LC-CDR2, or LC-CDR3 are substituted with another amino acid.
(52) (D011-7016-LD1; D011-7016-LD2; D011-7016-LD2 LCON; D011-7016-LD2_L0; D011-7016-LD2_L1; D011-7016-LD2_L2; D011-7016-LD2_L3; D011-7016-LD2_L4) a VL region comprising:
   a LC-CDR1 having the amino acid sequence of SEQ ID NO: 144
   a LC-CDR2 having the amino acid sequence YAS
   a LC-CDR3 having the amino acid sequence of SEQ ID NO: 64,
   or a variant thereof in which one or two or three amino acids in one or more of the LC-CDR1, LC-CDR2, or LC-CDR3 are substituted with another amino acid.
(53) (D011-1E11p; D011-1E11-5T2 consensus) a VL region comprising:
   a LC-CDR1 having the amino acid sequence of SEQ ID NO: 162
   a LC-CDR2 having the amino acid sequence LVS
   a LC-CDR3 having the amino acid sequence of SEQ ID NO: 16,
   or a variant thereof in which one or two or three amino acids in one or more of the LC-CDR1, LC-CDR2, or LC-CDR3 are substituted with another amino acid.
(54) (D011-1E11p; D011-1E11-5T2) a VL region comprising:
   a LC-CDR1 having the amino acid sequence of SEQ ID NO: 14, 131 or 162
   a LC-CDR2 having the amino acid sequence LVS
   a LC-CDR3 having the amino acid sequence of SEQ ID NO: 16,
   or a variant thereof in which one or two or three amino acids in one or more of the LC-CDR1, LC-CDR2, or LC-CDR3 are substituted with another amino acid.
(55) (D011-7016; D011-7016-LD1; D011-7016-LD2 consensus) a VL region comprising:
   a LC-CDRT having the amino acid sequence of SEQ ID NO: 154
   a LC-CDR2 having the amino acid sequence YAS
   a LC-CDR3 having the amino acid sequence of SEQ ID NO: 64,
   or a variant thereof in which one or two or three amino acids in one or more of the LC-CDR1, LC-CDR2, or LC-CDR3 are substituted with another amino acid.
(56) (D011-7016; D011-7016-LD1; D011-7016-LD2) a VL region comprising:
   a LC-CDR1 having the amino acid sequence of SEQ ID NO: 62, 144 or 154
   a LC-CDR2 having the amino acid sequence YAS
   a LC-CDR3 having the amino acid sequence of SEQ ID NO: 64,
   or a variant thereof in which one or two or three amino acids in one or more of the LC-CDR1, LC-CDR2, or LC-CDR3 are substituted with another amino acid.

In some embodiments the VH4-34 antigen-binding molecule comprises a VL region according to one of (57) to (73) below:
(57) (D011-1E11p) a VL region comprising:
   a LC-FR1 having the amino acid sequence of SEQ ID NO: 17
   a LC-FR2 having the amino acid sequence of SEQ ID NO: 18
   a LC-FR3 having the amino acid sequence of SEQ ID NO: 19
   a LC-FR4 having the amino acid sequence of SEQ ID NO: 20,
   or a variant thereof in which one or two or three amino acids in one or more of the LC-FR1, LC-FR2, LC-FR3, or LC-FR4 are substituted with another amino acid.
(58) (D011-6039) a VL region comprising:
   a LC-FRT having the amino acid sequence of SEQ ID NO: 33
   a LC-FR2 having the amino acid sequence of SEQ ID NO: 34
   a LC-FR3 having the amino acid sequence of SEQ ID NO: 35
   a LC-FR4 having the amino acid sequence of SEQ ID NO: 36,
   or a variant thereof in which one or two or three amino acids in one or more of the LC-FR1, LC-FR2, LC-FR3, or LC-FR4 are substituted with another amino acid.
(59) (D011-6040) a VL region comprising:
   a LC-FR1 having the amino acid sequence of SEQ ID NO: 49
   a LC-FR2 having the amino acid sequence of SEQ ID NO: 50
   a LC-FR3 having the amino acid sequence of SEQ ID NO: 51
   a LC-FR4 having the amino acid sequence of SEQ ID NO: 52,
   or a variant thereof in which one or two or three amino acids in one or more of the LC-FR1, LC-FR2, LC-FR3, or LC-FR4 are substituted with another amino acid.
(60) (D011-7016) a VL region comprising:
   a LC-FRT having the amino acid sequence of SEQ ID NO: 65
   a LC-FR2 having the amino acid sequence of SEQ ID NO: 66
   a LC-FR3 having the amino acid sequence of SEQ ID NO: 67
   a LC-FR4 having the amino acid sequence of SEQ ID NO: 68,
   or a variant thereof in which one or two or three amino acids in one or more of the LC-FR1, LC-FR2, LC-FR3, or LC-FR4 are substituted with another amino acid.
(61) (D011-1E11-5T2) a VL region comprising:
   a LC-FRT having the amino acid sequence of SEQ ID NO: 132
   a LC-FR2 having the amino acid sequence of SEQ ID NO: 133
   a LC-FR3 having the amino acid sequence of SEQ ID NO: 134
   a LC-FR4 having the amino acid sequence of SEQ ID NO: 135,
   or a variant thereof in which one or two or three amino acids in one or more of the LC-FR1, LC-FR2, LC-FR3, or LC-FR4 are substituted with another amino acid.
(62) (D011-7016-LD1) a VL region comprising:
   a LC-FR1 having the amino acid sequence of SEQ ID NO: 145
   a LC-FR2 having the amino acid sequence of SEQ ID NO: 66
   a LC-FR3 having the amino acid sequence of SEQ ID NO: 67
   a LC-FR4 having the amino acid sequence of SEQ ID NO: 68,
   or a variant thereof in which one or two or three amino acids in one or more of the LC-FR1, LC-FR2, LC-FR3, or LC-FR4 are substituted with another amino acid.
(63) (D011-7016-LD2) a VL region comprising:
   a LC-FRT having the amino acid sequence of SEQ ID NO: 148
   a LC-FR2 having the amino acid sequence of SEQ ID NO: 66
   a LC-FR3 having the amino acid sequence of SEQ ID NO: 67
   a LC-FR4 having the amino acid sequence of SEQ ID NO: 68,
   or a variant thereof in which one or two or three amino acids in one or more of the LC-FR1, LC-FR2, LC-FR3, or LC-FR4 are substituted with another amino acid.
(64) (D011-1E11p; D011-11-5T2 consensus) a VL region comprising:
   a LC-FR1 having the amino acid sequence of SEQ ID NO: 163
   a LC-FR2 having the amino acid sequence of SEQ ID NO: 164
   a LC-FR3 having the amino acid sequence of SEQ ID NO: 165
   a LC-FR4 having the amino acid sequence of SEQ ID NO: 166.
   or a variant thereof in which one or two or three amino acids in one or more of the LC-FR1, LC-FR2, LC-FR3, or LC-FR4 are substituted with another amino acid.
(65) (D011-1E11p; D011-1E11-5T2) a VL region comprising:
   a LC-FRT having the amino acid sequence of SEQ ID NO: 17, 132, or 163
   a LC-FR2 having the amino acid sequence of SEQ ID NO: 18, 133, or 164
   a LC-FR3 having the amino acid sequence of SEQ ID NO: 19, 134, or 165
   a LC-FR4 having the amino acid sequence of SEQ ID NO: 20, 135, or 166,
   or a variant thereof in which one or two or three amino acids in one or more of the LC-FR1, LC-FR2, LC-FR3, or LC-FR4 are substituted with another amino acid.
(66) (D011-7016; D011-7016-LD1; D011-7016-LD2 consensus) a VL region comprising:
   a LC-FR1 having the amino acid sequence of SEQ ID NO: 155
   a LC-FR2 having the amino acid sequence of SEQ ID NO: 66
   a LC-FR3 having the amino acid sequence of SEQ ID NO: 67
   a LC-FR4 having the amino acid sequence of SEQ ID NO: 68,
   or a variant thereof in which one or two or three amino acids in one or more of the LC-FR1, LC-FR2, LC-FR3, or LC-FR4 are substituted with another amino acid.
(67) (D011-7016; D011-7016-LD1; D011-7016-LD2) a VL region comprising:
   a LC-FR1 having the amino acid sequence of SEQ ID NO: 65, 145, 148, or 155
   a LC-FR2 having the amino acid sequence of SEQ ID NO: 66
   a LC-FR3 having the amino acid sequence of SEQ ID NO: 67
   a LC-FR4 having the amino acid sequence of SEQ ID NO: 68,
   or a variant thereof in which one or two or three amino acids in one or more of the LC-FR1, LC-FR2, LC-FR3, or LC-FR4 are substituted with another amino acid.
(68) (D011-7016-LD2 LCON) a VL region comprising:
   a LC-FRT having the amino acid sequence of SEQ ID NO: 210
   a LC-FR2 having the amino acid sequence of SEQ ID NO: 211
   a LC-FR3 having the amino acid sequence of SEQ ID NO: 212
   a LC-FR4 having the amino acid sequence of SEQ ID NO: 213,
   or a variant thereof in which one or two or three amino acids in one or more of the LC-FR1, LC-FR2, LC-FR3, or LC-FR4 are substituted with another amino acid.
(69) (D011-7016-LD2 LO) a VL region comprising:
   a LC-FRT having the amino acid sequence of SEQ ID NO: 190
   a LC-FR2 having the amino acid sequence of SEQ ID NO: 191
   a LC-FR3 having the amino acid sequence of SEQ ID NO: 192
   a LC-FR4 having the amino acid sequence of SEQ ID NO: 135.
   or a variant thereof in which one or two or three amino acids in one or more of the LC-FR1, LC-FR2, LC-FR3, or LC-FR4 are substituted with another amino acid.
(70) (D011-7016-LD2_L1) a VL region comprising:
   a LC-FR1 having the amino acid sequence of SEQ ID NO: 194
   a LC-FR2 having the amino acid sequence of SEQ ID NO: 195
   a LC-FR3 having the amino acid sequence of SEQ ID NO: 196
   a LC-FR4 having the amino acid sequence of SEQ ID NO: 197,
   or a variant thereof in which one or two or three amino acids in one or more of the LC-FR1, LC-FR2, LC-FR3, or LC-FR4 are substituted with another amino acid.
(71) (D011-7016-LD2_L2) a VL region comprising:
   a LC-FR1 having the amino acid sequence of SEQ ID NO: 199
   a LC-FR2 having the amino acid sequence of SEQ ID NO: 195
   a LC-FR3 having the amino acid sequence of SEQ ID NO: 200
   a LC-FR4 having the amino acid sequence of SEQ ID NO: 197,
   or a variant thereof in which one or two or three amino acids in one or more of the LC-FR1, LC-FR2, LC-FR3, or LC-FR4 are substituted with another amino acid.
(72) (D011-7016-LD2_L3) a VL region comprising:
   a LC-FR1 having the amino acid sequence of SEQ ID NO: 202
   a LC-FR2 having the amino acid sequence of SEQ ID NO: 203
   a LC-FR3 having the amino acid sequence of SEQ ID NO: 204
   a LC-FR4 having the amino acid sequence of SEQ ID NO: 197,
   or a variant thereof in which one or two or three amino acids in one or more of the LC-FR1, LC-FR2, LC-FR3, or LC-FR4 are substituted with another amino acid.
(73) (D011-7016-LD2_L4) a VL region comprising:
   a LC-FR1 having the amino acid sequence of SEQ ID NO: 206
   a LC-FR2 having the amino acid sequence of SEQ ID NO: 207
   a LC-FR3 having the amino acid sequence of SEQ ID NO: 208
   a LC-FR4 having the amino acid sequence of SEQ ID NO: 68,
   or a variant thereof in which one or two or three amino acids in one or more of the LC-FR1, LC-FR2, LC-FR3, or LC-FR4 are substituted with another amino acid.

In some embodiments the VH4-34 antigen-binding molecule comprises a VL region comprising the CDRs according to one of (47) to (56) above, and the FRs according to one of (57) to (73) above.

In some embodiments the VH4-34 antigen-binding molecule comprises a VL region according to one of (74) to (85) below:
(74) (D011-1E11p) a VL region comprising the CDRs according to (47) and the FRs according to (57).
(75) (D011-6039) a VL region comprising the CDRs according to (48) and the FRs according to (58).
(76) (D011-6040) a VL region comprising the CDRs according to (49) and the FRs according to (59).
(77) (D011-7016) a VL region comprising the CDRs according to (50) and the FRs according to (60).
(78) (D011-1E11-5T2) a VL region comprising the CDRs according to (51) and the FRs according to (61).
(79) (D011-7016-LD1) a VL region comprising the CDRs according to (52) and the FRs according to (62).
(80) (D011-7016-LD2) a VL region comprising the CDRs according to (52) and the FRs according to (63).
(81) (D011-1E11p; D011-1E11-5T2 consensus) a VL region comprising the CDRs according to (53) and the FRs according to (64).
(82) (D011-1E11p; D011-1E11-5T2) a VL region comprising the CDRs according to (47), (51) or (53) and the FRs according to (57), (61) or (64).
(83) (D011-7016; D011-7016-LD1; D011-7016-LD2 consensus) a VL region comprising the CDRs according to (55) and the FRs according to (66).
(84) (D011-7016; D011-7016-LD1; D011-7016-LD2) a VL region comprising the CDRs according to (54), (52), (55), or (56) and the FRs according to (60), (62), (63), (66) or (67).
(85) (D011-7016-LD2 LCON; D011-7016-LD2_L0; D011-7016-LD2_L1; D011-7016-LD2_L2; D011-7016-LD2_L3; D011-7016-LD2 L4) a VL region comprising the CDRs according to (52) and the FRs according to any one of (68), (69), (70), (71), (72) or (73).

In some embodiments the VH4-34 antigen-binding molecule comprises a VL region according to one of (86) to (100) below:
(86) (D011-1E11p) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 13.
(87) (D011-6039) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 29.
(88) (D011-6040) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 45.
(89) (D011-7016) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 61.
(90) (D011-1E11-5T2) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 130.
(91) (D011-7016-LD1) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 143.
(92) (D011-7016-LD2) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 147.
(93) (D011-1E11p; D011-1E11-5T2 consensus) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 161.
(94) (D011-7016; D011-7016-LD1; D011-7016-LD2 consensus) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 153.
(95) (D011-7016-LD2 LCON) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 209.
(96) (D011-7016-LD2_L0) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 189.
(97) (D011-7016-LD2_L1) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 193.
(98) (D011-7016-LD2_L2) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 198.
(99) (D011-7016-LD2_L3) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 201.
(100) (D011-7016-LD2_L4) a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 205.

In some embodiments, the VH4-34 antigen-binding molecule comprises a VH region comprising: a HC-CDR1 having the amino acid sequence of SEQ ID NO: 6; a HC-CDR2 having the amino acid sequence of SEQ ID NO: 7; a HC-CDR3 having the amino acid sequence of SEQ ID NO: 8, or a variant thereof in which one or two or three amino acids in one or more of the HC-CDR1, HC-CDR2, or HC-CDR3 are substituted with another amino acid; and a VL region comprising: a LC-CDR1 having the amino acid sequence of SEQ ID NO: 14: a LC-CDR2 having the amino acid sequence LVS; a LC-CDR3 having the amino acid sequence of SEQ ID NO: 16, or a variant thereof in which one or two or three amino acids in one or more of the LC-CDR1, LC-CDR2, or LC-CDR3 are substituted with another amino acid.

In some embodiments, the VH4-34 antigen-binding molecule comprises a VH region comprising: a HC-CDR1 having the amino acid sequence of SEQ ID NO: 22: a HC-CDR2 having the amino acid sequence of SEQ ID NO: 23; a HC-CDR3 having the amino acid sequence of SEQ ID NO: 24, or a variant thereof in which one or two or three amino acids in one or more of the HC-CDR1, HC-CDR2, or HC-CDR3 are substituted with another amino acid; a VL region comprising: a LC-CDR1 having the amino acid sequence of SEQ ID NO: 30; a LC-CDR2 having the amino acid sequence YTS: a LC-CDR3 having the amino acid sequence of SEQ ID NO: 32, or a variant thereof in which one or two or three amino acids in one or more of the LC-CDR1, LC-CDR2, or LC-CDR3 are substituted with another amino acid.

In some embodiments, the VH4-34 antigen-binding molecule comprises a VH region comprising: a HC-CDR1 having the amino acid sequence of SEQ ID NO: 38; a HC-CDR2 having the amino acid sequence of SEQ ID NO: 39; a HC-CDR3 having the amino acid sequence of SEQ ID NO: 40, or a variant thereof in which one or two or three amino acids in one or more of the HC-CDR1, HC-CDR2, or HC-CDR3 are substituted with another amino acid; and a VL region comprising: a LC-CDR1 having the amino acid sequence of SEQ ID NO: 46; a LC-CDR2 having the amino acid sequence AAS; a LC-CDR3 having the amino acid sequence of SEQ ID NO: 48, or a variant thereof in which one or two or three amino acids in one or more of the LC-CDR1, LC-CDR2, or LC-CDR3 are substituted with another amino acid.

In some embodiments, the VH4-34 antigen-binding molecule comprises a VH region comprising: a HC-CDR1 having the amino acid sequence of SEQ ID NO: 54; a HC-CDR2 having the amino acid sequence of SEQ ID NO: 55; a HC-CDR3 having the amino acid sequence of SEQ ID NO: 56, or a variant thereof in which one or two or three amino acids in one or more of the HC-CDR1, HC-CDR2, or HC-CDR3 are substituted with another amino acid; and a VL region comprising: a LC-CDR1 having the amino acid sequence of SEQ ID NO: 62; a LC-CDR2 having the amino acid sequence YAS; a LC-CDR3 having the amino acid sequence of SEQ ID NO: 64, or a variant thereof in which one or two or three amino acids in one or more of the LC-CDR1, LC-CDR2, or LC-CDR3 are substituted with another amino acid.

In some embodiments, the VH4-34 antigen-binding molecule comprises a VH region comprising: a HC-CDR1 having the amino acid sequence of SEQ ID NO: 6; a HC-CDR2 having the amino acid sequence of SEQ ID NO: 7; a HC-CDR3 having the amino acid sequence of SEQ ID NO: 8, or a variant thereof in which one or two or three amino acids in one or more of the HC-CDR1. HC-CDR2, or HC-CDR3 are substituted with another amino acid; and a VL region comprising: a LC-CDR1 having the amino acid sequence of SEQ ID NO: 131; a LC-CDR2 having the amino acid sequence LVS; a LC-CDR3 having the amino acid sequence of SEQ ID NO: 16, or a variant thereof in which one or two or three amino acids in one or more of the LC-CDR1, LC-CDR2, or LC-CDR3 are substituted with another amino acid.

In some embodiments, the VH4-34 antigen-binding molecule comprises a VH region comprising: a HC-CDR1 having the amino acid sequence of SEQ ID NO: 142; a HC-CDR2 having the amino acid sequence of SEQ ID NO: 55; a HC-CDR3 having the amino acid sequence of SEQ ID NO: 56, or a variant thereof in which one or two or three amino acids in one or more of the HC-CDR1, HC-CDR2, or HC-CDR3 are substituted with another amino acid; and a VL region comprising: a LC-CDR1 having the amino acid sequence of SEQ ID NO: 144; a LC-CDR2 having the amino acid sequence YAS; a LC-CDR3 having the amino acid sequence of SEQ ID NO: 64, or a variant thereof in which one or two or three amino acids in one or more of the LC-CDR1, LC-CDR2, or LC-CDR3 are substituted with another amino acid.

In some embodiments, the VH4-34 antigen-binding molecule comprises a VH region comprising: a HC-CDR1 having the amino acid sequence of SEQ ID NO: 6; a HC-CDR2 having the amino acid sequence of SEQ ID NO: 7; a HC-CDR3 having the amino acid sequence of SEQ ID NO: 8, or a variant thereof in which one or two or three amino acids in one or more of the HC-CDR1, HC-CDR2, or HC-CDR3 are substituted with another amino acid; and a VL region comprising: a LC-CDR1 having the amino acid sequence of SEQ ID NO: 162; a LC-CDR2 having the amino acid sequence LVS; a LC-CDR3 having the amino acid sequence of SEQ ID NO: 16, or a variant thereof in which one or two or three amino acids in one or more of the LC-CDR1, LC-CDR2, or LC-CDR3 are substituted with another amino acid.

In some embodiments, the VH4-34 antigen-binding molecule comprises a VH region comprising: a HC-CDR1 having the amino acid sequence of SEQ ID NO: 152; a HC-CDR2 having the amino acid sequence of SEQ ID NO: 55: a HC-CDR3 having the amino acid sequence of SEQ ID NO: 56, or a variant thereof in which one or two or three amino acids in one or more of the HC-CDR1, HC-CDR2, or HC-CDR3 are substituted with another amino acid; and a VL region comprising: a LC-CDR1 having the amino acid sequence of SEQ ID NO: 154; a LC-CDR2 having the amino acid sequence YAS; a LC-CDR3 having the amino acid sequence of SEQ ID NO: 64, or a variant thereof in which one or two or three amino acids in one or more of the LC-CDR1, LC-CDR2, or LC-CDR3 are substituted with another amino acid.

In some embodiments, the VH4-34 antigen-binding molecule comprises a VH region comprising: a HC-CDR1 having the amino acid sequence of SEQ ID NO: 185; a HC-CDR2 having the amino acid sequence of SEQ ID NO: 55: a HC-CDR3 having the amino acid sequence of SEQ ID NO: 56, or a variant thereof in which one or two or three amino acids in one or more of the HC-CDR1, HC-CDR2, or HC-CDR3 are substituted with another amino acid; and a VL region comprising: a LC-CDR1 having the amino acid sequence of SEQ ID NO: 144; a LC-CDR2 having the amino acid sequence YAS; a LC-CDR3 having the amino acid sequence of SEQ ID NO: 64, or a variant thereof in which one or two or three amino acids in one or more of the LC-CDR1, LC-CDR2, or LC-CDR3 are substituted with another amino acid.

In some embodiments, the VH4-34 antigen-binding molecule comprises a VH region comprising: a HC-CDR1 having the amino acid sequence of SEQ ID NO: 172; a HC-CDR2 having the amino acid sequence of SEQ ID NO: 55; a HC-CDR3 having the amino acid sequence of SEQ ID NO: 56 or a variant thereof in which one or two or three amino acids in one or more of the HC-CDR1, HC-CDR2, or HC-CDR3 are substituted with another amino acid; and a VL region comprising: a LC-CDR1 having the amino acid sequence of SEQ ID NO: 144; a LC-CDR2 having the amino acid sequence YAS; a LC-CDR3 having the amino acid sequence of SEQ ID NO: 64, or a variant thereof in which one or two or three amino acids in one or more of the LC-CDR1, LC-CDR2, or LC-CDR3 are substituted with another amino acid.

In some embodiments the VH4-34 antigen-binding molecule comprises a VH region according to any one of (1) to (46) above, and a VL region according to any one of (47) to (100) above.

In some embodiments, the VH4-34 antigen-binding molecule comprises a heavy chain variable region (VH) that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the heavy chain variable region (VH) of a VH4-34 antigen-binding molecule disclosed in Table C, and a light chain variable region (VL) that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the light chain variable region (VL) of the same the VH4-34 antigen-binding molecule disclosed in Table C.

In some embodiments, the VH4-34 antigen-binding molecule comprises a heavy chain variable region comprising an amino acid sequence having at least 80% sequence identity with an amino acid sequence according to any one of SEQ ID NOs: 5, 21, 37, 53, 125, 141, 156, 151, 184, 167, 171, 176, 181, and 182; and a light chain variable region comprising an amino acid sequence having at least 80% sequence identity with an amino acid sequence according to any one of SEQ ID NOs: 13, 29, 45, 61, 130, 143, 147, 161, 153, 209, 189, 193, 198, 201, and 205. In some embodiments, the VH4-34 antigen-binding molecule comprises a heavy chain variable region comprising an amino acid sequence having at least 85% sequence identity with an amino acid sequence according to any one of SEQ ID NOs: 75, 21, 37, 53, 125, 141, 156, 151, 184, 167, 171, 176, 181, and 182; and a light chain variable region comprising an amino acid sequence having at least 85% sequence identity with an amino acid sequence according to any one of SEQ ID NOs: 13, 29, 45, 61, 130, 143, 147, 161, 153, 209, 189, 193, 198, 201, and 205. In some embodiments, the VH4-34 antigen-binding molecule comprises a heavy chain variable region comprising an amino acid sequence having at least 90% sequence identity with an amino acid sequence according to any one of SEQ ID NOs: 75, 21, 37, 53, 125, 141, 156, 151, 184, 167, 171, 176, 181, and 182; and a light chain variable region comprising an amino acid sequence having at least 90% sequence identity with an amino acid sequence according to any one of SEQ ID NOs: 13, 29, 45, 61, 130, 143, 147, 161, 153, 209, 189, 193, 198, 201, and 205. In some embodiments, the VH4-34 antigen-binding molecule comprises a heavy chain variable region comprising an amino acid sequence having at least 95% sequence identity with an amino acid sequence according to any one of SEQ ID NOs: 75, 21, 37, 53, 125, 141, 156, 151, 184, 167, 171, 176, 181, and 182; and a light chain variable region comprising an amino acid sequence having at least 95% sequence identity with an amino acid sequence according to any one of SEQ ID NOs: 13, 29, 45, 61, 130, 143, 147, 161, 153, 209, 189, 193, 198, 201, and 205. In some embodiments, the VH4-34 antigen-binding molecule comprises a heavy chain variable region comprising an amino acid sequence having at least 96% sequence identity with an amino acid sequence according to any one of SEQ ID NOs: 75, 21, 37, 53, 125, 141, 156, 151, 184, 167, 171, 176, 181, and 182; and a light chain variable region comprising an amino acid sequence having at least 96% sequence identity with an amino acid sequence according to any one of SEQ ID NOs: 13, 29, 45, 61, 130, 143, 147, 161, 153, 209, 189, 193, 198, 201, and 205. In some embodiments, the VH4-34 antigen-binding molecule comprises a heavy chain variable region comprising an amino acid sequence having at least 97% sequence identity with an amino acid sequence according to any one of SEQ ID NOs: 75, 21, 37, 53, 125, 141, 156, 151, 184, 167, 171, 176, 181, and 182; and a light chain variable region comprising an amino acid sequence having at least 97% sequence identity with an amino acid sequence according to any one of SEQ ID NOs: 13, 29, 45, 61, 130, 143, 147, 161, 153, 209, 189, 193, 198, 201, and 205. In some embodiments, the VH4-34 antigen-binding molecule comprises a heavy chain variable region comprising an amino acid sequence having at least 98% sequence identity with an amino acid sequence according to any one of SEQ ID NOs: 75, 21, 37, 53, 125, 141, 156, 151, 184, 167, 171, 176, 181, and 182; and a light chain variable region comprising an amino acid sequence having at least 98% sequence identity with an amino acid sequence according to any one of SEQ ID NOs: 13, 29, 45, 61, 130, 143, 147, 161, 153, 209, 189, 193, 198, 201, and 205. In some embodiments, the VH4-34 antigen-binding molecule comprises a heavy chain variable region comprising an amino acid sequence having at least 99% sequence identity with an amino acid sequence according to any one of SEQ ID NOs: 75, 21, 37, 53, 125, 141, 156, 151, 184, 167, 171, 176, 181, and 182; and a light chain variable region comprising an amino acid sequence having at least 99% sequence identity with an amino acid sequence according any one of SEQ ID NOs: 13, 29, 45, 61, 130, 143, 147, 161, 153, 209, 189, 193, 198, 201, and 205. In some embodiments, the VH4-34 antigen-binding molecule comprises a heavy chain variable region comprising an amino acid sequence according to any one of SEQ ID NOs: 75, 21, 37, 53, 125, 141, 156, 151, 184, 167, 171, 176, 181, and 182; and a light chain variable region comprising an amino acid sequence according to any one of SEQ ID NOs: 13, 29, 45, 61, 130, 143, 147, 161, 153, 209, 189, 193, 198, 201, and 205.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 5, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 13.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 21, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 29.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 37, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 45.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 53, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 61.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 125, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 130.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 141, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 143.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 141, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 147.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 156, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 161.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 151, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 153.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 184, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 209.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 184, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 189.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 184, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 193.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 184, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 198.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 184, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 201.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 184, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 205.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 167, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 209.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 167, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 189.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 167, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 193.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 167, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 198.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 167, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 201.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 167, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 205.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 171, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 209.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 171, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 189.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 171, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 193.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 171, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 198.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 910%, at least 92%, at least 93%, at least 94%, at least 95%, at least 96%, at least 97%, at least 98%, at least 99%, or 100%) identity with an amino acid sequence according to SEQ ID NO: 171, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 201.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 171, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 205.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 176, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 209.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 176, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 189.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 176, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 193.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 176, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 198.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 176, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 201.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 176, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 205.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 181, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 209.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 181, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 189.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 181, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 193.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 181, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 198.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 181, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 201.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 181, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 205.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 182, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 209.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 182, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 189.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 182, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 193.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 182, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 198.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 182, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 201.

In some embodiments, the VH4-34 antigen-binding molecule comprises a heavy chain variable region (VH) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 182, and a light chain variable region (VL) comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 205.

In some embodiments, the VH4-34 antigen-binding molecule comprises a heavy chain comprising an amino acid sequence having at least 70% (e.g., at least 70%, at least 80%, at least 85%, at least 90%, at least 91%, at least 92%, at least 93%, at least 94%, at least 95%, at least 96%, at least 97%, at least 98%, at least 99%, or 100%) sequence identity with an amino acid sequence according to any one of SEQ ID NOs: 5, 21, 37, 53, 125, 141, 156, 151, 184, 167, 171, 176, 181, and 182; and a light chain comprising an amino acid sequence having at least 75% sequence identity with an amino acid sequence according to any one of SEQ ID NOs: 86-89, 136, 139, and 214-219. In some embodiments, the VH4-34 antigen-binding molecule comprises a heavy chain comprising an amino acid sequence having at least 80% sequence identity with an amino acid sequence according to any one of SEQ ID NOs: 5, 21, 37, 53, 125, 141, 156, 151, 184, 167, 171, 176, 181, and 182; and a light chain comprising an amino acid sequence having at least 80% sequence identity with an amino acid sequence according to any one of SEQ ID NOs: 86-89, 136, 139, and 214-219. In some embodiments, the VH4-34 antigen-binding molecule comprises a heavy chain comprising an amino acid sequence having at least 80% sequence identity with an amino acid sequence according to any one of SEQ ID NOs: 5, 21, 37, 53, 125, 141, 156, 151, 184, 167, 171, 176, 181, and 182; and a light chain comprising an amino acid sequence having at least 80% sequence identity with an amino acid sequence according to any one of SEQ ID NOs: 86-89, 136, 139, and 214-219. In some embodiments, the VH4-34 antigen-binding molecule comprises a heavy chain comprising an amino acid sequence having at least 85% sequence identity with an amino acid sequence according to any one of SEQ ID NOs: 90-93, 137, 140, 146, and 220-225; and a light chain comprising an amino acid sequence having at least 85% sequence identity with an amino acid sequence according to any one of SEQ ID NOs: 86-89, 136, 139, and 214-219. In some embodiments, the VH4-34 antigen-binding molecule comprises a heavy chain comprising an amino acid sequence having at least 90% sequence identity with an amino acid sequence according to any one of SEQ ID NOs: 90-93, 137, 140, 146, and 220-225; and a light chain comprising an amino acid sequence having at least 90% sequence identity with an amino acid sequence according to any one of SEQ ID NOs: 86-89, 136, 139, and 214-219. In some embodiments, the VH4-34 antigen-binding molecule comprises a heavy chain comprising an amino acid sequence having at least 95% sequence identity with an amino acid sequence according to any one of SEQ ID NOs: 90-93, 137, 140, 146, and 220-225; and a light chain comprising an amino acid sequence having at least 95% sequence identity with an amino acid sequence according to any one of SEQ ID NOs: 86-89, 136, 139, and 214-219. In some embodiments, the VH4-34 antigen-binding molecule comprises a heavy chain comprising an amino acid sequence having at least 96% sequence identity with an amino acid sequence according to any one of SEQ ID NOs: 90-93, 137, 140, 146, and 220-225; and a light chain comprising an amino acid sequence having at least 96% sequence identity with an amino acid sequence according to any one of SEQ ID NOs: 86-89, 136, 139, and 214-219. In some embodiments, the VH4-34 antigen-binding molecule comprises a heavy chain comprising an amino acid sequence having at least 97% sequence identity with an amino acid sequence according to any one of SEQ ID NOs: 90-93, 137, 140, 146, and 220-225; and a light chain comprising an amino acid sequence having at least 97% sequence identity with an amino acid sequence according to any one of SEQ ID NOs: 86-89, 136, 139, and 214-219. In some embodiments, the VH4-34 antigen-binding molecule comprises a heavy chain comprising an amino acid sequence having at least 98% sequence identity with an amino acid sequence according to any one of SEQ ID NOs: 90-93, 137, 140, 146, and 220-225; and a light chain comprising an amino acid sequence having at least 98% sequence identity with an amino acid sequence according to any one of SEQ ID NOs: 86-89, 136, 139, and 214-219. In some embodiments, the VH4-34 antigen-binding molecule comprises a heavy chain comprising an amino acid sequence having at least 99% sequence identity with an amino acid sequence according to any one of SEQ ID NOs: 90-93, 137, 140, 146, and 220-225; and a light chain comprising an amino acid sequence having at least 99% sequence identity with an amino acid sequence according any one of SEQ ID NOs: 86-89, 136, 139, and 214-219. In some embodiments, the VH4-34 antigen-binding molecule comprises a heavy chain comprising an amino acid sequence according to any one of SEQ ID NOs: 90-93, 137, 140, 146, and 220-225; and a light chain comprising an amino acid sequence according to any one of SEQ ID NOs: 86-89, 136, 139, and 214-219. See Table D for heavy chain and light chain combinations.

In some embodiments, the VH4-34 antigen-binding molecule comprises a heavy chain comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 215, and a light chain comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 221.

In some embodiments, the VH4-34 antigen-binding molecule comprises a heavy chain comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 230, and a light chain comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 221.

In some embodiments, the VH4-34 antigen-binding molecule comprises a heavy chain comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 219, and a light chain comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 222.

In some embodiments, the VH4-34 antigen-binding molecule comprises a heavy chain comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 234, and a light chain comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 222.

In some embodiments, the VH4-34 antigen-binding molecule comprises a heavy chain comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 219, and a light chain comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 223.

In some embodiments, the VH4-34 antigen-binding molecule comprises a heavy chain comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 234, and a light chain comprising an amino acid sequence having at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identity with an amino acid sequence according to SEQ ID NO: 223.

In embodiments in accordance with the present disclosure in which one or more amino acids are substituted with another amino acid, the substitutions may be conservative substitutions, for example according to the following Table. In some embodiments, amino acids in the same block in the middle column are substituted. In some embodiments, amino acids in the same line in the rightmost column are substituted:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

In some embodiments, substitution(s) is functionally conservative. That is, in some embodiments the substitution may not affect (or may not substantially affect) one or more functional properties (e.g. target binding) of the VH4-34 antigen-binding molecule comprising the substitution as compared to the equivalent unsubstituted molecule.

The VH and VL regions of an antigen-binding region of an antibody together constitute the Fv region. In some embodiments, the VH4-34 antigen-binding molecule comprises, or consists of, an Fv region which binds to VH4-34. In some embodiments the VH and VL regions of the Fv are provided as single polypeptide joined by a linker region, i.e. a single chain Fv (scFv).

The VL and light chain constant (CL) region, and the VH region and heavy chain constant 1 (CH1) region of an antigen-binding region of an antibody together constitute the Fab region. In some embodiments the VH4-34 antigen-binding molecule comprises a Fab region comprising a VH, a CH1, a VL and a CL (e.g. CK or Ck). In some embodiments the Fab region comprises a polypeptide comprising a VH and a CH1 (e.g. a VH-CH1 fusion polypeptide), and a polypeptide comprising a VL and a CL (e.g. a VL-CL fusion polypeptide). In some embodiments the Fab region comprises a polypeptide comprising a VH and a CL (e.g. a VH-CL fusion polypeptide) and a polypeptide comprising a VL and a CH (e.g. a VL-CH1 fusion polypeptide); that is, in some embodiments the Fab region is a CrossFab region. In some embodiments the VH, CH1, VL and CL regions of the Fab or CrossFab are provided as single polypeptide joined by linker regions, i.e. as a single chain Fab (scFab) or a single chain CrossFab (scCrossFab).

In some embodiments, the VH4-34 antigen-binding molecule of the present disclosure comprises, or consists of, a Fab region which binds to VH4-34.

In some embodiments, the VH4-34 antigen-binding molecule described herein comprises, or consists of, a whole antibody which binds to VH4-34. As used herein, “whole antibody” refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125(202): S41-S52, which is hereby incorporated by reference in its entirety.

Immunoglobulins of type G (i.e. IgG) are ~150 kDa glycoproteins comprising two heavy chains and two light chains. From N- to C-terminus, the heavy chains comprise a VH followed by a heavy chain constant region comprising three constant domains (CH1, CH2, and CH3), and similarly the light chains comprise a VL followed by a CL. Depending on the heavy chain, immunoglobulins may be classed as IgG (e.g. IgG1, IgG2, IgG3. IgG4), IgA (e.g. IgAQ1. IgA2), IgD, IgE, or IgM. The light chain may be kappa (x) or lambda (Q).

In some embodiments, the VH4-34 antigen-binding molecule described herein comprises, or consists of, an IgG (e.g. IgG1, IgG2, IgG3, IgG4), IgA (e.g. IgAQ1, IgA2), IgD, IgE, or IgM which binds to VH4-34.

In some embodiments the VH4-34 antigen-binding molecule of the present disclosure comprises one or more regions (e.g. CH1, CH2, CH3, etc.) of an immunoglobulin heavy chain constant sequence. In some embodiments the immunoglobulin heavy chain constant sequence is, or is derived from, the heavy chain constant sequence of an IgG (e.g. IgG1, IgG2, IgG3, IgG4), IgA (e.g. IgA1, IgA2), IgD, IgE or IgM, e.g. a human IgG (e.g. hIgG1, hIgG2, hIgG3, hIgG4), hIgA (e.g. hIgA1, hIgA2), hIgD, hIgE or hIgM. In some embodiments the immunoglobulin heavy chain constant sequence is, or is derived from, the heavy chain constant sequence of a human IgG1 allotype (e.g. G1m1, G1m2, G1m3 or G1m17).

In some embodiments the VH4-34 antigen-binding molecule comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 71 or 76.

In some embodiments the VH4-34 antigen-binding molecule comprises a CH1 region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 72 or 77. In some embodiments the VH4-34 antigen-binding molecule comprises a hinge region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 73 or 85. In some embodiments the VH4-34 antigen-binding molecule comprises a CH2 region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 74. In some embodiments the VH4-34 antigen-binding molecule comprises a CH3 region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 75 or 78.

It will be appreciated that CH2 and/or CH3 regions may be provided with further substitutions in accordance with modification to an Fc region of the VH4-34 antigen-binding molecule as described herein.

In some embodiments the VH4-34 antigen-binding molecule of the present disclosure comprises one or more regions of an immunoglobulin light chain constant sequence. In some embodiments the immunoglobulin light chain constant sequence is human immunoglobulin kappa constant (IGKC; Cκ). In some embodiments the immunoglobulin light chain constant sequence is a human immunoglobulin lambda constant (IGLC; Cλ). e.g. IGLC1, IGLC2. IGLC3, IGLC6 or IGLC7.

In some embodiments the VH4-34 antigen-binding molecule comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO: 79. In some embodiments the VH4-34 antigen-binding molecule comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of one of SEQ ID NOs: 80 to 84.

In some embodiments, the VH4-34 antigen-binding molecule is or comprises a fully human antibody/antibody fragment. A fully human antibody/antibody fragment may be encoded by human nucleic acid sequence(s). A fully human antibody/antibody fragment may be devoid of non-human amino acid sequences. Commonly employed techniques for the production of fully human antibodies include (i) phage display, in which human antibody genes are expressed in phage display libraries, and (ii) production of antibodies in transgenic mice engineered to have human antibody genes (described in Park and Smolen, Advances in Protein Chemistry (2001) 56: 369-421). Briefly, in the human antibody gene-phage display technique, genes encoding the VH and VL chains are generated by PCR amplification and cloning from “naive” human lymphocytes, and assembled into a library from which they can be expressed either as disulfide-linked Fab fragments or as single-chain Fv (scFv) fragments. The Fab- or scFv-encoding genes are fused to a surface coat protein of filamentous bacteriophage and Fab or scFv capable of binding to the target of interest can then be identified by screening the library with antigen. Molecular evolution or affinity maturation procedures can be employed to enhance the affinity of the Fab/scFv fragment. In the transgenic mouse technique, mice in which the endogenous murine lg gene loci have been replaced by homologous recombination with their human homologues are immunized with antigen, and monoclonal antibody is prepared by conventional hybridoma technology, to yield a fully human monoclonal antibody.

In some embodiments, the VH4-34 antigen-binding molecule of the present disclosure is a mouse antibody/antibody fragment. In some embodiments the antibody/antibody fragment is obtained from phage display using a human naïve antibody gene library.

In some embodiments, the VH4-34 antigen-binding molecule is a mouse/human chimeric antibody/antibody fragment (i.e. an antigen-binding molecule comprising mouse antibody variable domains and human antibody constant regions). In some embodiments, the VH4-34 antigen-binding molecule is a humanised antibody/antibody fragment. In some embodiments, the VH4-34 antigen-binding molecule comprises mouse antibody CDRs and human antibody framework and constant regions. In some embodiments, the VH4-34 antigen-binding molecule comprises human antibody CDRs and human antibody framework and constant regions.

Mouse/human chimeric antigen-binding molecules can be prepared from mouse antibodies by the process of chimerisation, e.g. as described in Human Monoclonal Antibodies: Methods and Protocols, Michael Steinitz (Editor), Methods in Molecular Biology 1060, Springer Protocols, Humana Press (2014), in Chapter 8 thereof, in particular section 3 of Chapter 8.

Humanised antigen-binding molecules can be designed and prepared from mouse antibodies by the process of humanisation, e.g. as described in Human Monoclonal Antibodies: Methods and Protocols, Michael Steinitz (Editor), Methods in Molecular Biology 1060, Springer Protocols, Humana Press (2014), in Chapter 7 thereof, in particular section 3.1 of Chapter 7 entitled ‘Antibody Humanization’. Techniques for antibody humanisation are also described e.g. in Safdari et al., Biotechnol Genet Eng Rev (2013) 29:175-86.

Aspects of the present disclosure relate to multispecific antigen-binding molecules. By “multispecific” it is meant that the VH4-34 antigen-binding molecule displays specific binding to more than one target. In some embodiments the VH4-34 antigen-binding molecule is a bispecific antigen-binding molecule. In some embodiments the VH4-34 antigen-binding molecule comprises at least two different antigen-binding domains (i.e. at least two antigen-binding domains, e.g. comprising non-identical VHs and VLs).

In some embodiments the VH4-34 antigen-binding molecule binds to VH4-34 and another target (e.g. an antigen other than VH4-34), and so is at least bispecific. The term “bispecific” means that the VH4-34 antigen-binding molecule is able to bind specifically to at least two distinct antigenic determinants.

It will be appreciated that an antigen-binding molecule (e.g. a multispecific antigen-binding molecule) may comprise antigen-binding molecules capable of binding to the targets for which the VH4-34 antigen-binding molecule is specific. For example, an antigen-binding molecule which binds to VH4-34 and an antigen other than VH4-34 may comprise: (i) an antigen-binding molecule which binds to VH4-34, and (ii) an antigen-binding molecule which binds to an antigen other than VH4-34.

It will also be appreciated that an antigen-binding molecule (e.g. a multispecific antigen-binding molecule) may comprise antigen-binding polypeptides or antigen-binding polypeptide complexes capable of binding to the targets for which the VH4-34 antigen-binding molecule is specific.

In some embodiments, a component antigen-binding molecule of a larger antigen-binding molecule (e.g. a multispecific antigen-binding molecule) may be referred to e.g. as an “antigen-binding domain” or “antigen-binding region” of the larger antigen-binding molecule.

In some embodiments the antigen other than VH4-34 in a multispecific antigen-binding molecule is an immune cell surface molecule. In some embodiments, the antigen is a cancer cell antigen. In some embodiments the antigen is a receptor molecule, e.g. a cell surface receptor. In some embodiments the antigen is a cell signalling molecule, e.g. a cytokine, chemokine, interferon, interleukin or lymphokine. In some embodiments the antigen is a growth factor or a hormone.

A cancer cell antigen is an antigen which is expressed or over-expressed by a cancer cell. A cancer cell antigen may be any peptide/polypeptide, glycoprotein, lipoprotein, glycan, glycolipid, lipid, or fragment thereof. A cancer cell antigen's expression may be associated with a cancer. A cancer cell antigen may be abnormally expressed by a cancer cell (e.g. the cancer cell antigen may be expressed with abnormal localisation), or may be expressed with an abnormal structure by a cancer cell. A cancer cell antigen may be capable of eliciting an immune response. In some embodiments, the antigen is expressed at the cell surface of the cancer cell (i.e. the cancer cell antigen is a cancer cell surface antigen). In some embodiments, the part of the antigen which is bound by the VH4-34 antigen-binding molecule described herein is displayed on the external surface of the cancer cell (i.e. is extracellular). The cancer cell antigen may be a cancer-associated antigen. In some embodiments the cancer cell antigen is an antigen whose expression is associated with the development, progression or severity of symptoms of a cancer. The cancer-associated antigen may be associated with the cause or pathology of the cancer, or may be expressed abnormally as a consequence of the cancer. In some embodiments, the cancer cell antigen is an antigen whose expression is upregulated (e.g. at the RNA and/or protein level) by cells of a cancer, e.g. as compared to the level of expression by comparable non-cancerous cells (e.g. non-cancerous cells derived from the same tissue/cell type). In some embodiments, the cancer-associated antigen is preferentially expressed by cancerous cells, and not expressed by comparable non-cancerous cells (e.g. non-cancerous cells derived from the same tissue/cell type). In some embodiments, the cancer-associated antigen is the product of a mutated oncogene or mutated tumor suppressor gene. In some embodiments, the cancer-associated antigen is the product of an overexpressed cellular protein, a cancer antigen produced by an oncogenic virus, an oncofetal antigen, or a cell surface glycolipid or glycoprotein.

An immune cell surface molecule may be any peptide/polypeptide, glycoprotein, lipoprotein, glycan, glycolipid, lipid, or fragment thereof expressed at or on the cell surface of an immune cell. In some embodiments, the part of the immune cell surface molecule which is bound by the VH4-34 antigen-binding molecule of the present disclosure is on the external surface of the immune cell (i.e. is extracellular). The immune cell surface molecule may be expressed at the cell surface of any immune cell. In some embodiments, the immune cell is a cell of hematopoietic origin, e.g. a neutrophil, eosinophil, basophil, dendritic cell, lymphocyte, or monocyte. The lymphocyte may be e.g. a T cell, B cell, natural killer (NK) cell, NKT cell or innate lymphoid cell (ILC), or a precursor thereof (e.g. a thymocyte or pre-B cell).

In some embodiments the antigen is selected from: BCMA, TACI, CD47, CD33, CD123, Wilms' tumor protein (WT1), CD13, CD15, CD30, CD45, C-type lectin-like molecule 1 (CLL1), Fms-like tyrosine kinase 3 (FLT-3), VEGF and angiopoietin-2 (Ang-2). In some embodiments the antigen is a CD3 polypeptide (e.g. CD3ε, CD3δ, CD3γ or CD3ζ).

In some embodiments, multispecific antigen-binding molecules described herein display at least monovalent binding with respect to VH4-34, and also display at least monovalent binding with respect to an antigen other than VH4-34.

In some embodiments the VH4-34 antigen-binding molecule comprises an antigen-binding region (e.g. a polypeptide. Fv. Fab or antibody) capable of binding to an antigen other than VH4-34, and an antigen-binding region (e.g. a polypeptide, Fv, Fab or antibody) capable of binding to an antigen other than VH4-34. In some embodiments the VH4-34 antigen-binding molecule comprises the VH and VL of an antibody capable of binding to VH4-34 and the VH and VL of an antibody capable of binding to an antigen other than VH4-34. Binding valency refers to the number of binding sites in an antigen-binding molecule for a given antigenic determinant.

In some embodiments, the VH4-34 antigen-binding molecule is an immune cell engager. Immune cell engagers are reviewed e.g. in Goebeler and Bargou, Nat. Rev. Clin. Oncol. (2020) 17: 418-434 and Ellerman. Methods (2019) 154:102-117, both of which are hereby incorporated by reference in their entirety.

Immune cell engager molecules comprise an antigen-binding region for a target antigen of interest, and an antigen-binding region for recruiting/engaging an immune cell of interest. Immune cell engagers recruit/engage immune cells through an antigen-binding region specific for an immune cell surface molecule.

The best studied immune cells engagers are bispecific T cell engagers (BiTEs), which comprise a target antigen binding domain, and a CD3 polypeptide (typically CD3E)-binding domain, through which the BiTE recruits T cells. Binding of the BiTE to its target antigen and to the CD3 polypeptide expressed by the T cell results in activation of the T cell, and ultimately directs T cell effector activity against cells expressing the target antigen. Other kinds of immune cell engagers are well known in the art, and include natural killer cell engagers such as bispecific killer engagers (BiKEs), which recruit and activate NK cells.

In some embodiments, the immune cell engaged by the immune cell engager is a T cell or an NK cell. In some embodiments, the immune cell engager is a T cell-engager.

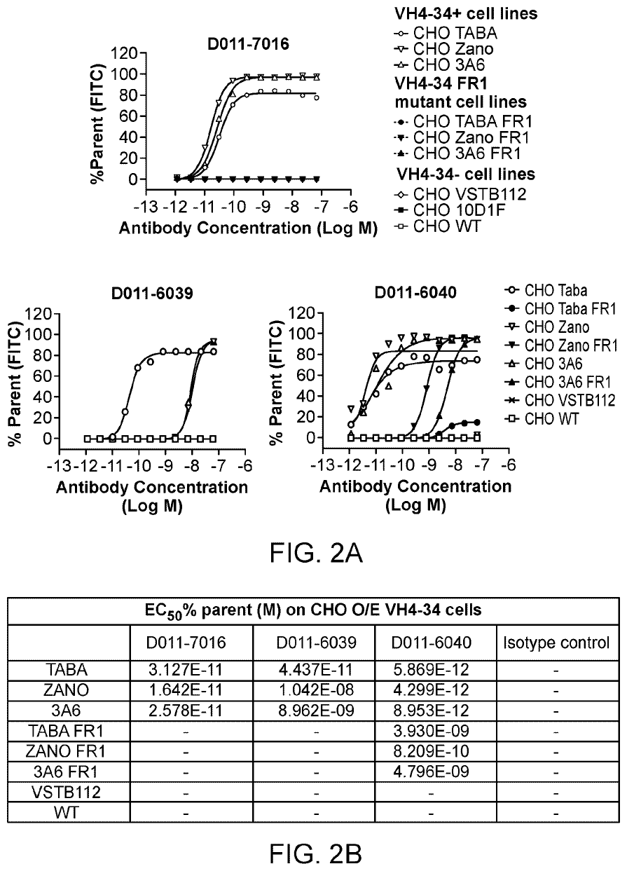

Multispecific antigen-binding molecules may be provided in any suitable format, such as those formats described in described in Brinkmann and Kontermann. MAbs (2017) 9(2): 182-212, which is hereby incorporated by reference in its entirety. Suitable formats include those shown in FIG. 2 of Brinkmann and Kontermann, MAbs (2017) 9(2): 182-212; antibody conjugates, e.g. IgG₂, F(ab′)₂ or CovX-Body; IgG or IgG-like molecules, e.g. IgG, chimeric IgG, κλ-body common HC; CH1/CL fusion proteins, e.g. scFv2-CH1/CL, VHH2-CH1/CL; ‘variable domain only’ bispecific antigen-binding molecules, e.g. tandem scFv (taFV), triplebodies, diabodies (Db), dsDb, Db(kih), DART, scDB, dsFv-dsFv, tandAbs, triple heads, tandem dAb/VHH, tertravalent dAb.VHH; Non-Ig fusion proteins, e.g. scFv2-albumin, scDb-albumin, taFv-albumin, taFv-toxin, miniantibody, DNL-Fab₂, DNL-Fab₂-scFv, DNL-Fab₂-IgG-cytokine₂, ImmTAC (TCR-scFv); modified Fc and CH3 fusion proteins, e.g. scFv-Fc(kih), scFv-Fc(CH3 charge pairs), scFv-Fc (EW-RVT), scFv-fc (HA-TF), scFv-Fc (SEEDbody), taFv-Fc(kih), scFv-Fc(kih)-Fv, Fab-Fc(kih)-scFv, Fab-scFv-Fc(kih), Fab-scFv-Fc(BEAT), Fab-scFv-Fc (SEEDbody), DART-Fc, scFv-CH3(kih), TriFabs; Fc fusions, e.g. Di-diabody, scDb-Fc, taFv-Fc, scFv-Fc-scFv, HCAb-VHH, Fab-scFv-Fc, scFv4-Ig, scFv2-Fcab; CH3 fusions, e.g. Dia-diabody, scDb-CH3; IgE/IgM CH2 fusions, e.g. scFv-EHD2-scFv, scFvMHD2-scFv; Fab fusion proteins, e.g. Fab-scFv (bibody), Fab-scFv₂ (tribody), Fab-Fv, Fab-dsFv, Fab-VHH, orthogonal Fab-Fab; non-Ig fusion proteins, e.g. DNL-Fab₃, DNL-Fab₂-scFv, DNL-Fab₂-IgG-cytokine₂; asymmetric IgG or IgG-like molecules, e.g. IgG(kih), IgG(kih) common LC, ZW1 IgG common LC. Biclonics common LC, CrossMab, CrossMab(kih), scFab-IgG(kih), Fab-scFab-IgG(kih), orthogonal Fab IgG(kih). DuetMab, CH3 charge pairs+CH1/CL charge pairs, hinge/CH3 charge pairs, SEED-body, Duobody, four-in-one-CrossMab(kih), LUZ-Y common LC; LUZ-Y scFab-IgG, FcFc*; appended and Fc-modified IgGs, e.g. IgG(kih)-Fv, IgG HA-TF-Fv, IgG(kih)scFab, scFab-Fc(kih)-scFv2, scFab-Fc(kih)-scFv, half DVD-Ig, DVI-Ig (four-in-one), CrossMab-Fab: modified Fc and CH3 fusion proteins. e.g. Fab-Fc(kih)-scFv, Fab-scFv-Fc(kih), Fab-scFv-Fc(BEAT), Fab-scFv-Fc-SEEDbody, TriFab; appended IgGs-HC fusions, e.g. IgG-HC, scFv, IgG-dAb, IgG-taFV, IgG-CrossFab, IgG-orthogonal Fab, IgG-(CαCβ) Fab, scFv-HC-IgG, tandem Fab-IgG (orthogonal Fab), Fab-IgG(CαCβ Fab), Fab-IgG(CR3), Fab-hinge-IgG(CR3); appended IgGs-LC fusions, e.g. IgG-scFv(LC), scFv(LC)-IgG, dAb-IgG; appended IgGs-HC and LC fusions, e.g. DVD-Ig, TVD-Ig, CODV-Ig, scFv₄-IgG, Zybody; Fc fusions, e.g. Fab-scFv-Fc, scFv₄-Ig; F(ab′)2 fusions, e.g. F(ab′)₂-scFv₂; CH1/CL fusion proteins e.g. scFv₂-CH1-hinge/CL; modified IgGs, e.g. DAF (two-in one-IgG), DutaMab, Mab²; and non-Ig fusions, e.g. DNL-Fab₄-IgG.

The skilled person is able to design and prepare bispecific antigen-binding molecules. Methods for producing bispecific antigen-binding molecules include chemically crosslinking antigen-binding molecules or antibody fragments, e.g. with reducible disulphide or non-reducible thioether bonds, for example as described in Segal and Bast, 2001. Production of Bispecific Antigen-binding molecules. Current Protocols in Immunology. 14:IV:2.13:2.13.1-2.13.16, which is hereby incorporated by reference in its entirety. For example, N-succinimidyl-3-(-2-pyridyldithio)-propionate (SPDP) can be used to chemically crosslink e.g. Fab fragments via hinge region SH-groups, to create disulfide-linked bispecific F(ab)₂ heterodimers.

Other methods for producing bispecific antigen-binding molecules include fusing antibody-producing hybridomas e.g. with polyethylene glycol, to produce a quadroma cell capable of secreting bispecific antibody, for example as described in D. M. and Bast, B. J. 2001. Production of Bispecific Antigen-binding molecules. Current Protocols in Immunology. 14:IV:2.13:2.13.1-2.13.16.

Bispecific antigen-binding molecules can also be produced recombinantly, by expression from e.g. a nucleic acid construct encoding polypeptides for the VH4-34 antigen-binding molecules, for example as described in Antibody Engineering: Methods and Protocols, Second Edition (Humana Press, 2012), at Chapter 40: Production of Bispecific Antigen-binding molecules: Diabodies and Tandem scFv (Hornig and Farber-Schwarz), or French, How to make bispecific antigen-binding molecules, Methods Mol. Med. 2000; 40:333-339, the entire contents of both of which are hereby incorporated by reference.

For example, a DNA construct encoding the light and heavy chain variable domains for the two antigen-binding fragments (i.e. the light and heavy chain variable domains for the antigen-binding fragment capable of binding VH4-34, and the light and heavy chain variable domains for the antigen-binding fragment capable of binding to another target protein), and including sequences encoding a suitable linker or dimerization domain between the antigen-binding fragments can be prepared by molecular cloning techniques. Recombinant bispecific antibody can thereafter be produced by expression (e.g. in vitro) of the construct in a suitable host cell (e.g. a mammalian host cell), and expressed recombinant bispecific antibody can then optionally be purified.

### Fc Regions

In some embodiments the VH4-34 antigen-binding molecules of the present disclosure comprise an Fc region.

An Fc region is composed of CH2 and CH3 regions from one polypeptide, and CH2 and CH3 regions from another polypeptide. The CH2 and CH3 regions from the two polypeptides together form the Fc region.

Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

Modifications to antibody Fc regions that influence Fc-mediated functions are known in the art, such as those described e.g. in Wang et al., Protein Cell (2018) 9(1):63-73, which is hereby incorporated by reference in its entirety. Exemplary Fc region modifications known to influence antibody effector function are summarised in Table 1 of Wang et al., Protein Cell (2018) 9(1):63-73.

In some embodiments the VH4-34 antigen-binding molecule of the present disclosure comprises an Fc region comprising modification to increase or reduce an Fc-mediated function as compared to an antigen-binding molecule comprising the corresponding unmodified Fc region.

The combination of substitutions F243L/R292P/Y300L/V3051/P396L is described in Stavenhagen et al. Cancer Res. (2007) to increase binding to FcγRIIIa, and thereby enhance ADCC. The combination of substitutions S239D/I332E or S239D/I332E/A330L is described in Lazar et al., Proc Natl Acad Sci USA. (2006)103:4005-4010 to increase binding to FcγRIIIa, and thereby increase ADCC. The combination of substitutions S239D/I332E/A330L is also described to decrease binding to FcγRIIb, and thereby increase ADCC. The combination of substitutions S298A/E333A/K334A is described in Shields et al., J Biol Chem. (2001) 276:6591-6604 to increase binding to FcγRIIIa. and thereby increase ADCC. The combination of substitutions L234Y/L235Q/G236W/S239M/H268D/D270E/S298A in one heavy chain, and the combination of substitutions D270E/K326D/A330M/K334E in the other heavy chain, is described in Mimoto et al., MAbs. (2013): 5:229-236 to increase binding to FcγRIIIa, and thereby increase ADCC. The combination of substitutions G236A/S239D/I332E is described in Richards et al., Mol Cancer Ther. (2008) 7:2517-2527 to increase binding to FcγRIIa and to increase binding to FcγRIIIa, and thereby increase ADCP.

The combination of substitutions K326W/E333S is described in Idusogie et al. J Immunol. (2001) 166(4):2571-5 to increase binding to C1q, and thereby increase CDC. The combination of substitutions S267E/H268F/S324T is described in Moore et al. MAbs. (2010) 2(2):181-9 to increase binding to C1q, and thereby increase CDC. The combination of substitutions described in Natsume et al., Cancer Res. (2008) 68(10):3863-72 is reported to increase binding to C1q, and thereby increase CDC. The combination of substitutions E345R/E430G/S440Y is described in Diebolder et al. Science (2014) 343(6176):1260-3 to increase hexamerisation, and thereby increase CDC.

The combination of substitutions M252Y/S254T/T256E is described in Dall'Acqua et al. J Immunol. (2002) 169:5171-5180 to increase binding to FcRn at pH 6.0, and thereby increase antigen-binding molecule half-life. The combination of substitutions M428L/N434S is described in Zalevsky et al. Nat Biotechnol. (2010) 28:157-159 to increase binding to FcRn at pH 6.0, and thereby increase antigen-binding molecule half-life.

Where a heavy chain constant region/Fc region/CH2-CH3 region/CH2 region/CH3 region is described herein as comprising position(s)/substitution(s) “corresponding to” reference position(s)/substitution(s), equivalent position(s)/substitution(s) in homologous heavy chain constant regions/Fc regions/CH2-CH3 regions/CH2 regions/CH3 regions are contemplated.

Where an Fc region is described as comprising specific position(s)/substitution(s), the position(s)/substitution(s) may be present in one or both of the polypeptide chains which together form the Fc region.

Unless otherwise specified, positions herein refer to positions of human immunoglobulin constant region amino acid sequences numbered according to the EU numbering system as described in Kabat et al., Sequences of Proteins of Immunological Interest. 5th Ed. Public Health Service, National Institutes of Health, Bethesda, MD, 1991. By way of illustration, the substitutions L242C and K334C in human IgG1 correspond to L>C substitution at position 125, and K>C substitution at position 217 of the human IgG1 constant region numbered according to SEQ ID NO: 71.

Homologous heavy chain constant regions are heavy chain constant regions comprising an amino acid sequence having at least 60%, preferably one of 70%, 75%, 80%, 85%, 90%, 91%, 92%, 93%, 94%, 95%, 96%, 97%, 98%, 99% or 100% amino acid sequence identity to the heavy chain constant region of Human IgG1 (i.e. the amino acid sequence shown in SEQ ID NO: 71). Homologous Fc regions are Fc regions comprised of polypeptides comprising an amino acid sequence having at least 60%, preferably one of 70%, 75%, 80%, 85%, 90%, 91%, 92%, 93%, 94%, 95%, 96%, 97%, 98%, 99% or 100% amino acid sequence identity to CH2-CH3 region of Human IgG1 (i.e. the amino acid sequences shown in SEQ ID NO: 74 and 75). Homologous CH2 regions are CH2 regions comprising an amino acid sequence having at least 60%, preferably one of 70%, 75%, 80%, 85%, 90%, 91%, 92%, 93%, 94%, 95%, 96%, 97%, 98%, 99% or 100% amino acid sequence identity to CH2 region of Human IgG1 (i.e. the amino acid sequence shown in SEQ ID NO: 74). Homologous CH3 regions are CH3 regions comprising an amino acid sequence having at least 60%, preferably one of 70%, 75%, 80%, 85%, 90%, 91%, 92%. 9³%. 94%, 95%, 96%, 97%, 98%, 99% or 100% amino acid sequence identity to CH3 region of Human IgG1 (i.e. the amino acid sequence shown in SEQ ID NO: 75).

Corresponding positions to those identified in human IgG1 can be identified by sequence alignment which can be performed e.g. using sequence alignment software such as ClustalOmega (Soding, J. 2005, Bioinformatics 21, 951-960).

In some embodiments the VH4-34 antigen-binding molecule of the present disclosure comprises an Fc region comprising modification to increase an Fc-mediated function. In some embodiments the Fc region comprises modification to increase ADCC. In some embodiments the Fc region comprises modification to increase ADCP. In some embodiments the Fc region comprises modification to increase CDC. An antigen-binding molecule comprising an Fc region comprising modification to increase an Fc-mediated function (e.g ADCC, ADCP, CDC) induces an increased level of the relevant effector function as compared to an antigen-binding molecule comprising the corresponding unmodified Fc region.

In some embodiments, the VH4-34 antigen-binding molecule of the present disclosure comprises an Fc region comprising modification to increase affinity for one or more Fc receptors (e.g. FcγRIIa, FcγRIIIa). Modifications increasing affinity for Fc receptors can increase Fc-mediated effector function such as antibody-dependent cellular cytotoxicity (ADCC) and/or antibody-dependent cellular phagocytosis (ADCP). In some embodiments, the VH4-34 antigen-binding molecule of the present disclosure comprises an Fc region comprising modification to reduce affinity for C1q; such modification reducing complement-dependent cytotoxicity (CDC), which can be desirable. In some embodiments, the VH4-34 antigen-binding molecule of the present disclosure comprises an Fc region comprising modification to increase hexamer formation. Modifications to the Fc region capable of increasing affinity for one or more Fc receptors, reducing affinity for C1q and/or increasing hexamer formation are described e.g. in Saxena and Wu Front Immunol. (2016) 7:580, which is hereby incorporated by reference in its entirety. In some embodiments the VH4-34 antigen-binding molecule of the present disclosure comprises an Fc region comprising CH2/CH3 comprising one or more of the substitutions shown in Table 1 of Saxena and Wu Front Immunol. (2016) 7:580.

In some embodiments the VH4-34 antigen-binding molecule of the present disclosure comprises an Fc comprising modification to increase binding to an Fc receptor. In some embodiments the Fc region comprises modification to increase binding to an Fcγ receptor. In some embodiments the Fc region comprises modification to increase binding to one or more of FcγRI, FcγRIIa, FcγRIIb, FcγRIIc, FcγRIIIa and FcγRIIIb. In some embodiments the Fc region comprises modification to increase binding to FcγRIIIa. In some embodiments the Fc region comprises modification to increase binding to FcγRIIa. In some embodiments the Fc region comprises modification to increase binding to FcγRIIb. In some embodiments the Fc region comprises modification to increase binding to FcRn. In some embodiments the Fc region comprises modification to increase binding to a complement protein. In some embodiments the Fc region comprises modification to increase or reduce binding to C1q. In some embodiments the Fc region comprises modification to promote hexamerisation of the VH4-34 antigen-binding molecule. In some embodiments the Fc region comprises modification to increase antigen-binding molecule serum half-life. In some embodiments the Fc region comprises modification to increase co-engagement.

In this specification an “Fcγ receptor” may be from any species, and includes isoforms, fragments, variants (including mutants) or homologues from any species. Similarly, “FcγRI”, “FcγRIIa”, “FcγRIIb”, “FcγRIIc”, “FcγRIIIa” and “FcγRIIIb” refer respectively to FcγRI/FcγRIIa/FcγRIIb/FcγRIIc/FcγRIIIa/FcγRIIIb from any species, and include isoforms, fragments, variants (including mutants) or homologues from any species. Humans have six different classes of Fc γ receptor (mouse orthologues are shown in brackets): FcγRI (mFcγRI). FcγRIIa (mFcγRIII), FcγRIIb (mFcγRIIb), FcγRIIc, FcγRIIIa (mFcγRIV) and FcγRIIIb.

Variant Fc γ receptors include e.g. the 158V and 158F polymorphs of human FcγRIIIa, and the 167H and 167R polymorphs of human FcγRIIa.

In some embodiments, the VH4-34 antigen-binding molecule of the present disclosure comprises an Fc region comprising (e.g. comprising one more polypeptides comprising a heavy chain constant region, or a CH2-CH3 region, comprising) one or more (e.g. 1, 2, 3, 4, 5, 6, 7 or 8) of the following: C at the position corresponding to position 242; C at the position corresponding to position 334; A at the position corresponding to position 236; D at the position corresponding to position 239: E at the position corresponding to position 332; L at the position corresponding to position 330; K at the position corresponding to position 345; and G at the position corresponding to position 430.

In some embodiments the VH4-34 antigen-binding molecule of the present disclosure comprises an Fc region comprising (e.g. comprising one more polypeptides comprising a heavy chain constant region, or a CH2-CH3 region, comprising) one or more (e.g. 1, 2, 3, 4, 5, 6, 7 or 8) of the following substitutions (or corresponding substitutions): L242C, K334C, G236A, S239D, I332E, A330L. E345K, and E430G.

In some embodiments the VH4-34 antigen-binding molecule comprises an Fc region comprising (e.g. comprising one more polypeptides comprising a heavy chain constant region, a CH2-CH3 region, or a CH2 region, comprising) a C at the position corresponding to position 242. In some embodiments the Fc region comprises (e.g. comprises one more polypeptides comprising a heavy chain constant region, a CH2-CH3 region, or a CH2 region, comprising) a C at the position corresponding to position 334. In some embodiments the Fc region comprises (e.g. comprises one more polypeptides comprising a heavy chain constant region, a CH2-CH3 region, or a CH2 region, comprising) a C at the position corresponding to position 242 and a C at the position corresponding to position 334.

In some embodiments the VH4-34 antigen-binding molecule comprises an Fc region comprising (e.g. comprising one more polypeptides comprising a heavy chain constant region, a CH2-CH3 region, or a CH2 region, comprising) an A at the position corresponding to position 236. In some embodiments the Fc region comprises (e.g. comprises one more polypeptides comprising a heavy chain constant region, a CH2-CH3 region, or a CH2 region, comprising) a D at the position corresponding to position 239. In some embodiments the Fc region comprises (e.g. comprises one more polypeptides comprising a heavy chain constant region, a CH2-CH3 region, or a CH2 region, comprising) an A at the position corresponding to position 236, and a D at the position corresponding to position 239.

In some embodiments the VH4-34 antigen-binding molecule comprises an Fc region comprising (e.g. comprising one more polypeptides comprising a heavy chain constant region, a CH2-CH3 region, or a CH2 region, comprising) an E at the position corresponding to position 332. In some embodiments the Fc region comprises (e.g. comprises one more polypeptides comprising a heavy chain constant region, a CH2-CH3 region, or a CH2 region, comprising) an A at the position corresponding to position 236, a D at the position corresponding to position 239, and an E at the position corresponding to position 332.

In some embodiments the VH4-34 antigen-binding molecule comprises an Fc region comprising (e.g. comprising one more polypeptides comprising a heavy chain constant region, a CH2-CH3 region, or a CH2 region, comprising) an L at the position corresponding to position 330. In some embodiments the Fc region comprises (e.g. comprises one more polypeptides comprising a heavy chain constant region, a CH2-CH3 region, or a CH2 region, comprising) an A at the position corresponding to position 236, a D at the position corresponding to position 239, an E at the position corresponding to position 332, and an L at the position corresponding to position 330.

In some embodiments the VH4-34 antigen-binding molecule comprises an Fc region comprising (e.g. comprising one more polypeptides comprising a heavy chain constant region, a CH2-CH3 region, or a CH3 region, comprising) a K at the position corresponding to position 345. In some embodiments the Fc region comprises (e.g. comprises one more polypeptides comprising a heavy chain constant region, a CH2-CH3 region, or a CH3 region, comprising) a G at the position corresponding to position 430. In some embodiments the Fc region comprises (e.g. comprises one more polypeptides comprising a heavy chain constant region, a CH2-CH3 region, or a CH2 region, comprising) a K at the position corresponding to position 345, and a G at the position corresponding to position 430.

In some embodiments the VH4-34 antigen-binding molecule comprises an Fc region comprising (e.g. comprising one more polypeptides comprising a heavy chain constant region, a CH2-CH3 region, or a CH2 region, comprising) a C at the position corresponding to position 242, a C at the position corresponding to position 334, an A at the position corresponding to position 236, and a D at the position corresponding to position 239.

In some embodiments the VH4-34 antigen-binding molecule comprises an Fc region comprising (e.g. comprising one more polypeptides comprising a heavy chain constant region, a CH2-CH3 region, or a CH2 region, comprising) a C at the position corresponding to position 242, a C at the position corresponding to position 334, an A at the position corresponding to position 236, a D at the position corresponding to position 239, and an E at the position corresponding to position 332.

In some embodiments the VH4-34 antigen-binding molecule comprises an Fc region comprising (e.g. comprising one more polypeptides comprising a heavy chain constant region, a CH2-CH3 region, or a CH2 region, comprising) a C at the position corresponding to position 242, a C at the position corresponding to position 334, an A at the position corresponding to position 236, a D at the position corresponding to position 239, an E at the position corresponding to position 332, and an L at the position corresponding to position 330.

In some embodiments the VH4-34 antigen-binding molecule comprises an Fc region comprising (e.g. comprising one more polypeptides comprising a heavy chain constant region, or a CH2-CH3 region, comprising) a C at the position corresponding to position 242, a C at the position corresponding to position 334, a K at the position corresponding to position 345, and a G at the position corresponding to position 430.

In some embodiments the VH4-34 antigen-binding molecule comprises an Fc region comprising (e.g. comprising one more polypeptides comprising a heavy chain constant region, a CH2-CH3 region, or a CH2 region, comprising) the substitution L242C (or an equivalent substitution). In some embodiments the Fc region comprises (e.g. comprises one more polypeptides comprising a heavy chain constant region, a CH2-CH3 region, or a CH2 region, comprising) the substitution K334C (or an equivalent substitution). In some embodiments the Fc region comprises (e.g. comprises one more polypeptides comprising a heavy chain constant region, a CH2-CH3 region, or a CH2 region, comprising) the substitution L242C (or an equivalent substitution) and the substitution K334C (or an equivalent substitution).

In some embodiments the VH4-34 antigen-binding molecule comprises an Fc region comprising (e.g. comprising one more polypeptides comprising a heavy chain constant region, a CH2-CH3 region, or a CH2 region, comprising) the substitution G236A (or an equivalent substitution). In some embodiments the Fc region comprises (e.g. comprises one more polypeptides comprising a heavy chain constant region, a CH2-CH3 region, or a CH2 region, comprising) the substitution S239D (or an equivalent substitution). In some embodiments the Fc region comprises (e.g. comprises one more polypeptides comprising a heavy chain constant region, a CH2-CH3 region, or a CH2 region, comprising) the substitution G236A (or an equivalent substitution), and the substitution S239D (or an equivalent substitution).

In some embodiments the VH4-34 antigen-binding molecule comprises an Fc region comprising (e.g. comprising one more polypeptides comprising a heavy chain constant region, a CH2-CH3 region, or a CH2 region, comprising) the substitution I332E (or an equivalent substitution). In some embodiments the Fc region comprises (e.g. comprises one more polypeptides comprising a heavy chain constant region, a CH2-CH3 region, or a CH2 region, comprising) the substitution G236A (or an equivalent substitution), the substitution S239D (or an equivalent substitution), and the substitution I332E (or an equivalent substitution).

In some embodiments the VH4-34 antigen-binding molecule comprises an Fc region comprising (e.g. comprising one more polypeptides comprising a heavy chain constant region, a CH2-CH3 region, or a CH2 region, comprising) the substitution A330L (or an equivalent substitution). In some embodiments the Fc region comprises (e.g. comprises one more polypeptides comprising a heavy chain constant region, a CH2-CH3 region, or a CH2 region, comprising) the substitution G236A (or an equivalent substitution), the substitution S239D (or an equivalent substitution), the substitution I332E (or an equivalent substitution), and the substitution A330L (or an equivalent substitution).

In some embodiments the VH4-34 antigen-binding molecule comprises an Fc region comprising (e.g. comprising one more polypeptides comprising a heavy chain constant region, a CH2-CH3 region, or a CH3 region, comprising) the substitution E345K (or an equivalent substitution). In some embodiments the Fc region comprises (e.g. comprises one more polypeptides comprising a heavy chain constant region, a CH2-CH3 region, or a CH3 region, comprising) the substitution E430G (or an equivalent substitution). In some embodiments the Fc region comprises (e.g. comprises one more polypeptides comprising a heavy chain constant region, a CH2-CH3 region, or a CH2 region, comprising) the substitution E345K (or an equivalent substitution), and the substitution E430G (or an equivalent substitution).

In some embodiments the VH4-34 antigen-binding molecule comprises an Fc region comprising (e.g. comprising one more polypeptides comprising a heavy chain constant region, a CH2-CH3 region, or a CH2 region, comprising) the substitution L242C (or an equivalent substitution), the substitution K334C (or an equivalent substitution), the substitution G236A (or an equivalent substitution), and the substitution S239D (or an equivalent substitution).

In some embodiments the VH4-34 antigen-binding molecule comprises an Fc region comprising (e.g. comprising one more polypeptides comprising a heavy chain constant region, a CH2-CH3 region, or a CH2 region, comprising) the substitution L242C (or an equivalent substitution), the substitution K334C (or an equivalent substitution), the substitution G236A (or an equivalent substitution), the substitution S239D (or an equivalent substitution), and the substitution I332E (or an equivalent substitution).

In some embodiments the VH4-34 antigen-binding molecule comprises an Fc region comprising (e.g. comprising one more polypeptides comprising a heavy chain constant region, a CH2-CH3 region, or a CH2 region, comprising) the substitution L242C (or an equivalent substitution), the substitution K334C (or an equivalent substitution), the substitution G236A (or an equivalent substitution), the substitution S239D (or an equivalent substitution), the substitution 1³32E (or an equivalent substitution), and the substitution A330L (or an equivalent substitution).

In some embodiments the VH4-34 antigen-binding molecule comprises an Fc region comprising (e.g. comprising one more polypeptides comprising a heavy chain constant region, or a CH2-CH3 region, comprising) the substitution L242C (or an equivalent substitution), the substitution K334C (or an equivalent substitution), the substitution E345K (or an equivalent substitution), and the substitution E430G (or an equivalent substitution).

In some embodiments the VH4-34 antigen-binding molecule comprises an Fc region comprising (e.g. comprising one more polypeptides comprising a heavy chain constant region, or a CH2-CH3 region, comprising) one or more (e.g. 1, 2, 3, 4, 5, 6, 7, 8, 9, 10, 11 or 12) of the following: L at the position corresponding to position 243, P at the position corresponding to position 292, L at the position corresponding to position 300, I at the position corresponding to position 305 and L at the position corresponding to position 396; D at the position corresponding to position 239 and E at the position corresponding to position 332; D at the position corresponding to position 239, E at the position corresponding to position 332 and L at the position corresponding to position 330: A at the position corresponding to position 298. A at the position corresponding to position 333 and A at the position corresponding to position 334; Y at the position corresponding to position 234, Q at the position corresponding to position 235, W at the position corresponding to position 236, M at the position corresponding to position 239, D at the position corresponding to position 268, E at the position corresponding to position 270 and A at the position corresponding to position 298; E at the position corresponding to position 270, D at the position corresponding to position 326, M at the position corresponding to position 330 and E at the position corresponding to position 334; A at the position corresponding to position 236, D at the position corresponding to position 239 and E at the position corresponding to position 332; W at the position corresponding to position 326 and S at the position corresponding to position 333: E at the position corresponding to position 267, F at the position corresponding to position 268 and T at the position corresponding to position 324; R at the position corresponding to position 345, G at the position corresponding to position 430 and Y at the position corresponding to position 440; Y at the position corresponding to position 252, T at the position corresponding to position 254 and E at the position corresponding to position 256; and L at the position corresponding to position 428 and S at the position corresponding to position 434.

In some embodiments the VH4-34 antigen-binding molecule comprises an Fc region comprising (e.g. comprising one more polypeptides comprising a heavy chain constant region, or a CH2-CH3 region, comprising) one or more (e.g. 1, 2, 3, 4, 5, 6, 7, 8, 9, 10, 11 or 12) of the following combinations of substitutions (or corresponding substitutions): F243L/R292P/Y300L/V3051/P396L; S239D/I332E; S239D/I332E/A330L; S298A/E333A/K334A; L234Y/L235Q/G236W/S239M/H268D/D270E/S298A; D270E/K326D/A330M/K334E; G236A/S239D/I332E; K326W/E333S; S267E/H268F/S324T; E345R/E430G/S440Y; M252Y/S254T/T256E; and M428L/N434S.

In some embodiments, the VH4-34 antigen-binding molecule of the present disclosure comprises an Fc region comprising modification in one or more of the CH2 and CH3 regions promoting association of the Fc region. Recombinant co-expression of constituent polypeptides of an antigen-binding molecule and subsequent association leads to several possible combinations. To improve the yield of the desired combinations of polypeptides in antigen-binding molecules in recombinant production, it is advantageous to introduce in the Fc regions modification(s) promoting association of the desired combination of heavy chain polypeptides. Modifications may promote e.g. hydrophobic and/or electrostatic interaction between CH2 and/or CH3 regions of different polypeptide chains. Suitable modifications are described e.g. in Ha et al., Front. Immnol (2016) 7:394, which is hereby incorporated by reference in its entirety.

In some embodiments the antigen antigen-binding molecule of the present disclosure comprises an Fc region comprising paired substitutions in the CH3 regions of the Fc region according to one of the following formats, as shown in Table 1 of Ha et al., Front. Immnol (2016) 7:394: KiH, KiHₛ₋ₛ, HA-TF, ZW1, 7.8.60, DD-KK, EW-RVT, EW-RVTₛ₋ₛ, SEED or A107.

### Polypeptides

The present disclosure also provides polypeptide constituents of antigen-binding molecules. The polypeptides may be provided in isolated or substantially purified form.

The VH4-34 antigen-binding molecule of the present disclosure may be, or may comprise, a complex of polypeptides.

In the present specification where a polypeptide comprises more than one domain or region, it will be appreciated that the plural domains/regions are preferably present in the same polypeptide chain. That is, the polypeptide comprising more than one domain or region is a fusion polypeptide comprising the domains/regions.

In some embodiments a polypeptide comprises, or consists of, a VH as described herein. In some embodiments a polypeptide comprises, or consists of, a VL as described herein.

In some embodiments, the polypeptide additionally comprises one or more antibody heavy chain constant regions (CH). In some embodiments, the polypeptide additionally comprises one or more antibody light chain constant regions (CL). In some embodiments, the polypeptide comprises a CH1, CH2 region and/or a CH3 region of an immunoglobulin (Ig).

In some embodiments the polypeptide comprises one or more regions of an immunoglobulin heavy chain constant sequence. In some embodiments the polypeptide comprises a CH1 region as described herein. In some embodiments the polypeptide comprises a CH1-CH2 hinge region as described herein. In some embodiments the polypeptide comprises a CH2 region as described herein. In some embodiments the polypeptide comprises a CH3 region as described herein.

In some embodiments the polypeptide comprises one or more regions of an immunoglobulin light chain constant sequence. In some embodiments the polypeptide comprises a CL region as described herein.

In some embodiments, the polypeptide comprises a structure from N- to C-terminus according to one of the following:

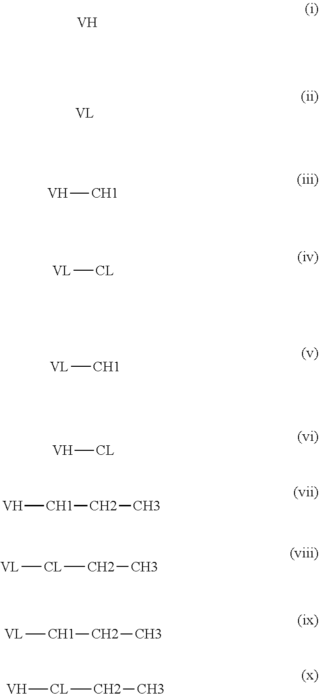

Also provided by the present disclosure are antigen-binding molecules composed of the polypeptides of the present disclosure. In some embodiments, the VH4-34 antigen-binding molecule of the present disclosure comprises one of the following combinations of polypeptides:

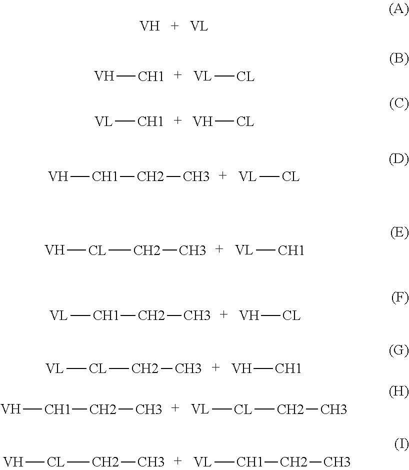

In some embodiments the VH4-34 antigen-binding molecule comprises more than polypeptide of the combinations shown in (A) to (I) above. By way of example, with reference to (D) above, in some embodiments the VH4-34 antigen-binding molecule comprises two polypeptides comprising the structure VH-CH1-CH2-CH3, and two polypeptides comprising the structure VL-CL.

In some embodiments, the VH4-34 antigen-binding molecule of the present disclosure comprises one of the following combinations of polypeptides:

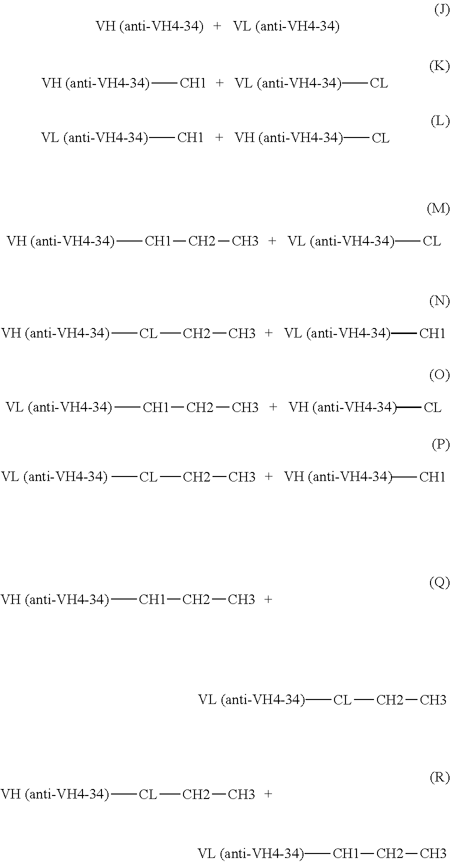

Wherein: “VH(anti-VH4-34)” refers to the VH of an antigen-binding molecule capable of binding to VH4-34 as described herein, e.g as defined in one of (1) to (31), and “VL(anti-VH4-34)” refers to the VL of an antigen-binding molecule capable of binding to VH4-34 as described herein, e.g. as defined in one of (32) to (66).

In some embodiments, the VH4-34 antigen-binding molecule of the present disclosure comprises a polypeptide which comprises or consists of an amino acid sequence having at least 70%, preferably one of 80%, 85%, 90%, 91%, 92%, 93%, 94%, 95%, 96%, 97%, 98%, 99% or 100% amino acid sequence identity to any one of SEQ ID NOs: 5, 21, 37, 53, 69, 70, 125, 141, 151, 156, 167, 171, 176, 181, 182, or 184.

In some embodiments, the VH4-34 antigen-binding molecule of the present disclosure comprises a polypeptide which comprises or consists of an amino acid sequence having at least 70%, preferably one of 80%, 85%, 90%, 91%, 92%, 93%, 94%, 95%, 96%, 97%, 98%, 99% or 100% amino acid sequence identity to any one of SEQ ID NOs: 13, 29, 45, 61, 130, 143, 147, 153, 161, 189, 193, 198, 201, 205, or 209.

In some embodiments, the VH4-34 antigen-binding molecule of the present disclosure comprises a polypeptide which comprises or consists of an amino acid sequence having at least 70%, preferably one of 80%, 85%, 90%, 91%, 92%, 93%, 94%, 95%, 96%, 97%, 98%, 99% or 100% amino acid sequence identity to any one of SEQ ID NOs: 86 to 89, 136, 139, or 214 to 219.

In some embodiments, the VH4-34 antigen-binding molecule of the present disclosure comprises a polypeptide which comprises or consists of an amino acid sequence having at least 70%, preferably one of 80%, 85%, 90%, 91%, 92%, 93%, 94%, 95%, 96%, 97%, 98%, 99% or 100% amino acid sequence identity to any one of SEQ ID NOs: 90 to 93, 137, 140, 146, or 220 to 225.

In some embodiments, the VH4-34 antigen-binding molecule of the present disclosure comprises a polypeptide which comprises or consists of an amino acid sequence having at least 70%, preferably one of 80%, 85%, 90%, 91%, 92%, 93%, 94%, 95%, 96%, 97%, 98%, 99% or 100% amino acid sequence identity to any one of SEQ ID NOs: 95 to 99, or 138.

In some embodiments, the VH4-34 antigen-binding molecule of the present disclosure comprises a polypeptide which comprises or consists of an amino acid sequence having at least 70%, preferably one of 80%, 85%, 90%, 91%, 92%, 93%, 94%, 95%, 96%, 97%, 98%, 99% or 100% amino acid sequence identity to any one of SEQ ID NOs: 101 to 105, 149 or 150.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 156, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 13 or 130.
   In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 5 or 125, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 161.
   In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 151, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 61, 143 or 147.
   In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 53 or 141, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 153.

### Particular Exemplary Embodiments of the VH4-34 Antigen-Binding Molecules

In some embodiments, the VH4-34 antigen-binding molecule of the present disclosure comprises a polypeptide or polypeptides comprising a VH region comprising the heavy chain CDRs, and a VL region comprising the light chain CDRs, of a clone selected from those shown in Table A herein. That is, in some embodiments, the VH4-34 antigen-binding molecule comprises a polypeptide or polypeptides comprising: (i) a VH region comprising a HC-CDR1, a HC-CDR2 and a HC-CDR3 as indicated in column A of Table A, and (ii) a VL region comprising a LC-CDR1, a LC-CDR2 and a LC-CDR3 as indicated in column B of Table A, wherein the sequences of columns A and B are selected from the same row of Table A.

In some embodiments, the VH4-34 antigen-binding molecule of the present disclosure comprises a polypeptide or polypeptides comprising a VH region comprising the heavy chain FRs, and a VL region comprising the light chain FRs, of a clone selected from those shown in Table B herein. That is, in some embodiments, the VH4-34 antigen-binding molecule comprises a polypeptide or polypeptides comprising: (i) a VH region comprising a HC-FR1, a HC-FR2, a HC-FR3 and a HC-FR4 as indicated in column A of Table B. and (ii) a VL region comprising a LC-FR1, a LC-FR2, a LC-FR3, and a LC-FR4 as indicated in column B of Table B, wherein the sequences of columns A and B are selected from the same row of Table B.

In some embodiments, the VH4-34 antigen-binding molecule of the present disclosure comprises a polypeptide or polypeptides comprising: (i) an amino acid sequence having at least 70%, preferably one of 80%, 85%, 90%, 91%, 92%, 93%, 94%, 95%, 96%, 97%, 98%, 99% or 100% amino acid sequence identity to an amino acid sequence indicated in column A of Table C, and (ii) an amino acid sequence having at least 70%, preferably one of 80%, 85%, 90%, 91%, 92%, 93%, 94%, 95%, 96%, 97%, 98%, 99% or 100% amino acid sequence identity to an amino acid sequence indicated in column B of Table C, wherein the sequences of columns A and B are selected from the same row of Table C.

In some embodiments, the VH4-34 antigen-binding molecule of the present disclosure comprises a polypeptide or polypeptides comprising a VH region and a VL region of a clone selected from those shown in Table C herein. That is, in some embodiments, the VH4-34 antigen-binding molecule comprises a polypeptide or polypeptides comprising: (i) an amino acid sequence indicated in column A of Table C, and (ii) an amino acid sequence indicated in column B of Table C, wherein the sequences of columns A and B are selected from the same row of Table C.

In some embodiments, the VH4-34 antigen-binding molecule of the present disclosure comprises: (i) a polypeptide comprising or consisting of an amino acid sequence having at least 70%, preferably one of 80%, 85%, 90%, 91%, 92%, 93%, 94%, 95%, 96%, 97%, 98%, 99% or 100% amino acid sequence identity to an amino acid sequence indicated in column A of Table D, and (ii) a polypeptide comprising or consisting of an amino acid sequence having at least 70%, preferably one of 80%, 85%, 90%, 91%, 92%, 93%, 94%, 95%, 96%, 97%, 98%, 99% or 100% amino acid sequence identity to an amino acid sequence indicated in column B of Table D, wherein the sequences of columns A and B are selected from the same row of Table D.

In some embodiments, the VH4-34 antigen-binding molecule of the present disclosure comprises the polypeptides of an antigen-binding molecule according to Table D herein. That is, in some embodiments, the VH4-34 antigen-binding molecule comprises: (i) a polypeptide comprising or consisting of an amino acid sequence indicated in column A of Table D, and (ii) a polypeptide comprising or consisting of an amino acid sequence indicated in column B of Table D, wherein the sequences of columns A and B are selected from the same row of Table D.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 86, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 90.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 87, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 91.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 88, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 92.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 89, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 93.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 136, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 137.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 139, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 140.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 139, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 146.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 95, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 90.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 96, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 91.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 97, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 92.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 98, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 93.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 99, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 137.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 138, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 140.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 139, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 146.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 228, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 90, 137, or 229.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 86, 136 or 228, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 229.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 226, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 93, 140, 146 or 227.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 89, 139 or 226, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 227.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 214, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 220, 221, 222, 223, 224, or 225.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 215, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 220, 221, 222, 223, 224, or 225.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 216, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 220, 221, 222, 223, 224, or 225.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 217, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 220, 221, 222, 223, 224, or 225.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 218, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 220, 221, 222, 223, 224, or 225.

In some embodiments, the VH4-34 antigen-binding molecule of the present disclosure comprises:
(i) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 219, and
(ii) one or more (e.g. 1, 2) polypeptide(s) comprising, or consisting of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO: 220, 221, 222, 223, 224, or 225.

It will be appreciated that in some embodiments, an antigen-binding molecule may comprise a polypeptide comprising polypeptides according to (i) and (ii) defined in accordance with the preceding paragraphs. For example, in embodiments wherein the VH4-34 antigen-binding molecule comprises or consists of a single-chain Fv, polypeptides according to (i) and (ii) are provided in tandem in the same polypeptide, e.g. joined by a linker sequence.

### Linkers and Additional Sequences

In some embodiments the VH4-34 antigen-binding molecules and polypeptides of the present disclosure comprise one or more linker sequences between amino acid sequences. A linker sequence may be provided at one or both ends of one or more of a VH. VL, CH1-CH2 hinge region. CH2 region and a CH3 region of the VH4-34 antigen-binding molecule/polypeptide.

Linker sequences are known to the skilled person, and are described, for example in Chen et al., Adv Drug Deliv Rev (2013) 65(10): 1357-1369, which is hereby incorporated by reference in its entirety. In some embodiments, a linker sequence is a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues.

In some embodiments, the linker sequence comprises at least one glycine residue and/or at least one serine residue. In some embodiments the linker sequence consists of glycine and serine residues. In some embodiments, the linker sequence comprises one or more copies (e.g. in tandem) of the sequence motif GaS. In some embodiments, the linker sequence has a length of 1-2, 1-3, 1-4, 1-5, 1-10, 1-15, 1-20, 1-25, or 1-30 amino acids.

The VH4-34 antigen-binding molecules and polypeptides of the present disclosure may additionally comprise further amino acids or sequences of amino acids. For example, the VH4-34 antigen-binding molecules and polypeptides may comprise amino acid sequence(s) to facilitate expression, folding, trafficking, processing, purification or detection of the VH4-34 antigen-binding molecule/polypeptide. For example, the VH4-34 antigen-binding molecule/polypeptide may comprise a sequence encoding a His, (e.g. 6×His (SEQ ID NO: 236)), Myc, GST, MBP, FLAG, HA, E, or Biotin tag, optionally at the N- or C-terminus of the VH4-34 antigen-binding molecule/polypeptide. In some embodiments the VH4-34 antigen-binding molecule/polypeptide comprises a detectable moiety, e.g. a fluorescent, luminescent, immuno-detectable, radio, chemical, nucleic acid or enzymatic label.

The VH4-34 antigen-binding molecules and polypeptides of the present disclosure may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides.

The signal peptide may be present at the N-terminus of the VH4-34 antigen-binding molecule/polypeptide, and may be present in the newly synthesized antigen-binding molecule/polypeptide. The signal peptide provides for efficient trafficking and secretion of the VH4-34 antigen-binding molecule/polypeptide. Signal peptides are often removed by cleavage, and thus are not comprised in the mature antigen-binding molecule/polypeptide secreted from the cell expressing the VH4-34 antigen-binding molecule/polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8: 785-786) or Signal-BLAST (Frank and Sippl, 2008 Bioinformatics 24: 2172-2176).

### Labels and Conjugates

In some embodiments the VH4-34 antigen-binding molecules of the present disclosure additionally comprise a detectable moiety.

In some embodiments the VH4-34 antigen-binding molecule comprises a detectable moiety, e.g. a fluorescent label, phosphorescent label, luminescent label, immuno-detectable label (e.g. an epitope tag), radiolabel, chemical, nucleic acid or enzymatic label. The VH4-34 antigen-binding molecule may be covalently or non-covalently labelled with the detectable moiety.

Fluorescent labels include e.g. fluorescein, rhodamine, allophycocyanin, eosine and NDB, green fluorescent protein (GFP), chelates of rare earths such as europium (Eu), terbium (Tb) and samarium (Sm), tetramethyl rhodamine, Texas Red, 4-methyl umbelliferone, 7-amino-4-methyl coumarin, Cy3, and Cy5. Radiolabels include radioisotopes such as Iodine¹²³, Iodine¹²⁵, Iodine¹²⁶, Iodine¹³¹, Iodine¹³³, Bromine⁷⁷, Technetium^{99m}, Indium¹¹¹, Indium^{113m}, Gallium⁶⁷, Gallium⁶⁸, Ruthenium⁹⁵, Ruthenium⁹⁷, Ruthenium¹⁰³, Ruthenium¹⁰⁵, Mercury²⁰⁷, Mercury²⁰³, Rhenium^{99m}, Rhenium¹⁰¹, Rhenium¹⁰⁵. Scandium⁴⁷, Tellurium^{121m}, Tellurium^{122m}, Tellurium^{122m}, Thulium¹⁶⁵, Thuliuml¹⁶⁷, Thulium¹⁶⁸, Copper⁶⁷, Fluorine¹⁸, Yttrium⁹⁰. Palladium¹⁰⁰, Bismuth²¹⁷ and Antimony²¹¹. Luminescent labels include as radioluminescent, chemiluminescent (e.g. acridinium ester, luminol, isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers. Enzymatic labels include e.g. peroxidase, alkaline phosphatase, glucose oxidase, beta-galactosidase and luciferase.

In some embodiments the VH4-34 antigen-binding molecules of the present disclosure are conjugated to a chemical moiety. The chemical moiety may be a moiety for providing a therapeutic effect. The VH4-34 antigen-binding molecule may be provided as an antibody-drug conjugate. Antibody-drug conjugates are reviewed e.g. in Parslow et al., Biomedicines. 2016 September; 4(3):14 (hereby incorporated by reference in its entirety). In some embodiments, the chemical moiety is a drug moiety (e.g. a cytotoxic moiety). In some embodiments, the drug moiety is a chemotherapeutic agent.

### Functional Properties of the VH4-34 Antigen-Binding Molecules

The VH4-34 antigen-binding molecules described herein may be characterised by reference to certain functional properties. In some embodiments, the VH4-34 antigen-binding molecule described herein possesses one or more of the following properties:
binds to VH4-34 and/or a peptide/polypeptide/polypeptide complex (e.g. an antigen-binding molecule) comprising VH4-34:
binds to cells expressing VH4-34 and/or a peptide/polypeptide/polypeptide complex (e.g. an antigen-binding molecule) comprising VH4-34;
inhibits interaction between VH4-34 and/or a peptide/polypeptide/polypeptide complex (e.g. an antigen-binding molecule) comprising VH4-34 and an interaction partner for VH4-34 (e.g. I/i carbohydrate, a glycoprotein bearing N-acetyl lactosamine (e.g. CD45 isoform B220), a lactosamine moiety and/or antibody 9G4);
reduces the number/proportion of cells (e.g. B cells) expressing VH4-34 and/or a peptide/polypeptide/polypeptide complex (e.g. an antigen-binding molecule) comprising VH4-34;
increases cell killing of cells (e.g. B cells) expressing VH4-34 and/or a peptide/polypeptide/polypeptide complex (e.g. an antigen-binding molecule) comprising VH4-34;
increases cell killing of cells expressing VH4-34 and/or a peptide/polypeptide/polypeptide complex (e.g. an antigen-binding molecule) comprising VH4-34 via antibody dependent cell-mediated cvtotoxicity (ADCC);
increases cell killing of cells expressing VH4-34 and/or a peptide/polypeptide/polypeptide complex (e.g. an antigen-binding molecule) comprising VH4-34 via antibody dependent cell-mediated phagocytosis (ADCP);
reduces the level of VH4-34 and/or peptide/polypeptide/polypeptide complex (e.g. an antigen-binding molecule) comprising VH4-34 in the blood/serum/plasma; and/or
reduces the number/proportion of cells expressing VH4-34 and/or a peptide/polypeptide/polypeptide complex (e.g. an antigen-binding molecule) comprising VH4-34 in the blood/serum/plasma.

It will be appreciated that a given antigen-binding molecule may display more than one of the properties recited in the preceding paragraph. A given antigen-binding molecule may be evaluated for the properties recited in the preceding paragraph using suitable assays. The assays may be e.g. in vitro assays, which may be cell-free or cell-based assays. Alternatively, the assays may be e.g. in vivo assays, i.e. performed in non-human animals.

Where assays are cell-based assays, they may comprise contacting cells with a given antigen-binding molecule in order to determine whether the VH4-34 antigen-binding molecule displays one or more of the recited properties. Assays may employ species labelled with detectable entities in order to facilitate their detection. Assays may comprise evaluating the recited properties following treatment of cells separately with a range of quantities/concentrations of antigen-binding molecule (e.g. a dilution series). It will be appreciated that the cells are preferably cells that express VH4-34, e.g. B cells expressing an antigen-binding molecule comprising VH4-34.

Analysis of the results of such assays may comprise determining the concentration at which 50% of the maximal level of the relevant activity is attained. The concentration of antigen-binding molecule at which 50% of the maximal level of the relevant activity is attained may be referred to as the ‘half-maximal effective concentration’ of the VH4-34 antigen-binding molecule in relation to the relevant activity, which may also be referred to as the ‘EC₅₀’. By way of illustration, the EC₅₀ of a given antigen-binding molecule for binding to VH4-34 may be the concentration at which 50% of the maximal level of binding to the relevant species is achieved.

Depending on the property, the EC₅₀ may also be referred to as the ‘half-maximal inhibitory concentration’ or ‘IC₅₀’, this being the concentration of antigen-binding molecule at which 50% of the maximal level of inhibition of a given property is observed. By way of illustration, the IC₅₀ of a given antigen-binding molecule for inhibiting interaction between VH4-34 and a glycoprotein bearing N-acetyl lactosamine (e.g. CD45 isoform B220) may be the concentration at which 50% of the maximal level of inhibition is achieved.

The VH4-34 antigen-binding molecules described herein bind to VH4-34. In some embodiments, the VH4-34 antigen-binding molecules display specific binding to VH4-34. As used herein, “specific binding” refers to binding which is selective for the antigen, and which can be discriminated from non-specific binding to non-target antigen. An antigen-binding molecule that specifically binds to VH4-34 preferably binds to VH4-34 with greater affinity, and/or with greater duration than it binds to other, non-target molecules.

The ability of a given polypeptide to bind specifically to a given molecule can be determined by analysis according to methods known in the art, such as by ELISA, Surface Plasmon Resonance (SPR; see e.g. Hearty et al., Methods Mol Biol (2012) 907:411-442), Bio-Layer Interferometry (see e.g. Lad et al., (2015) J Biomol Screen 20(4): 498-507), flow cytometry, or by a radiolabelled antigen-binding assay (RIA) enzyme-linked immunosorbent assay. Through such analysis binding to a given molecule can be measured and quantified. In some embodiments, the binding is the response detected in a given assay.

In some embodiments, the extent of binding of the VH4-34 antigen-binding molecule to a non-target molecule is less than about 10% of the binding of the antibody to the target molecule as measured, e.g. by ELISA, SPR, Bio-Layer Interferometry or by RIA. Alternatively, binding specificity may be reflected in terms of binding affinity where the VH4-34 antigen-binding molecule binds with a dissociation constant (K_{D}) that is at least 0.1 order of magnitude (i.e. 0.1×10ⁿ, where n is an integer representing the order of magnitude) greater than the K_{D} of the VH4-34 antigen-binding molecule towards a non-target molecule. This may optionally be one of at least 0.2, 0.3, 0.4, 0.5, 0.6, 0.7, 0.8, 0.9, 1.0, 1.5, or 2.0.

Binding to VH4-34 may be determined by ELISA, e.g. as described in the Examples of the present disclosure.

In some embodiments, an antigen-binding molecule binds to VH4-34. In some embodiments, an antigen-binding molecule binds to a peptide or polypeptide comprising VH4-34. In some embodiments, an antigen-binding molecule binds to an immunoglobulin heavy chain variable region (i.e. a VH) comprising VH4-34. In some embodiments, an antigen-binding molecule binds to a peptide/polypeptide comprising a VH comprising VH4-34. In some embodiments, an antigen-binding molecule binds to an immunoglobulin heavy chain comprising VH4-34. In some embodiments, an antigen-binding molecule binds to the VH of zanolimumab, patritumab and/or tabalumab. In some embodiments, an antigen-binding molecule binds to the heavy chain of zanolimumab, patritumab and/or tabalumab.

In some embodiments, an antigen-binding molecule binds to a peptide or polypeptide complex comprising VH4-34. In some embodiments, an antigen-binding molecule binds to a peptide or polypeptide complex comprising a peptide or polypeptide comprising VH4-34. In some embodiments, an antigen-binding molecule binds to a peptide or polypeptide complex comprising a peptide/polypeptide comprising a VH comprising VH4-34. In some embodiments, an antigen-binding molecule binds to an immunoglobulin comprising an immunoglobulin heavy chain comprising VH4-34. In some embodiments, an antigen-binding molecule binds to an immunoglobulin formed of immunoglobulin heavy and light chains, wherein the VH4-34 antigen-binding molecule comprises an immunoglobulin heavy chain comprising VH4-34. In some embodiments, an antigen-binding molecule binds to zanolimumab, patritumab and/or tabalumab.

Zanolimumab comprises a heavy chain of SEQ ID NO: 106 and a light chain of SEQ ID NO: 109. Patritumab comprises a heavy chain of SEQ ID NO: 107 and a light chain of SEQ ID NO: 110. Tabalumab comprises a heavy chain of SEQ ID NO: 108 and a light chain of SEQ ID NO: 111.

In some embodiments, an antigen-binding molecule binds to VH4-34 with an affinity in the micromolar range. i.e. K_{D}=9.9×10⁻⁴ to 1×10⁻⁶ M. In some embodiments, an antigen-binding molecule binds to VH4-34 with sub-micromolar affinity, i.e. K_{D}<1×10⁻⁶ M. In some embodiments, an antigen-binding molecule binds to VH4-34 with an affinity in the nanomolar range, i.e. K_{D}=9.9×10⁻⁷ to 1×10⁻⁹ M. In some embodiments, an antigen-binding molecule binds to VH4-34 with sub-nanomolar affinity. i.e. K_{D}<1×10⁻⁹ M. In some embodiments, an antigen-binding molecule binds to VH4-34 with an affinity in the picomolar range, i.e. K_{D}=9.9×10⁻¹⁰ to 1×10⁻¹² M. In some embodiments, an antigen-binding molecule binds to VH4-34 with sub-picomolar affinity, i.e. K_{D}<1×10⁻¹² M.

In some embodiments, an antigen-binding molecule binds to VH4-34 with a K_{D} of 10 μM or less, preferably one of ≤5 pM, ≤2 pM, ≤1 pM, ≤500 nM, ≤100 nM, ≤75 nM, ≤50 nM, ≤40 nM, ≤30 nM, 20 nM≤15 nM, ≤12.5 nM, ≤10 nM, ≤9 nM, ≤8 nM, ≤7 nM, ≤6 nM, ≤5 nM, ≤4 nM≤3 nM, ≤2 nM, ≤1 nM, ≤500 pM, ≤400 pM, ≤300 pM, ≤200 pM, ≤100 pM, ≤50 pM, ≤40 pM, ≤30 pM, ≤20 pM, ≤10 pM or ≤1 pM.

In some embodiments, an antigen-binding molecule binds to VH4-34 with an EC₅₀ (e.g. as determined by ELISA, e.g. an ELISA as described in the Examples of the present disclosure) of 0.1 μg/ml or less preferably one of ≤0.05 μg/ml, ≤0.04 μg/ml, ≤0.03 μg/ml, ≤0.02 μg/ml, ≤0.01 μg/ml, ≤0.009 μg/ml, ≤0.008 μg/ml, ≤0.007 μg/ml, ≤0.006 μg/ml, ≤0.005 μg/ml, ≤0.004 μg/ml, ≤0.003 μg/ml, ≤0.002 μg/ml or ≤0.001 μg/ml.

The VH4-34 antigen-binding molecules of the present disclosure may bind to a particular region of interest of VH4-34. The antigen-binding region of an antigen-binding molecule may bind to linear epitope of VH4-34, consisting of a contiguous sequence of amino acids (i.e. an amino acid primary sequence). In some embodiments, the antigen-binding region of an antigen-binding molecule may bind to a conformational epitope of VH4-34, consisting of a discontinuous sequence of amino acids of the amino acid sequence.

The region of a peptide/polypeptide to which an antigen-binding molecule binds can be determined by the skilled person using various methods well known in the art, including X-ray co-crystallography analysis of antibody-antigen complexes, peptide scanning, mutagenesis mapping, hydrogen-deuterium exchange analysis by mass spectrometry, phage display, competition ELISA and proteolysis-based ‘protection’ methods. Such methods are described, for example, in Gershoni et al., BioDrugs, 2007, 21(3):145-156, which is hereby incorporated by reference in its entirety.

In some embodiments, an antigen-binding molecule binds to the region of VH4-34 corresponding to the region shown in SEQ ID NO: 3. In some embodiments, an antigen-binding molecule binds to a polypeptide comprising or consisting of the amino acid sequence shown in SEQ ID NO: 3.

In some embodiments, an antigen-binding molecule binds to the region of VH4-34 corresponding to the region shown in SEQ ID NO: 4. In some embodiments, the epitope of an antigen-binding molecule comprises one or more amino acids of the region shown in SEQ ID NO: 4. In some embodiments, an antigen-binding molecule binds to VH4-34 via contact with one or more amino acids of the region shown in SEQ ID NO: 4. In some embodiments, an antigen-binding molecule binds to a polypeptide comprising or consisting of the amino acid sequence shown in SEQ ID NO: 4.

In some embodiments, the VH4-34 antigen-binding molecule may inhibit the interaction between VH4-34 and an antigen, such as molecule comprising an N-acetyl lactosamine moiety.

In some embodiments, an antigen-binding molecule binds to the region of VH4-34 through which VH4-34 binds to I/i carbohydrate. In some embodiments, an antigen-binding molecule binds to the region of VH4-34 through which VH4-34 binds to glycoproteins bearing N-acetyl lactosamine. In some embodiments, an antigen-binding molecule to the region of VH4-34 through which VH4-34 binds to a lactosamine moiety.

In some embodiments, the epitope of the VH4-34 antigen-binding molecule comprises one or more of (e.g. 1, 2, 3, 4 or all of); Q at the position corresponding to position 32, W at the position corresponding to position 33, A at the position corresponding to position 49. V at the position corresponding to position 50 and Y at the position corresponding to position 51 of SEQ ID NO: 1. In some embodiments, an antigen-binding molecule binds to VH4-34 via contact with one or more of (e.g. 1, 2, 3, 4 or all of); Q at the position corresponding to position 32, W at the position corresponding to position 33, A at the position corresponding to position 49. V at the position corresponding to position 50 and Y at the position corresponding to position 51 of SEQ ID NO: 1.

In some embodiments, the VH4-34 antigen-binding molecule binds to a polypeptide comprising or consisting of the amino acid sequence shown in SEQ ID NO: 3 with greater affinity than the affinity with which an antigen-binding molecule binds to a polypeptide comprising or consisting of the amino acid sequence shown in SEQ ID NO: 112. In some embodiments, the level of binding of an antigen-binding molecule to a polypeptide comprising or consisting of the amino acid sequence shown in SEQ ID NO: 112 is less than 50% of the level of binding to a polypeptide comprising or consisting of the amino acid sequence shown in SEQ ID NO: 3. In some embodiments, an antigen-binding molecule displays substantially no binding to a polypeptide comprising or consisting of the amino acid sequence shown in SEQ ID NO: 112.

In some embodiments, an antigen-binding molecule of the present disclosure binds to an epitope of VH4-34 which is non-identical to the epitope of VH4-34 bound by anti-VH4-34 antibody 9G4 (described e.g. in Stevenson, et al. Blood (1986) 68: 430, which is hereby incorporated by reference in its entirety).

The ability of an antigen-binding molecule to bind to a given peptide/polypeptide can be analysed by methods well known to the skilled person, including analysis by ELISA, immunoblot (e.g. western blot), immunoprecipitation, surface plasmon resonance and biolayer interferometry.

In some embodiments an antigen-binding molecule binds to VH4-34 in a region which is accessible to an antigen-binding molecule (i.e., an extracellular antigen-binding molecule) when VH4-34 (e.g. present in a peptide/polypeptide or polypeptide complex comprising VH4-34 comprising VH4-34) is expressed at the cell surface (i.e. in or at the cell membrane) of a cell expressing VH4-34 or a peptide/polypeptide or polypeptide complex comprising VH4-34. In some embodiments an antigen-binding molecule binds to cells expressing VH4-34 or a peptide/polypeptide or polypeptide complex comprising VH4-34. Cells expressing VH4-34 or a peptide/polypeptide or polypeptide complex comprising VH4-34 include e.g. B cell lineage cells encoding/expressing immunoglobulins comprising a VH comprising VH4-34. Cells expressing VH4-34 or a peptide/polypeptide or polypeptide complex comprising VH4-34 may be referred to herein as VH4-34-positive (i.e. VH4-34+) cells.

The ability of an antigen-binding molecule to bind to a given cell type can be analysed by contacting cells with the VH4-34 antigen-binding molecule, and detecting antigen-binding molecule bound to the cells, e.g. after a washing step to remove unbound antigen-binding molecule. The ability of an antigen-binding molecule to bind to immune cell surface molecule-expressing cells and/or cancer cell antigen-expressing cells can be analysed by methods such as flow cytometry and immunofluorescence microscopy.

In some embodiments, an antigen-binding molecule binds to the same region of VH4-34, or an overlapping region of VH4-34, to the region of VH4-34 which is bound by an antigen-binding molecule comprising the VH and VL regions of one of clones DOI-1E11p, D011-6039. D011-6040. D011-7016, D011-1E11-5T2, D011-7016-LD1, D011-7016-LD2, D011-7016-LD2_H0L0, D011-7016-LD2_H1L1, D011-7016-LD2_H1L2, D011-7016-LD2_H1L3, D011-7016-LD2_H1L4, D011-7016-LD2_H3L1, D011-7016-LD2_H3L2, D011-7016-LD2_H3L3, D011-7016-LD2_H3L4, D011-7016-LD2_H4L1, D011-7016-LD2_H4L2, D011-7016-LD2_H4L3, D011-7016-LD2_H4L4, D011-7016-LD2_H5L1, D011-7016-LD2_H5L2, D011-7016-LD2_H5L3, and D011-7016-LD2_H5L4 described herein.

Whether a test antigen-binding molecule binds to the same or an overlapping region of a given target as a reference antigen-binding molecule can be evaluated, for example, by analysis of (i) interaction between the test antigen-binding molecule and the target in the absence of the reference binding molecule, and (ii) interaction between the test antigen-binding molecule in the presence of the reference antigen-binding molecule, or following incubation of the target with the reference antigen-binding molecule. Determination of a reduced level of interaction between the test antigen-binding molecule and the target following analysis according to (ii) as compared to (i) might support an inference that the test and reference antigen-binding molecule bind to the same or an overlapping region of the target. Suitable assays for such analysis include e.g. competition ELISA assays and epitope binning assays.

In some embodiments, an antigen-binding molecule binds to VH4-34 in the region which is bound by an interaction partner for VH4-34 (e.g. I/i carbohydrate, a glycoprotein bearing N-acetyl lactosamine (e.g. CD45 isoform B220), a lactosamine moiety and/or antibody 9G4). In some embodiments, the VH4-34 antigen-binding molecule inhibits interaction between an interaction partner for VH4-34 (e.g. I/i carbohydrate, a glycoprotein bearing N-acetyl lactosamine (e.g. CD45 isoform B220), a lactosamine moiety and/or antibody 9G4) and VH4-34. In some embodiments, the VH4-34 antigen-binding molecule is a competitive inhibitor of binding of an interaction partner for VH4-34 (e.g. I/i carbohydrate, a glycoprotein bearing N-acetyl lactosamine (e.g. CD45 isoform B220), a lactosamine moiety and/or antibody 9G4) to VH4-34. In some embodiments, the VH4-34 antigen-binding molecule is an allosteric inhibitor of binding of an interaction partner for VH4-34 (e.g. I/i carbohydrate, a glycoprotein bearing N-acetyl lactosamine (e.g. CD45 isoform B220), a lactosamine moiety and/or antibody 9G4) to VH4-34. In some embodiments, the VH4-34 antigen-binding molecule displaces an interaction partner for VH4-34 (e.g. I/i carbohydrate, a glycoprotein bearing N-acetyl lactosamine (e.g. CD45 isoform B220), a lactosamine moiety and/or antibody 9G4) from a complex comprising VH4-34 and the interaction partner for VH4-34. In some embodiments, the VH4-34 antigen-binding molecule does not bind to a complex comprising VH4-34 and an interaction partner for VH4-34 (e.g. I/i carbohydrate, a glycoprotein bearing N-acetyl lactosamine (e.g. CD45 isoform B220), a lactosamine moiety and/or antibody 9G4).

The ability of a given antigen-binding molecule to inhibit interaction between two species can be determined for example by analysis of interaction in the presence of, or following incubation of one or both of the interaction partners with, the given antigen-binding molecule. An example of a suitable assay to determine whether a given antigen-binding molecule is capable of inhibiting interaction between two interaction partners is a competition ELISA assay. An antigen-binding molecule which is capable of inhibiting a given interaction (e.g. between VH4-34 and an interaction partner for VH4-34) is identified by the observation of a reduction/decrease in the level of interaction between the interaction partners in the presence of—or following incubation of one or both of the interaction partners with—the VH4-34 antigen-binding molecule, as compared to the level of interaction in the absence of the VH4-34 antigen-binding molecule (or in the presence of an appropriate control antigen-binding molecule known not to inhibit the relevant interaction). Suitable analysis can be performed in vitro, e.g. using recombinant interaction partners or using cells expressing the interaction partners. Cells expressing an interaction partner may do so endogenously, or may do so from nucleic acid introduced into the cell. For the purposes of such assays, one or both of the interaction partners and/or the VH4-34 antigen-binding molecule may be labelled or used in conjunction with a detectable entity for the purposes of detecting and/or measuring the level of interaction.

In some embodiments, an antigen-binding molecule inhibits interaction between VH4-34 and/or a peptide/polypeptide/polypeptide complex (e.g. an antigen-binding molecule) comprising VH4-34 and an interaction partner for VH4-34 (e.g. I/i carbohydrate, a glycoprotein bearing N-acetvl lactosamine (e.g. CD45 isoform B220), a lactosamine moiety and/or antibody 9G4) to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of interaction observed in the absence of the VH4-34 antigen-binding molecule, or in the presence of the same quantity of an appropriate control antigen-binding molecule, in a given assay.

In some embodiments, an antigen-binding molecule potentiates (i.e. upregulates, enhances) cell killing of cells encoding/comprising/expressing VH4-34 and/or a peptide/polypeptide/polypeptide complex (e.g. an antigen-binding molecule) comprising VH4-34.

In some embodiments an antigen-binding molecule is capable of reducing the number/proportion of cells encoding/comprising/expressing VH4-34 and/or a peptide/polypeptide/polypeptide complex (e.g an antigen-binding molecule) comprising VH4-34. In some embodiments, an antigen-binding molecule is capable of depleting/enhancing depletion of such cells. Such cells may be B cell lineage cells.

In some embodiments, an antigen-binding molecule reduces the number/proportion of cells encoding/comprising/expressing VH4-34 and/or a peptide/polypeptide/polypeptide complex (e.g. an antigen-binding molecule) comprising VH4-34 to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the number/proportion of such cells observed in the absence of the VH4-34 antigen-binding molecule, or in the presence of the same quantity of an appropriate control antigen-binding molecule, in a given assay.

Antigen-binding molecules may comprise one or more moieties for potentiating a reduction in the number/proportion of cells encoding/comprising/expressing VH4-34 and/or a peptide/polypeptide/polypeptide complex (e.g. an antigen-binding molecule) comprising VH4-34. For example, an antigen-binding molecule may e.g. comprise an Fc region and/or a drug moiety.

Fc regions provide for interaction with Fc receptors and other molecules of the immune system to bring about functional effects. IgG Fc-mediated effector functions are reviewed e.g. in Jefferis et al., Immunol Rev 1998 163:59-76 (hereby incorporated by reference in its entirety), and are brought about through Fc-mediated recruitment and activation of immune cells (e.g. macrophages, dendritic cells, neutrophils, basophils, eosinophils, platelets, mast cells, NK cells and T cells) through interaction between the Fc region and Fc receptors expressed by the immune cells, recruitment of complement pathway components through binding of the Fc region to complement protein C1q, and consequent activation of the complement cascade. Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

In some embodiments, an antigen-binding molecule comprises an Fc region capable of potentiating/directing one or more of ADCC, ADCP, CDC against, and/or potentiating formation of a MAC on or cell degranulation of, a cell encoding/comprising/expressing VH4-34 and/or a peptide/polypeptide/polypeptide complex (e.g. an antigen-binding molecule) comprising VH4-34 (e.g. a cell encoding/comprising/expressing VH4-34 and/or a peptide/polypeptide/polypeptide complex (e.g. an antigen-binding molecule) at the cell surface).

In some embodiments, an antigen-binding molecule is capable of potentiating/directing ADCC against a cell encoding/comprising/expressing VH4-34 and/or a peptide/polypeptide/polypeptide complex (e.g. an antigen-binding molecule) comprising VH4-34.

In some embodiments, an antigen-binding molecule is capable of potentiating/directing ADCP against a cell encoding/comprising/expressing VH4-34 and/or a peptide/polypeptide/polypeptide complex (e.g. an antigen-binding molecule) comprising VH4-34.

The ability of an antigen-binding molecule to potentiate/direct ADCC and/or ADCP can be assessed, for example, by incubating target cells with the VH4-34 antigen-binding molecule and Jurkat-Lucia™ NFAT cells (InvivoGen) or macrophages. Suitable methods are described herein.

In some embodiments, an antigen-binding molecule comprises a drug moiety. The VH4-34 antigen-binding molecule may be conjugated to the drug moiety. Antibody-drug conjugates are reviewed e.g. in Parslow et al., Biomedicines. 2016 September; 4(3):14 (hereby incorporated by reference in its entirety). In some embodiments, the drug moiety is or comprises a cytotoxic agent, such that the VH4-34 antigen-binding molecule displays cytotoxicity to a cell encoding/comprising/expressing VH4-34 and/or a peptide/polypeptide/polypeptide complex (e.g. an antigen-binding molecule) comprising VH4-34 (e.g. a cell encoding/comprising/expressing VH4-34 and/or a peptide/polypeptide/polypeptide complex (e.g. an antigen-binding molecule) at the cell surface). In some embodiments the drug moiety is or comprises a chemotherapeutic agent.

In some embodiments, an antigen-binding molecule comprises an immune cell-engaging moiety. In some embodiments, the VH4-34 antigen-binding molecule comprises a CD3 polypeptide-binding moiety (e.g. an antigen-binding domain capable of binding to a CD3 polypeptide).

In some embodiments, an antigen-binding molecule is capable of potentiating/directing T cell-mediated cytolytic activity against a cell encoding/comprising/expressing VH4-34 and/or a peptide/polypeptide/polypeptide complex (e.g. an antigen-binding molecule) comprising VH4-34.

In some embodiments, an antigen-binding molecule reduces the number/proportion of cells encoding/comprising/expressing VH4-34 and/or a peptide/polypeptide/polypeptide complex (e.g. an antigen-binding molecule) comprising VH4-34 to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the number/proportion of such cells observed in the absence of the VH4-34 antigen-binding molecule, or in the presence of the same quantity of an appropriate control antigen-binding molecule, in a given assay.

In some embodiments, an antigen-binding molecule increases the level of killing of cells encoding/comprising/expressing VH4-34 and/or a peptide/polypeptide/polypeptide complex (e.g. an antigen-binding molecule) comprising VH4-34 to greater than 1 times, e.g. ≥21.5 times, ≥2 times, ≥3 times, ≥4 times, ≥5 times, ≥6 times, ≥7 times, ≥8 times, ≥9 times, ≥10 times, ≥15 times, ≥20 times, ≥30 times, ≥40 times or ≥50 times the level of killing of such cells observed in the absence of the VH4-34 antigen-binding molecule, or in the presence of the same quantity of an appropriate control antigen-binding molecule, in a given assay.

In some embodiments, an antigen-binding molecule is capable of reducing/decreasing the level of VH4-34 and/or a peptide/polypeptide/polypeptide complex (e.g. an antigen-binding molecule) comprising VH4-34 (or cells expressing such articles) in a subject to which the VH4-34 antigen-binding molecule is administered. In some embodiments, an antigen-binding molecule is capable of reducing/decreasing the level of VH4-34 and/or a peptide/polypeptide/polypeptide complex (e.g. an antigen-binding molecule) comprising VH4-34 (or cells expressing such articles) in the blood/plasma/serum, e.g. in a subject to which the VH4-34 antigen-binding molecule is administered.

In some embodiments, an antigen-binding molecule reduces the serum half-life of VH4-34 and/or a peptide/polypeptide/polypeptide complex (e.g. an antigen-binding molecule) comprising VH4-34 (or cells expressing such articles). In some embodiments, an antigen-binding molecule enhances/accelerates clearance/elimination of VH4-34 and/or a peptide/polypeptide/polypeptide complex (e.g. an antigen-binding molecule) comprising VH4-34 (or cells expressing such articles).

In some embodiments, an antigen-binding molecule reduces the level of VH4-34 and/or a peptide/polypeptide/polypeptide complex (e.g. an antigen-binding molecule) comprising VH4-34 (or cells expressing such articles) in the blood/plasma/serum of a subject to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the VH4-34 antigen-binding molecule, or following administration of the same quantity of an appropriate control antigen-binding molecule.

### Chimeric Antigen Receptors (CARs)

The present disclosure also provides Chimeric Antigen Receptors (CARs) comprising the antigen-binding polypeptides or polypeptides of the present disclosure.

CARs are recombinant receptors that provide both antigen-binding and T cell activating functions. CAR structure and engineering is reviewed, for example, in Dotti et al., Immunol Rev (2014) 257(1), hereby incorporated by reference in its entirety. CARs comprise an antigen-binding region linked to a cell membrane anchor region and a signalling region. An optional hinge region may provide separation between the antigen-binding region and cell membrane anchor region, and may act as a flexible linker.

The CAR of the present disclosure comprises an antigen-binding region which comprises or consists of the VH4-34 antigen-binding molecule of the present disclosure, or which comprises or consists of a polypeptide according to the present disclosure.

The cell membrane anchor region is provided between the antigen-binding region and the signalling region of the CAR and provides for anchoring the CAR to the cell membrane of a cell expressing a CAR, with the antigen-binding region in the extracellular space, and signalling region inside the cell. In some embodiments, the CAR comprises a cell membrane anchor region comprising or consisting of an amino acid sequence which comprises, consists of, or is derived from, the transmembrane region amino acid sequence for one of CD3-ζ, CD4, CD8 or CD28. As used herein, a region which is ‘derived from’ a reference amino acid sequence comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the reference sequence.

The signalling region of a CAR allows for activation of the T cell. The CAR signalling regions may comprise the amino acid sequence of the intracellular domain of CD3−, which provides immunoreceptor tyrosine-based activation motifs (ITAMs) for phosphorylation and activation of the CAR-expressing T cell. Signalling regions comprising sequences of other ITAM-containing proteins such as FcγRI have also been employed in CARs (Haynes et al., 2001 J Immunol 166(1):182-187). Signalling regions of CARs may also comprise co-stimulatory sequences derived from the signalling region of co-stimulatory molecules, to facilitate activation of CAR-expressing T cells upon binding to the target protein. Suitable co-stimulatory molecules include CD28, OX40, 4-1BB, ICOS and CD27. In some cases, CARs are engineered to provide for co-stimulation of different intracellular signalling pathways. For example, signalling associated with CD28 co-stimulation preferentially activates the phosphatidylinositol 3-kinase (PI3K) pathway, whereas the 4-1BB-mediated signalling is through TNF receptor associated factor (TRAF) adaptor proteins. Signalling regions of CARs therefore sometimes contain co-stimulatory sequences derived from signalling regions of more than one co-stimulatory molecule. In some embodiments, the CAR of the present disclosure comprises one or more co-stimulatory sequences comprising or consisting of an amino acid sequence which comprises, consists of, or is derived from, the amino acid sequence of the intracellular domain of one or more of CD28, OX40, 4-1BB, ICOS and CD27.

An optional hinge region may provide separation between the antigen-binding domain and the transmembrane domain, and may act as a flexible linker. Hinge regions may be derived from IgG1. In some embodiments, the CAR of the present disclosure comprises a hinge region comprising or consisting of an amino acid sequence which comprises, consists of, or is derived from, the amino acid sequence of the hinge region of IgG1.

Also provided is a cell comprising a CAR according to the present disclosure. The CAR may be used to generate CAR-expressing immune cells, e.g. CAR-T or CAR-NK cells. Engineering of CARs into immune cells may be performed during culture, in vitro.

The antigen-binding region of the CAR of the present disclosure may be provided with any suitable format, e.g. scFv, scFab, etc.

### Nucleic Acids and Vectors

The present disclosure provides a nucleic acid, or a plurality of nucleic acids, encoding an antigen-binding molecule, polypeptide or CAR according to the present disclosure. In some embodiments the nucleic acid(s) comprise or consist of DNA and/or RNA.

The present disclosure also provides a vector, or plurality of vectors, comprising the nucleic acid or plurality of nucleic acids according to the present disclosure.

Nucleic acids and vectors may be provided in purified or isolated form, i.e. from other nucleic acid, or naturally-occurring biological material.

The nucleotide sequence may be contained in a vector, e.g. an expression vector. A “vector” as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector according to the present disclosure.

The term “operably linked” may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. The resulting transcript(s) may then be translated into a desired peptide(s)/polypeptide(s).

Suitable vectors include plasmids, binary vectors. DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes).

In some embodiments, the vector is a eukaryotic vector, e.g. a vector comprising the elements necessary for expression of protein from the vector in a eukaryotic cell. In some embodiments, the vector is a mammalian vector, e.g. comprising a cytomegalovirus (CMV) or SV40 promoter to drive protein expression.

Constituent polypeptides of an antigen-binding molecule may be encoded by different nucleic acids of the plurality of nucleic acids, or by different vectors of the plurality of vectors.

### Cells Encoding/Comprising/Expressing the VH4-34 Antigen-Binding Molecules and Polypeptides

The present disclosure also provides a cell comprising or expressing an antigen-binding molecule, polypeptide or CAR according to the present disclosure. Also provided is a cell comprising or expressing a nucleic acid, a plurality of nucleic acids, a vector or a plurality of vectors according to the present disclosure.

The cell may be a eukaryotic cell. e.g. a mammalian cell. The mammal may be a primate (rhesus, cynomolgus, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell is, or is derived from, a cell type commonly used for the expression of polypeptides for use in therapy in humans. Exemplary cells are described e.g. in Kunert and Reinhart, Appl Microbiol Biotechnol. (2016) 100:3451-3461 (hereby incorporated by reference in its entirety), and include e.g. CHO, HEK 293, PER.C6, NSO and BHK cells. In some embodiments, the cell is, or is derived from, a CHO cell.

The present disclosure also provides a method for producing a cell comprising a nucleic acid(s) or vector(s), comprising introducing a nucleic acid, a plurality of nucleic acids, a vector or a plurality of vectors into a cell. In some embodiments, introducing an isolated nucleic acid(s) or vector(s) into a cell comprises transformation, transfection, electroporation or transduction (e.g. retroviral transduction).

The present disclosure also provides a method for producing a cell expressing/comprising an antigen-binding molecule, polypeptide or CAR according to the present disclosure, comprising introducing a nucleic acid, a plurality of nucleic acids, a vector or a plurality of vectors in a cell. In some embodiments, the methods additionally comprise culturing the cell under conditions suitable for expression of the nucleic acid(s) or vector(s) by the cell. In some embodiments, the methods are performed in vitro.

The present disclosure also provides cells obtained or obtainable by the methods according to the present disclosure.

### Producing the VH4-34 Antigen-Binding Molecules and Polypeptides

Antigen-binding molecules and polypeptides may be prepared according to methods for the production of polypeptides known to the skilled person.

Polypeptides may be prepared by chemical synthesis, e.g. liquid or solid phase synthesis. For example, peptides/polypeptides can be synthesized using the methods described in, for example, Chandrudu et al., Molecules (2013), 18: 4373-4388, which is hereby incorporated by reference in its entirety.

Alternatively, antigen-binding molecules and polypeptides may be produced by recombinant expression. Molecular biology techniques suitable for recombinant production of polypeptides are well known in the art, such as those set out in Green and Sambrook, Molecular Cloning: A Laboratory Manual (4th Edition), Cold Spring Harbor Press, 2012, and in Nat Methods. (2008): 5(2): 135-146 both of which are hereby incorporated by reference in their entirety. Methods for the recombinant production of antigen-binding molecules are also described in Frenzel et al., Front Immunol. (2013); 4: 217 and Kunert and Reinhart, Appl Microbiol Biotechnol. (2016) 100: 3451-3461, both of which are hereby incorporated by reference in their entirety.

In some cases, the VH4-34 antigen-binding molecules of the present disclosure are comprised of more than one polypeptide chain. In such cases, production of the VH4-34 antigen-binding molecules may comprise transcription and translation of more than one polypeptide, and subsequent association of the polypeptide chains to form the VH4-34 antigen-binding molecule.

For recombinant production according to the present disclosure, any cell suitable for the expression of polypeptides may be used. The cell may be a prokaryote or eukaryote. In some embodiments the cell is a prokaryotic cell, such as a cell of archaea or bacteria. In some embodiments the bacteria may be Gram-negative bacteria such as bacteria of the family Enterobacteriaceae, for example Escherichia coli. In some embodiments, the cell is a eukaryotic cell such as a yeast cell, a plant cell, insect cell or a mammalian cell, e.g. a cell described hereinabove.

In some cases, the cell is not a prokaryotic cell because some prokaryotic cells do not allow for the same folding or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

In some embodiments, polypeptides are prepared by cell-free-protein synthesis (CFPS), e.g. according to a system described in Zemella et al. Chembiochem (2015) 16(17): 2420-2431, which is hereby incorporated by reference in its entirety.

Production may involve culture or fermentation of a eukaryotic cell modified to express the polypeptide(s) of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polvpeptide(s). Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the VH4-34 antigen-binding molecule/polypeptide(s), the polypeptide(s) of interest may be isolated. Any suitable method for separating proteins from cells known in the art may be used. In order to isolate the polypeptide, it may be necessary to separate the cells from nutrient medium. If the polypeptide(s) are secreted from the cells, the cells may be separated by centrifugation from the culture media that contains the secreted polypeptide(s) of interest. If the polypeptide(s) of interest collect within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polvpeptide(s) of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography. These may be used as an alternative to precipitation or may be performed subsequently to precipitation.

Once the polypeptide(s) of interest have been isolated from culture it may be desired or necessary to concentrate the polypeptide(s). A number of methods for concentrating proteins are known in the art, such as ultrafiltration or lyophilisation.

### Compositions

The present disclosure also provides compositions comprising the VH4-34 antigen-binding molecules, polypeptides. CARs, nucleic acids, expression vectors and cells described herein.

The VH4-34 antigen-binding molecules, polypeptides, CARs, nucleic acids, expression vectors and cells described herein may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion.

Suitable formulations may comprise the VH4-34 antigen-binding molecule in a sterile or isotonic medium. Medicaments and pharmaceutical compositions may be formulated in fluid, including gel, form. Fluid formulations may be formulated for administration by injection or infusion (e.g. via cannula) to a selected region of the human or animal body.

In some embodiments the composition is formulated for injection or infusion, e.g. into a blood vessel, tissue/organ of interest.

The present disclosure also provides methods for the production of pharmaceutically useful compositions, such methods of production may comprise one or more steps selected from: producing an antigen-binding molecule, polypeptide, CAR, nucleic acid (or plurality thereof), expression vector (or plurality thereof) or cell described herein; isolating an antigen-binding molecule, polypeptide, CAR, nucleic acid (or plurality thereof), expression vector (or plurality thereof) or cell described herein; and/or mixing an antigen-binding molecule, polypeptide, CAR, nucleic acid (or plurality thereof), expression vector (or plurality thereof) or cell described herein with a pharmaceutically acceptable carrier, adjuvant, excipient or diluent.

For example, a further aspect the present disclosure relates to a method of formulating or producing a medicament or pharmaceutical composition for use in the treatment of a disease or condition (e.g. a cancer), the method comprising formulating a pharmaceutical composition or medicament by mixing an antigen-binding molecule, polypeptide. CAR, nucleic acid (or plurality thereof), expression vector (or plurality thereof) or cell described herein with a pharmaceutically acceptable carrier, adjuvant, excipient or diluent.

### Therapeutic and Prophylactic Applications

The VH4-34 antigen-binding molecules, polypeptides, CARs, nucleic acids, expression vectors, cells and compositions described herein find use in therapeutic and prophylactic methods.

The present disclosure provides an antigen-binding molecule, polypeptide, CAR, nucleic acid (or plurality thereof), expression vector (or plurality thereof), cell or composition described herein for use in a method of medical treatment or prophylaxis. Also provided is the use of an antigen-binding molecule, polypeptide, CAR, nucleic acid (or plurality thereof), expression vector (or plurality thereof), cell or composition described herein in the manufacture of a medicament for treating or preventing a disease or condition. Also provided is a method of treating or preventing a disease or condition, comprising administering to a subject a therapeutically or prophylactically effective amount of an antigen-binding molecule, polypeptide, CAR, nucleic acid (or plurality thereof), expression vector (or plurality thereof), cell or composition described herein.

Therapeutic or prophylactic intervention in accordance with the present disclosure may be effective to reduce the development or progression of a disease or condition, alleviate the symptoms of a disease or condition or reduce the pathology of a disease or condition. The intervention may be effective to prevent progression of the disease or condition, e.g. to prevent worsening of, or to slow the rate of development of, the disease or condition. In some embodiments the intervention may lead to an improvement in the disease or condition, e.g. a reduction in the symptoms of the disease or condition or reduction in some other correlate of the severity/activity of the disease or condition. In some embodiments the intervention may prevent development of the disease or condition to a later stage (e.g. a more severe stage, or a chronic stage).

The terms ‘develop’, ‘developing’, and ‘development’, e.g. of a disorder, as used herein refer both to the onset of a disease as well as the progression, exacerbation or worsening of a disease state/correlate thereof.

It will be appreciated that the articles of the present disclosure may be used for the treatment/prevention of any disease or condition that would derive therapeutic or prophylactic benefit from a reduction in the level of VH4-34 and/or a peptide/polypeptide/polypeptide complex (e.g. an antigen-binding molecule) comprising VH4-34, and/or a reduction in the number/proportion and/or activity of cells encoding/comprising/expressing VH4-34 and/or a peptide/polypeptide/polypeptide complex (e.g. an antigen-binding molecule) comprising VH4-34.

Therapy and prophylaxis in accordance with the aspects and embodiments disclosed herein are concerned primarily with diseases/conditions characterised by expression of VH4-34, e.g. in the form of a peptide/polypeptide/polypeptide complex (e.g. an antigen-binding molecule) comprising VH4-34.

A disease or condition characterised by expression of VH4-34 may be associated with VH4-34 and/or a peptide/polypeptide/polypeptide complex (e.g. an antigen-binding molecule) comprising VH4-34, e.g. in the blood/serum/plasma. A disease or condition characterised by expression of VH4-34 may be associated with IgG and/or IgM comprising VH4-34, e.g. in the blood/serum/plasma.

A disease or condition characterised by expression of VH4-34 may be associated with cells encoding/comprising/expressing VH4-34 and/or a peptide/polypeptide/polypeptide complex (e.g. an antigen-binding molecule) comprising VH4-34. A disease or condition characterised by expression of VH4-34 may be associated with cells encoding/comprising/expressing IgG and/or IgM comprising VH4-34.

In some embodiments, the disease or condition is a disease or condition in a subject comprising VH4-34 and/or a peptide/polypeptide/polypeptide complex (e.g. an antigen-binding molecule) comprising VH4-34, e.g. in their blood/serum/plasma. In some embodiments, the disease or condition is a disease or condition in a subject comprising IgG and/or IgM comprising VH4-34, e.g. in their blood/serum/plasma.

In some embodiments, the disease or condition is a disease or condition in a subject comprising cells encoding/comprising/expressing VH4-34 and/or a peptide/polypeptide/polypeptide complex (e.g. an antigen-binding molecule) comprising VH4-34 e.g. in their blood. In some embodiments, the disease or condition is a disease or condition in a subject comprising cells encoding/comprising/expressing IgG and/or IgM comprising VH4-34, e.g. in their blood.

For example, the disease or condition may be a disease or condition in which VH4-34 and/or a peptide/polypeptide/polypeptide complex (e.g. an antigen-binding molecule) comprising VH4-34, and/or cells encoding/comprising/expressing VH4-34 and/or a peptide/polypeptide/polypeptide complex (e.g. an antigen-binding molecule) comprising VH4-34 are pathologically implicated.

The disease or condition may be a disease or condition in which VH4-34 and/or a peptide/polypeptide/polypeptide complex (e.g. an antigen-binding molecule) comprising VH4-34 is positively associated with the onset, development or progression of the disease or condition, and/or severity of one or more symptoms of the disease or condition, or for which VH4-34 and/or a peptide/polypeptide/polypeptide complex (e.g. an antigen-binding molecule) comprising VH4-34 is a risk factor for the onset, development or progression of the disease or condition.

The disease or condition may be a disease or condition in which cells encoding/comprising/expressing VH4-34 and/or cells encoding/comprising/expressing a peptide/polypeptide/polypeptide complex (e.g. an antigen-binding molecule) comprising VH4-34 are positively associated with the onset, development or progression of the disease or condition, and/or severity of one or more symptoms of the disease or condition, or for which such cells are a risk factor for the onset, development or progression of the disease or condition.

In some embodiments, the disease or condition to be treated/prevented is a disease or condition characterised by an increased level of VH4-34 and/or a peptide/polypeptide/polypeptide complex (e.g. an antigen-binding molecule) comprising VH4-34, in a subject or a sample obtained therefrom (e.g. a blood-derived sample), e.g. as compared to the level in the absence of the disease or condition. In some embodiments, the disease or condition is characterised by an increased number/proportion of cells encoding/comprising/expressing VH4-34 and/or cells encoding/comprising/expressing a peptide/polypeptide/polypeptide complex (e.g. an antigen-binding molecule) comprising VH4-34, in a subject or a sample (e.g. a blood-derived sample) obtained therefrom, e.g. as compared to the number/proportion of such cells in the absence of the disease or condition.

The increased level of VH4-34 and/or a peptide/polypeptide/polypeptide complex (e.g. an antigen-binding molecule) comprising VH4-34 and/or the increased number/proportion of cells encoding/comprising/expressing VH4-34 and/or cells encoding/comprising/expressing a peptide/polypeptide/polypeptide complex (e.g. an antigen-binding molecule) comprising VH4-34 in accordance with the preceding paragraph may be in tissue and/or an organ in which one or more symptoms of the disease or condition manifest.

Therapeutic or prophylactic intervention in accordance with the present disclosure may achieve a reduction in the level of VH4-34 and/or a peptide/polypeptide/polypeptide complex (e.g. an antigen-binding molecule) comprising VH4-34, e.g. in the blood/plasma/serum of the treated subject. In some embodiments, the intervention may achieve a reduction in the level of IgG and/or IgM comprising VH4-34, e.g. in the blood/plasma/serum of the treated subject. In some embodiments, the intervention may achieve a reduction in the level of IgG comprising VH4-34, e.g. in the blood/plasma/serum of the treated subject. In some embodiments, the intervention may achieve a reduction in the level of IgM comprising VH4-34, e.g. in the blood/plasma/serum of the treated subject.

Therapeutic or prophylactic intervention in accordance with the present disclosure may achieve a reduction in the number/proportion of cells (e.g. B cells) encoding/comprising/expressing VH4-34 and/or cells (e.g. B cells) encoding/comprising/expressing a peptide/polypeptide/polypeptide complex (e.g. an antigen-binding molecule) comprising VH4-34, e.g. in the treated subject (e.g. in the peripheral blood of the treated subject). In some embodiments, the intervention may achieve a reduction in the number/proportion of cells (e.g. B cells) encoding/comprising/expressing IgG and/or IgM comprising VH4-34 in the treated subject. In some embodiments, the intervention may achieve a reduction in the number/proportion of cells (e.g. B cells) encoding/comprising/expressing IgG comprising VH4-34 in the treated subject. In some embodiments, the intervention may achieve a reduction in the number/proportion of cells (e.g. B cells) encoding/comprising/expressing IgM comprising VH4-34 in the treated subject.

In some embodiments, the disease or condition to be treated/prevented is a disease or condition characterised by a reduced number/proportion of cells expressing the B220 glycoform of CD45, e.g. as compared to the number/proportion of such cells in the absence of the disease or condition. In some embodiments, the disease or condition is characterised by B cell lymphopenia. In some embodiments, the disease or condition is characterised by a reduced number of erythrocytes, and/or increased agglutination of erythrocytes, e.g. as compared to the level observed in the absence of the disease or condition. In some embodiments, the disease or condition is characterised by hemolysis and/or hemolytic anemia.

Diseases/conditions characterised by expression of VH4-34 (e.g. in the form of a peptide/polypeptide/polypeptide complex (e.g. an antigen-binding molecule) comprising VH4-34) include systemic lupus erythematosus (SLE), hairy cell leukemia (HCL), chronic lymphocytic leukemia (CLL), diffuse large B-cell lymphoma (DLBCL), cold agglutinin disease, and Wiskott-Aldrich syndrome.

In SLE, antibodies comprising VH4-34 are thought to behave as anti-lymphocyte autoantibodies (see e.g. Cappione et al., J Immunol. (2004) 172(7):4298-307). VH4-34 binds to N-acetyl lactosamine in the B220 glycoform of CD45 expressed by certain naïve B cells, potentiating their depletion. The proportion of IgG comprising VH4-34 as a fraction of total IgG in the serum of SLE patients has been shown to increase dramatically during SLE flares (Pugh-Bernard et al., J Clin Invest. (2001) 108(7):1061-70. Jenks et al., Arthritis Rheum. (2013) 65(12): 3165-3175). In some embodiments, the VH4-34 antigen-binding molecules of the present disclosure are capable of alleviating the symptoms of SLE, and can e.g. prevent/reduce the depletion of B cells. In some embodiments, the VH4-34 antigen-binding molecules of the present disclosure are capable of alleviating/preventing/reducing fatigue, skin rashes, fever, pain, swelling and/or inflammation associated with SLE.

In cold agglutinin disease (CAD), VH4-34 binds N-acetyl lactosamine in I/i carbohydrates expressed by erythrocytes, giving rise to their agglutination and destruction. VH4-34 antibodies are also implicated in the pathogenesis of Wiskott-Aldrich syndrome (see e.g. Kolhatkar et al., J Exp Med. (2015) 212(10):1663-1677). In some embodiments, the VH4-34 antigen-binding molecules of the present disclosure are capable of alleviating the symptoms of CAD, e.g. by reducing/preventing agglutination and/or destruction of erythrocytes. In some embodiments, the VH4-34 antigen-binding molecules of the present disclosure are capable of alleviating/preventing/reducing fatigue, dizziness, headaches, sweating, shortness of breath (dyspnea), fast heartbeat (tachycardia) and/or jaundice associated with CAD.

VH4-34+hairy cell leukemia (HCL) B cells have been shown to define a subtype of HCL known as hairy cell leukemia variant (HCL-V). HCL-V is characterised by particularly poor prognosis, with a median progression-free survival of just 5.1 months, compared with 22.8 months for classic HCL (Arons et al., Blood (2009) 114(21):4687-95). See also Xi et al., Blood (2012) 119(14): 3330-3332.

IGHV4-34 is the most frequently used IGHV gene in chronic lymphocytic leukemia (CLL) cases expressing B cell receptor immunoglobulin (BCR IG) with somatically hvpermutated IGHV genes (M-CLL: Xochelli et al., Clin Cancer Res. (2017) 23(17):5292-5301).

Approximately 30% of activated B cell diffuse large B-cell lymphoma (ABC-DLBCL) B cells encode VH4-34 BCRs, compared to ~10% of germinal center B cell diffuse large B-cell lymphoma (GBC-DLBCL), and fewer than 4% of normal B cells (Young et al., PNAS USA (2015) 112(44):13447-54).

Described herein, in certain embodiments, are methods of treating/preventing a disease or condition comprising administering a therapeutically effective amount of a VH4-34 antibody described herein. In some embodiments, the methods of treating/preventing a disease or condition comprise administering a therapeutically effective amount of a VH4-34 binding molecule that specifically binds to an epitope of VH4-34 comprising SEQ ID NO: 1 at any one of amino acid residues Q at the position corresponding to position 32, W at the position corresponding to position 33, A at the position corresponding to position 49, V at the position corresponding to position 50 and Y at the position corresponding to position 51 of SEQ ID NO: 1. In some embodiments, the methods of treating/preventing a disease or condition comprise administering a therapeutically effective amount of a VH4-34 binding molecule that specifically binds to an epitope of VH4-34 comprising SEQ ID NO: 1 at amino acid residues Q at the position corresponding to position 32, W at the position corresponding to position 33, A at the position corresponding to position 49, V at the position corresponding to position 50 and Y at the position corresponding to position 51 of SEQ ID NO: 1.

In some embodiments, the disease or condition to be treated/prevented is an autoimmune disease. In some embodiments, the autoimmune disease is selected from the group consisting of: autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD), and Wiskott-Aldrich syndrome. In some embodiments, the autoimmune disease is cold agglutinin disease (CAD). In some embodiments, the autoimmune disease is systemic lupus erythematosus (SLE).

In some embodiments, the disease or condition to be treated/prevented is cancer. In some embodiments, the cancer is a B cell malignancy. In some embodiments, the B cell malignancy is selected from the group consisting of: a B cell leukemia, hairy cell leukemia (HCL), hairy cell leukemia variant (HCL-V), chronic lymphocytic leukemia (CLL), mutated chronic lvmphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, and mantle cell lymphoma.

In some embodiments, the disease or condition characterised by expression of VH4-34 is selected from: a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, mantle cell lymphoma, autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD), and Wiskott-Aldrich syndrome.

In some embodiments, the disease or condition to be treated/prevented is selected from: a B cell malignancy, a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, mantle cell lymphoma, autoimmune hemolytic anemia (AIHA), common variable immunodeficiencv (CVID), rheumatoid arthritis, systemic lupus erythematosus (SLE), lupus nephritis, central nervous system (CNS) lupus, cold agglutinin disease and Wiskott-Aldrich syndrome. In some embodiments, the disease or condition is systemic lupus erythematosus (SLE), hairy cell leukemia (HCL), chronic lymphocytic leukemia (CLL) or diffuse large B-cell lymphoma (DLBCL).

Administration of the articles of the present disclosure is preferably in a “therapeutically effective” or “prophylactically effective” amount, this being sufficient to show therapeutic or prophylactic benefit to the subject. The actual amount administered, and rate and time-course of administration, will depend on the nature and severity of the disease or condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

In some embodiments, the articles of the present disclosure are administered following an exacerbation/worsening/progression of one or more symptoms of the disease or condition. That is, articles of the present disclosure may be administered following a flare in one or more symptoms of the disease or condition.

Administration of an article of the present disclosure may be alone or in combination with other treatments, either simultaneously or sequentially dependent upon the condition to be treated. Simultaneous administration refers to administration with another therapeutic agent together, for example as a pharmaceutical composition containing both agents (combined preparation), or immediately after each other and optionally via the same route of administration (e.g. to the same tissue, artery, vein or other blood vessel). Sequential administration refers to administration of one agent followed after a given time interval by separate administration of another agent. It is not required that the two agents are administered by the same route, although this is the case in some embodiments. The time interval may be any time interval.

In some embodiments, the articles are employed in combination with another therapy for a disease or condition described herein (e.g. a disease condition selected from systemic lupus erythematosus (SLE), hairy cell leukemia (HCL), chronic lymphocytic leukemia (CLL) and diffuse large B-cell lymphoma (DLBCL)).

In some embodiments, the articles are employed in combination with immunosuppressive therapy. In some embodiments, the immunosuppressive therapy comprises treatment with one or more agents selected from: hydroxychloroquine or chloroquine, prednisone, azathioprine and methotrexate.

In some embodiments, the articles are employed in combination with a B cell depleting agent. In some embodiments, a B cell depleting agent is an antibody to a surface antigen expressed by a B cell.

In some embodiments, the B cell depleting agent depletes BLyS-expressing cells. In some embodiments, the B cell depleting agent is a BLyS-targeting agent (e.g. a BLyS-targeting antibody, e.g. belimumab (CAS No. 356547-88-1)). In some embodiments, the B cell depleting agent depletes CD20-expressing cells. In some embodiments, the B cell depleting agent is a CD20-targeting antibody, e.g. rituximab (CAS No. 174722-31-7)). In some embodiments, the B cell depleting agent depletes CD38-expressing cells. In some embodiments, the B cell depleting agent is a CD38-targeting antibody, e.g. daratumumab (CAS No. 945721-28-8)).

Multiple doses of the VH4-34 antigen-binding molecule, polypeptide, CAR, nucleic acid (or plurality thereof), expression vector (or plurality thereof), cell or composition may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

In accordance with various aspects of the present disclosure, a method of treating and/or preventing a disease or condition may comprise one or more of the following: reducing the number/proportion of cells expressing VH4-34 and/or a peptide/polypeptide/polypeptide complex (e.g. an antigen-binding molecule) comprising VH4-34; increasing cell killing of cells expressing VH4-34 and/or a peptide/polypeptide/polypeptide complex (e.g. an antigen-binding molecule) comprising VH4-34; reducing the level of VH4-34 and/or peptide/polypeptide/polypeptide complex (e.g. an antigen-binding molecule) comprising VH4-34 in the blood/serum/plasma; reducing the number/proportion of cells (e.g. B cells) encoding/comprising/expressing IgG and/or IgM comprising VH4-34; reducing the level of IgG and/or IgM comprising VH4-34 in the blood/serum/plasma.

### Methods of Detection

The present disclosure also provides the articles of the present disclosure for use in methods for detecting, localizing or imaging VH4-34, peptides/polypeptides/polypeptide complexes (e.g. an antigen-binding molecules) comprising VH4-34, or cells encoding/comprising/expressing the same.

The VH4-34 antigen-binding molecules described herein may be used in methods that involve detecting binding of the VH4-34 antigen-binding molecule to VH4-34. Such methods may involve detection of the bound complex of the VH4-34 antigen-binding molecule and VH4-34.

As such, a method is provided, comprising contacting a sample containing, or suspected to contain. VH4-34 and/or a peptide/polypeptide/polypeptide complex (e.g. an antigen-binding molecule) comprising VH4-34, and detecting the formation of a complex of the VH4-34 antigen-binding molecule and the relevant species. Also provided is a method comprising contacting a sample containing, or suspected to contain, a cell encoding/comprising/expressing VH4-34 and/or a peptide/polypeptide/polypeptide complex (e.g. an antigen-binding molecule) comprising VH4-34, and detecting the formation of a complex of the VH4-34 antigen-binding molecule and such a cell.

Suitable method formats are well known in the art, including immunoassays such as sandwich assays, e.g. ELISA. The methods may involve labelling the VH4-34 antigen-binding molecule, or target(s), or both, with a detectable moiety, e.g. a fluorescent label, phosphorescent label, luminescent label, immuno-detectable label, radiolabel, chemical, nucleic acid or enzymatic label as described herein. Detection techniques are well known to those of skill in the art and can be selected to correspond with the labelling agent.

Methods comprising detecting VH4-34, peptides/polypeptides/polypeptide complexes (e.g. an antigen-binding molecules) comprising VH4-34, or cells encoding/comprising/expressing the same include methods for diagnosing/prognosing a disease or condition described herein.

Methods of this kind may be performed in vitro on a patient sample, or following processing of a patient sample. Once the sample is collected, the patient is not required to be present for the in vitro method to be performed, and therefore the method may be one which is not practised on the human or animal body. In some embodiments the method is performed in vivo.

Such methods may involve detecting or quantifying one or more of VH4-34, a peptide/polypeptide/polypeptide complex (e.g. an antigen-binding molecule) comprising VH4-34 and/or cells encoding/comprising/expressing VH4-34 or a peptide/polypeptide/polypeptide complex (e.g. an antigen-binding molecule) comprising VH4-34 e.g. in a patient sample. Where the method comprises quantifying the relevant factor, the method may further comprise comparing the determined amount against a standard or reference value as part of the diagnostic or prognostic evaluation. Other diagnostic/prognostic tests may be used in conjunction with those described herein to enhance the accuracy of the diagnosis or prognosis or to confirm a result obtained by using the tests described herein.

Detection in a sample may be used for the purpose of diagnosis of a disease or condition, predisposition to a disease or condition, or for providing a prognosis (prognosticating) for a disease or condition, e.g. a disease or condition described herein. The diagnosis or prognosis may relate to an existing (previously diagnosed) disease or condition.

A sample may be taken from any tissue or bodily fluid. The sample may comprise or may be derived from: a quantity of blood; a quantity of plasma, which may comprise the fluid portion of the blood obtained after removal of the blood cells: a quantity of serum, which may comprise the fluid portion of the blood obtained after removal of the fibrin clot and blood cells; a tissue sample or biopsy; pleural fluid; cerebrospinal fluid (CSF); or cells isolated from a subject. In some embodiments, the sample may be obtained or derived from a tissue or tissues which are affected by the disease or condition (e.g. tissue or tissues in which symptoms of the disease manifest, or which are involved in the pathogenesis of the disease or condition).

A subject may be selected for diagnostic/prognostic evaluation based on the presence of symptoms indicative of a disease or condition described herein, or based on the subject being considered to be at risk of developing a disease or condition described herein.

The present disclosure also provides methods for selecting/stratifying a subject for treatment with a VH4-34-targeted agent. In some embodiments a subject is selected for treatment/prevention in accordance with the methods of the present disclosure, or is identified as a subject which would benefit from such treatment/prevention, based on detection/quantification of VH4-34, peptides/polypeptides/polypeptide complexes (e.g. an antigen-binding molecules) comprising VH4-34, or cells encoding/comprising/expressing the same, e.g. in a sample obtained from the subject.

### Subjects

The subject in accordance with aspects of the present disclosure may be any animal or human. The subject is preferably mammalian, more preferably human. The subject may be a non-human mammal, but is more preferably human. The subject may be male or female. The subject may be a patient. A subject may have been diagnosed with a disease or condition requiring treatment (e.g. a disease or condition described herein such as systemic lupus erythematosus (SLE), hairy cell leukemia (HCL), chronic lymphocytic leukemia (CLL) or diffuse large B-cell lymphoma (DLBCL)), may be suspected of having such a disease or condition, or may be at risk of developing/contracting such a disease or condition.

In embodiments the subject is preferably a human subject. In some embodiments, the subject to be treated according to a therapeutic or prophylactic method of the present disclosure is a subject having, or at risk of developing, a disease described herein (e.g. a disease or condition described herein such as systemic lupus erythematosus (SLE), hairy cell leukemia (HCL), chronic lvmphocytic leukemia (CLL) or diffuse large B-cell lymphoma (DLBCL)). In embodiments according to the present disclosure, a subject may be selected for treatment according to the methods based on characterisation for certain markers of such disease or condition.

In some embodiments, the subject comprises VH4-34 and/or a peptide/polypeptide/polypeptide complex (e.g. an antigen-binding molecule) comprising VH4-34, e.g. in their blood/serum/plasma. In some embodiments, the subject comprises IgG and/or IgM comprising VH4-34, e.g. in their blood/serum/plasma.

In some embodiments, the subject comprises cells encoding/comprising/expressing VH4-34 and/or a peptide/polypeptide/polypeptide complex (e.g. an antigen-binding molecule) comprising VH4-34 e.g. in their blood. In some embodiments, the subject comprises cells encoding/comprising/expressing IgG and/or IgM comprising VH4-34, e.g. in their blood.

### Kits

In some aspects of the present disclosure a kit of parts is provided. In some embodiments, the kit may have at least one container having a predetermined quantity of an antigen-binding molecule, polypeptide, CAR, nucleic acid (or plurality thereof), expression vector (or plurality thereof), cell or composition described herein.

In some embodiments, the kit may comprise materials for producing an antigen-binding molecule, polypeptide. CAR, nucleic acid (or plurality thereof), expression vector (or plurality thereof), cell or composition described herein.

The kit may provide the VH4-34 antigen-binding molecule, polypeptide, CAR, nucleic acid (or plurality thereof), expression vector (or plurality thereof), cell or composition together with instructions for administration to a patient in order to treat a specified disease or condition.

In some embodiments, the kit may further comprise at least one container having a predetermined quantity of another therapeutic agent. In such embodiments, the kit may also comprise a second medicament or pharmaceutical composition such that the two medicaments or pharmaceutical compositions may be administered simultaneously or separately such that they provide a combined treatment for the specific disease or condition. The therapeutic agent may also be formulated so as to be suitable for injection or infusion to a tissue/organ, or to the blood.

### Sequence Identity

As used herein, “sequence identity” refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Soding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013. Molecular Biology and Evolution. 30(4) 772-780) software. When using such software, the default parameters, e.g. for gap penalty and extension penalty, are preferably used.

As used herein, an amino acid sequence, or a region of a polypeptide which ‘corresponds’ to a specified reference amino acid sequence or region of a polypeptide has at least 60%. e.g. one of at least 65%, 70%, 75%, 80%, 85%, 90%, 91%, 92%, 93%, 94%, 95%, 96%, 97%, 98%, 99% or 100% sequence identity to the amino acid sequence of the amino acid sequence/polypeptide/region. An amino acid sequence/region/position of a polypeptide/amino acid sequence which ‘corresponds’ to a specified reference amino acid sequence/region/position of a polypeptide/amino acid sequence can be identified by sequence alignment of the subject sequence to the reference sequence, e.g. using sequence alignment software such as ClustalOmega (Soding, J. 2005, Bioinformatics 21, 951-960).

Sequences

| SEQ | | |
|---|---|---|
| ID | | |
| NO: | DESCRIPTION | SEQUENCE |
| 1 | Human V4-34 | MDLLHKNMKHLWFFLLLVAAPRWVLSQVQLQQWGAGLLKPS |
| | (UniProtKB: P06331- | ETLSLTCAVYGGSFSGYYWSWIRQPPGKGLEWIGEINHSGSTNY |
| | 1, v2) | NPSLKSRVTISVDTSKNQFSLKLSSVTAADTAVYYCAR |
| 2 | Human V4-34 signal | MDLLHKNMKHLWFFLLLVAAPRWVLS |
| | peptide | |
| 3 | Human V4-34 mature | QVQLQQWGAGLLKPSETLSLTCAVYGGSFSGYYWSWIRQPPGK |
| | form | GLEWIGEINHSGSTNYNPSLKSRVTISVDTSKNQFSLKLSSVTAA |
| | | DTAVYYCAR |
| 4 | V4-34 FR1 | QVQLQQWGAGLLKPSETLSLTCAVY |
| 5 | D011-1E11p VH | EVQLQQSGPELVKPGASMKISCKASGYSFTGSTMNWVKQSPGK |
| | | NLEWIGLINPYNGGTSYNQKFEGKATLTVDKSSSTAYMELLSLT |
| | | SVDSAVYYCASRGKNGYDGRDFDCWGQGTSVTVSS |
| 6 | D011-1E11p; D011- | GYSFTGST |
| | 1E11-5T2 HC-CDR1 | |
| 7 | D011-1E11p; D011- | INPYNGGT |
| | 1E11-5T2 HC-CDR2 | |
| 8 | D011-1E11p; D011- | ASRGKNGYDGRDFDC |
| | 1E11-5T2 HC-CDR3 | |
| 9 | D011-1E11p HC-FR1 | EVQLQQSGPELVKPGASMKISCKAS |
| 10 | D011-1E11p HC-FR2 | MNWVKQSPGKNLEWIGL |
| 11 | DO11-1E11p HC-FR3 | SYNQKFEGKATLTVDKSSSTAYMELLSLTSVDSAVYYC |
| 12 | D011-1E11p HC-FR4 | WGQGTSVTVSS |
| 13 | D011-1E11p VL | DIVLTQSPLSLPVNIGDQASISCKSTKSLLNSGGFTYLDWYLQKP |
| | | GQSPQLLIYLVSNRFSGVPDRFSGSGSGTDFTLKISRVEAEDLGV |
| | | YYCFQSNYFPFTFGSGTKLEIK |
| 14 | D011-1E11p LC- | KSLLNSGGFTY |
| | CDR1 | |
| | D011-1E11p; D011- | LVS |
| | 1E11-5T2 LC-CDR2 | |
| 16 | D011-1E11p; D011- | FQSNYFPFT |
| | 1E11-5T2 LC-CDR3 | |
| 17 | D011-1E11p LC-FR1 | DIVLTQSPLSLPVNIGDQASISCKST |
| 18 | D011-1E11p LC-FR2 | LDWYLQKPGQSPQLLIY |
| 19 | D011-1E11p LC-FR3 | NRFSGVPDRFSGSGSGTDFTLKISRVEAEDLGVYYC |
| 20 | D011-1E11p LC-FR4 | FGSGTKLEIK |
| 21 | D011-6039 VH | EVMLVESGGGLVQPGGSLKLSCAASGFTFSSYGMSWVRQTPDK |
| | | RLELVATINSNGVNTYYPDSVKGRFTISRDNAKNTLYLQMSSLK |
| | | SEDTAMYYCARVPFDYWGQGTSLTVSS |
| 22 | D011-6039 HC-CDR1 | GFTFSSYG |
| 23 | D011-6039 HC-CDR2 | INSNGVNT |
| 24 | D011-6039 HC-CDR3 | ARVPFDY |
| 25 | D011-6039 HC-FR1 | EVMLVESGGGLVQPGGSLKLSCAAS |
| 26 | D011-6039 HC-FR2 | MSWVRQTPDKRLELVAT |
| 27 | D011-6039 HC-FR3 | YYPDSVKGRFTISRDNAKNTLYLQMSSLKSEDTAMYYC |
| 28 | D011-6039 HC-FR4 | WGQGTSLTVSS |
| 29 | D011-6039 VL | DIVMTQSPAIMSASLGEKVTMSCRASSSVDYIYWYQQKSDASP |
| | | KLWIYYTSTLAPGVPARFSGSGSGNSYSLTISSMEGEDAATYYC |
| | | QQFTSSPWTFGGGTRLEIK |
| 30 | D011-6039 LC-CDR1 | SSVDY |
| | D011-6039 LC-CDR2 | YTS |
| 32 | D011-6039 LC-CDR3 | QQFTSSPWT |
| 33 | D011-6039 LC-FR1 | DIVMTQSPAIMSASLGEKVTMSCRAS |
| 34 | D011-6039 LC-FR2 | IYWYQQKSDASPKLWIY |
| 35 | D011-6039 LC-FR3 | TLAPGVPARFSGSGSGNSYSLTISSMEGEDAATYYC |
| 36 | D011-6039 LC-FR4 | FGGGTRLEIK |
| 37 | D011-6040 VH | EVQLQQSGAEFVRSGASVKLSCTASGFNIKDYYMHWVKQRPE |
| | | QGLDWIGWIDPENGDADYDPKFQGKATMTADTSSNTAYLHLSS |
| | | LTSEDAAVYYCNACHYYGYEDAMDYWGQGTSVTVSS |
| 38 | D011-6040 HC-CDR1 | GFNIKDYY |
| 39 | D011-6040 HC-CDR2 | IDPENGDA |
| 40 | D011-6040 HC-CDR3 | NACHYYGYEDAMDY |
| 41 | D011-6040 HC-FR1 | EVQLQQSGAEFVRSGASVKLSCTAS |
| 42 | D011-6040 HC-FR2 | MHWVKQRPEQGLDWIGW |
| 43 | D011-6040 HC-FR3 | DYDPKFQGKATMTADTSSNTAYLHLSSLTSEDAAVYYC |
| 44 | D011-6040 HC-FR4 | WGQGTSVTVSS |
| 45 | D011-6040 VL | DIQMTQSPSSLSASLGERVSLTCRASQEISGYLSWLQQKPDGTIK |
| | | RLIYAASTLDSGVPKRFSGSRSGSDYSLTISSLESEDFADYYCLQ |
| | | YAGYPRTFGGGTRLEIK |
| 46 | D011-6040 LC-CDR1 | QEISGY |
| | D011-6040 LC-CDR2 | AAS |
| 48 | D011-6040 LC-CDR3 | LQYAGYPRT |
| 49 | D011-6040 LC-FR1 | DIQMTQSPSSLSASLGERVSLTCRAS |
| 50 | D011-6040 LC-FR2 | LSWLQQKPDGTIKRLIY |
| 51 | D011-6040 LC-FR3 | TLDSGVPKRFSGSRSGSDYSLTISSLESEDFADYYC |
| 52 | D011-6040 LC-FR4 | FGGGTRLEIK |
| 53 | D011-7016 VH | EVQLVESGPSLVKPSQTLSLICSVTGDSITSGYWNWVRKFPGNK |
| | | LEYMGYISYSGSAHYNPSLKSRISITRDTSKNQYYLQLNSMTTE |
| | | DTATYYCASYSFAYWGQGTLVTVSA |
| 54 | D011-7016 HC-CDR1 | GDSITSGY |
| 55 | D011-7016; D011- | ISYSGSA |
| | 7016-LD1; D011- | |
| | 7016-LD2; D011- | |
| | 7016-LD2_H0; D011- | |
| | 7016-LD2_H1; D011- | |
| | 7016-LD2_H3; D011- | |
| | 7016-LD2_H4: D011- | |
| | 7016-LD2_H5; D011- | |
| | 7016-LD2_HCON | |
| | HC-CDR2 | |
| 56 | D011-7016; D011- | ASYSFAY |
| | 7016-LD1; D011- | |
| | 7016-LD2; D011- | |
| | 7016-LD2_H0; D011- | |
| | 7016-LD2_H1; D011- | |
| | 7016-LD2_H3; D011- | |
| | 7016-LD2_H4; D011- | |
| | 7016-LD2_H5; D011- | |
| | 7016-LD2_HCON | |
| | HC-CDR3 | |
| 57 | D011-7016; D011- | EVQLVESGPSLVKPSQTLSLICSVT |
| | 7016-LD1; D011- | |
| | 7016-LD2 HC-FR1 | |
| 58 | D011-7016; D011- | WNWVRKFPGNKLEYMGY |
| | 7016-LD1; D011- | |
| | 7016-LD2 HC-FR2 | |
| 59 | D011-7016; D011- | HYNPSLKSRISITRDTSKNQYYLQLNSMTTEDTATYYC |
| | 7016-LD1; D011- | |
| | 7016-LD2 HC-FR3 | |
| 60 | D011-7016; D011- | WGQGTLVTVSA |
| | 7016-LD1; D011- | |
| | 7016-LD2 HC-FR4 | |
| 61 | D011-7016 VL | DIVMTQSPATLSVTPGDSVSLSCRANQSIDNNLHWYQQRSHESP |
| | | RLLINYASQSISGIPSRFSGSGSGTDFTLSISSVETEDFGMYFCQQS |
| | | YGWPYTFGGGTKLEIK |
| 62 | D011-7016 LC-CDR1 | QSIDNN |
| | D011-7016; D011- | YAS |
| | 7016-LD1; D011- | |
| | 7016-LD2; D011- | |
| | 7016-LD2_L0; D011- | |
| | 7016-LD2_L1; D011- | |
| | 7016-LD2_L2; D011- | |
| | 7016-LD2_L3; D011- | |
| | 7016-LD2_L4; D011- | |
| | 7016-LD2_LCON LC- | |
| | CDR2 | |
| 64 | D011-7016; D011- | QQSYGWPYT |
| | 7016-LD1; D011- | |
| | 7016-LD2; D011- | |
| | 7016-LD2_L0; D011- | |
| | 7016-LD2_L1; D011- | |
| | 7016-LD2_L2; D011- | |
| | 7016-LD2_L3; D011- | |
| | 7016-LD2_L4; D011- | |
| | 7016-LD2_LCON LC- | |
| | CDR3 | |
| 65 | D011-7016 LC-FR1 | DIVMTQSPATLSVTPGDSVSLSCRAN |
| 66 | D011-7016; D011- | LHWYQQRSHESPRLLIN |
| | 7016-LD1; D011- | |
| | 7016-LD2 LC-FR2 | |
| 67 | D011-7016; D011- | QSISGIPSRFSGSGSGTDFTLSISSVETEDFGMYFC |
| | 7016-LD1; D011- | |
| | 7016-LD2 LC-FR3 | |
| 68 | D011-7016; D011- | FGGGTKLEIK |
| | 7016-LD1; D011- | |
| | 7016-LD2; D011- | |
| | 7016-LD2_L4 LC- | |
| | FR4 | |
| 69 | 1E11P, 6040 | EVQLQQSGX₂EX₃VX₄X₅GASX₆KX₇SCX₈ASGX₉X₁₀X₁₁X₁₂X₁₃X₁₄X₁₅ |
| | consensus VH | MX₁₆WVKQX₁₇PX₁₈X₁₉X₂₀LX₂₁WIGX₂₂IX₂₃PX₂₄NGX₂₅X₂₆X₂₇YX₂₈ |
| | | X₂₉KFX₃₀GKATX₃₁TX₃₂DX₃₃SSX₃₄TAYX₃₅X₃₆LX₃₇SLTSX₃₈DX₃₉AV |
| | | YYCX₄₀X₄₁X₄₂X₄₃X₄₄X₄₅GYX₄₆X₄₇X₄₈X₄₉X₅₀DX₅₁WGQGTSVTVSS |
| | | Wherein X₂ = P or A, X₃ = L or F, X₄ = K or R, X₅ = P or |
| | | S, X₆ = M or V, X₇ = L or I, X₈ = K or T, X₉ = Y or F, |
| | | X₁₀ = S or N, X₁₁ = F or I, X₁₂ = T or K, X₁₃ = Gor D, |
| | | X₁₄ = S or Y, X₁₅ = T or Y, X₁₆ = N or H, X₁₇ = S or R, |
| | | X₁₈ = G or E, X₁₉ = K or Q, X₂₀ = N or G, X₂₁ = E or D, |
| | | X₂₂ = L or W, X₂₃ = N or D, X₂₄ = Y or E, X₂₅ = Gor D. |
| | | X₂₆ = T or A, X₂₇ = S or D, X₂₈ = N or D, X₂₉ = Q or P, |
| | | X₃₀ = E or Q, X₃₁ = L or M, X₃₂ = V or A, X₃₃ = K or T, |
| | | X₃₄ = S or N, X₃₅ = M or L, X₃₆ = E or H, X₃₇ = L or S, |
| | | X₃₈ = V or E, X₃₉ = S or A, X₄₀ = A or N, X₄₁ = S or A, |
| | | X₄₂ = R or C, X₄₃ = G or H, X₄₄ = K or Y, X₄₅ = N or Y, |
| | | X₄₆ = D or E, X₄₇ = G or D, X₄₈ = R or absent, |
| | | X₄₉ = D or A, X₅₀ = F or M, X₅₁ = C or Y. |
| 70 | 6039, 7016 consensus | EVX₅₂LVESGX₅₄X₅₅LVX₅₆PX₅₇X₅₈X₅₉LX₆₀LX₆₁CX₆₂X₆₃X₆₄GX₆₅X₆₆ |
| | VH | X₆₇X₆₈SX₆₉X₇₀X₇₁X₇₂WVRX₇₃X₇₄PX₇₅X₇₆X₇₇LEX₇₈X₇₉X₈₀X₈₁IX₈₂X₈₃ |
| | | X₈₄GX₈₅X₈₆X₈₇YX₈₈X₈₉X₉₀SX₉₁KX₉₂RX₉₃X₉₄IX₉₅RDX₉₆X₉₇KNX₉₈X₉₉YL |
| | | QX₁₀₀X₁₀₁SX₁₀₂X₁₀₃X₁₀₄EDTAX₁₀₅YYCAX₁₀₆X₁₀₇X₁₀₈FX₁₀₉YWGQGT |
| | | X₁₁₀X₁₁₁TVSX₁₁₂ |
| | | Wherein X₅₂ = M or Q, X₅₄ = G or P, X₅₅ = G or S, X₅₆ = Q |
| | | or K, X₅₇ = G or S, X₅₈ = G or Q, X₅₉ = S or T, X₆₀ = |
| | | K or S, X₆₁ = S or I, X₆₂ = A or S, X63 = A or V, X64 = |
| | | S or T, X₆₅ = F or D, X₆₆ = T or S, X₆₇ = F or I, X₆₈ = S |
| | | or T, X₆₉ = Y or G, X₇₀ = Gor Y, X₇₁ = M or W, X₇₂ = S or |
| | | N, X₇₃ = Q or K, X₇₄ = T or F, X₇₅ = D or G, X₇₆ = K or N, |
| | | X₇₇ = R or K, X₇₈ = L or Y, X₇₉ = V or M, X₈₀ = A or G, |
| | | X₈₁ = T or Y, X₈₂ = N or S, X₈₃ = S or Y, X₈₄ = N or S, |
| | | X₈₅ = V or S, X₈₆ = N or A, X₈₇ = T or H, X₈₈ = Y or N, |
| | | X₈₉ = P or absent, X₉₀ = D or P, X₉₁ = V or L, X₉₂ = G or |
| | | S, X₉₃ = F or I, X₉₄ = T or S, X₉₅ = S or T, X₉₆ = N or T, |
| | | X₉₇ = A or S, X₉₈ = T or Q, X₉₉ = L or Y, X₁₀₀ = M or L, |
| | | X₁₀₁ = S or N, X₁₀₂ = L or M, X₁₀₃ = K or T, X₁₀₄ = S or T, |
| | | X₁₀₅ = M or T, X₁₀₆ = R or S, X₁₀₇ = V or Y, X₁₀₈ = P or S, |
| | | X₁₀₉ = D or A, X₁₁₀ = S or L, X₁₁₁ = L or V, X₁₁₂ = S or A. |
| 71 | Human IgG1 constant | ASTKGPSVFPLAPSSKSTSGGTAALGCLVKDYFPEPVTVSWNSG |
| | region (IGHG1; | ALTSGVHTFPAVLQSSGLYSLSSVVTVPSSSLGTQTYICNVNHKP |
| | UniProt:P01857-1, v1) | SNTKVDKKVEPKSCDKTHTCPPCPAPELLGGPSVFLFPPKPKDT |
| | | LMISRTPEVTCVVVDVSHEDPEVKFNWYVDGVEVHNAKTKPR |
| | | EEQYNSTYRVVSVLTVLHQDWLNGKEYKCKVSNKALPAPIEKT |
| | | ISKAKGQPREPQVYTLPPSRDELTKNQVSLTCLVKGFYPSDIAVE |
| | | WESNGQPENNYKTTPPVLDSDGSFFLYSKLTVDKSRWQQGNVF |
| | | SCSVMHEALHNHYTQKSLSLSPGK |
| 72 | CH1 IgG1 (positions | ASTKGPSVFPLAPSSKSTSGGTAALGCLVKDYFPEPVTVSWNSG |
| | 1-98 of P01857-1, v1) | ALTSGVHTFPAVLQSSGLYSLSSVVTVPSSSLGTQTYICNVNHKP |
| | | SNTKVDKKV |
| 73 | Hinge IgG1 (positions | EPKSCDKTHTCP |
| | 99-110 of P01857-1, | |
| | v1) | |
| 74 | CH2 IgG1 (positions | PCPAPELLGGPSVFLFPPKPKDTLMISRTPEVTCVVVDVSHEDPE |
| | 111-223 of P01857-1, | VKFNWYVDGVEVHNAKTKPREEQYNSTYRVVSVLTVLHQDW |
| | v1) | LNGKEYKCKVSNKALPAPIEKTISKAK |
| 75 | CH3 IgG1 (positions | GQPREPQVYTLPPSRDELTKNQVSLTCLVKGFYPSDIAVEWESN |
| | 224-330 of P01857-1, | GQPENNYKTTPPVLDSDGSFFLYSKLTVDKSRWQQGNVFSCSV |
| | v1) | MHEALHNHYTQKSLSLSPGK |
| 76 | Human IgG1 constant | ASTKGPSVFPLAPSSKSTSGGTAALGCLVKDYFPEPVTVSWNSG |
| | region G1m3 allotype | ALTSGVHTFPAVLQSSGLYSLSSVVTVPSSSLGTQTYICNVNHKP |
| | (K214R, D356E and | SNTKVDKRVEPKSCDKTHTCPPCPAPELLGGPSVFLFPPKPKDTL |
| | L358M (EU | MISRTPEVTCVVVDVSHEDPEVKFNWYVDGVEVHNAKTKPRE |
| | numbering) relative to | EQYNSTYRVVSVLTVLHQDWLNGKEYKCKVSNKALPAPIEKTI |
| | P01857-1) | SKAKGQPREPQVYTLPPSREEMTKNQVSLTCLVKGFYPSDIAVE |
| | | WESNGQPENNYKTTPPVLDSDGSFFLYSKLTVDKSRWQQGNVF |
| | | SCSVMHEALHNHYTQKSLSLSPGK |
| 77 | CH1 IgG1 G1m3 | ASTKGPSVFPLAPSSKSTSGGTAALGCLVKDYFPEPVTVSWNSG |
| | allotype | ALTSGVHTFPAVLQSSGLYSLSSVVTVPSSSLGTQTYICNVNHKP |
| | | SNTKVDKRV |
| 78 | CH3 IgGI G1m3 | GQPREPQVYTLPPSREEMTKNQVSLTCLVKGFYPSDIAVEWESN |
| | allotype | GQPENNYKTTPPVLDSDGSFFLYSKLTVDKSRWQQGNVFSCSV |
| | | MHEALHNHYTQKSLSLSPGK |
| 79 | Cκ CL (IGKC; | RTVAAPSVFIFPPSDEQLKSGTASVVCLLNNFYPREAKVQWKVD |
| | UniProt: P01834-1, | NALQSGNSQESVTEQDSKDSTYSLSSTLTLSKADYEKHKVYAC |
| | v2) | EVTHQGLSSPVTKSFNRGEC |
| 80 | Cλ CL1 (IGLC1; | GQPKANPTVTLFPPSSEELQANKATLVCLISDFYPGAVTVAWKA |
| | UniProt: P0CG04-1, | DGSPVKAGVETTKPSKQSNNKYAASSYLSLTPEQWKSHRSYSC |
| | v1) | QVTHEGSTVEKTVAPTECS |
| 81 | Cλ CL2 (IGLC2; | GQPKAAPSVTLFPPSSEELQANKATLVCLISDFYPGAVTVAWKA |
| | UniProt: PODOY2-1, | DSSPVKAGVETTTPSKQSNNKYAASSYLSLTPEQWKSHRSYSCQ |
| | v1) | VTHEGSTVEKTVAPTECS |
| 82 | Cλ CL3 (IGLC3; | GQPKAAPSVTLFPPSSEELQANKATLVCLISDFYPGAVTVAWKA |
| | UniProt: PODOY3-1, | DSSPVKAGVETTTPSKQSNNKYAASSYLSLTPEQWKSHKSYSC |
| | v1) | QVTHEGSTVEKTVAPTECS |
| 83 | Cλ CL6 (IGLC6; | GQPKAAPSVTLFPPSSEELQANKATLVCLISDFYPGAVKVAWKA |
| | UniProt: POCF74-1, | DGSPVNTGVETTTPSKQSNNKYAASSYLSLTPEQWKSHRSYSC |
| | v1) | QVTHEGSTVEKTVAPAECS |
| 84 | Cλ CL7 (IGLC7; | GQPKAAPSVTLFPPSSEELQANKATLVCLVSDFNPGAVTVAWK |
| | UniProt: A0M8Q6-1, | ADGSPVKVGVETTKPSKQSNNKYAASSYLSLTPEQWKSHRSYS |
| | v3) | CRVTHEGSTVEKTVAPAECS |
| 85 | C220S Hinge | EPKSSDKTHTCP |
| 86 | D011-1E11p hIgG1 | EVQLQQSGPELVKPGASMKISCKASGYSFTGSTMNWVKQSPGK |
| | HC | NLEWIGLINPYNGGTSYNQKFEGKATLTVDKSSSTAYMELLSLT |
| | | SVDSAVYYCASRGKNGYDGRDFDCWGQGTSVTVSSASTKGPS |
| | | VFPLAPSSKSTSGGTAALGCLVKDYFPEPVTVSWNSGALTSGVH |
| | | TFPAVLQSSGLYSLSSVVTVPSSSLGTQTYICNVNHKPSNTKVD |
| | | KKVEPKSCDKTHTCPPCPAPELLGGPSVFLFPPKPKDTLMISRTP |
| | | EVTCVVVDVSHEDPEVKFNWYVDGVEVHNAKTKPREEQYNST |
| | | YRVVSVLTVLHQDWLNGKEYKCKVSNKALPAPIEKTISKAKGQ |
| | | PREPQVYTLPPSRDELTKNQVSLTCLVKGFYPSDIAVEWESNGQ |
| | | PENNYKTTPPVLDSDGSFFLYSKLTVDKSRWQQGNVFSCSVMH |
| | | EALHNHYTQKSLSLSPGK |
| 87 | D011-6039 hIgG1 HC | EVMLVESGGGLVQPGGSLKLSCAASGFTFSSYGMSWVRQTPDK |
| | | RLELVATINSNGVNTYYPDSVKGRFTISRDNAKNTLYLQMSSLK |
| | | SEDTAMYYCARVPFDYWGQGTSLTVSSASTKGPSVFPLAPSSKS |
| | | TSGGTAALGCLVKDYFPEPVTVSWNSGALTSGVHTFPAVLQSS |
| | | GLYSLSSVVTVPSSSLGTQTYICNVNHKPSNTKVDKKVEPKSCD |
| | | KTHTCPPCPAPELLGGPSVFLFPPKPKDTLMISRTPEVTCVVVDV |
| | | SHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSRDELTKNQVSLTCLVKGFYPSDIAVEWESNGQPENNYKTTP |
| | | PVLDSDGSFFLYSKLTVDKSRWQQGNVFSCSVMHEALHNHYT |
| | | QKSLSLSPGK |
| 88 | D011-6040 hIgG1 HC | EVQLQQSGAEFVRSGASVKLSCTASGFNIKDYYMHWVKQRPE |
| | | QGLDWIGWIDPENGDADYDPKFQGKATMTADTSSNTAYLHLSS |
| | | LTSEDAAVYYCNACHYYGYEDAMDYWGQGTSVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFLFPPKPKDTLMISRT |
| | | PEVTCVVVDVSHEDPEVKFNWYVDGVEVHNAKTKPREEQYNS |
| | | TYRVVSVLTVLHQDWLNGKEYKCKVSNKALPAPIEKTISKAKG |
| | | QPREPQVYTLPPSRDELTKNQVSLTCLVKGFYPSDIAVEWESNG |
| | | QPENNYKTTPPVLDSDGSFFLYSKLTVDKSRWQQGNVFSCSVM |
| | | HEALHNHYTQKSLSLSPGK |
| 89 | D011-7016 hIgG1 HC | EVQLVESGPSLVKPSQTLSLICSVTGDSITSGYWNWVRKFPGNK |
| | | LEYMGYISYSGSAHYNPSLKSRISITRDTSKNQYYLQLNSMTTE |
| | | DTATYYCASYSFAYWGQGTLVTVSAASTKGPSVFPLAPSSKSTS |
| | | GGTAALGCLVKDYFPEPVTVSWNSGALTSGVHTFPAVLQSSGL |
| | | YSLSSVVTVPSSSLGTQTYICNVNHKPSNTKVDKKVEPKSCDKT |
| | | HTCPPCPAPELLGGPSVFLFPPKPKDTLMISRTPEVTCVVVDVSH |
| | | EDPEVKFNWYVDGVEVHNAKTKPREEQYNSTYRVVSVLTVLH |
| | | QDWLNGKEYKCKVSNKALPAPIEKTISKAKGQPREPQVYTLPPS |
| | | RDELTKNQVSLTCLVKGFYPSDIAVEWESNGQPENNYKTTPPVL |
| | | DSDGSFFLYSKLTVDKSRWQQGNVFSCSVMHEALHNHYTQKS |
| | | LSLSPGK |
| 90 | D011-1E11p κLC | DIVLTQSPLSLPVNIGDQASISCKSTKSLLNSGGFTYLDWYLQKP |
| | | GQSPQLLIYLVSNRFSGVPDRFSGSGSGTDFTLKISRVEAEDLGV |
| | | YYCFQSNYFPFTFGSGTKLEIKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 91 | D011-6039 κLC | DIVMTQSPAIMSASLGEKVTMSCRASSSVDYIYWYQQKSDASP |
| | | KLWIYYTSTLAPGVPARFSGSGSGNSYSLTISSMEGEDAATYYC |
| | | QQFTSSPWTFGGGTRLEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |
| 92 | D011-6040 κLC | DIQMTQSPSSLSASLGERVSLTCRASQEISGYLSWLQQKPDGTIK |
| | | RLIYAASTLDSGVPKRFSGSRSGSDYSLTISSLESEDFADYYCLQ |
| | | YAGYPRTFGGGTRLEIKRTVAAPSVFIFPPSDEQLKSGTASVVCL |
| | | LNNFYPREAKVQWKVDNALQSGNSQESVTEQDSKDSTYSLSST |
| | | LTLSKADYEKHKVYACEVTHQGLSSPVTKSFNRGEC |
| 93 | D011-7016 κLC | DIVMTQSPATLSVTPGDSVSLSCRANQSIDNNLHWYQQRSHESP |
| | | RLLINYASQSISGIPSRFSGSGSGTDFTLSISSVETEDFGMYFCQQS |
| | | YGWPYTFGGGTKLEIKRTVAAPSVFIFPPSDEQLKSGTASVVCL |
| | | LNNFYPREAKVQWKVDNALQSGNSQESVTEQDSKDSTYSLSST |
| | | LTLSKADYEKHKVYACEVTHQGLSSPVTKSFNRGEC |
| 94 | Mouse IgG2 constant | AKTTAPSVYPLAPVCGDTTGSSVTLGCLVKGYFPEPVTLTWNS |
| | region, A allele | GSLSSGVHTFPAVLQSDLYTLSSSVTVTSSTWPSQSITCNVAHPA |
| | (IGHG2a; UniProt: | SSTKVDKKIEPRGPTIKPCPPCKCPAPNLLGGPSVFIFPPKIKDVL |
| | P01863, v1) | MISLSPIVTCVVVDVSEDDPDVQISWFVNNVEVHTAQTQTHRED |
| | | YNSTLRVVSALPIQHQDWMSGKEFKCKVNNKDLPAPIERTISKP |
| | | KGSVRAPQVYVLPPPEEEMTKKQVTLTCMVTDFMPEDIYVEWT |
| | | NNGKTELNYKNTEPVLDSDGSYFMYSKLRVEKKNWVERNSYS |
| | | CSVVHEGLHNHHTTKSFSRTPGK |
| 95 | D011-1E11p IgG2a | EVQLQQSGPELVKPGASMKISCKASGYSFTGSTMNWVKQSPGK |
| | HC | NLEWIGLINPYNGGTSYNQKFEGKATLTVDKSSSTAYMELLSLT |
| | | SVDSAVYYCASRGKNGYDGRDFDCWGQGTSVTVSSASTKGPS |
| | | VFPLAPSSKSTSGGTAALGCLVKDYFPEPVTVSWNSGALTSGVH |
| | | TFPAVLQSSGLYSLSSVVTVPSSSLGTQTYICNVNHKPSNTKVD |
| | | KKVEPKSCDKTHTCPPCPAPELLGGPSVFIFPPKIKDVLMISLSPI |
| | | VTCVVVDVSEDDPDVQISWFVNNVEVHTAQTQTHREDYNSTL |
| | | RVVSALPIQHQDWMSGKEFKCKVNNKDLPAPIERTISKPKGSVR |
| | | APQVYVLPPPEEEMTKKQVTLTCMVTDFMPEDIYVEWTNNGK |
| | | TELNYKNTEPVLDSDGSYFMYSKLRVEKKNWVERNSYSCSVV |
| | | HEGLHNHHTTKSFSRTPGK |
| 96 | D011-6039 IgG2a HC | EVMLVESGGGLVQPGGSLKLSCAASGFTFSSYGMSWVRQTPDK |
| | | RLELVATINSNGVNTYYPDSVKGRFTISRDNAKNTLYLQMSSLK |
| | | SEDTAMYYCARVPFDYWGQGTSLTVSSASTKGPSVFPLAPSSKS |
| | | TSGGTAALGCLVKDYFPEPVTVSWNSGALTSGVHTFPAVLQSS |
| | | GLYSLSSVVTVPSSSLGTQTYICNVNHKPSNTKVDKKVEPKSCD |
| | | KTHTCPPCPAPELLGGPSVFIFPPKIKDVLMISLSPIVTCVVVDVS |
| | | EDDPDVQISWFVNNVEVHTAQTQTHREDYNSTLRVVSALPIQH |
| | | QDWMSGKEFKCKVNNKDLPAPIERTISKPKGSVRAPQVYVLPPP |
| | | EEEMTKKQVTLTCMVTDFMPEDIYVEWTNNGKTELNYKNTEP |
| | | VLDSDGSYFMYSKLRVEKKNWVERNSYSCSVVHEGLHNHHTT |
| | | KSFSRTPGK |
| 97 | D011-6040 IgG2a HC | EVQLQQSGAEFVRSGASVKLSCTASGFNIKDYYMHWVKQRPE |
| | | QGLDWIGWIDPENGDADYDPKFQGKATMTADTSSNTAYLHLSS |
| | | LTSEDAAVYYCNACHYYGYEDAMDYWGQGTSVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKKVEPKSCDKTHTCPPCPAPELLGGPSVFIFPPKIKDVLMISLSP |
| | | IVTCVVVDVSEDDPDVQISWFVNNVEVHTAQTQTHREDYNSTL |
| | | RVVSALPIQHQDWMSGKEFKCKVNNKDLPAPIERTISKPKGSVR |
| | | APQVYVLPPPEEEMTKKQVTLTCMVTDFMPEDIYVEWTNNGK |
| | | TELNYKNTEPVLDSDGSYFMYSKLRVEKKNWVERNSYSCSVV |
| | | HEGLHNHHTTKSFSRTPGK |
| 98 | D011-7016 IgG2a HC | EVQLVESGPSLVKPSQTLSLICSVTGDSITSGYWNWVRKFPGNK |
| | | LEYMGYISYSGSAHYNPSLKSRISITRDTSKNQYYLQLNSMTTE |
| | | DTATYYCASYSFAYWGQGTLVTVSAASTKGPSVFPLAPSSKSTS |
| | | GGTAALGCLVKDYFPEPVTVSWNSGALTSGVHTFPAVLQSSGL |
| | | YSLSSVVTVPSSSLGTQTYICNVNHKPSNTKVDKKVEPKSCDKT |
| | | HTCPPCPAPELLGGPSVFIFPPKIKDVLMISLSPIVTCVVVDVSED |
| | | DPDVQISWFVNNVEVHTAQTQTHREDYNSTLRVVSALPIQHQD |
| | | WMSGKEFKCKVNNKDLPAPIERTISKPKGSVRAPQVYVLPPPEE |
| | | EMTKKQVTLTCMVTDFMPEDIYVEWTNNGKTELNYKNTEPVL |
| | | DSDGSYFMYSKLRVEKKNWVERNSYSCSVVHEGLHNHHTTKS |
| | | FSRTPGK |
| 99 | D011-1E11-5T2 | QVQLVQSGAEVKKPGASMKVSCKASGYSFTGSTMNWVRQAPG |
| | IgG2a HC | QGLEWMGWINPYNGGTNYAQKFQGRVTMTRDTSISTAYMELS |
| | | RLRSDDTAVYYCASRGKNGYDGRDFDCWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKKVEPKSCDKTHTCPPCPAPELLGGPSVFIFPPKIKDVLMIS |
| | | LSPIVTCVVVDVSEDDPDVQISWFVNNVEVHTAQTQTHREDYN |
| | | STLRVVSALPIQHQDWMSGKEFKCKVNNKDLPAPIERTISKPKG |
| | | SVRAPQVYVLPPPEEEMTKKQVTLTCMVTDFMPEDIYVEWTNN |
| | | GKTELNYKNTEPVLDSDGSYFMYSKLRVEKKNWVERNSYSCS |
| | | VVHEGLHNHHTTKSFSRTPGK |
| 100 | Mouse Igkc (UniProt: | ELVMTQSPLSLSVSLGDQASISCRSSQSLVHTNGNTYLHWYLQK |
| | A2P1G9_MOUSE, | PGLSPKLLIYIVSNRFSGVPDRFSGSGSGTDFTLKISRVEAEDLGV |
| | v1) | YFCSQVTHVPWTFGGGTKLEIKRADAAPTVSIFPPSSEQLTSGGA |
| | | SVVCFLNNFYPKDINVKWKIDGSERQNGVLNSWTDQDSKDSTY |
| | | SMSSTLTLTKDEYERHNSYTCEATHKTSTSPIVKSFNRGEC |
| 101 | D011-1E11p mκLC | DIVLTQSPLSLPVNIGDQASISCKSTKSLLNSGGFTYLDWYLQKP |
| | | GQSPQLLIYLVSNRFSGVPDRFSGSGSGTDFTLKISRVEAEDLGV |
| | | YYCFQSNYFPFTFGSGTKLEIKELVMTQSPLSLSVSLGDQASISC |
| | | RSSQSLVHTNGNTYLHWYLQKPGLSPKLLIYIVSNRFSGVPDRF |
| | | SGSGSGTDFTLKISRVEAEDLGVYFCSQVTHVPWTFGGGTKLEI |
| | | KRADAAPTVSIFPPSSEQLTSGGASVVCFLNNFYPKDINVKWKI |
| | | DGSERQNGVLNSWTDQDSKDSTYSMSSTLTLTKDEYERHNSYT |
| | | CEATHKTSTSPIVKSFNRGEC |
| 102 | D011-6039 mκLC | DIVMTQSPAIMSASLGEKVTMSCRASSSVDYIYWYQQKSDASP |
| | | KLWIYYTSTLAPGVPARFSGSGSGNSYSLTISSMEGEDAATYYC |
| | | QQFTSSPWTFGGGTRLEIKELVMTQSPLSLSVSLGDQASISCRSS |
| | | QSLVHTNGNTYLHWYLQKPGLSPKLLIYIVSNRFSGVPDRFSGS |
| | | GSGTDFTLKISRVEAEDLGVYFCSQVTHVPWTFGGGTKLEIKRA |
| | | DAAPTVSIFPPSSEQLTSGGASVVCFLNNFYPKDINVKWKIDGSE |
| | | RQNGVLNSWTDQDSKDSTYSMSSTLTLTKDEYERHNSYTCEAT |
| | | HKTSTSPIVKSFNRGEC |
| 103 | D011-6040 mκLC | DIQMTQSPSSLSASLGERVSLTCRASQEISGYLSWLQQKPDGTIK |
| | | RLIYAASTLDSGVPKRFSGSRSGSDYSLTISSLESEDFADYYCLQ |
| | | YAGYPRTFGGGTRLEIKELVMTQSPLSLSVSLGDQASISCRSSQS |
| | | LVHTNGNTYLHWYLQKPGLSPKLLIYIVSNRFSGVPDRFSGSGS |
| | | GTDFTLKISRVEAEDLGVYFCSQVTHVPWTFGGGTKLEIKRADA |
| | | APTVSIFPPSSEQLTSGGASVVCFLNNFYPKDINVKWKIDGSERQ |
| | | NGVLNSWTDQDSKDSTYSMSSTLTLTKDEYERHNSYTCEATHK |
| | | TSTSPIVKSFNRGEC |
| 104 | D011-7016 mκLC | DIVMTQSPATLSVTPGDSVSLSCRANQSIDNNLHWYQQRSHESP |
| | | RLLINYASQSISGIPSRFSGSGSGTDFTLSISSVETEDFGMYFCQQS |
| | | YGWPYTFGGGTKLEIKELVMTQSPLSLSVSLGDQASISCRSSQSL |
| | | VHTNGNTYLHWYLQKPGLSPKLLIYIVSNRFSGVPDRFSGSGSG |
| | | TDFTLKISRVEAEDLGVYFCSQVTHVPWTFGGGTKLEIKRADAA |
| | | PTVSIFPPSSEQLTSGGASVVCFLNNFYPKDINVKWKIDGSERQN |
| | | GVLNSWTDQDSKDSTYSMSSTLTLTKDEYERHNSYTCEATHKT |
| | | STSPIVKSFNRGEC |
| 105 | D011-1E11-5T2 | EIVLTQSPATLSLSPGERATLSCKSTKSLLNSGGFSYLDWYQQKP |
| | mκLC | GQAPRLLIYL VSNRFTGIPARFSGSGSGTDFTLTISSLEPEDFAVY |
| | | YCFQSNYFPFTFGQGTKLEIKELVMTQSPLSLSVSLGDQASISCR |
| | | SSQSLVHTNGNTYLHWYLQKPGLSPKLLIYIVSNRFSGVPDRFS |
| | | GSGSGTDFTLKISRVEAEDLGVYFCSQVTHVPWTFGGGTKLEIK |
| | | RADAAPTVSIFPPSSEQLTSGGASVVCFLNNFYPKDINVKWKID |
| | | GSERQNGVLNSWTDQDSKDSTYSMSSTLTLTKDEYERHNSYTC |
| | | EATHKTSTSPIVKSFNRGEC |
| 106 | zanolimumab hIgG1 | QVQLQQWGAGLLKPSETLSLTCAVYGGSFSGYYWSWIRQPPGK |
| | HC | GLEWIGEINHSGSTNYNPSLKSRVTISVDTSKNQFSLKLSSVTAA |
| | | DTAVYYCARVINWFDPWGQGTLVTVSSASTKGPSVFPLAPSSK |
| | | STSGGTAALGCLVKDYFPEPVTVSWNSGALTSGVHTFPAVLQSS |
| | | GLYSLSSVVTVPSSSLGTQTYICNVNHKPSNTKVDKKVEPKSCD |
| | | KTHTCPPCPAPELLGGPSVFLFPPKPKDTLMISRTPEVTCVVVDV |
| | | SHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSRDELTKNQVSLTCLVKGFYPSDIAVEWESNGQPENNYKTTP |
| | | PVLDSDGSFFLYSKLTVDKSRWQQGNVFSCSVMHEALHNHYT |
| | | QKSLSLSPGK |
| 107 | patritumab hIgG1 HC | QVQLQQWGAGLLKPSETLSLTCAVYGGSFSGYYWSWIRQPPGK |
| | | GLEWIGEINHSGSTNYNPSLKSRVTISVETSKNQFSLKLSSVTAA |
| | | DTAVYYCARDKWTWYFDLWGRGTLVTVSSASTKGPSVFPLAP |
| | | SSKSTSGGTAALGCLVKDYFPEPVTVSWNSGALTSGVHTFPAVL |
| | | QSSGLYSLSSVVTVPSSSLGTQTYICNVNHKPSNTKVDKKVEPK |
| | | SCDKTHTCPPCPAPELLGGPSVFLFPPKPKDTLMISRTPEVTCVV |
| | | VDVSHEDPEVKFNWYVDGVEVHNAKTKPREEQYNSTYRVVSV |
| | | LTVLHQDWLNGKEYKCKVSNKALPAPIEKTISKAKGQPREPQV |
| | | YTLPPSRDELTKNQVSLTCLVKGFYPSDIAVEWESNGQPENNYK |
| | | TTPPVLDSDGSFFLYSKLTVDKSRWQQGNVFSCSVMHEALHNH |
| | | YTQKSLSLSPGK |
| 108 | tabalumab hIgG4 HC | QVQLQQWGAGLLKPSETLSLTCAVYGGSFSGYYWSWIRQPPGK |
| | | GLEWIGEINHSGSTNYNPSLKSRVTISVDTSKNQFSLKLSSVTAA |
| | | DTAVYYCARGYYDILTGYYYYFDYWGQGTLVTVSSASTKGPS |
| | | VFPLAPCSRSTSESTAALGCLVKDYFPEPVTVSWNSGALTSGVH |
| | | TFPAVLQSSGLYSLSSVVTVPSSSLGTKTYTCNVDHKPSNTKVD |
| | | KRVESKYGPPCPSCPAPEFLGGPSVFLFPPKPKDTLMISRTPEVT |
| | | CVVVDVSQEDPEVQFNWYVDGVEVHNAKTKPREEQFNSTYRV |
| | | VSVLTVLHQDWLNGKEYKCKVSNKGLPSSIEKTISKAKGQPREP |
| | | QVYTLPPSQEEMTKNQVSLTCLVKGFYPSDIAVEWESNGQPEN |
| | | NYKTTPPVLDSDGSFFLYSRLTVDKSRWQEGNVFSCSVMHEAL |
| | | HNHYTQKSLSLSLGK |
| 109 | zanolimumab κLC | DIQMTQSPSSVSASVGDRVTITCRASQDISSWLAWYQHKPGKAP |
| | | KLLIYAASSLQSGVPSRFSGSGSGTDFTLTISSLQPEDFATYYCQ |
| | | QANSFPYTFGQGTKLEIKRTVAAPSVFIFPPSDEQLKSGTASVVC |
| | | LLNNFYPREAKVQWKVDNALQSGNSQESVTEQDSKDSTYSLSS |
| | | TLTLSKADYEKHKVYACEVTHQGLSSPVTKSFNRGEC |
| 110 | patritumab κLC | DIEMTQSPDSLAVSLGERATINCRSSQSVLYSSSNRNYLAWYQQ |
| | | NPGQPPKLLIYWASTRESGVPDRFSGSGSGTDFTLTISSLQAEDV |
| | | AVYYCQQYYSTPRTFGQGTKVEIKRTVAAPSVFIFPPSDEQLKS |
| | | GTASVVCLLNNFYPREAKVQWKVDNALQSGNSQESVTEQDSK |
| | | DSTYSLSSTLTLSKADYEKHKVYACEVTHQGLSSPVTKSFNRGE |
| | | C |
| 111 | tabalumab κLC | EIVLTQSPATLSLSPGERATLSCRASQSVSRYLAWYQQKPGQAP |
| | | RLLIYDASNRATGIPARFSGSGSGTDSTLTISSLEPEDFAVYYCQ |
| | | QRSNWPRTFGQGTKVEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |
| 112 | zanolimumab mutant | QVQLQGAGAGLLKPSETLSLTCGASGGSFSGYYWSWIRQPPGK |
| | FR1 VH | GLEWIGEINHSGSTNYNPSLKSRVTISVDTSKNQFSLKLSSVTAA |
| | | DTAVYYCARVINWFDPWGQGTLVTVSS |
| 113 | zanolimumab mutant | QVQLQGAGAGLLKPSETLSLTCGASGGSFSGYYWSWIRQPPGK |
| | FR1 hIgG1 HC | GLEWIGEINHSGSTNYNPSLKSRVTISVDTSKNQFSLKLSSVTAA |
| | | DTAVYYCARVINWFDPWGQGTLVTVSSASTKGPSVFPLAPSSK |
| | | STSGGTAALGCLVKDYFPEPVTVSWNSGALTSGVHTFPAVLQSS |
| | | GLYSLSSVVTVPSSSLGTQTYICNVNHKPSNTKVDKKVEPKSCD |
| | | KTHTCPPCPAPELLGGPSVFLFPPKPKDTLMISRTPEVTCVVVDV |
| | | SHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSRDELTKNQVSLTCLVKGFYPSDIAVEWESNGQPENNYKTTP |
| | | PVLDSDGSFFLYSKLTVDKSRWQQGNVFSCSVMHEALHNHYT |
| | | QKSLSLSPGK |
| 114 | 3A6 hIgG1 HC | QVQLQQWGAGLLKPSETLSLTCAVYGGSFSGYYWSWIRQPPGK |
| | | GLEWIGEINHSGSTNYNPSLKSRVTISVDTSKNQFSLKLSSVTAA |
| | | DTAVYYCARGLGYSGSYYAPNWGQGTLVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPELLGGPSVFLFPPKPKDTLMISRTPEVTC |
| | | VVVDVSHEDPEVKFNWYVDGVEVHNAKTKPREEQYNSTYRVV |
| | | SVLTVLHQDWLNGKEYKCKVSNKALPAPIEKTISKAKGQPREP |
| | | QVYTLPPSRDELTKNQVSLTCLVKGFYPSDIAVEWESNGQPENN |
| | | YKTTPPVLDSDGSFFLYSKLTVDKSRWQQGNVFSCSVMHEALH |
| | | NHYTQKSLSLSPGK |
| 115 | 3A6 κLC | DVVMTQSPLSLPVTPGEPASISCRSSQSLLHSNGYNYLDWYLQK |
| | | PGQSPQLLIYLGSNRASGVPDRFSGSGSGTDFTLKISRVEAEDVG |
| | | VYYCMQGTHWPRTFGQGTKVEIKRTVAAPSVFIFPPSDEQLKSG |
| | | TASVVCLLNNFYPREAKVQWKVDNALQSGNSQESVTEQDSKD |
| | | STYSLSSTLTLSKADYEKHKVYACEVTHQGLSSPVTKSFNRGEC |
| 116 | 3A6 mutant FR1 VH | QVQLQGAGAGLLKPSETLSLTCGASGGSFSGYYWSWIRQPPGK |
| | | GLEWIGEINHSGSTNYNPSLKSRVTISVDTSKNQFSLKLSSVTAA |
| | | DTAVYYCARGLGYSGSYYAPNWGQGTLVTVSS |
| 117 | 3A6 mutant FR1 | QVQLQGAGAGLLKPSETLSLTCGASGGSFSGYYWSWIRQPPGK |
| | hIgG1 HC | GLEWIGEINHSGSTNYNPSLKSRVTISVDTSKNQFSLKLSSVTAA |
| | | DTAVYYCARGLGYSGSYYAPNWGQGTLVTVSSASTKGPSVFPL |
| | | APSSKSTSGGTAALGCLVKDYFPEPVTVSWNSGALTSGVHTFPA |
| | | VLQSSGLYSLSSVVTVPSSSLGTQTYICNVNHKPSNTKVDKKVE |
| | | PKSCDKTHTCPPCPAPELLGGPSVFLFPPKPKDTLMISRTPEVTC |
| | | VVVDVSHEDPEVKFNWYVDGVEVHNAKTKPREEQYNSTYRVV |
| | | SVLTVLHQDWLNGKEYKCKVSNKALPAPIEKTISKAKGQPREP |
| | | QVYTLPPSRDELTKNQVSLTCLVKGFYPSDIAVEWESNGQPENN |
| | | YKTTPPVLDSDGSFFLYSKLTVDKSRWQQGNVFSCSVMHEALH |
| | | NHYTQKSLSLSPGK |
| 118 | tabalumab mutant FR1 | QVQLQGAGAGLLKPSETLSLTCGASGGSFSGYYWSWIRQPPGK |
| | VH | GLEWIGEINHSGSTNYNPSLKSRVTISVDTSKNQFSLKLSSVTAA |
| | | DTAVYYCARGYYDILTGYYYYFDYWGQGTLVTVSS |
| 119 | tabalumab mutant FR1 | QVQLQGAGAGLLKPSETLSLTCGASGGSFSGYYWSWIRQPPGK |
| | hIgG1 HC | GLEWIGEINHSGSTNYNPSLKSRVTISVDTSKNQFSLKLSSVTAA |
| | | DTAVYYCARGYYDILTGYYYYFDYWGQGTLVTVSSASTKGPS |
| | | VFPLAPSSKSTSGGTAALGCLVKDYFPEPVTVSWNSGALTSGVH |
| | | TFPAVLQSSGLYSLSSVVTVPSSSLGTQTYICNVNHKPSNTKVD |
| | | KKVEPKSCDKTHTCPPCPAPELLGGPSVFLFPPKPKDTLMISRTP |
| | | EVTCVVVDVSHEDPEVKFNWYVDGVEVHNAKTKPREEQYNST |
| | | YRVVSVLTVLHQDWLNGKEYKCKVSNKALPAPIEKTISKAKGQ |
| | | PREPQVYTLPPSRDELTKNQVSLTCLVKGFYPSDIAVEWESNGQ |
| | | PENNYKTTPPVLDSDGSFFLYSKLTVDKSRWQQGNVFSCSVMH |
| | | EALHNHYTQKSLSLSPGK |
| 120 | tabalumab hIgG1 HC | QVQLQQWGAGLLKPSETLSLTCAVYGGSFSGYYWSWIRQPPGK |
| | | GLEWIGEINHSGSTNYNPSLKSRVTISVDTSKNQFSLKLSSVTAA |
| | | DTAVYYCARGYYDILTGYYYYFDYWGQGTLVTVSSASTKGPS |
| | | VFPLAPSSKSTSGGTAALGCLVKDYFPEPVTVSWNSGALTSGVH |
| | | TFPAVLQSSGLYSLSSVVTVPSSSLGTQTYICNVNHKPSNTKVD |
| | | KKVEPKSCDKTHTCPPCPAPELLGGPSVFLFPPKPKDTLMISRTP |
| | | EVTCVVVDVSHEDPEVKFNWYVDGVEVHNAKTKPREEQYNST |
| | | YRVVSVLTVLHQDWLNGKEYKCKVSNKALPAPIEKTISKAKGQ |
| | | PREPQVYTLPPSRDELTKNQVSLTCLVKGFYPSDIAVEWESNGQ |
| | | PENNYKTTPPVLDSDGSFFLYSKLTVDKSRWQQGNVFSCSVMH |
| | | EALHNHYTQKSLSLSPGK |
| 121 | Human CH2 IgG1 | PCPAPE |
| | fragment (positions | |
| | 111-116 of P01857-1, | |
| | v1) | |
| 122 | Mouse CH2 IgG2a | LLGGPSVFIFPPKIKDVLMISLSPIVTCVVVDVSEDDPDVQISWFV |
| | (positions 117-223 of | NNVEVHTAQTQTHREDYNSTLRVVSALPIQHQDWMSGKEFKC |
| | UniProt: P01863, v1) | KVNNKDLPAPIERTISKPK |
| 123 | Mouse CH3 IgG2a | GSVRAPQVYVLPPPEEEMTKKQVTLTCMVTDFMPEDIYVEWTN |
| | (positions 224-330 of | NGKTELNYKNTEPVLDSDGSYFMYSKLRVEKKNWVERNSYSC |
| | UniProt: P01863, v1) | SVVHEGLHNHHTTKSFSRTPGK |
| 124 | Human/Mouse hybrid | ASTKGPSVFPLAPSSKSTSGGTAALGCLVKDYFPEPVTVSWNSG |
| | HC constant | ALTSGVHTFPAVLQSSGLYSLSSVVTVPSSSLGTQTYICNVNHKP |
| | (Human CH1 IgG1- | SNTKVDKKVEPKSCDKTHTCPPCPAPELLGGPSVFIFPPKIKDVL |
| | Human Hinge IgG1- | MISLSPIVTCVVVDVSEDDPDVQISWFVNNVEVHTAQTQTHRED |
| | Human CH2 IgG1- | YNSTLRVVSALPIQHQDWMSGKEFKCKVNNKDLPAPIERTISKP |
| | Mouse CH2 IgG2a- | KGSVRAPQVYVLPPPEEEMTKKQVTLTCMVTDFMPEDIYVEWT |
| | Mouse CH3 IgG2a) | NNGKTELNYKNTEPVLDSDGSYFMYSKLRVEKKNWVERNSYS |
| | | CSVVHEGLHNHHTTKSFSRTPGK |
| 125 | D011-1E11-5T2 VH | QVQLVQSGAEVKKPGASMKVSCKASGYSFTGSTMNWVRQAPG |
| | | QGLEWMGWINPYNGGTNYAQKFQGRVTMTRDTSISTAYMELS |
| | | RLRSDDTAVYYCASRGKNGYDGRDFDCWGQGTLVTVSS |
| 126 | D011-1E11-5T2 HC- | QVQLVQSGAEVKKPGASMKVSCKAS |
| | FR1 | |
| 127 | D011-1E11-5T2 HC- | MNWVRQAPGQGLEWMGW |
| | FR2 | |
| 128 | D011-1E11-5T2 HC- | NYAQKFQGRVTMTRDTSISTAYMELSRLRSDDTAVYYC |
| | FR3 | |
| 129 | D011-1E11-5T2; | WGQGTLVTVSS |
| | D011-7016-LD2_H0; | |
| | D011-7016-LD2_H1; | |
| | D011-7016-LD2_H3; | |
| | D011-7016-LD2_H4; | |
| | D011-7016-LD2_H5; | |
| | D011-7016- | |
| | LD2_HCON HC-FR4 | |
| 130 | D011-1E11-5T2 VL | EIVLTQSPATLSLSPGERATLSCKSTKSLLNSGGFSYLDWYQQKP |
| | | GQAPRLLIYLVSNRFTGIPARFSGSGSGTDFTLTISSLEPEDFAVY |
| | | YCFQSNYFPFTFGQGTKLEIK |
| 131 | D011-1E11-5T2 LC- | KSLLNSGGFSY |
| | CDR1 | |
| 132 | D011-1E11-5T2 LC- | EIVLTQSPATLSLSPGERATLSCKST |
| | FR1 | |
| 133 | D011-1E11-5T2 LC- | LDWYQQKPGQAPRLLIY |
| | FR2 | |
| 134 | D011-1E11-5T2 LC- | NRFTGIPARFSGSGSGTDFTLTISSLEPEDFAVYYC |
| | FR3 | |
| 135 | D011-1E11-5T2; | FGQGTKLEIK |
| | D011-7016-LD2_L0 | |
| | LC-FR4 | |
| 136 | D011-1E11-5T2 | QVQLVQSGAEVKKPGASMKVSCKASGYSFTGSTMNWVRQAPG |
| | hIgG1 HC | QGLEWMGWINPYNGGTNYAQKFQGRVTMTRDTSISTAYMELS |
| | | RLRSDDTAVYYCASRGKNGYDGRDFDCWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKKVEPKSCDKTHTCPPCPAPELLGGPSVFLFPPKPKDTLMIS |
| | | RTPEVTCVVVDVSHEDPEVKFNWYVDGVEVHNAKTKPREEQY |
| | | NSTYRVVSVLTVLHQDWLNGKEYKCKVSNKALPAPIEKTISKA |
| | | KGQPREPQVYTLPPSRDELTKNQVSLTCLVKGFYPSDIAVEWES |
| | | NGQPENNYKTTPPVLDSDGSFFLYSKLTVDKSRWQQGNVFSCS |
| | | VMHEALHNHYTQKSLSLSPGK |
| 137 | D011-1E11-5T2 κLC | EIVLTQSPATLSLSPGERATLSCKSTKSLLNSGGFSYLDWYQQKP |
| | | GQAPRLLIYLVSNRFTGIPARFSGSGSGTDFTLTISSLEPEDFAVY |
| | | YCFQSNYFPFTFGQGTKLEIKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 138 | D011-7016-LD1; | EVQLVESGPSLVKPSQTLSLICSVTGDAITSGYWNWVRKFPGNK |
| | D011-7016-LD2 | LEYMGYISYSGSAHYNPSLKSRISITRDTSKNQYYLQLNSMTTE |
| | IgG2a HC | DTATYYCASYSFAYWGQGTLVTVSAASTKGPSVFPLAPSSKSTS |
| | | GGTAALGCLVKDYFPEPVTVSWNSGALTSGVHTFPAVLQSSGL |
| | | YSLSSVVTVPSSSLGTQTYICNVNHKPSNTKVDKKVEPKSCDKT |
| | | HTCPPCPAPELLGGPSVFIFPPKIKDVLMISLSPIVTCVVVDVSED |
| | | DPDVQISWFVNNVEVHTAQTQTHREDYNSTLRVVSALPIQHQD |
| | | WMSGKEFKCKVNNKDLPAPIERTISKPKGSVRAPQVYVLPPPEE |
| | | EMTKKQVTLTCMVTDFMPEDIYVEWTNNGKTELNYKNTEPVL |
| | | DSDGSYFMYSKLRVEKKNWVERNSYSCSVVHEGLHNHHTTKS |
| | | FSRTPGK |
| 139 | D011-7016-LD1; | EVQLVESGPSLVKPSQTLSLICSVTGDAITSGYWNWVRKFPGNK |
| | D011-7016-LD2 | LEYMGYISYSGSAHYNPSLKSRISITRDTSKNQYYLQLNSMTTE |
| | hIgG1 HC | DTATYYCASYSFAYWGQGTLVTVSAASTKGPSVFPLAPSSKSTS |
| | | GGTAALGCLVKDYFPEPVTVSWNSGALTSGVHTFPAVLQSSGL |
| | | YSLSSVVTVPSSSLGTQTYICNVNHKPSNTKVDKKVEPKSCDKT |
| | | HTCPPCPAPELLGGPSVFLFPPKPKDTLMISRTPEVTCVVVDVSH |
| | | EDPEVKFNWYVDGVEVHNAKTKPREEQYNSTYRVVSVLTVLH |
| | | QDWLNGKEYKCKVSNKALPAPIEKTISKAKGQPREPQVYTLPPS |
| | | RDELTKNQVSLTCLVKGFYPSDIAVEWESNGQPENNYKTTPPVL |
| | | DSDGSFFLYSKLTVDKSRWQQGNVFSCSVMHEALHNHYTQKS |
| | | LSLSPGK |
| 140 | D011-7016-LD1 κLC | DIVMTQSPATLSVTPGDSVSLSCRAQQSIDNQLHWYQQRSHESP |
| | | RLLINYASQSISGIPSRFSGSGSGTDFTLSISSVETEDFGMYFCQQS |
| | | YGWPYTFGGGTKLEIKRTVAAPSVFIFPPSDEQLKSGTASVVCL |
| | | LNNFYPREAKVQWKVDNALQSGNSQESVTEQDSKDSTYSLSST |
| | | LTLSKADYEKHKVYACEVTHQGLSSPVTKSFNRGEC |
| 141 | D011-7016-LD1; | EVQLVESGPSLVKPSQTLSLICSVTGDAITSGYWNWVRKFPGNK |
| | D011-7016-LD2 VH | LEYMGYISYSGSAHYNPSLKSRISITRDTSKNQYYLQLNSMTTE |
| | | DTATYYCASYSFAYWGQGTLVTVSA |
| 142 | D011-7016-LD1; | GDAITSGY |
| | D011-7016-LD2; | |
| | D011-7016-LD2_H0 | |
| | HC-CDR1 | |
| 143 | D011-7016-LD1 VL | DIVMTQSPATLSVTPGDSVSLSCRAQQSIDNQLHWYQQRSHESP |
| | | RLLINYASQSISGIPSRFSGSGSGTDFTLSISSVETEDFGMYFCQQS |
| | | YGWPYTFGGGTKLEIK |
| 144 | D011-7016-LD1; | QSIDNQ |
| | D011-7016-LD2; | |
| | D011-7016-LD2_L0; | |
| | D011-7016-LD2_L1; | |
| | D011-7016-LD2_L2; | |
| | D011-7016-LD2_L3; | |
| | D011-7016-LD2_L4; | |
| | D011-7016- | |
| | LD2_LCON LC- | |
| | CDR1 | |
| 145 | D011-7016-LD1 LC- | DIVMTQSPATLSVTPGDSVSLSCRAQ |
| | FR1 | |
| 146 | D011-7016-LD2 κLC | DIVMTQSPATLSVTPGDSVSLSCRASQSIDNQLHWYQQRSHESP |
| | | RLLINYASQSISGIPSRFSGSGSGTDFTLSISSVETEDFGMYFCQQS |
| | | YGWPYTFGGGTKLEIKRTVAAPSVFIFPPSDEQLKSGTASVVCL |
| | | LNNFYPREAKVQWKVDNALQSGNSQESVTEQDSKDSTYSLSST |
| | | LTLSKADYEKHKVYACEVTHQGLSSPVTKSFNRGEC |
| 147 | D011-7016-LD2 VL | DIVMTQSPATLSVTPGDSVSLSCRASQSIDNQLHWYQQRSHESP |
| | | RLLINYASQSISGIPSRFSGSGSGTDFTLSISSVETEDFGMYFCQQS |
| | | YGWPYTFGGGTKLEIK |
| 148 | D011-7016-LD2 LC- | DIVMTQSPATLSVTPGDSVSLSCRAS |
| | FR1 | |
| 149 | D011-7016-LD1 | DIVMTQSPATLSVTPGDSVSLSCRAQQSIDNQLHWYQQRSHESP |
| | mκLC | RLLINYASQSISGIPSRFSGSGSGTDFTLSISSVETEDFGMYFCQQS |
| | | YGWPYTFGGGTKLEIKELVMTQSPLSLSVSLGDQASISCRSSQSL |
| | | VHTNGNTYLHWYLQKPGLSPKLLIYIVSNRFSGVPDRFSGSGSG |
| | | TDFTLKISRVEAEDLGVYFCSQVTHVPWTFGGGTKLEIKRADAA |
| | | PTVSIFPPSSEQLTSGGASVVCFLNNFYPKDINVKWKIDGSERQN |
| | | GVLNSWTDQDSKDSTYSMSSTLTLTKDEYERHNSYTCEATHKT |
| | | STSPIVKSFNRGEC |
| 150 | D011-7016-LD2 | DIVMTQSPATLSVTPGDSVSLSCRASQSIDNQLHWYQQRSHESP |
| | mκLC | RLLINYASQSISGIPSRFSGSGSGTDFTLSISSVETEDFGMYFCQQS |
| | | YGWPYTFGGGTKLEIKELVMTQSPLSLSVSLGDQASISCRSSQSL |
| | | VHTNGNTYLHWYLQKPGLSPKLLIYIVSNRFSGVPDRFSGSGSG |
| | | TDFTLKISRVEAEDLGVYFCSQVTHVPWTFGGGTKLEIKRADAA |
| | | PTVSIFPPSSEQLTSGGASVVCFLNNFYPKDINVKWKIDGSERQN |
| | | GVLNSWTDQDSKDSTYSMSSTLTLTKDEYERHNSYTCEATHKT |
| | | STSPIVKSFNRGEC |
| 151 | 7016 vs LD1/LD2 VH | EVQLVESGPSLVKPSQTLSLICSVTGDX114ITSGYWNWVRKFPG |
| | consensus | NKLEYMGYISYSGSAHYNPSLKSRISITRDTSKNQYYLQLNSMT |
| | | TEDTATYYCASYSFAYWGQGTLVTVSA |
| | | Wherein X₁₁₄ = S or A |
| 152 | 7016 vs LD1/LD2 | GDX₁₁₅ITSGY |
| | HC-CDR1 consensus | Wherein X₁₁₅ = S or A |
| 153 | 7016 vs LD1 vs LD2 | DIVMTQSPATLSVTPGDSVSLSCRAX₁₁₇QSIDNX₁₁₈LHWYQQRSH |
| | VL consensus | ESPRLLINYASQSISGIPSRFSGSGSGTDFTLSISSVETEDFGMYFC |
| | | QQSYGWPYTFGGGTKLEIK |
| | | Wherein X₁₁₇ = N, Q or S, X₁₁₈ = N or Q |
| 154 | 7016 vs LD1/LD2 LC- | QSIDNX₁₁₉ |
| | CDR1 consensus | Wherein X₁₁₉ = N or Q |
| 155 | 7016 vs LD1 vs LD2 | DIVMTQSPATLSVTPGDSVSLSCRAX₁₂₀ |
| | LC-FR1 consensus | Wherein X₁₂₀ = N, Q or S |
| 156 | 5T2 vs 1E11p VH | X₁₂₁VQLX₁₂₂QSGX₁₂₃EX₁₂₄X₁₂₅KPGASMKX₁₂₆SCKASGYSFTGSTM |
| | consensus | NWVX₁₂₇QX₁₂₈PGX₁₂₉X₁₃₀LEWX₁₃₁GX₁₃₂INPYNGGTX₁₃₃YX₁₃₄QKF |
| | | X₁₃₅GX₁₃₆X₁₃₇TX₁₃₈TX₁₃₉DX₁₄₀SX₁₄₁STAYMELX₁₄₂X₁₄₃LX₁₄₄SX₁₄₅D |
| | | X₁₄₆AVYYCASRGKNGYDGRDFDCWGQGTX₁₄₇VTVSS |
| | | Wherein X₁₂₁ = E or Q, X₁₂₂ = Q or V, X₁₂₃ = P or A, |
| | | X₁₂₄ = L or V, X₁₂₅ = V or K, X₁₂₆ = I or V, X₁₂₇ = K or R, |
| | | X₁₂₈ = S or A, X₁₂₉ = K or Q, X₁₃₀ = N or G, X₁₃₁ = I or M, |
| | | X₁₃₂ = L or W, X₁₃₃ = S or N, X₁₃₄ = N or A, X₁₃₅ = E or Q, |
| | | X₁₃₆ = K or R, X₁₃₇ = A or V, X₁₃₈ = L or M, X₁₃₉ = V or R, |
| | | X₁₄₀ = K or T, X₁₄₁ = S or I, X₁₄₂ = L or S, X₁₄₃ = S or R, |
| | | X₁₄₄ = T or R, X₁₄₅ = V or D, X₁₄₆ = S or T, X₁₄₇ = S or L |
| 157 | 5T2 vs 1E11p HC- | X₁₄₈VQLX₁₄₉QSGX₁₅₀EX₁₅₁X₁₅₂KPGASMKX₁₅₃SCKAS |
| | FR1 consensus | Wherein X₁₄₈ = E or Q, X₁₄₉ = Q or V, X₁₅₀ = P or A, X₁₅₁ = |
| | | L or V, X₁₅₂ = Vor K, X₁₅₃ = I or V |
| 158 | 5T2 vs 1E11p HC- | MNWVX₁₅₄QX₁₅₅PGX₁₅₆X₁₅₇LEWX₁₅₈GX₁₅₉ |
| | FR2 consensus | Wherein X₁₅₄ = K or R, X₁₅₅ = S or A, X₁₅₆ = K or Q, X₁₅₇ = |
| | | N or G, X₁₅₈ = I or M, X₁₅₉ = L or W |
| 159 | 5T2 vs 1E11p HC- | X₁₆₀YX₁₆₁QKFX₁₆₂GX₁₆₃X₁₆₄TX₁₆₅TX₁₆₆DX₁₆₇SX₁₆₈STAYMELX₁₆₉X₁₇₀ |
| | FR3 consensus | LX₁₇₁SX₁₇₂DX₁₇₃AVYYC |
| | | Wherein X₁₆₀ = S or N, X₁₆₁ = N or A, X₁₆₂ = E or Q, X₁₆₃ = |
| | | K or R, X₁₆₄ = A or V, X₁₆₅ = L or M, X₁₆₆ = Vor R, X₁₆₇ = |
| | | K or T, X₁₆₈ = S or I, X₁₆₉ = L or S, X₁₇₀ = S or R, |
| | | X₁₇₁ = T or R, X₁₇₂ = V or D, X₁₇₃ = S, A, or T |
| 160 | 5T2 vs 1E11p HC- | WGQGTX₁₇₄VTVSS |
| | FR4 consensus | Wherein X₁₇₄ = S or L |
| 161 | 5T2 vs 1E11p VL | X₁₇₅IVLTQSPX₁₇₆X₁₇₇LX₁₇₈X₁₇₉X₁₈₀X₁₈₁GX₁₈₂X₁₈₃AX₁₈₄X₁₈₅SCKSTK |
| | consensus | SLLNSGGFX₁₈₆YLDWYX₁₈₇QKPGQX₁₈₈PX₁₈₉LLIYLVSNRFX₁₉₀GX₁₉₁ |
| | | PX₁₉₂RFSGSGSGTDFTLX₁₉₃ISX₁₉₄X₁₉₅EX₁₉₆EDX₁₉₇X₁₉₈VYYCFQ |
| | | SNYFPFTFGX₁₉₉GTKLEIK |
| | | Wherein X₁₇₅ = D or E, X₁₇₆ = L or A, X₁₇₇ = S or T, X₁₇₈ = |
| | | P or S, X₁₇₉ = V or L, X₁₈₀ = N or S, X₁₈₁ = I or P, X₁₈₂ = |
| | | D or E, X₁₈₃ = Q or R, X₁₈₄ = S or T, X₁₈₅ = I or L, X₁₈₆ = |
| | | T or S, X₁₈₇ = L or Q, X₁₈₈ = S or A, X₁₈₉ = Q or R, X₁₉₀ = |
| | | S or T, X₁₉₁ = V or I, X₁₉₂ = D or A, X₁₉₃ = K or T, X₁₉₄ = |
| | | R or S, X₁₉₅ = V or L, X₁₉₆ = A or P, X₁₉₇ = L or F, X₁₉₈ = |
| | | G or A, X₁₉₉ = S or Q |
| 162 | 5T2 vs 1E1lp LC- | KSLLNSGGFX₂₀₀Y |
| | CDR1 consensus | Wherein X₂₀₀ = T or S |
| 163 | 5T2 vs 1E11p LC-FR1 | X₂₀₁IVLTQSPX₂₀₂X₂₀₃LX₂₀₄X₂₀₅X₂₀₆X₂₀₇GX₂₀₈X₂₀₉AX₂₁₀X₂₁₁SCKST |
| | consensus | Wherein X₂₀₁ = D or E, X₂₀₂ = L or A, X₂₀₃ = S or T, X₂₀₄ = |
| | | P or S, X₂₀₅ = V or L, X₂₀₆ = N or S, X₂₀₇ = I or P, X₂₀₈ = |
| | | D or E, X₂₀₉ = Q or R, X₂₁₀ = S or T, X₂₁₁ = I or L |
| 164 | 5T2 vs 1E11p LC-FR2 | LDWYX₂₁₂QKPGQX₂₁₃PX₂₁₄LLIY |
| | consensus | Wherein X₂₁₂ = L or Q, X₂₁₃ = S or A, X₂₁₄ = Q or R |
| 165 | 5T2 vs 1E11p LC-FR3 | NRFX₂₁₅GX₂₁₆PX₂₁₇RFSGSGSGTDFTLX₂₁₈ISX₂₁₉X₂₂₀EX₂₂₁EDX₂₂₂ |
| | consensus | X₂₂₃VYYC |
| | | Wherein X₂₁₅ = S or T, X₂₁₆ = V or I, X₂₁₇ = D or A, X₂₁₈ = |
| | | K or T, X₂₁₉= R or S, X₂₂₀ = V or L, X₂₂₁ = A or P, X₂₂₂ = |
| | | L or F, X₂₂₃ = G or A |
| 166 | 5T2 vs 1E11p LC-FR4 | FGX₂₂₄GTKLEIK |
| | consensus | Wherein X₂₂₄ = S or Q |
| 167 | D011-7016-LD2_H0 | QVQLQESGPGLVKPSETLSLTCTVSGDAITSGYWNWIRQPPGKG |
| | VH | LEYMGYISYSGSAHYNPSLKSRITISRDTSKNQYSLKLSSVTAAD |
| | | TAVYYCASYSFAYWGQGTLVTVSS |
| 168 | D011-7016-LD2_H0; | QVQLQESGPGLVKPSETLSLTCTVS |
| | D011-7016-LD2_H4 | |
| | HC-FR1 | |
| 169 | D011-7016-LD2_H0 | WNWIRQPPGKGLEYMGY |
| | HC-FR2 | |
| 170 | D011-7016-LD2_H0; | HYNPSLKSRITISRDTSKNQYSLKLSSVTAADTAVYYC |
| | D011-7016-LD2_H4; | |
| | D011-7016-LD2_H5 | |
| | HC-FR3 | |
| 171 | D011-7016-LD2_H1 | QVQLQESGPGLVKPSQTLSLTCTVSWDAITSGYWNWVRQHPG |
| | VH | KGLEYMGYISYSGSAHYNPSLKSRITISRDTSKNQYSLKLSSVTA |
| | | ADTATYYCASYSFAYWGQGTLVTVSS |
| 172 | D011-7016-LD2_H1; | WDAITSGY |
| | D011-7016-LD2_H3; | |
| | D011-7016-LD2_H4; | |
| | D011-7016-LD2_H5 | |
| | HC-CDR1 | |
| 173 | D011-7016-LD2_H1; | QVQLQESGPGLVKPSQTLSLTCTVS |
| | D011-7016-LD2_H5 | |
| | HC-FR1 | |
| 174 | D011-7016-LD2_H1 | WNWVRQHPGKGLEYMGY |
| | HC-FR2 | |
| 175 | D011-7016-LD2_H1 | HYNPSLKSRITISRDTSKNQYSLKLSSVTAADTATYYC |
| | HC-FR3 | |
| 176 | D011-7016-LD2_H3 | QVQLQESGPGLVKPSETLSLTCSVSWDAITSGYWNWVRQPPGN |
| | VH | KLEYMGYISYSGSAHYNPSLKSRITISRDTSKNQYSLTLTSMTAA |
| | | DTALYYCASYSFAYWGQGTLVTVSS |
| 178 | D011-7016-LD2_H3 | QVQLQESGPGLVKPSETLSLTCSVS |
| | HC-FR1 | |
| 179 | D011-7016-LD2_H3; | WNWVRQPPGNKLEYMGY |
| | D011-7016-LD2_H4 | |
| | HC-FR2 | |
| 180 | D011-7016-LD2_H3 | HYNPSLKSRITISRDTSKNQYSLTLTSMTAADTALYYC |
| | HC-FR3 | |
| 181 | D011-7016-LD2_H4 | QVQLQESGPGLVKPSETLSLTCTVSWDAITSGYWNWVRQPPGN |
| | VH | KLEYMGYISYSGSAHYNPSLKSRITISRDTSKNQYSLKLSSVTAA |
| | | DTAVYYCASYSFAYWGQGTLVTVSS |
| 182 | D011-7016-LD2_H5 | QVQLQESGPGLVKPSQTLSLTCTVSWDAITSGYWNWVRQHPG |
| | VH | NKLEYMGYISYSGSAHYNPSLKSRITISRDTSKNQYSLKLSSVTA |
| | | ADTAVYYCASYSFAYWGQGTLVTVSS |
| 183 | D011-7016-LD2_H5 | WNWVRQHPGNKLEYMGY |
| | HC-FR2 | |
| 184 | D011-7016- | QVQLQESGPGLVKPSX₂₂₅TLSLTCX₂₂₆VSX₂₂₇DAITSGYWNWX₂₂₈ |
| | LD2_HCON VH | RQX₂₂₉PGX₂₃₀X₂₃₁LEYMGYISYSGSAHYNPSLKSRITISRDTSKNQ |
| | | YSLKLSSVTAADTAX₂₃₂YYCASYSFAYWGQGTLVTVSS |
| | | Wherein X₂₂₅ = E or Q, X₂₂₆ = T or S, X₂₂₇ = W or G, X₂₂₈ = |
| | | V or I, X₂₂₉ = P or H, X₂₃₀ = K or N, X₂₃₁ = Go r K, X₂₃₂ = |
| | | V or T |
| 185 | D011-7016- | X₂₃₃DAITSGY |
| | LD2_HCON HC- | Wherein X₂₃₃ = W or G |
| | CDR1 | |
| 186 | D011-7016- | QVQLQESGPGLVKPSX₂₃₄TLSLTCX₂₃₅VS |
| | LD2_HCON HC-FR1 | Wherein X₂₃₄ = E or Q, X₂₃₅ = T or S |
| 187 | D011-7016- | WNWX₂₃₆RQX₂₃₇PGX₂₃₈X₂₃₉LEYMGY |
| | LD2_HCON HC-FR2 | Wherein X₂₃₆ = V or I, X₂₃₇ = P or H, X₂₃₈ = K or N, X₂₃₉ = |
| | | G or K |
| 188 | D011-7016- | HYNPSLKSRITISRDTSKNQYSLKLSSVTAADTAX₂₄₀YYC |
| | LD2_HCON HC-FR3 | Wherein X₂₄₀ = V or T |
| 189 | D011-7016-LD2_L0 | EIVMTQSPDFQSVTPKEKVTITCRASQSIDNQLHWYQQKPDQSP |
| | VL | KLLINYASQSISGVPSRFSGSGSGTDFTLTISSLEAEDAATYYCQQ |
| | | SYGWPYTFGQGTKLEIK |
| 190 | D011-7016-LD2_L0 | EIVMTQSPDFQSVTPKEKVTITCRAS |
| | LC-FRI | |
| 191 | D011-7016-LD2_L0 | LHWYQQKPDQSPKLLIN |
| | LC-FR2 | |
| 192 | D011-7016-LD2_L0 | QSISGVPSRFSGSGSGTDFTLTISSLEAEDAATYYC |
| | LC-FR3 | |
| 193 | D011-7016-LD2_L1 | DIVMTQSPPSLSVTPGEPASLSCRASQSIDNQLHWYLQKPGQSP |
| | VL | QLLINYASQSISGVTDRFSGSGSGTDFTLKISRVEAEDVGVYFCQ |
| | | QSYGWPYTFGGGTKVEIK |
| 194 | D011-7016-LD2_L1 | DIVMTQSPPSLSVTPGEPASLSCRAS |
| | LC-FR1 | |
| 195 | D011-7016-LD2_L1; | LHWYLQKPGQSPQLLIN |
| | D011-7016-LD2_L2 | |
| | LC-FR2 | |
| 196 | D011-7016-LD2_L1 | QSISGVTDRFSGSGSGTDFTLKISRVEAEDVGVYFC |
| | LC-FR3 | |
| 197 | D011-7016-LD2_L1; | FGGGTKVEIK |
| | D011-7016-LD2_L2; | |
| | D011-7016-LD2_L3 | |
| | LC-FR4 | |
| 198 | D011-7016-LD2_L2 | DIVMTQSPPSLSVTPGESASISCRASQSIDNQLHWYLQKPGQSPQ |
| | VL | LLINYASQSISGVTDRFSGSGSGTDFTLKISRVEAEDVGMYFCQQ |
| | | SYGWPYTFGGGTKVEIK |
| 199 | D011-7016-LD2_L2 | DIVMTQSPPSLSVTPGESASISCRAS |
| | LC-FR1 | |
| 200 | D011-7016-LD2_L2 | QSISGVTDRFSGSGSGTDFTLKISRVEAEDVGMYFC |
| | LC-FR3 | |
| 201 | D011-7016-LD2_L3 | DIQMTQSPSSLSASVGDRVTITCRASQSIDNQLHWYQQKPGKSP |
| | VL | NLLINYASQSISGVPSRFSGSGSGTDFTLTISSLQPEDFATYFCQQ |
| | | SYGWPYTFGGGTKVEIK |
| 202 | D011-7016-LD2_L3 | DIQMTQSPSSLSASVGDRVTITCRAS |
| | LC-FR1 | |
| 203 | D011-7016-LD2_L3 | LHWYQQKPGKSPNLLIN |
| | LC-FR2 | |
| 204 | D011-7016-LD2_L3 | QSISGVPSRFSGSGSGTDFTLTISSLQPEDFATYFC |
| | LC-FR3 | |
| 205 | D011-7016-LD2_L4 | EIVLTQSPDFQSVTPKEKVTITCRASQSIDNQLHWYQQKPDQSP |
| | VL | KLLIKYASQSISGVPSRFSGSGSGTDFTLTISSLEAEDAATYFCQQ |
| | | SYGWPYTFGGGTKLEIK |
| 206 | D011-7016-LD2_L4 | EIVLTQSPDFQSVTPKEKVTITCRAS |
| | LC-FR1 | |
| 207 | D011-7016-LD2_L4 | LHWYQQKPDQSPKLLIK |
| | LC-FR2 | |
| 208 | D011-7016-LD2_L4 | QSISGVPSRFSGSGSGTDFTLTISSLEAEDAATYFC |
| | LC-FR3 | |
| 209 | D011-7016- | X₂₄₁IX₂₄₂X₂₄₃TQSPX₂₄₄X₂₄₅X₂₄₆SX₂₄₇X₂₄₈X₂₄₉X₂₅₀X₂₅₁X₂₅₂X₂₅₃ |
| | LD2_LCON VL | X₂₅₄X₂₅₅X₂₅₆CRASQSIDNQLHWYX₂₅₇QKPX₂₅₈X₂₅₉SPX₂₆₀LLIX₂₆₁ |
| | | YASQSISGVX₂₆₂X₂₆₃RFSGSGSGTDFTLX₂₆₄ISX₂₆₅X₂₆₆X₂₆₇X₂₆₈ |
| | | EDX₂₆₉X₂₇₀X₂₇₁YX₂₇₂CQQSYGWPYTFGX₂₇₃GTKX₂₇₄EIK |
| | | Wherein X₂₄₁ = D or E, X₂₄₂ = V or Q, X₂₄₃ = M or L, X₂₄₄ = |
| | | P, D or S, X₂₄₅ = S or F, X₂₄₆ = L or Q, X₂₄₇ = V or A, |
| | | X₂₄₈ = T or S, X₂₄₉ = P or V, X₂₅₀ = G or K, X₂₅₁ = E or D, |
| | | X₂₅₂ = K, R, S or P, X₂₅₃ = V or A, X₂₅₄ = T or S, X₂₅₅ = L |
| | | or I, X₂₅₆ = T or S, X₂₅₇ = Q or L, X₂₅₈ = G or D, X₂₅₉ = Q |
| | | or K, X₂₆₀ = K, N or Q, X₂₆₁ = N or K, X₂₆₂ = P or T, X₂₆₃ = |
| | | S or D, X₂₆₄ = T or K, X₂₆₅ = R or S, X₂₆₆ = L or V, X₂₆₇ = |
| | | E or Q, X₂₆₈ = A or P, X₂₆₉ = V, For A, X₂₇₀ = A or G, |
| | | X₂₇₁ = T, V or M, X₂₇₂ = F or Y, X₂₇₃ = G or Q, X₂₇₄ = |
| | | L or V |
| 210 | D011-7016- | X₂₇₅IX₂₇₆X₂₇₇TQSPX₂₇₈X₂₇₉X₂₈₀SX₂₈₁X₂₈₂X₂₈₃X₂₈₄X₂₈₅X₂₈₆X₂₈₇X₂₈₈X₂₈₉ |
| | LD2_LCON LC-FR1 | X₂₉₀CRAS |
| | | Where in X₂₇₅ = D or E, X₂₇₆ = V or Q, X₂₇₇ = M or L, X₂₇₈ = |
| | | P, D or S, X₂₇₉ = S or F, X₂₈₀ = L or Q, X₂₈₁ = V or A, |
| | | X282 = T or S, X₂₈₃ = P or V, X₂₈₄ = G or K, X₂₈₅ = E or D, |
| | | X₂₈₆ = K, R, S or P, X₂₈₇ = V or A, X₂₈₈ = T or S, X₂₈₉ = |
| | | L or I, X₂₉₀ = T or S |
| 211 | D011-7016- | LHWYX₂₉₁QKPX₂₉₂X₂₉₃SPX₂₉₄LLIX₂₉₅ |
| | LD2_LCON LC-FR2 | Wherein X₂₉₁ = Q or L, X₂₉₂ = Gor D, X₂₉₃ = Q or K, X₂₉₄ = |
| | | K, N or Q, X₂₉₅ = N or K |
| 212 | D011-7016- | QSISGVX₂₉₆X₂₉₇RFSGSGSGTDFTLX₂₉₈ISX₂₉₉X₃₀₀X₃₀₁X₃₀₂EDX₃₀₃X₃₀₄ |
| | LD2_LCON LC-FR3 | X₃₀₅YX₃₀₆C |
| | | Wherein X₂₉₆ = P or T, X₂₉₇ = S or D, X₂₉₈ = T or K, X₂₉₉ = |
| | | R or S, X₃₀₀ = L or V, X₃₀₁ = E or Q, X₃₀₂ = A or P, X₃₀₃ = |
| | | V, F or A, X₃₀₄ = A or G, X₃₀₅ = T, V or M, X₃₀₆ = F or Y |
| 213 | D011-7016- | FGX₃₀₇GTKX₃₀₈EIK |
| | LD2_LCON LC-FR4 | Wherein X307 = G or Q, X308 = L or V |
| 214 | D011-7016-LD2 | QVQLQESGPGLVKPSX₃₀₉TLSLTCX₃₁₀VSX₃₁₁DAITSGYWNWX₃₁2 |
| | HCON hIgG1 HC | RQX₃₁₃PGX₃₁₄X₃₁₅LEYMGYISYSGSAHYNPSLKSRITISRDTSKNQ |
| | | YSLKLSSVTAADTAX₃₁₆YYCASYSFAYWGQGTLVTVSSASTKG |
| | | PSVFPLAPSSKSTSGGTAALGCLVKDYFPEPVTVSWNSGALTSG |
| | | VHTFPAVLQSSGLYSLSSVVTVPSSSLGTQTYICNVNHKPSNTK |
| | | VDKKVEPKSCDKTHTCPPCPAPELLGGPSVFLFPPKPKDTLMISR |
| | | TPEVTCVVVDVSHEDPEVKFNWYVDGVEVHNAKTKPREEQYN |
| | | STYRVVSVLTVLHQDWLNGKEYKCKVSNKALPAPIEKTISKAK |
| | | GQPREPQVYTLPPSRDELTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGK |
| | | Wherein X₃₀₉ = E or Q, X₃₁₀ = T or S, X₃₁₁ = W or G, X₃₁₂ = |
| | | V or I, X₃₁₃ = P or H, X₃₁₄ = K or N, X₃₁₅ = Gor K, X₃₁₆ = |
| | | V or T |
| 215 | D011-7016-LD2 H0 | QVQLQESGPGLVKPSETLSLTCTVSGDAITSGYWNWIRQPPGKG |
| | hIgGI HC | LEYMGYISYSGSAHYNPSLKSRITISRDTSKNQYSLKLSSVTAAD |
| | | TAVYYCASYSFAYWGQGTLVTVSSASTKGPSVFPLAPSSKSTSG |
| | | GTAALGCLVKDYFPEPVTVSWNSGALTSGVHTFPAVLQSSGLY |
| | | SLSSVVTVPSSSLGTQTYICNVNHKPSNTKVDKKVEPKSCDKTH |
| | | TCPPCPAPELLGGPSVFLFPPKPKDTLMISRTPEVTCVVVDVSHE |
| | | DPEVKFNWYVDGVEVHNAKTKPREEQYNSTYRVVSVLTVLHQ |
| | | DWLNGKEYKCKVSNKALPAPIEKTISKAKGQPREPQVYTLPPSR |
| | | DELTKNQVSLTCLVKGFYPSDIAVEWESNGQPENNYKTTPPVL |
| | | DSDGSFFLYSKLTVDKSRWQQGNVFSCSVMHEALHNHYTQKS |
| | | LSLSPGK |
| 216 | D011-7016-LD2 H1 | QVQLQESGPGLVKPSQTLSLTCTVSWDAITSGYWNWVRQHPG |
| | hIgG1 HC | KGLEYMGYISYSGSAHYNPSLKSRITISRDTSKNQYSLKLSSVTA |
| | | ADTATYYCASYSFAYWGQGTLVTVSSASTKGPSVFPLAPSSKST |
| | | SGGTAALGCLVKDYFPEPVTVSWNSGALTSGVHTFPAVLQSSG |
| | | LYSLSSVVTVPSSSLGTQTYICNVNHKPSNTKVDKKVEPKSCDK |
| | | THTCPPCPAPELLGGPSVFLFPPKPKDTLMISRTPEVTCVVVDVS |
| | | HEDPEVKFNWYVDGVEVHNAKTKPREEQYNSTYRVVSVLTVL |
| | | HQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVMHEALHNHYTQ |
| | | KSLSLSPGK |
| 217 | D011-7016-LD2 H3 | QVQLQESGPGLVKPSETLSLTCSVSWDAITSGYWNWVRQPPGN |
| | hIgG1 HC | KLEYMGYISYSGSAHYNPSLKSRITISRDTSKNQYSLTLTSMTAA |
| | | DTALYYCASYSFAYWGQGTLVTVSSASTKGPSVFPLAPSSKSTS |
| | | GGTAALGCLVKDYFPEPVTVSWNSGALTSGVHTFPAVLQSSGL |
| | | YSLSSVVTVPSSSLGTQTYICNVNHKPSNTKVDKKVEPKSCDKT |
| | | HTCPPCPAPELLGGPSVFLFPPKPKDTLMISRTPEVTCVVVDVSH |
| | | EDPEVKFNWYVDGVEVHNAKTKPREEQYNSTYRVVSVLTVLH |
| | | QDWLNGKEYKCKVSNKALPAPIEKTISKAKGQPREPQVYTLPPS |
| | | RDELTKNQVSLTCLVKGFYPSDIAVEWESNGQPENNYKTTPPVL |
| | | DSDGSFFLYSKLTVDKSRWQQGNVFSCSVMHEALHNHYTQKS |
| | | LSLSPGK |
| 218 | D011-7016-LD2 H4 | QVQLQESGPGLVKPSETLSLTCTVSWDAITSGYWNWVRQPPGN |
| | hIgG1 HC | KLEYMGYISYSGSAHYNPSLKSRITISRDTSKNQYSLKLSSVTAA |
| | | DTAVYYCASYSFAYWGQGTLVTVSSASTKGPSVFPLAPSSKSTS |
| | | GGTAALGCLVKDYFPEPVTVSWNSGALTSGVHTFPAVLQSSGL |
| | | YSLSSVVTVPSSSLGTQTYICNVNHKPSNTKVDKKVEPKSCDKT |
| | | HTCPPCPAPELLGGPSVFLFPPKPKDTLMISRTPEVTCVVVDVSH |
| | | EDPEVKFNWYVDGVEVHNAKTKPREEQYNSTYRVVSVLTVLH |
| | | QDWLNGKEYKCKVSNKALPAPIEKTISKAKGQPREPQVYTLPPS |
| | | RDELTKNQVSLTCLVKGFYPSDIAVEWESNGQPENNYKTTPPVL |
| | | DSDGSFFLYSKLTVDKSRWQQGNVFSCSVMHEALHNHYTQKS |
| | | LSLSPGK |
| 219 | D011-7016-LD2 H5 | QVQLQESGPGLVKPSQTLSLTCTVSWDAITSGYWNWVRQHPG |
| | hIgG1 HC | NKLEYMGYISYSGSAHYNPSLKSRITISRDTSKNQYSLKLSSVTA |
| | | ADTAVYYCASYSFAYWGQGTLVTVSSASTKGPSVFPLAPSSKST |
| | | SGGTAALGCLVKDYFPEPVTVSWNSGALTSGVHTFPAVLQSSG |
| | | LYSLSSVVTVPSSSLGTQTYICNVNHKPSNTKVDKKVEPKSCDK |
| | | THTCPPCPAPELLGGPSVFLFPPKPKDTLMISRTPEVTCVVVDVS |
| | | HEDPEVKFNWYVDGVEVHNAKTKPREEQYNSTYRVVSVLTVL |
| | | HQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVMHEALHNHYTQ |
| | | KSLSLSPGK |
| 220 | D011-7016-LD2 | X₃₁₇IX₃₁₈X₃₁₉TQSPX₃₂₀X₃₂₁X₃₂₂SX₃₂₃X₃₂₄X₃₂₅X₃₂₆X₃₂₇X₃₂₈X₃₂₉ |
| | LCON LC | X₃₃₀X₃₃₁X₃₃₂CRASQSIDNQLHWYX₃₃₃QKPX₃₃₄X₃₃₅SPX₃₃₆LLIX₃₃₇ |
| | | YASQSISGVX₃₃₈X₃₃₉RFSGSGSGTDFTLX₃₄₀ISX₃₄₁X₃₄₂X₃₄₃X₃₄₄EDX₃₄₅ |
| | | X₃₄₆X₃₄₇YX₃₄₈CQQSYGWPYTFGX₃₄₉GTKX₃₅₀EIKRTVAAPSVFIFPPS |
| | | DEQLKSGTASVVCLLNNFYPREAKVQWKVDNALQSGNSQESVTEQD |
| | | SKDSTYSLSSTLTLSKADYEKHKVYACEVTHQGLSSPVTKSFNRGEC |
| | | Wherein X₃₁₇ = D or E, X₃₁₈ = V or Q, X₃₁₉ = M or L, X₃₂₀ = |
| | | P, D or S, X₃₂₁ = S or F, X₃₂₂ = L or Q, X₃₂₃ = V or A, |
| | | X₃₂₄ = T or S, X₃₂₅ = P or V, X₃₂₆ = Gor K, X₃₂₇ = E or D, |
| | | X₃₂₈ = K, R, S or P, X₃₂₉ = V or A, X₃₃₀ = T or S, X₃₃₁ = |
| | | L or I, X₃₃₂ = T or S, X₃₃₃ = Q or L, X₃₃₄ = Gor D, X₃₃₅ = |
| | | Q or K, X₃₃₆ = K, N or Q, X₃₃₇ = N or K, X₃₃₈ = P or T, |
| | | X₃₃₉ = S or D, X₃₄₀ = T or K, X₃₄₁ = R or S, X₃₄₂ = L or V, |
| | | X₃₄₃ = E or Q, X₃₄₄ = A or P, X₃₄₅ = V, For A, X₃₄₆ = |
| | | A or G, X₃₄₇ = T, V or M, X₃₄₈ = F or Y, X₃₄₉ = G or Q, |
| | | X₃₅₀ = L or V |
| 221 | D011-7016-LD2 L0 | EIVMTQSPDFQSVTPKEKVTITCRASQSIDNQLHWYQQKPDQSP |
| | LC | KLLINYASQSISGVPSRFSGSGSGTDFTLTISSLEAEDAATYYCQQ |
| | | SYGWPYTFGQGTKLEIKRTVAAPSVFIFPPSDEQLKSGTASVVCL |
| | | LNNFYPREAKVQWKVDNALQSGNSQESVTEQDSKDSTYSLSST |
| | | LTLSKADYEKHKVYACEVTHQGLSSPVTKSFNRGEC |
| 222 | D011-7016-LD2 L1 | DIVMTQSPPSLSVTPGEPASLSCRASQSIDNQLHWYLQKPGQSP |
| | LC | QLLINYASQSISGVTDRFSGSGSGTDFTLKISRVEAEDVGVYFCQ |
| | | QSYGWPYTFGGGTKVEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |
| 223 | D011-7016-LD2 L2 | DIVMTQSPPSLSVTPGESASISCRASQSIDNQLHWYLQKPGQSPQ |
| | LC | LLINYASQSISGVTDRFSGSGSGTDFTLKISRVEAEDVGMYFCQQ |
| | | SYGWPYTFGGGTKVEIKRTVAAPSVFIFPPSDEQLKSGTASVVC |
| | | LLNNFYPREAKVQWKVDNALQSGNSQESVTEQDSKDSTYSLSS |
| | | TLTLSKADYEKHKVYACEVTHQGLSSPVTKSFNRGEC |
| 224 | D011-7016-LD2 L3 | DIQMTQSPSSLSASVGDRVTITCRASQSIDNQLHWYQQKPGKSP |
| | LC | NLLINYASQSISGVPSRFSGSGSGTDFTLTISSLQPEDFATYFCQQ |
| | | SYGWPYTFGGGTKVEIKRTVAAPSVFIFPPSDEQLKSGTASVVC |
| | | LLNNFYPREAKVQWKVDNALQSGNSQESVTEQDSKDSTYSLSS |
| | | TLTLSKADYEKHKVYACEVTHQGLSSPVTKSFNRGEC |
| 225 | D011-7016-LD2 L4 | EIVLTQSPDFQSVTPKEKVTITCRASQSIDNQLHWYQQKPDQSP |
| | LC | KLLIKYASQSISGVPSRFSGSGSGTDFTLTISSLEAEDAATYFCQQ |
| | | SYGWPYTFGGGTKLEIKRTVAAPSVFIFPPSDEQLKSGTASVVCL |
| | | LNNFYPREAKVQWKVDNALQSGNSQESVTEQDSKDSTYSLSST |
| | | LTLSKADYEKHKVYACEVTHQGLSSPVTKSFNRGEC |
| 226 | D011-7016; D011- | EVQLVESGPSLVKPSQTLSLICSVTGDX₁₁₄ITSGYWNWVRKFPG |
| | 7016-LD1; D011- | NKLEYMGYISYSGSAHYNPSLKSRISITRDTSKNQYYLQLNSMT |
| | 7016-LD2 consensus | TEDTATYYCASYSFAYWGQGTLVTVSAASTKGPSVFPLAPSSKS |
| | hIgG1 HC | TSGGTAALGCLVKDYFPEPVTVSWNSGALTSGVHTFPAVLQSS |
| | | GLYSLSSVVTVPSSSLGTQTYICNVNHKPSNTKVDKKVEPKSCD |
| | | KTHTCPPCPAPELLGGPSVFLFPPKPKDTLMISRTPEVTCVVVDV |
| | | SHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSRDELTKNQVSLTCLVKGFYPSDIAVEWESNGQPENNYKTTP |
| | | PVLDSDGSFFLYSKLTVDKSRWQQGNVFSCSVMHEALHNHYT |
| | | QKSLSLSPGK |
| | | Wherein X₁₁₄ = S or A |
| 227 | D011-7016; D011- | DIVMTQSPATLSVTPGDSVSLSCRAX₁₁₇QSIDNX₁₁₈LHWYQQRSH |
| | 7016-LD1; D011- | ESPRLLINYASQSISGIPSRFSGSGSGTDFTLSISSVETEDFGMYFC |
| | 7016-LD2 consensus | QQSYGWPYTFGGGTKLEIKRTVAAPSVFIFPPSDEQLKSGTASV |
| | LC | VCLLNNFYPREAKVQWKVDNALQSGNSQESVTEQDSKDSTYSL |
| | | SSTLTLSKADYEKHKVYACEVTHQGLSSPVTKSFNRGEC |
| | | Wherein X₁₁₇ = N, Q or S, X₁₁₈ = N or Q |
| 228 | D011-1E11p; D011- | X₁₂1VQLX₁₂₂QSGX₁₂₃EX₁₂₄X₁₂₅KPGASMKX₁₂₆SCKASGYSFTGSTM |
| | 1E11-5T2 consensus | NWVX₁₂₇QX₁₂₈PGX₁₂₉X₁₃₀LEWX₁₃₁GX₁₃₂INPYNGGTX₁₃₃YX₁₃₄QKF |
| | hIgG1 HC | X₁₃₅GX₁₃₆X₁₃₇TX₁₃₈TX₁₃₉DX₁₄₀SX₁₄₁STAYMELX₁₄₂X₁₄₃LX₁₄₄SX₁₄₅D |
| | | X₁₄₆AVYYCASRGKNGYDGRDFDCWGQGTX₁₄₇VTVSSASTKGPS |
| | | VFPLAPSSKSTSGGTAALGCLVKDYFPEPVTVSWNSGALTSGVH |
| | | TFPAVLQSSGLYSLSSVVTVPSSSLGTQTYICNVNHKPSNTKVD |
| | | KKVEPKSCDKTHTCPPCPAPELLGGPSVFLFPPKPKDTLMISRTP |
| | | EVTCVVVDVSHEDPEVKFNWYVDGVEVHNAKTKPREEQYNST |
| | | YRVVSVLTVLHQDWLNGKEYKCKVSNKALPAPIEKTISKAKGQ |
| | | PREPQVYTLPPSRDELTKNQVSLTCLVKGFYPSDIAVEWESNGQ |
| | | PENNYKTTPPVLDSDGSFFLYSKLTVDKSRWQQGNVFSCSVMH |
| | | EALHNHYTQKSLSLSPGK |
| | | Wherein X₁₂₁ = E or Q, X₁₂₂ = Q or V, X₁₂₃ = P or A, X₁₂₄ = |
| | | L or V, X₁₂₅ = V or K, X₁₂₆ = I or V, X₁₂₇ = K or R, X₁₂₈ = |
| | | S or A, X₁₂₉ = K or Q, X₁₃₀ = N or G, X₁₃₁ = I or M, X₁₃₂ = |
| | | L or W, X₁₃₃ = S or N, X₁₃₄ = N or A, X₁₃₅ = E or Q, X₁₃₆ = |
| | | K or R, X₁₃₇ = A or V, X₁₃₈ = L or M, X₁₃₉ = V or R, X₁₄₀ = |
| | | K or T, X₁₄₁ = S or I, X₁₄₂ = L or S, X₁₄₃ = S or R, X₁₄₄ = |
| | | T or R, X₁₄₅ = V or D, X₁₄₆ = S or T, X₁₄₇ = S or L |
| 229 | D011-1E11p: D011- | X₁₇₅IVLTQSPX₁₇₆X₁₇₇LX₁₇₈X₁₇₉X₁₈₀X₁₈₁GX₁₈₂X₁₈₃AX₁₈₄X₁₈₅SCKSTK |
| | 1E11-5T2 consensus | SLLNSGGFX₁₈₆YLDWYX₁₈₇QKPGQX₁₈₈PX₁₈₉LLIYLVSNRFX₁₉₀GX₁₉₁ |
| | LC | PX₁₉₂RFSGSGSGTDFTLX₁₉₃ISX₁₉₄X₁₉₅EX₁₉₆EDX₁₉₇X₁₉₈VYYCFQ |
| | | SNYFPFTFGX₁₉₉GTKLEIKRTVAAPSVFIFPPSDEQLKSGTASVVC |
| | | LLNNFYPREAKVQWKVDNALQSGNSQESVTEQDSKDSTYSLSS |
| | | TLTLSKADYEKHKVYACEVTHQGLSSPVTKSFNRGEC |
| | | Wherein X₁₇₅ = D or E, X₁₇₆ = L or A, X₁₇₇ = S or T, X₁₇₈ = |
| | | P or S, X₁₇₉ = V or L, X₁₈₀ = N or S, X₁₈₁ = I or P, X₁₈₂ = |
| | | D or E, X₁₈₃ = Q or R, X₁₈₄ = S or T, X₁₈₅ = I or L, X₁₈₆ = |
| | | T or S, X₁₈₇ = L or Q, X₁₈₈ = S or A, X₁₈₉ = Q or R, X₁₉₀ = |
| | | S or T, X₁₉₁ = V or I, X₁₉₂ = D or A, X₁₉₃ = K or T, X₁₉₄ = |
| | | R or S, X₁₉₅ = V or L, X₁₉₆ = A or P, X₁₉₇ = L or F, X₁₉₈ = |
| | | G or A, X₁₉₉ = S or Q |
| 230 | D011-7016-LD2 H0 | QVQLQESGPGLVKPSETLSLTCTVSGDAITSGYWNWIRQPPGKG |
| | IgG2a HC | LEYMGYISYSGSAHYNPSLKSRITISRDTSKNQYSLKLSSVTAAD |
| | | TAVYYCASYSFAYWGQGTLVTVSSASTKGPSVFPLAPSSKSTSG |
| | | GTAALGCLVKDYFPEPVTVSWNSGALTSGVHTFPAVLQSSGLY |
| | | SLSSVVTVPSSSLGTQTYICNVNHKPSNTKVDKKVEPKSCDKTH |
| | | TCPPCPAPELLGGPSVFIFPPKIKDVLMISLSPIVTCVVVDVSEDD |
| | | PDVQISWFVNNVEVHTAQTQTHREDYNSTLRVVSALPIQHQDW |
| | | MSGKEFKCKVNNKDLPAPIERTISKPKGSVRAPQVYVLPPPEEE |
| | | MTKKQVTLTCMVTDFMPEDIYVEWTNNGKTELNYKNTEPVLD |
| | | SDGSYFMYSKLRVEKKNWVERNSYSCSVVHEGLHNHHTTKSFS |
| | | RTPGK |
| 231 | D011-7016-LD2 H1 | QVQLQESGPGLVKPSQTLSLTCTVSWDAITSGYWNWVRQHPG |
| | IgG2a HC | KGLEYMGYISYSGSAHYNPSLKSRITISRDTSKNQYSLKLSSVTA |
| | | ADTATYYCASYSFAYWGQGTLVTVSSASTKGPSVFPLAPSSKST |
| | | SGGTAALGCLVKDYFPEPVTVSWNSGALTSGVHTFPAVLQSSG |
| | | LYSLSSVVTVPSSSLGTQTYICNVNHKPSNTKVDKKVEPKSCDK |
| | | THTCPPCPAPELLGGPSVFIFPPKIKDVLMISLSPIVTCVVVDVSE |
| | | DDPDVQISWFVNNVEVHTAQTQTHREDYNSTLRVVSALPIQHQ |
| | | DWMSGKEFKCKVNNKDLPAPIERTISKPKGSVRAPQVYVLPPPE |
| | | EEMTKKQVTLTCMVTDFMPEDIYVEWTNNGKTELNYKNTEPV |
| | | LDSDGSYFMYSKLRVEKKNWVERNSYSCSVVHEGLHNHHTTK |
| | | SFSRTPGK |
| 232 | D011-7016-LD2 H3 | QVQLQESGPGLVKPSETLSLTCSVSWDAITSGYWNWVRQPPGN |
| | IgG2a HC | KLEYMGYISYSGSAHYNPSLKSRITISRDTSKNQYSLTLTSMTAA |
| | | DTALYYCASYSFAYWGQGTLVTVSSASTKGPSVFPLAPSSKSTS |
| | | GGTAALGCLVKDYFPEPVTVSWNSGALTSGVHTFPAVLQSSGL |
| | | YSLSSVVTVPSSSLGTQTYICNVNHKPSNTKVDKKVEPKSCDKT |
| | | HTCPPCPAPELLGGPSVFIFPPKIKDVLMISLSPIVTCVVVDVSED |
| | | DPDVQISWFVNNVEVHTAQTQTHREDYNSTLRVVSALPIQHQD |
| | | WMSGKEFKCKVNNKDLPAPIERTISKPKGSVRAPQVYVLPPPEE |
| | | EMTKKQVTLTCMVTDFMPEDIYVEWTNNGKTELNYKNTEPVL |
| | | DSDGSYFMYSKLRVEKKNWVERNSYSCSVVHEGLHNHHTTKS |
| | | FSRTPGK |
| 233 | D011-7016-LD2 H4 | QVQLQESGPGLVKPSETLSLTCTVSWDAITSGYWNWVRQPPGN |
| | IgG2a HC | KLEYMGYISYSGSAHYNPSLKSRITISRDTSKNQYSLKLSSVTAA |
| | | DTAVYYCASYSFAYWGQGTLVTVSSASTKGPSVFPLAPSSKSTS |
| | | GGTAALGCLVKDYFPEPVTVSWNSGALTSGVHTFPAVLQSSGL |
| | | YSLSSVVTVPSSSLGTQTYICNVNHKPSNTKVDKKVEPKSCDKT |
| | | HTCPPCPAPELLGGPSVFIFPPKIKDVLMISLSPIVTCVVVDVSED |
| | | DPDVQISWFVNNVEVHTAQTQTHREDYNSTLRVVSALPIQHQD |
| | | WMSGKEFKCKVNNKDLPAPIERTISKPKGSVRAPQVYVLPPPEE |
| | | EMTKKQVTLTCMVTDFMPEDIYVEWTNNGKTELNYKNTEPVL |
| | | DSDGSYFMYSKLRVEKKNWVERNSYSCSVVHEGLHNHHTTKS |
| | | FSRTPGK |
| 234 | D011-7016-LD2 H5 | QVQLQESGPGLVKPSQTLSLTCTVSWDAITSGYWNWVRQHPG |
| | IgG2a HC | NKLEYMGYISYSGSAHYNPSLKSRITISRDTSKNQYSLKLSSVTA |
| | | ADTAVYYCASYSFAYWGQGTLVTVSSASTKGPSVFPLAPSSKST |
| | | SGGTAALGCLVKDYFPEPVTVSWNSGALTSGVHTFPAVLQSSG |
| | | LYSLSSVVTVPSSSLGTQTYICNVNHKPSNTKVDKKVEPKSCDK |
| | | THTCPPCPAPELLGGPSVFIFPPKIKDVLMISLSPIVTCVVVDVSE |
| | | DDPDVQISWFVNNVEVHTAQTQTHREDYNSTLRVVSALPIQHQ |
| | | DWMSGKEFKCKVNNKDLPAPIERTISKPKGSVRAPQVYVLPPPE |
| | | EEMTKKQVTLTCMVTDFMPEDIYVEWTNNGKTELNYKNTEPV |
| | | LDSDGSYFMYSKLRVEKKNWVERNSYSCSVVHEGLHNHHTTK |
| | | SFSRTPGK |
| 235 | D011-7016-LD2 | QVQLQESGPGLVKPSX₂₂₅TLSLTCX₂₂₆VSX₂₂₇DAITSGYWNWX₂₂₈ |
| | HCON IgG2a HC | RQX₂₂₉PGX₂₃₀X₂₃₁LEYMGYISYSGSAHYNPSLKSRITISRDTSKNQ |
| | | YSLKLSSVTAADTAX₂₃₂YYCASYSFAYWGQGTLVTVSSASTKG |
| | | PSVFPLAPSSKSTSGGTAALGCLVKDYFPEPVTVSWNSGALTSG |
| | | VHTFPAVLQSSGLYSLSSVVTVPSSSLGTQTYICNVNHKPSNTK |
| | | VDKKVEPKSCDKTHTCPPCPAPELLGGPSVFIFPPKIKDVLMISLS |
| | | PIVTCVVVDVSEDDPDVQISWFVNNVEVHTAQTQTHREDYNST |
| | | LRVVSALPIQHQDWMSGKEFKCKVNNKDLPAPIERTISKPKGSV |
| | | RAPQVYVLPPPEEEMTKKQVTLTCMVTDFMPEDIYVEWTNNG |
| | | KTELNYKNTEPVLDSDGSYFMYSKLRVEKKNWVERNSYSCSV |
| | | VHEGLHNHHTTKSFSRTPGK |
| | | Wherein X₂₂₅ = E or Q, X₂₂₆ = T or S, X₂₂₇ = W or G, X₂₂₈ = |
| | | V or I, X₂₂₉ = P or H, X₂₃₀ = K or N, X₂₃₁ = G or K, X₂₃₂ = |
| | | V or T |

TABLE A

| | Column A | | | Column B | | |
|---|---|---|---|---|---|---|
| | VH | | | VL | | |
| Antibody | HC-CDR1 | HC-CDR2 | HC-CDR3 | LC-CDR1 | LC-CDR2 | LC-CDR3 |
| D011-1E11p | SEQ ID NO: 6 | SEQ ID NO: 7 | SEQ ID NO: 8 | SEQ ID NO: 14 | LVS | SEQ ID NO: 16 |
| D011-6039 | SEQ ID NO: 22 | SEQ ID NO: 23 | SEQ ID NO: 24 | SEQ ID NO: 30 | YTS | SEQ ID NO: 32 |
| D011-6040 | SEQ ID NO: 38 | SEQ ID NO: 39 | SEQ ID NO: 40 | SEQ ID NO: 46 | AAS | SEQ ID NO: 48 |
| D011-7016 | SEQ ID NO: 54 | SEQ ID NO: 55 | SEQ ID NO: 56 | SEQ ID NO: 62 | YAS | SEQ ID NO: 64 |
| D011-1E11-5T2 | SEQ ID NO: 6 | SEQ ID NO: 7 | SEQ ID NO: 8 | SEQ ID NO: 131 | LVS | SEQ ID NO: 16 |
| D011-7016-LD1 | SEQ ID NO: 142 | SEQ ID NO: 55 | SEQ ID NO: 56 | SEQ ID NO: 144 | YAS | SEQ ID NO: 64 |
| D011-7016-LD2 | SEQ ID NO: 142 | SEQ ID NO: 55 | SEQ ID NO: 56 | SEQ ID NO: 144 | YAS | SEQ ID NO: 64 |
| D011-1E11p; D011-1E11- | SEQ ID NO: 6 | SEQ ID NO: 7 | SEQ ID NO: 8 | SEQ ID NO: 162 | LVS | SEQ ID NO: 16 |
| 5T2 consensus | | | | | | |
| D011-7016; D011-7016- | SEQ ID NO: 152 | SEQ ID NO: 55 | SEQ ID NO: 56 | SEQ ID NO: 154 | YAS | SEQ ID NO: 64 |
| LDI: D011-7016-LD2 | | | | | | |
| consensus | | | | | | |
| D011-7016-LD2 | SEQ ID NO: 185 | SEQ ID NO: 55 | SEQ ID NO: 56 | SEQ ID NO: 144 | YAS | SEQ ID NO: 64 |
| HCON/LCON | | | | | | |
| D011-7016-LD2 HCON/L0 | SEQ ID NO: 185 | SEQ ID NO: 55 | SEQ ID NO: 56 | SEQ ID NO: 144 | YAS | SEQ ID NO: 64 |
| D011-7016-LD2 HCON/L1 | SEQ ID NO: 185 | SEQ ID NO: 55 | SEQ ID NO: 56 | SEQ ID NO: 144 | YAS | SEQ ID NO: 64 |
| D011-7016-LD2 HCON/L2 | SEQ ID NO: 185 | SEQ ID NO: 55 | SEQ ID NO: 56 | SEQ ID NO: 144 | YAS | SEQ ID NO: 64 |
| D011-7016-LD2 HCON/L3 | SEQ ID NO: 185 | SEQ ID NO: 55 | SEQ ID NO: 56 | SEQ ID NO: 144 | YAS | SEQ ID NO: 64 |
| D011-7016-LD2 HCON/L4 | SEQ ID NO: 185 | SEQ ID NO: 55 | SEQ ID NO: 56 | SEQ ID NO: 144 | YAS | SEQ ID NO: 64 |
| D011-7016-LD2 H0/LCON | SEQ ID NO: 142 | SEQ ID NO: 55 | SEQ ID NO: 56 | SEQ ID NO: 144 | YAS | SEQ ID NO: 64 |
| D011-7016-LD2 H0/L0 | SEQ ID NO: 142 | SEQ ID NO: 55 | SEQ ID NO: 56 | SEQ ID NO: 144 | YAS | SEQ ID NO: 64 |
| D011-7016-LD2 H0/L1 | SEQ ID NO: 142 | SEQ ID NO: 55 | SEQ ID NO: 56 | SEQ ID NO: 144 | YAS | SEQ ID NO: 64 |
| D011-7016-LD2 H0/L2 | SEQ ID NO: 142 | SEQ ID NO: 55 | SEQ ID NO: 56 | SEQ ID NO: 144 | YAS | SEQ ID NO: 64 |
| D011-7016-LD2 H0/L3 | SEQ ID NO: 142 | SEQ ID NO: 55 | SEQ ID NO: 56 | SEQ ID NO: 144 | YAS | SEQ ID NO: 64 |
| D011-7016-LD2 H0/L4 | SEQ ID NO: 142 | SEQ ID NO: 55 | SEQ ID NO: 56 | SEQ ID NO: 144 | YAS | SEQ ID NO: 64 |
| D011-7016-LD2 H1/LCON | SEQ ID NO: 172 | SEQ ID NO: 55 | SEQ ID NO: 56 | SEQ ID NO: 144 | YAS | SEQ ID NO: 64 |
| D011-7016-LD2 H1/L0 | SEQ ID NO: 172 | SEQ ID NO: 55 | SEQ ID NO: 56 | SEQ ID NO: 144 | YAS | SEQ ID NO: 64 |
| D011-7016-LD2 H1/L1 | SEQ ID NO: 172 | SEQ ID NO: 55 | SEQ ID NO: 56 | SEQ ID NO: 144 | YAS | SEQ ID NO: 64 |
| D011-7016-LD2 H1/L2 | SEQ ID NO: 172 | SEQ ID NO: 55 | SEQ ID NO: 56 | SEQ ID NO: 144 | YAS | SEQ ID NO: 64 |
| D011-7016-LD2 H1/L3 | SEQ ID NO: 172 | SEQ ID NO: 55 | SEQ ID NO: 56 | SEQ ID NO: 144 | YAS | SEQ ID NO: 64 |
| D011-7016-LD2 H1/L4 | SEQ ID NO: 172 | SEQ ID NO: 55 | SEQ ID NO: 56 | SEQ ID NO: 144 | YAS | SEQ ID NO: 64 |
| D011-7016-LD2 H3/LCON | SEQ ID NO: 172 | SEQ ID NO: 55 | SEQ ID NO: 56 | SEQ ID NO: 144 | YAS | SEQ ID NO: 64 |
| D011-7016-LD2 H3/L0 | SEQ ID NO: 172 | SEQ ID NO: 55 | SEQ ID NO: 56 | SEQ ID NO: 144 | YAS | SEQ ID NO: 64 |
| D011-7016-LD2 H3/L1 | SEQ ID NO: 172 | SEQ ID NO: 55 | SEQ ID NO: 56 | SEQ ID NO: 144 | YAS | SEQ ID NO: 64 |
| D011-7016-LD2 H3/L2 | SEQ ID NO: 172 | SEQ ID NO: 55 | SEQ ID NO: 56 | SEQ ID NO: 144 | YAS | SEQ ID NO: 64 |
| D011-7016-LD2 H3/L3 | SEQ ID NO: 172 | SEQ ID NO: 55 | SEQ ID NO: 56 | SEQ ID NO: 144 | YAS | SEQ ID NO: 64 |
| D011-7016-LD2 H3/L4 | SEQ ID NO: 172 | SEQ ID NO: 55 | SEQ ID NO: 56 | SEQ ID NO: 144 | YAS | SEQ ID NO: 64 |
| D011-7016-LD2 H4/LCON | SEQ ID NO: 172 | SEQ ID NO: 55 | SEQ ID NO: 56 | SEQ ID NO: 144 | YAS | SEQ ID NO: 64 |
| D011-7016-LD2 H4/L0 | SEQ ID NO: 172 | SEQ ID NO: 55 | SEQ ID NO: 56 | SEQ ID NO: 144 | YAS | SEQ ID NO: 64 |
| D011-7016-LD2 H4/L1 | SEQ ID NO: 172 | SEQ ID NO: 55 | SEQ ID NO: 56 | SEQ ID NO: 144 | YAS | SEQ ID NO: 64 |
| D011-7016-LD2 H4/L2 | SEQ ID NO: 172 | SEQ ID NO: 55 | SEQ ID NO: 56 | SEQ ID NO: 144 | YAS | SEQ ID NO: 64 |
| D011-7016-LD2 H4/L3 | SEQ ID NO: 172 | SEQ ID NO: 55 | SEQ ID NO: 56 | SEQ ID NO: 144 | YAS | SEQ ID NO: 64 |
| D011-7016-LD2 H4/L4 | SEQ ID NO: 172 | SEQ ID NO: 55 | SEQ ID NO: 56 | SEQ ID NO: 144 | YAS | SEQ ID NO: 64 |
| D011-7016-LD2 H5/LCON | SEQ ID NO: 172 | SEQ ID NO: 55 | SEQ ID NO: 56 | SEQ ID NO: 144 | YAS | SEQ ID NO: 64 |
| D011-7016-LD2 H5/L0 | SEQ ID NO: 172 | SEQ ID NO: 55 | SEQ ID NO: 56 | SEQ ID NO: 144 | YAS | SEQ ID NO: 64 |
| D011-7016-LD2 H5/L1 | SEQ ID NO: 172 | SEQ ID NO: 55 | SEQ ID NO: 56 | SEQ ID NO: 144 | YAS | SEQ ID NO: 64 |
| D011-7016-LD2 H5/L2 | SEQ ID NO: 172 | SEQ ID NO: 55 | SEQ ID NO: 56 | SEQ ID NO: 144 | YAS | SEQ ID NO: 64 |
| D011-7016-LD2 H5/L3 | SEQ ID NO: 172 | SEQ ID NO: 55 | SEQ ID NO: 56 | SEQ ID NO: 144 | YAS | SEQ ID NO: 64 |
| D011-7016-LD2 H5/L4 | SEQ ID NO: 172 | SEQ ID NO: 55 | SEQ ID NO: 56 | SEQ ID NO: 144 | YAS | SEQ ID NO: 64 |

TABLE B

| | Column A | | | | Column B | | | |
|---|---|---|---|---|---|---|---|---|
| | VH | | | | VL | | | |
| Antibody | HC-FR1 | HC-FR2 | HC-FR3 | HC-FR4 | LC-FR1 | LC-FR2 | LC-FR3 | LC-FR4 |
| D011-1E11p | SEQ ID NO: 9 | SEQ ID NO: 10 | SEQ ID NO: 11 | SEQ ID NO: 12 | SEQ ID NO: 17 | SEQ ID NO: 18 | SEQ ID NO: 19 | SEQ ID NO: |
| | | | | | | | | 20 |
| D011-6039 | SEQ ID NO: 25 | SEQ ID NO: 26 | SEQ ID NO: 27 | SEQ ID NO: 28 | SEQ ID NO: 33 | SEQ ID NO: 34 | SEQ ID NO: 35 | SEQ ID NO: |
| | | | | | | | | 36 |
| D011-6040 | SEQ ID NO: 41 | SEQ ID NO: 42 | SEQ ID NO: 43 | SEQ ID NO: 44 | SEQ ID NO: 49 | SEQ ID NO: 50 | SEQ ID NO: 51 | SEQ ID NO: |
| | | | | | | | | 52 |
| D011-7016 | SEQ ID NO: 57 | SEQ ID NO: 58 | SEQ ID NO: 59 | SEQ ID NO: 60 | SEQ ID NO: 65 | SEQ ID NO: 66 | SEQ ID NO: 67 | SEQ ID NO: |
| | | | | | | | | 68 |
| D011-1E11-5T2 | SEQ ID NO: | SEQ ID NO: | SEQ ID NO: | SEQ ID NO: | SEQ ID NO: | SEQ ID NO: | SEQ ID NO: | SEQ ID NO: |
| | 126 | 127 | 128 | 129 | 132 | 133 | 134 | 135 |
| D011-1E11p; | SEQ ID NO: | SEQ ID NO: | SEQ ID NO: | SEQ ID NO: | SEQ ID NO: | SEQ ID NO: | SEQ ID NO: | SEQ ID NO: |
| D011-1E11-5T2 | 157 | 158 | 159 | 160 | 163 | 164 | 165 | 166 |
| consensus | | | | | | | | |
| D011-7016-LD1 | SEQ ID NO: 57 | SEQ ID NO: 58 | SEQ ID NO: 59 | SEQ ID NO: 60 | SEQ ID NO: | SEQ ID NO: 66 | SEQ ID NO: 67 | SEQ ID NO: |
| | | | | | 145 | | | 68 |
| D011-7016-LD2 | SEQ ID NO: 57 | SEQ ID NO: 58 | SEQ ID NO: 59 | SEQ ID NO: 60 | SEQ ID NO: | SEQ ID NO: 66 | SEQ ID NO: 67 | SEQ ID NO: |
| | | | | | 148 | | | 68 |
| D011-7016: | SEQ ID NO: 57 | SEQ ID NO: 58 | SEQ ID NO: 59 | SEQ ID NO: 60 | SEQ ID NO: | SEQ ID NO: 66 | SEQ ID NO: 67 | SEQ ID NO |
| D011-7016-LD1; | | | | | 155 | | | 68 |
| D011-7016-LD2 | | | | | | | | |
| consensus | | | | | | | | |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| HCON/LCON | 186 | 187 | 188 | 129 | 210 | 211 | 212 | 213 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| HCON/L0 | 186 | 187 | 188 | 129 | 190 | 191 | 192 | 135 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| HCON/L1 | 186 | 187 | 188 | 129 | 194 | 195 | 196 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| HCON/L2 | 186 | 187 | 188 | 129 | 199 | 195 | 200 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| HCON/L3 | 186 | 187 | 188 | 129 | 202 | 203 | 204 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| HCON/L4 | 186 | 187 | 188 | 129 | 206 | 207 | 208 | 68 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H0/LCON | 168 | 169 | 170 | 129 | 210 | 211 | 212 | 213 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H0/L0 | 168 | 169 | 170 | 129 | 190 | 191 | 192 | 135 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H0/L1 | 168 | 169 | 170 | 129 | 194 | 195 | 196 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H0/L2 | 168 | 169 | 170 | 129 | 199 | 195 | 200 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H0/L3 | 168 | 169 | 170 | 129 | 202 | 203 | 204 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H0/L4 | 168 | 169 | 170 | 129 | 206 | 207 | 208 | 68 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H1/LCON | 173 | 174 | 175 | 129 | 210 | 211 | 212 | 213 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H1/L0 | 173 | 174 | 175 | 129 | 190 | 191 | 192 | 135 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H1/L1 | 173 | 174 | 175 | 129 | 194 | 195 | 196 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H1/L2 | 173 | 174 | 175 | 129 | 199 | 195 | 200 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H1/L3 | 173 | 174 | 175 | 129 | 202 | 203 | 204 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H1/L4 | 173 | 174 | 175 | 129 | 206 | 207 | 208 | 68 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H3/LCON | 178 | 179 | 180 | 129 | 210 | 211 | 212 | 213 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H3/L0 | 178 | 179 | 180 | 129 | 190 | 191 | 192 | 135 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H3/L1 | 178 | 179 | 180 | 129 | 194 | 195 | 196 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H3/L2 | 178 | 179 | 180 | 129 | 199 | 195 | 200 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H3/L3 | 178 | 179 | 180 | 129 | 202 | 203 | 204 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H3/L4 | 178 | 179 | 180 | 129 | 206 | 207 | 208 | 68 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H4/LCON | 168 | 179 | 170 | 129 | 210 | 211 | 212 | 213 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H4/L0 | 168 | 179 | 170 | 129 | 190 | 191 | 192 | 135 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H4/L1 | 168 | 179 | 170 | 129 | 194 | 195 | 196 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H4/L2 | 168 | 179 | 170 | 129 | 199 | 195 | 200 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H4/L3 | 168 | 179 | 170 | 129 | 202 | 203 | 204 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H4/L4 | 168 | 179 | 170 | 129 | 206 | 207 | 208 | 68 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H5/LCON | 173 | 183 | 170 | 129 | 210 | 211 | 212 | 213 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H5/L0 | 173 | 183 | 170 | 129 | 190 | 191 | 192 | 135 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H5/L1 | 173 | 183 | 170 | 129 | 194 | 195 | 196 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H5/L2 | 173 | 183 | 170 | 129 | 199 | 195 | 200 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H5/L3 | 173 | 183 | 170 | 129 | 202 | 203 | 204 | 197 |
| D011-7016-LD2 | SEQ ID NO: | SEQ ID NO: | SEQ ID NO: | SEQ ID NO: | SEQ ID NO: | SEQ ID NO: | SEQ ID NO: | SEQ ID NO: |
| H5/L4 | 173 | 183 | 170 | 129 | 206 | 207 | 208 | 68 |

TABLE C

| | Column A | Column B |
|---|---|---|
| Antibody | VH | VL |
| D011-1E11p | SEQ ID NO: 5 | SEQ ID NO: 13 |
| D011-6039 | SEQ ID NO: 21 | SEQ ID NO: 29 |
| D011-6040 | SEQ ID NO: 37 | SEQ ID NO: 45 |
| D011-7016 | SEQ ID NO: 53 | SEQ ID NO: 61 |
| D011-1E11-5T2 | SEQ ID NO: 125 | SEQ ID NO: 130 |
| D011-7016-LD1 | SEQ ID NO: 141 | SEQ ID NO: 143 |
| D011-7016-LD2 | SEQ ID NO: 141 | SEQ ID NO: 147 |
| D011-1E11p; D011-1E11-5T2 | SEQ ID NO: 156 | SEQ ID NO: 161 |
| consensus | | |
| D011-7016; D011-7016-LD1; | SEQ ID NO: 151 | SEQ ID NO: 153 |
| D011-7016-LD2 consensus | | |
| D011-7016-LD2 HCON/LCON | SEQ ID NO: 184 | SEQ ID NO: 209 |
| D011-7016-LD2 HCON/L0 | SEQ ID NO: 184 | SEQ ID NO: 189 |
| D011-7016-LD2 HCON/L1 | SEQ ID NO: 184 | SEQ ID NO: 193 |
| D011-7016-LD2 HCON/L2 | SEQ ID NO: 184 | SEQ ID NO: 198 |
| D011-7016-LD2 HCON/L3 | SEQ ID NO: 184 | SEQ ID NO: 201 |
| D011-7016-LD2 HCON/L4 | SEQ ID NO: 184 | SEQ ID NO: 205 |
| D011-7016-LD2 H0/LCON | SEQ ID NO: 167 | SEQ ID NO: 209 |
| D011-7016-LD2 H0/L0 | SEQ ID NO: 167 | SEQ ID NO: 189 |
| D011-7016-LD2 H0/L1 | SEQ ID NO: 167 | SEQ ID NO: 193 |
| D011-7016-LD2 H0/L2 | SEQ ID NO: 167 | SEQ ID NO: 198 |
| D011-7016-LD2 H0/L3 | SEQ ID NO: 167 | SEQ ID NO: 201 |
| D011-7016-LD2 H0/L4 | SEQ ID NO: 167 | SEQ ID NO: 205 |
| D011-7016-LD2 H1/LCON | SEQ ID NO: 171 | SEQ ID NO: 209 |
| D011-7016-LD2 H1/L0 | SEQ ID NO: 171 | SEQ ID NO: 189 |
| D011-7016-LD2 H1/L1 | SEQ ID NO: 171 | SEQ ID NO: 193 |
| D011-7016-LD2 H1/L2 | SEQ ID NO: 171 | SEQ ID NO: 198 |
| D011-7016-LD2 H1/L3 | SEQ ID NO: 171 | SEQ ID NO: 201 |
| D011-7016-LD2 H1/L4 | SEQ ID NO: 171 | SEQ ID NO: 205 |
| D011-7016-LD2 H3/LCON | SEQ ID NO: 176 | SEQ ID NO: 209 |
| D011-7016-LD2 H3/L0 | SEQ ID NO: 176 | SEQ ID NO: 189 |
| D011-7016-LD2 H3/L1 | SEQ ID NO: 176 | SEQ ID NO: 193 |
| D011-7016-LD2 H3/L2 | SEQ ID NO: 176 | SEQ ID NO: 198 |
| D011-7016-LD2 H3/L3 | SEQ ID NO: 176 | SEQ ID NO: 201 |
| D011-7016-LD2 H3/L4 | SEQ ID NO: 176 | SEQ ID NO: 205 |
| D011-7016-LD2 H4/LCON | SEQ ID NO: 181 | SEQ ID NO: 209 |
| D011-7016-LD2 H4/L0 | SEQ ID NO: 181 | SEQ ID NO: 189 |
| D011-7016-LD2 H4/L1 | SEQ ID NO: 181 | SEQ ID NO: 193 |
| D011-7016-LD2 H4/L2 | SEQ ID NO: 181 | SEQ ID NO: 198 |
| D011-7016-LD2 H4/L3 | SEQ ID NO: 181 | SEQ ID NO: 201 |
| D011-7016-LD2 H4/L4 | SEQ ID NO: 181 | SEQ ID NO: 205 |
| D011-7016-LD2 H5/LCON | SEQ ID NO: 182 | SEQ ID NO: 209 |
| D011-7016-LD2 H5/L0 | SEQ ID NO: 182 | SEQ ID NO: 189 |
| D011-7016-LD2 H5/L1 | SEQ ID NO: 182 | SEQ ID NO: 193 |
| D011-7016-LD2 H5/L2 | SEQ ID NO: 182 | SEQ ID NO: 198 |
| D011-7016-LD2 H5/L3 | SEQ ID NO: 182 | SEQ ID NO: 201 |
| D011-7016-LD2 H5/L4 | SEQ ID NO: 182 | SEQ ID NO: 205 |

TABLE D

| | Column A | Column B |
|---|---|---|
| Name | Heavy Chain | Light Chain |
| D011-1E11p hIgG1 | SEQ ID NO: 86 | SEQ ID NO: 90 |
| D011-1E11p IgG2a | SEQ ID NO: 95 | SEQ ID NO: 90 |
| D011-6039 hIgG1 | SEQ ID NO: 87 | SEQ ID NO: 91 |
| D011-6039 IgG2a | SEQ ID NO: 96 | SEQ ID NO: 91 |
| D011-6040 hIgG1 | SEQ ID NO: 88 | SEQ ID NO: 92 |
| D011-6040 IgG2a | SEQ ID NO: 97 | SEQ ID NO: 92 |
| D011-7016 hIgG1 | SEQ ID NO: 89 | SEQ ID NO: 93 |
| D011-7016 IgG2a | SEQ ID NO: 98 | SEQ ID NO: 93 |
| D011-1E11-5T2 hIgG1 | SEQ ID NO: 136 | SEQ ID NO: 137 |
| D011-1E11-5T2 IgG2a | SEQ ID NO: 99 | SEQ ID NO: 137 |
| D011-7016-LD1 hIgG1 | SEQ ID NO: 139 | SEQ ID NO: 140 |
| D011-7016-LD1 IgG2a | SEQ ID NO: 138 | SEQ ID NO: 140 |
| D011-7016-LD2 hIgG1 | SEQ ID NO: 139 | SEQ ID NO: 146 |
| D011-7016-LD2 IgG2a | SEQ ID NO: 138 | SEQ ID NO: 146 |
| D011-1E11p; D011-1E11-5T2 | SEQ ID NO: 228 | SEQ ID NO: 229 |
| consensus hIgG1 | | |
| D011-7016; D011-7016-LD1; | SEQ ID NO: 226 | SEQ ID NO: 227 |
| D011-7016-LD2 consensus hIgG1 | | |
| D011-7016-LD2 HCON/LCON | SEQ ID NO: 214 | SEQ ID NO: 220 |
| hIgG1 | | |
| D011-7016-LD2 HCON/LCON | SEQ ID NO: 235 | SEQ ID NO: 220 |
| IgG2a | | |
| D011-7016-LD2 HCON/L0 hIgG1 | SEQ ID NO: 214 | SEQ ID NO: 221 |
| D011-7016-LD2 HCON/L0 IgG2a | SEQ ID NO: 235 | SEQ ID NO: 221 |
| D011-7016-LD2 HCON/L1 hIgG1 | SEQ ID NO: 214 | SEQ ID NO: 222 |
| D011-7016-LD2 HCON/L1 IgG2a | SEQ ID NO: 235 | SEQ ID NO: 222 |
| D011-7016-LD2 HCON/L2 hIgG1 | SEQ ID NO: 214 | SEQ ID NO: 223 |
| D011-7016-LD2 HCON/L2 IgG2a | SEQ ID NO: 235 | SEQ ID NO: 223 |
| D011-7016-LD2 HCON/L3 hIgG1 | SEQ ID NO: 214 | SEQ ID NO: 224 |
| D011-7016-LD2 HCON/L3 IgG2a | SEQ ID NO: 235 | SEQ ID NO: 224 |
| D011-7016-LD2 HCON/L4 hIgG1 | SEQ ID NO: 214 | SEQ ID NO: 225 |
| D011-7016-LD2 HCON/L4 IgG2a | SEQ ID NO: 235 | SEQ ID NO: 225 |
| D011-7016-LD2 H0/LCON hIgG1 | SEQ ID NO: 215 | SEQ ID NO: 220 |
| D011-7016-LD2 H0/LCON IgG2a | SEQ ID NO: 230 | SEQ ID NO: 220 |
| D011-7016-LD2 H0/L0 hIgG1 | SEQ ID NO: 215 | SEQ ID NO: 221 |
| D011-7016-LD2 H0/L0 IgG2a | SEQ ID NO: 230 | SEQ ID NO: 221 |
| D011-7016-LD2 H0/L1 hIgG1 | SEQ ID NO: 215 | SEQ ID NO: 222 |
| D011-7016-LD2 H0/L1 IgG2a | SEQ ID NO: 230 | SEQ ID NO: 222 |
| D011-7016-LD2 H0/L2 hIgG1 | SEQ ID NO: 215 | SEQ ID NO: 223 |
| D011-7016-LD2 H0/L2 IgG2a | SEQ ID NO: 230 | SEQ ID NO: 223 |
| D011-7016-LD2 H0/L3 hIgG1 | SEQ ID NO: 215 | SEQ ID NO: 224 |
| D011-7016-LD2 H0/L3 IgG2a | SEQ ID NO: 230 | SEQ ID NO: 224 |
| D011-7016-LD2 H0/L4 hIgG1 | SEQ ID NO: 215 | SEQ ID NO: 225 |
| D011-7016-LD2 H0/L4 IgG2a | SEQ ID NO: 230 | SEQ ID NO: 225 |
| D011-7016-LD2 H1/LCON hIgG1 | SEQ ID NO: 216 | SEQ ID NO: 220 |
| D011-7016-LD2 H1/LCON IgG2a | SEQ ID NO: 231 | SEQ ID NO: 220 |
| D011-7016-LD2 H1/L0 hIgG1 | SEQ ID NO: 216 | SEQ ID NO: 221 |
| D011-7016-LD2 H1/L0 IgG2a | SEQ ID NO: 231 | SEQ ID NO: 221 |
| D011-7016-LD2 H1/L1 hIgG1 | SEQ ID NO: 216 | SEQ ID NO: 222 |
| D011-7016-LD2 H1/L1 IgG2a | SEQ ID NO: 231 | SEQ ID NO: 222 |
| D011-7016-LD2 H1/L2 hIgG1 | SEQ ID NO: 216 | SEQ ID NO: 223 |
| D011-7016-LD2 H1/L2 IgG2a | SEQ ID NO: 231 | SEQ ID NO: 223 |
| D011-7016-LD2 H1/L3 hIgG1 | SEQ ID NO: 216 | SEQ ID NO: 224 |
| D011-7016-LD2 H1/L3 IgG2a | SEQ ID NO: 231 | SEQ ID NO: 224 |
| D011-7016-LD2 H1/L4 hIgG1 | SEQ ID NO: 216 | SEQ ID NO: 225 |
| D011-7016-LD2 H1/L4 IgG2a | SEQ ID NO: 231 | SEQ ID NO: 225 |
| D011-7016-LD2 H3/LCON hIgG1 | SEQ ID NO: 217 | SEQ ID NO: 220 |
| D011-7016-LD2 H3/LCON IgG2a | SEQ ID NO: 232 | SEQ ID NO: 220 |
| D011-7016-LD2 H3/L0 hIgG1 | SEQ ID NO: 217 | SEQ ID NO: 221 |
| D011-7016-LD2 H3/L0 IgG2a | SEQ ID NO: 232 | SEQ ID NO: 221 |
| D011-7016-LD2 H3/L1 hIgG1 | SEQ ID NO: 217 | SEQ ID NO: 222 |
| D011-7016-LD2 H3/L1 IgG2a | SEQ ID NO: 232 | SEQ ID NO: 222 |
| D011-7016-LD2 H3/L2 hIgG1 | SEQ ID NO: 217 | SEQ ID NO: 223 |
| D011-7016-LD2 H3/L2 IgG2a | SEQ ID NO: 232 | SEQ ID NO: 223 |
| D011-7016-LD2 H3/L3 hIgG1 | SEQ ID NO: 217 | SEQ ID NO: 224 |
| D011-7016-LD2 H3/L3 IgG2a | SEQ ID NO: 232 | SEQ ID NO: 224 |
| D011-7016-LD2 H3/L4 hIgG1 | SEQ ID NO: 217 | SEQ ID NO: 225 |
| D011-7016-LD2 H3/L4 IgG2a | SEQ ID NO: 232 | SEQ ID NO: 225 |
| D011-7016-LD2 H4/LCON hIgG1 | SEQ ID NO: 218 | SEQ ID NO: 220 |
| D011-7016-LD2 H4/LCON IgG2a | SEQ ID NO: 233 | SEQ ID NO: 220 |
| D011-7016-LD2 H4/L0 hIgG1 | SEQ ID NO: 218 | SEQ ID NO: 221 |
| D011-7016-LD2 H4/L0 IgG2a | SEQ ID NO: 233 | SEQ ID NO: 221 |
| D011-7016-LD2 H4/L1 hIgG1 | SEQ ID NO: 218 | SEQ ID NO: 222 |
| D011-7016-LD2 H4/L1 IgG2a | SEQ ID NO: 233 | SEQ ID NO: 222 |
| D011-7016-LD2 H4/L2 hIgG1 | SEQ ID NO: 218 | SEQ ID NO: 223 |
| D011-7016-LD2 H4/L2 IgG2a | SEQ ID NO: 233 | SEQ ID NO: 223 |
| D011-7016-LD2 H4/L3 hIgG1 | SEQ ID NO: 218 | SEQ ID NO: 224 |
| D011-7016-LD2 H4/L3 IgG2a | SEQ ID NO: 233 | SEQ ID NO: 224 |
| D011-7016-LD2 H4/L4 hIgG1 | SEQ ID NO: 218 | SEQ ID NO: 225 |
| D011-7016-LD2 H4/L4 IgG2a | SEQ ID NO: 233 | SEQ ID NO: 225 |
| D011-7016-LD2 H5/LCON hIgG1 | SEQ ID NO: 219 | SEQ ID NO: 220 |
| D011-7016-LD2 H5/LCON IgG2a | SEQ ID NO: 234 | SEQ ID NO: 220 |
| D011-7016-LD2 H5/L0 hIgG1 | SEQ ID NO: 219 | SEQ ID NO: 221 |
| D011-7016-LD2 H5/L0 IgG2a | SEQ ID NO: 234 | SEQ ID NO: 221 |
| D011-7016-LD2 H5/L1 hIgG1 | SEQ ID NO: 219 | SEQ ID NO: 222 |
| D011-7016-LD2 H5/L1 IgG2a | SEQ ID NO: 234 | SEQ ID NO: 222 |
| D011-7016-LD2 H5/L2 hIgG1 | SEQ ID NO: 219 | SEQ ID NO: 223 |
| D011-7016-LD2 H5/L2 IgG2a | SEQ ID NO: 234 | SEQ ID NO: 223 |
| D011-7016-LD2 H5/L3 hIgG1 | SEQ ID NO: 219 | SEQ ID NO: 224 |
| D011-7016-LD2 H5/L3 IgG2a | SEQ ID NO: 234 | SEQ ID NO: 224 |
| D011-7016-LD2 H5/L4 hIgG1 | SEQ ID NO: 219 | SEQ ID NO: 225 |
| D011-7016-LD2 H5/L4 IgG2a | SEQ ID NO: 234 | SEQ ID NO: 225 |

### EXAMPLES

The disclosure now being generally described, will be more readily understood by reference to the following examples, which are included merely for purposes of illustration of certain aspects and embodiments of the present disclosure, and is not intended to limit the disclosure.

In the following Examples, the inventors describe the generation of novel VH4-34-binding antibodies, and the biophysical and functional characterisation thereof.

### Example 1: VH4-34-Binding Antibodies

### 1.1 Production of VH4-34-Binding Antibodies

VH4-34-binding antibodies were raised by immunisation of BALB/c mice with VH4-34 antigen. Hybridomas were produced by fusion of B cells from spleens and lymph nodes of immunised mice with cells of the myeloma cell line P3X63.Ag8.653. Fused cells were harvested and plated for screening. Cell culture supernatants were screened using flow cytometry to identify colonies producing VH4-34-binding antibodies.

Total RNA was isolated from 1 to 5×10⁵ cells of the identified colonies using an RNA extraction kit, in accordance with the manufacturer's instructions. The isolated RNA was reverse-transcribed into cDNA, and heavy and light chain variable regions were amplified separately using gene-specific anti-sense primers and degenerate sense primer mixes using a one-step RT-PCR kit, following the manufacturer's instructions. Heavy and light chain variable regions were further amplified using degenerate primer mixes using a hot-start Taq DNA polymerase, following manufacturer's instructions, before cloning into expression vectors for transformation. After sequencing analysis of the resulting bacterial colonies, the cloned expression vectors were used for antibody production.

Four monoclonal VH4-34-binding antibody clones were selected for further development (Table 1):

D011-lEl1p, D011-6039, D011-6040 and D011-7016.

TABLE 1

| Antibody clone | VH/VL sequences |
|---|---|
| D011-1E11p | VH = SEQ ID NO: 5 |
| | VL = SEQ ID NO: 13 |
| D011-6039 | VH = SEQ ID NO: 21 |
| | VL = SEQ ID NO: 29 |
| D011-6040 | VH = SEQ ID NO: 37 |
| | VL = SEQ ID NO: 45 |
| D011-7016 | VH = SEQ ID NO: 53 |
| | VL = SEQ ID NO: 61 |

A humanised version of anti-VH4-34 antibody clone D011-1E11p was also designed: D011-1E11-5T2 (VH=SEQ ID NO: 125, VL=SEQ ID NO: 130).

DNA sequence encoding the heavy or light chain variable regions of the anti-VH4-34 antibody clones were subcloned into eukarvotic expression vectors encoding an engineered human/mouse hybrid IgG2a heavy chain constant region (SEQ ID NO: 124), and the constant region of human kappa light chain (SEQ ID NO: 79) to produce the antibodies below (Table 2).

Anti-VH4-34 clone D011-7016 was also expressed in human IgG1 format, D011-7016.hIgG1, comprising the constant regions of human IgG1 (SEQ ID NO: 71), and the constant region of human kappa light chain (SEQ ID NO: 79), thus generating a mouse-human chimeric antibody (Table 2).

Two further variants of clone D011-7016 were generated, D011-7016-LD-1 and D011-7016-LD2, and were expressed to include the engineered human/mouse hybrid heavy chain constant region (SEQ ID NO: 124) (Table 2).

TABLE 2

| Antibody | Name | Heavy chain | Light chain |
|---|---|---|---|
| [1] | D011-1E11p.IgG2a | SEQ ID NO: 95 | SEQ ID NO: 90 |
| [2] | D011-6039.IgG2a | SEQ ID NO: 96 | SEQ ID NO: 91 |
| [3] | D011-6040.IgG2a | SEQ ID NO: 97 | SEQ ID NO: 92 |
| [4] | D011-7016.IgG2a | SEQ ID NO: 98 | SEQ ID NO: 93 |
| [5] | D011-1E11- | SEQ ID NO: 99 | SEQ ID NO: 137 |
| | 5T2.IgG2a | | |
| [6] | D011-7016.hIgG1 | SEQ ID NO: 89 | SEQ ID NO: 93 |
| [7] | D011-7016- | SEQ ID NO: 138 | SEQ ID NO: 140 |
| | LD1.IgG2a | | |
| [8] | D011-7016- | SEQ ID NO: 138 | SEQ ID NO: 146 |
| | LD2.IgG2a | | |

### 1.2 VH4-34-Binding Antibodies Bind to the FR1 Hydrophobic Patch of VH4-34 Overex Pressing Cells

FACS assays were performed to evaluate binding of VH4-34-binding antibodies [1], [2], [3] and [4] to cells overexpressing VH4-34 antibodies.

Binding of the VH4-34-specific antibodies to CHO cell lines overexpressing surface VH4-34 antibodies tabalumab (Taba), 3A6 or zanolimumab (Zano) was evaluated.

Tabalumab, 3A6, and zanolimumab each comprise VH4-34, and are formed of the following immunoglobulin heavy and light chains (Table 3):

TABLE 3

| Antibody | Heavy chain | Light chain |
|---|---|---|
| tabalumab | SEQ ID NO: 120 | SEQ ID NO: 111 |
| 3A6 | SEQ ID NO: 114 | SEQ ID NO: 115 |
| zanolimumab | SEQ ID NO: 106 | SEQ ID NO: 109 |

The Tabalumab, 3A6, and zanolimumab antibodies are also modified to mutate the FR1 hydrophobic patch of VH4-34.

Tabalumab mutant FR1 (Taba FR1) comprises a heavy chain of SEQ ID NO: 119 and a light chain of SEQ ID NO: 111. The VH region of tabalumab mutant FR1 (SEQ ID NO: 118) comprises the following substitutions (numbered relative to SEQ ID NO: 120), which are predicted to disrupt the hydrophobic patch of VH4-34: Q6G, W7A, A23G, V24A and Y25S.

3A6 mutant FR1 (3A6 FR1) comprises a heavy chain of SEQ ID NO: 117 and a light chain of SEQ ID NO: 115. The VH region of 3A6 mutant FR1 (SEQ ID NO: 116) comprises the same substitutions above (numbered relative to SEQ ID NO: 114).

Zanolimumab mutant FR1 (Zano FR1) comprises a heavy chain of SEQ ID NO: 113 and a light chain of SEQ ID NO: 109. The VH region of zanolimumab mutant FR1 (SEQ ID NO: 112) comprises the same substitutions above (numbered relative to SEQ ID NO: 106).

Binding was compared to negative control cell lines, including WT CHO cells, CHO cells expressing VH4-34 antibodies lacking the hydrophobic patch (Zano FR1/3A6 FR1/Taba FR1), and VH4-34 negative cell lines CHO 10D1F and/or VSTB112.

FACS was performed according to standard protocols. Briefly, cells were assayed in PBS with 0.5% BSA and 2 mM EDTA. 60 k cells per well were incubated with the VH4-34-binding antibody or isotype control for 60 mins at 4° C. Following this, cells were incubated with a secondary anti-mouse FITC antibody for 30 mins at 4° C. FITC readout was measured by FACS on a benchtop flow cytometer.

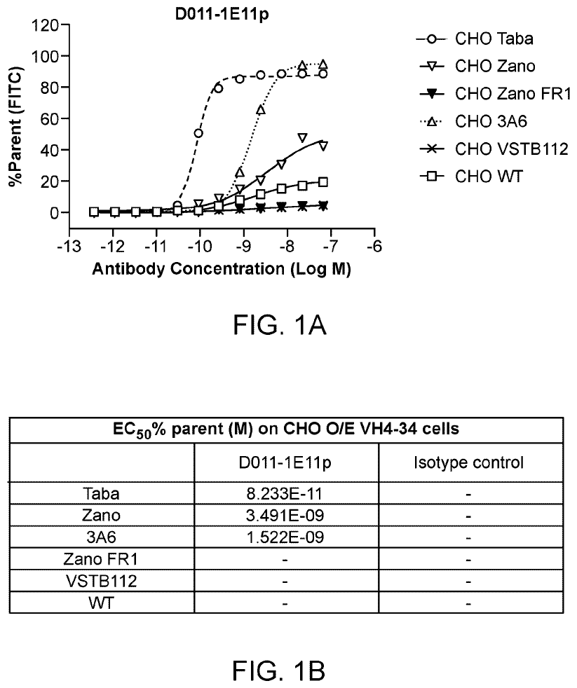

The resulting graphs are shown in FIG. 1A and FIG. 2A and EC₅₀% values are tabulated in FIGS. 1B and 2B. Anti-VH4-34 antibodies D011-1E11p [1], D011-7016 [4] and D011-6039 [2] demonstrated VH4-34-specific binding targeted to the VH4-34 FR1 hydrophobic patch on VH4-34 overexpressing cell lines, and not to the cells displaying control antibodies lacking VH4-34 or displaying mutant FR1. The hydrophobic patch of VH4-34 is important for binding of the antibodies to VH4-34.

Antibodies [7] and [8](variants of clone D011-7016) and D011-7016 were tested for their ability to bind to CHO cells overexpressing surface VH4-34 antibodies tabalumab (Taba), 3A6 or zanolimumab (Zano).

For FACS evaluation, cells were assayed in PBS with 0.5% BSA and 2 mM EDTA. 30 k cells per well were incubated with the VH4-34-binding antibody or isotype control for 45 mins at 4° C. Cells were incubated with a secondary anti-mouse FITC antibody for 20 mins at 4° C. FITC readout was measured by FACS.

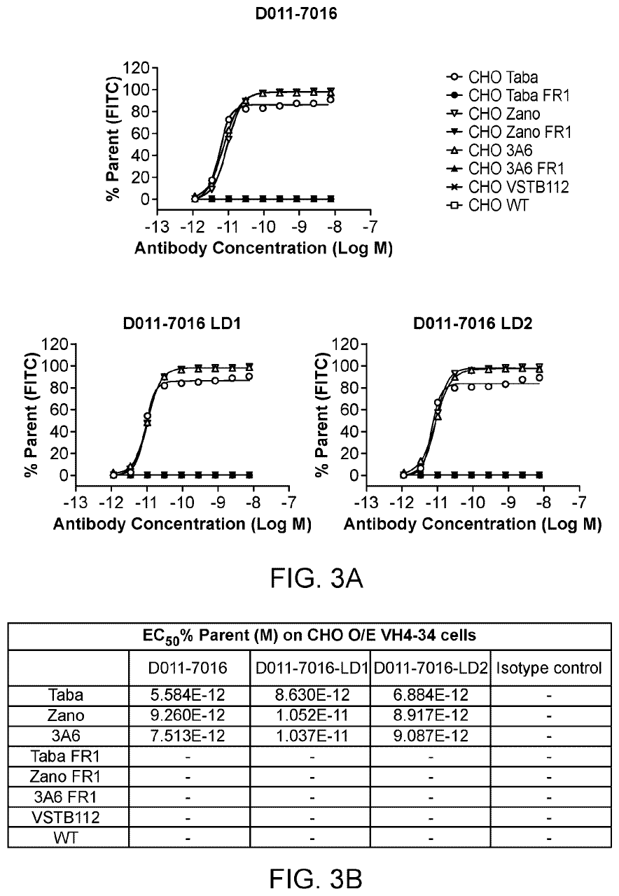

The graphical results are shown in FIG. 3A. EC50% values are tabulated in FIG. 3B. Anti-VH4-34 antibodies D011-7016 [4], D011-7016-LD1 [7] and D011-7016-LD2 [8] showed VH4-34-specific binding targeted to the VH4-34 FR1 hydrophobic patch on overexpressing cell lines. Anti-VH4-34 antibodies did not bind to the cells expressing FR1 mutants (Taba FR1, Zano FR1, 3A6 FR1) or antibodies that lack VH4-34 (e.g. VSTB112).

### 1.3 VH4-34-Binding Antibodies Bind to the FR1 Hydrophobic Patch of VH4-34 Overexpressing Cells

A FACS assay was performed with endogenously VH4-34-expressing cell line OCI-Ly3 to assess binding of VH4-34-binding antibodies [2], [3] and [4]. Binding was compared to a negative-control Pfeiffer cell line and an IgG isotype control.

FACS was performed according to standard protocols. Briefly, cells were assayed in PBS with 0.5% BSA and 2 mM EDTA. Live dead staining was performed with a live dead cell dye for 15 minutes at 4° C. 100 k cells per well were incubated with the VH4-34-binding antibody or isotype control for 60 mins at 4° C. Following this, cells were incubated with a secondary PE goat anti-mouse IgG for 30 mins at 4° C. and PE readout was measured.

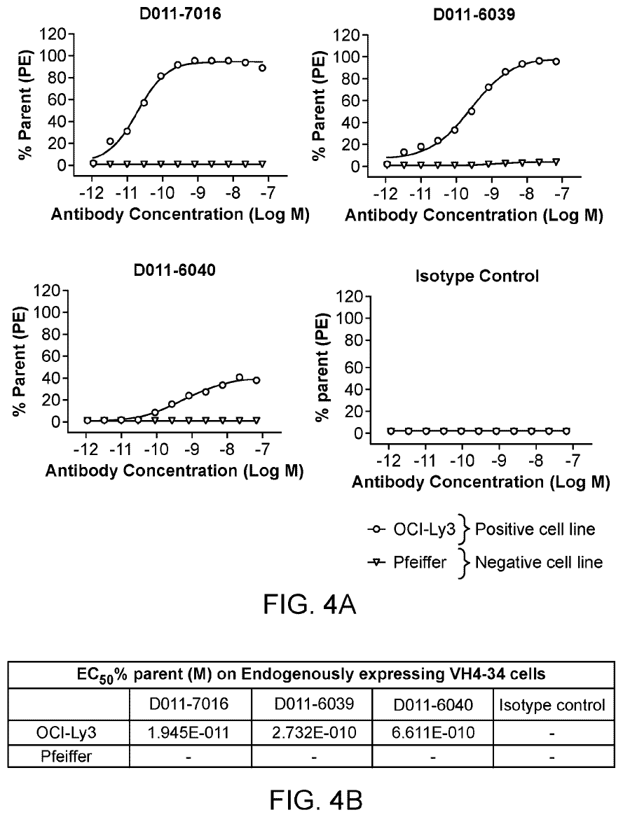

The results are shown in FIG. 4A. Antibodies D011-7016 [4] and D011-6039 [2] demonstrated VH4-34 specific binding targeted to the VH4-34 FR1 hydrophobic patch on the OCI-Ly3 cell line that endogenously expresses VH4-34, and did not bind to the control cell line lacking VH4-34. EC50% values are tabulated in FIG. 4B.

### 1.4 Binding of Humanised VH4-34-Binding Antibody to Cell Lines Expressing VH4-34

A FACS assay was performed to assess binding of the humanised VH4-34-binding antibody [5] to endogenously VH4-34-expressing cell lines HBL-1 and GA-10. Binding was compared to a negative-control MM.1S cell line and an IgG isotype control.

FACS was performed according to standard protocols. Briefly, cells were assayed in PBS with 0.5% BSA and 2 mM EDTA. Live dead staining was performed with a live dead cell dye for 15 minutes at 4° C. 100 k cells per well were incubated with the VH4-34-binding antibody or mIgG1 isotype control for 60 mins at 4° C. Following this, cells were incubated with an APC-conjugated secondary goat anti-mouse IgG (1:300) for 30 mins at 4° C. and the readout was measured by FACS.

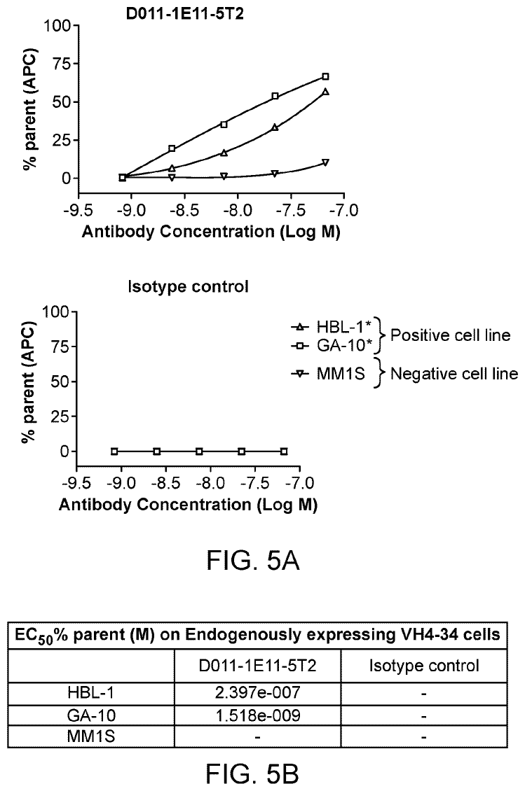

The results are shown in FIG. 5A. EC50% values are tabulated in FIG. 5B. Humanised clone D011-1E11-5T2 [5] showed dose dependent binding to VH4-34-expressing HBL-1 and GA-10 cell lines, and not to the control cell line lacking VH4-34 (MM1S).

### Example 2: In Vitro Studies Using VH4-34-Binding Antibodies

### 2.1 Killing of VH4-34 Positive Cells Via ADCC

The ability of VH4-34-binding antibodies to induce antibody dependent cell-mediated cytotoxicity (ADCC) was evaluated.

CHO cells overexpressing VH4-34-encoded antibody tabalumab or VH4-34-encoded antibody zanolimumab were used as the target cells. The Jurkat Lucia NFAT-CD16 reporter cell line was used as the effector cell.

Efficacy of antibody [6] was compared against an IgG1 isotype negative control (purified human IgG1 isotype, 1 mg/ml).

Target cells were seeded in a 96-well plate at 100 k cells per well and incubated with 3-fold serially diluted antibodies and controls (30 g/ml to 0.005 g/ml) for 1 hr at 37° C. under 5% CO₂. After 1 hour, 200 k effector cells were added to each well, and the plate was incubated overnight at 37° C. under 5% CO₂. The following day, 20 μl supernatant of the co-incubated target and effector cells was transferred to a 96-well white opaque plate and detection medium was added. Luminescence was measured luminescence reader.

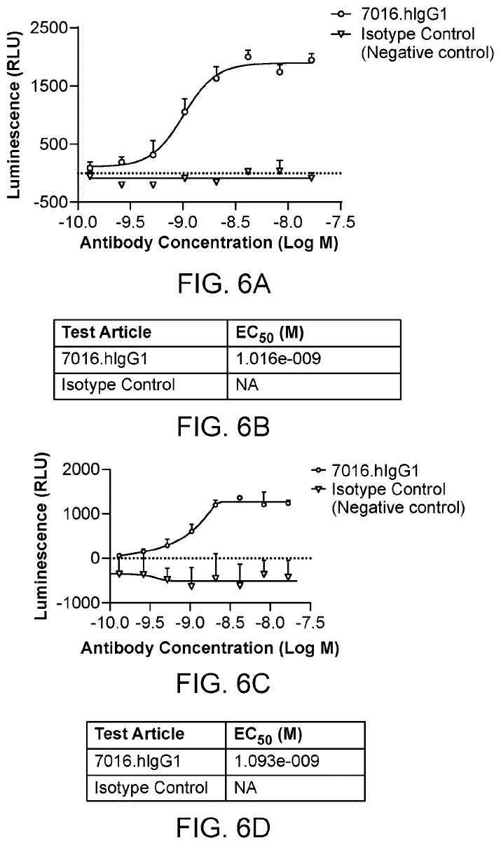

The results are shown in FIGS. 6A and 6C and EC50 values are tabulated in FIGS. 6B and 6D. D011-7016.hIgG1 [6] was shown to kill VH4-34-positive cells expressing either tabalumab (FIGS. 6A, 6B) or zanolimumab (FIGS. 6C, 6D) via ADCC.

### 2.2 Killing of VH4-34-Positive Cells Via ADCP

The ability of VH4-34-binding antibodies to induce antibody dependent cell-mediated phagocytosis (ADCP) was evaluated.

OCI-Ly3 cell endogenously expressing VH4-34 was used as the target cell line. Pfeiffer cell was used as the negative control cell line. Effector cells were MO macrophages, differentiated by culturing monocytes from fresh blood with 80 ng/ml M-CSF for 5 days.

Efficacy of antibody [6] was tested compared to one positive control, Rituximab.hIgG1, and one negative control, an IgG1 isotype control (purified human IgG1 isotype, 1 mg/ml).

Target cells labelled with 0.15 M carboxyfluoroscein succinimidyl ester (CFSE) were incubated with antibodies and controls described above for 1 hr at 37° C. under 5% CO₂. After 1 hr, macrophage effector cells were added in a 2:1 effector to target ratio, and the cells were incubated for 3 hrs at 37° C. under 5% CO₂. After incubation, macrophages were stained with CD14. Phagocytosis readout was measured by FACS, measuring % CD14⁺CFSE⁺ cells.

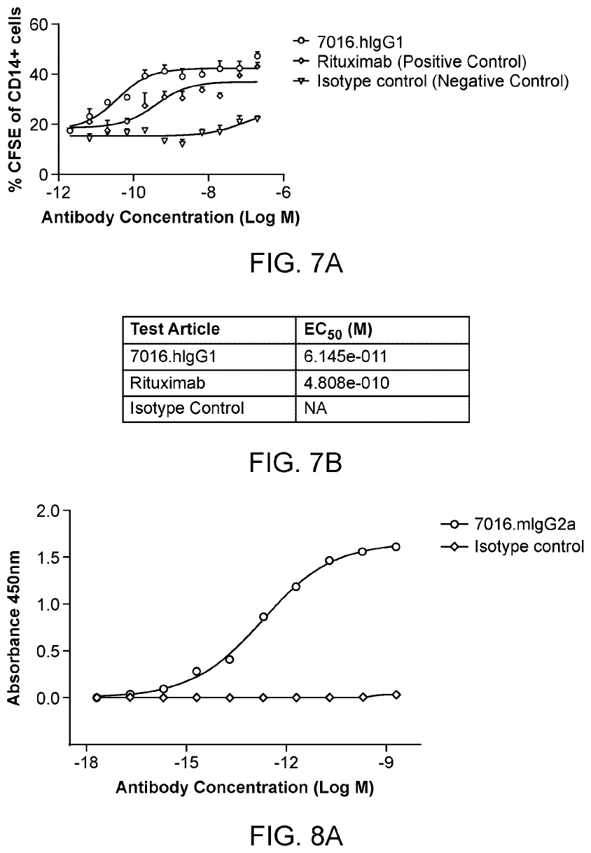

The results are shown in FIG. 7A, and EC50 values are tabulated in FIG. 7B. DOt 1-7016.hIgG1 [6] was shown to kill VH4-34-positive cells via ADCP. 2.3 VH4-34-binding antibody blocks cold agglutination

Anti-VH4-34 antibody [4] was tested for its ability to bind to VH4-34-encoded antibody IGM-55.5.

Plates were coated with 1 μg/ml of IGM-55.5 overnight at 4° C., then washed and blocked with PBS+1% BSA for 2 hr at room temperature (RT). Plate was washed 3× with TBS+0.1% Tween 20. Test antibody [4] and IgG1 isotype control (starting concentrations: 30 ug/ml) were added onto the plate and incubated for 1.5 hrs at RT. A secondary antibody (HRP-conjugated goat anti-mouse IgG) was prepared at 1:5000 dilution in blocking buffer and added to the plate with 1 hr incubation RT. TMB ELISA substrate was added and incubated for 15 mins at RT in the dark, before adding the stop solution. The absorbance was read at 450 nm.

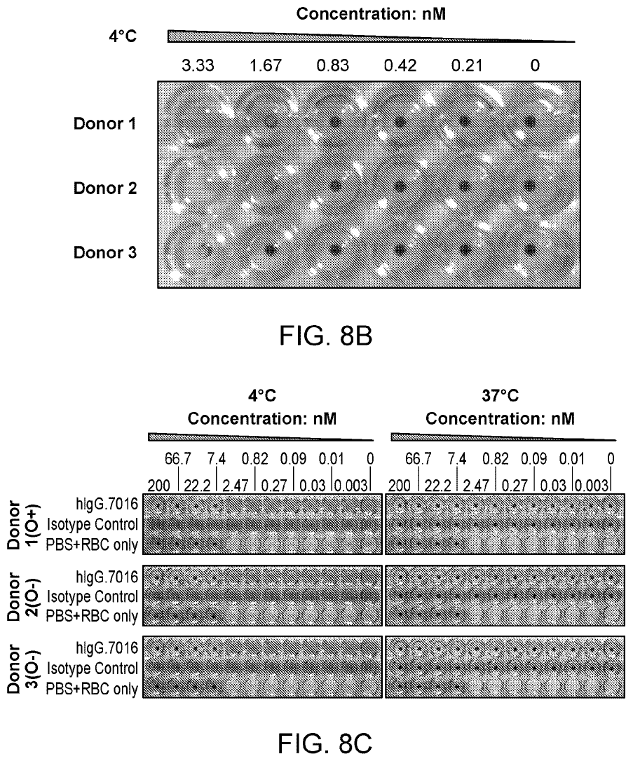

The results are shown in FIG. 8A. D011-7016.mlgG2a [4] demonstrated binding to VH4-34-encoded antibody IGM-55.5.

FIG. 8B shows that VH4-34-encoded antibody IGM-55.5 induces cold agglutination of red blood cells from 3 donors at 3.33 nM at 4° C. (relevant to cold agglutinin disease: a rare type of autoimmune hemolytic anemia in which the immune system attacks red blood cells at low body temperatures).

Anti-VH4-34 antibody [6] was tested for its ability to inhibit cold agglutination caused by antibody IGM-55.5.

Isolated red blood cells were obtained from three donors (two 0-, one 0+). Briefly, 4 mL of whole blood was topped up with PBS and centrifuged at 200×g for 10 min. The platelet fraction in supernatant was removed by aspiration and the cell pellet was washed with 15 mL PBS, mixed well by inverting the tube and centrifuged at 1500 rpm for 5 min. The wash step was repeated thrice. RBCs were resuspended with 10 mL of PBS. 2.5% RBCs was prepared by mixing 250 uL of resuspended RBCs with 9.75 mL of PBS.

Antibody [6] and IgG1 isotype control were serially diluted in PBS (200 nM to 0.003 nM). 50 uL of antibody [6] or isotype control was incubated with 50 uL of VH4-34-encoded IGM-55.5 for 30 minutes at 4° C. 100 uL of 2.5% donor RBCs was introduced into each well, with the IGM-55.5 concentration being 3.33 nM (3 g/mL) at 200 uL final volume. Plates were incubated at 4° C. overnight.

The results are shown in FIG. 8C. Cold agglutination caused by VH4-34-encoded antibody IGM-55.5 was blocked using anti-VH4-34 antibody D011-7016.hIgG1 [6] at a ~1:2 ratio (IgM-55.5:7016).

### Example 3: In Vivo Clearance of VH4-34-Positive Antibodies by VH4-34-Binding Antibodies

In vivo experiments were performed to evaluate the clearance of VH4-34-positive Zanolimumab (Zano.hIgG1) antibody in mouse circulation by VH4-34-binding antibody [4].

20-week BALB/c female mice were inoculated with one of five treatment arms (n=3) intravenously (tail vein) according to the schedule below (Table 4). Sera was collected from two groups of mice. Sera collection time for Group 1 mice was at 6 h. 96 h (4 days), 240 h (10 days). Sera collection time for Group 2 mice was at 24 h. 168 h (7 days). 336 h (14 days).

TABLE 4

| | VH4-34 mAb | |
|---|---|---|
| Arms | injection (i.v.) | Treatment (i.v.) |
| 1 | Zano.hIgG1 10 mg/kg | None |
| 2 | Zano.hIgG1 10 mg/kg | D011-7016.mIgG2a 10 mg/kg, at 6 h |
| 3 | Zano.hIgG1 10 mg/kg | D011-7016.mIgG2a 10 mg/kg, at 6 h |
| | | and 96 h |
| 4 | Zano.hIgG1 10 mg/kg | D011-7016.mIgG2a 20 mg/kg, at 6 h |
| 5 | None | D011-7016.mIgG2a 10 mg/kg at 6 h |

ELISA was performed on collected sera samples to evaluate clearance of the VH4-34-positive antibody Zanolimumab (Zano.hIgG1) from mouse circulation.

ELISA was performed according to standard protocol. Briefly, wells were coated with 1 g/ml His-tagged human CD4, incubated overnight at 4° C., washed and blocked with 1× phosphate-buffered saline (PBS) with 1% BSA for 2 hours at room temperature. The plate was washed three times with 1×PBS with 0.05% Tween 20 and dried in between each step. Mouse sera from groups and arms described above and standards spiked with a known concentration of Zanolimumab (serially diluted with 1×PBS+1% BSA) were added into the plate and incubated for 1.5 hr at room temperature. HRP goat anti-human secondary antibody was prepared at 1:7000 dilution with 1×PBS with 1% BSA and added to the plate. The plate was incubated in the dark, at room temperature, for 1 hr. Plates were developed with colorimetric detection substrate 3.3′.5.5′-tetramethylbenzidine. The reaction was stopped with an ELISA Stop Solution, and OD was measured at 450 nM using a microplate reader.

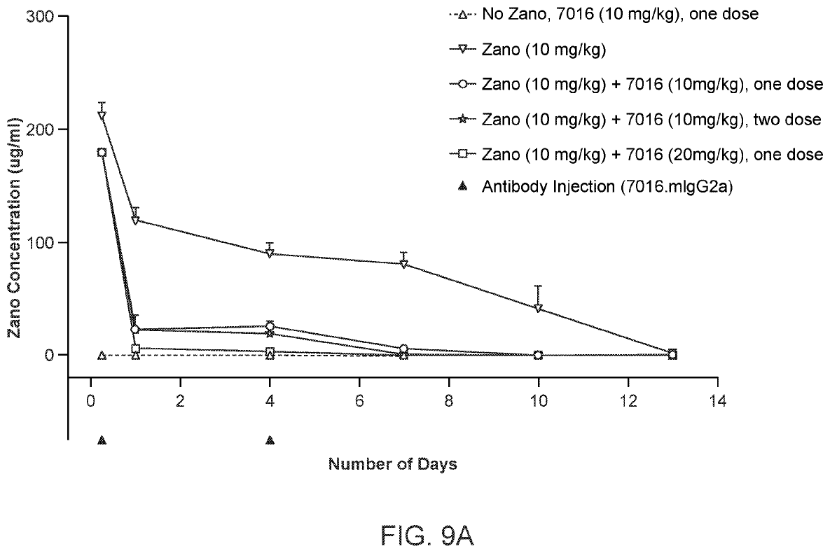

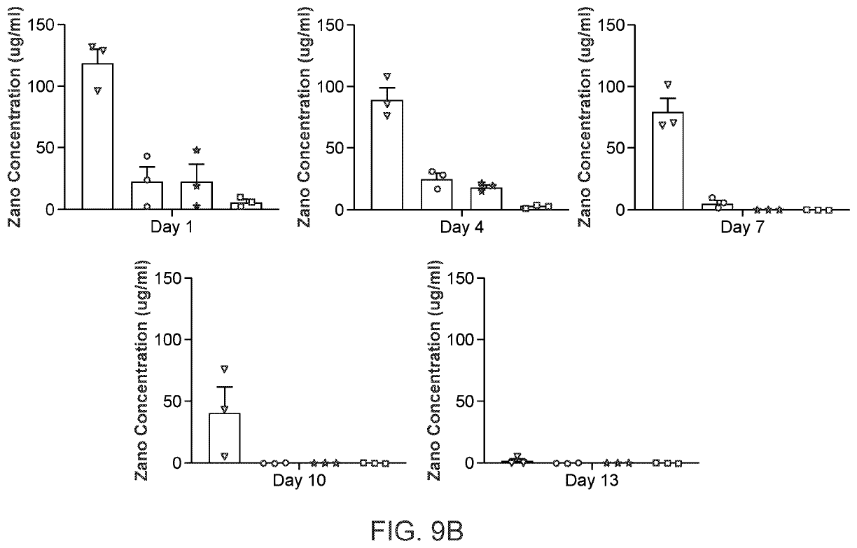

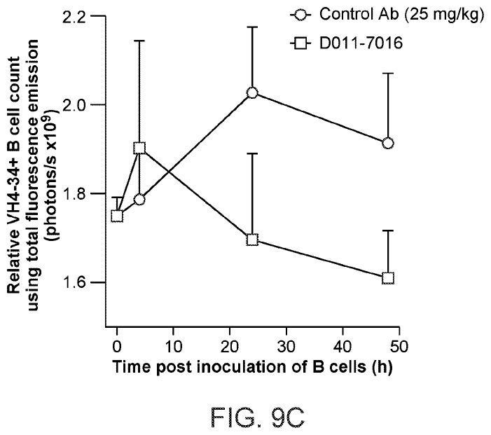

The results are shown in FIGS. 9A and 9B. Anti-VH4-34 antibody D011-7016.mIgG2a [4] was demonstrated to clear the VH4-34-encoded antibody Zano.hIgG1 from the mouse circulation within 24 hours.

In vivo experiments were performed to evaluate the clearance of VH4-34-positive B cells. Nude mice were i.p. injected with D011-7016, mIgG2athen injected again at 1 h with OCI-Ly3 that endogenously expresses antibodies containing VH4-34 and transiently expression mCardinal by i.v. injection. Mice were imaged at time points indicated in FIG. 9C. Anti-VH4-34 antibody D011-7016.mlgG2a [4] was demonstrated to clear the VH4-34-positive B cells from mouse circulation.

### Example 4: Generation and Assessment of Humanized Antibodies

Clone D011-7016-LD2 was selected for humanization. Five humanized VH and five humanized VL sequences were generated and paired in the combinations below (Table 5).

TABLE 5

| | D011-7016- | D011-7016- | D011-7016- | D011-7016- | D011-7016- |
|---|---|---|---|---|---|
| | LD2_L1 VL | LD2_L2 VL | LD2_L3 VL | LD2_L4 VL | LD2_L0 VL |
| | SEQ ID NO: | SEQ ID NO: | SEQ ID NO: | SEQ ID NO: | SEQ ID NO: |
| | 193 | 198 | 201 | 205 | 189 |
| D011-7016- | H1L1 | H1L2 | H1L3 | H1L4 | — |
| LD2_H1 VH | | | | | |
| SEQ ID NO: | | | | | |
| 171 | | | | | |
| D011-7016- | H3L1 | H3L2 | H3L3 | H3L4 | — |
| LD2_H3 VH | | | | | |
| SEQ ID NO: | | | | | |
| 176 | | | | | |
| D011-7016- | H4L1 | H4L2 | H4L3 | H4L4 | — |
| LD2_H4 VH | | | | | |
| SEQ ID NO: | | | | | |
| 181 | | | | | |
| D011-7016- | H5L1 | H5L2 | H5L3 | H5L4 | — |
| LD2_H5 VH | | | | | |
| SEQ ID NO: | | | | | |
| 182 | | | | | |
| D011-7016- | — | — | — | — | H0L0 |
| LD2_H0 VH | | | | | |
| SEQ ID NO: | | | | | |
| 167 | | | | | |

DNA sequence encoding the heavy or light chain variable regions of the anti-VH4-34 antibodN, clones w ere subcloned into eukaryotic expression vectors encoding an engineered human/mouse hybrid IgG2a heavy chain constant region (SEQ ID NO: 124), and the constant region of human kappa light chain (SEQ ID NO: 79) to produce the following antibodies (Table 6).

TABLE 6

| Antibody | Heavy chain | Light chain |
|---|---|---|
| H1L1.IgG2a | SEQ ID NO: 231 | SEQ ID NO: 222 |
| H1L2.IgG2a | SEQ ID NO: 231 | SEQ ID NO: 223 |
| H1L3.IgG2a | SEQ ID NO: 231 | SEQ ID NO: 224 |
| H1L4.IgG2a | SEQ ID NO: 231 | SEQ ID NO: 225 |
| H3L1.IgG2a | SEQ ID NO: 232 | SEQ ID NO: 222 |
| H3L2.IgG2a | SEQ ID NO: 232 | SEQ ID NO: 223 |
| H3L3.IgG2a | SEQ ID NO: 232 | SEQ ID NO: 224 |
| H3L4.IgG2a | SEQ ID NO: 232 | SEQ ID NO: 225 |
| H4L1.IgG2a | SEQ ID NO: 233 | SEQ ID NO: 222 |
| H4L2.IgG2a | SEQ ID NO: 233 | SEQ ID NO: 223 |
| H4L3.IgG2a | SEQ ID NO: 233 | SEQ ID NO: 224 |
| H4L4.IgG2a | SEQ ID NO: 233 | SEQ ID NO: 225 |
| H5L1.IgG2a | SEQ ID NO: 234 | SEQ ID NO: 222 |
| H5L2.IgG2a | SEQ ID NO: 234 | SEQ ID NO: 223 |
| H5L3.IgG2a | SEQ ID NO: 234 | SEQ ID NO: 224 |
| H5L4.IgG2a | SEQ ID NO: 234 | SEQ ID NO: 225 |
| H0L0.IgG2a | SEQ ID NO: 230 | SEQ ID NO: 221 |

The binding affinity of each humanized antibody for 3A6 antibody (containing VH4-34 antigen), 3A6-FR1 (VH4-34 FR1 mutant) and VSTB112 (negative control protein) was evaluated.

Plates were coated with 1 ug/ml of anti-human IgG Fc overnight at 4° C. Plates were washed thrice with PBS/0.05% Tween (PBST) before blocking with 5% BSA for 1 h at room temp (RT). Plates were then washed again before addition of 1 μg/ml of 3A6 (VH4-34 antigen), 3A6-FR1 (VH4-34 FR1 mutant) or VSTB112 (negative control protein) human IgG1 proteins. Plates were washed thrice. The indicated primary antibodies, 4× serially diluted from 1 μg/ml, were added and the plates were incubated th, RT. After washing the plates, anti-mouse IgG Fc/HRP diluted 1:5000 in 1% BSA was added and incubated for 1 h at RT. Plates were washed before detection using 1-step ELISA substrate.

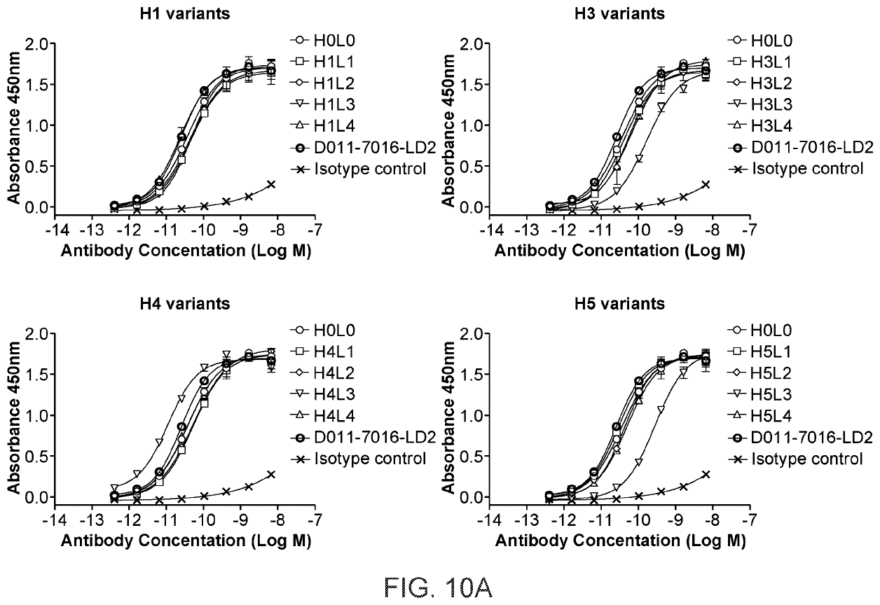

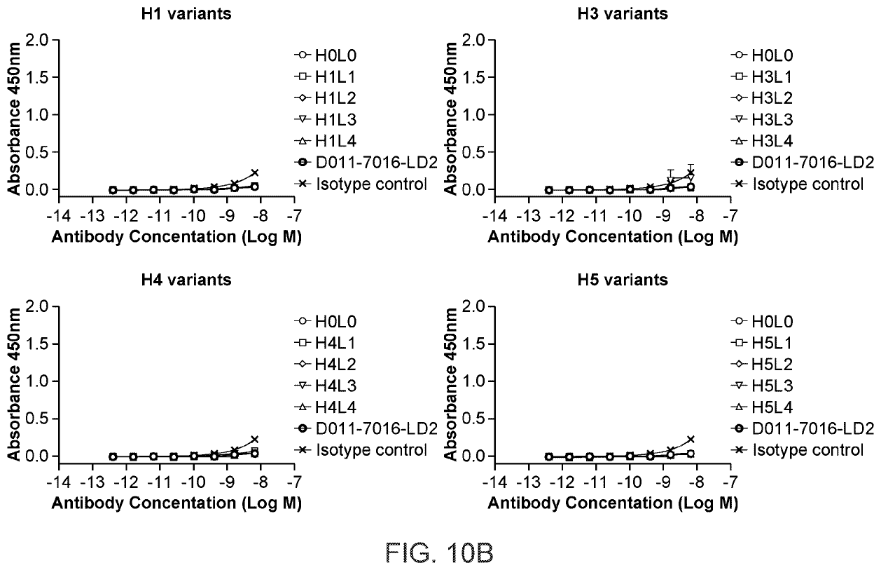

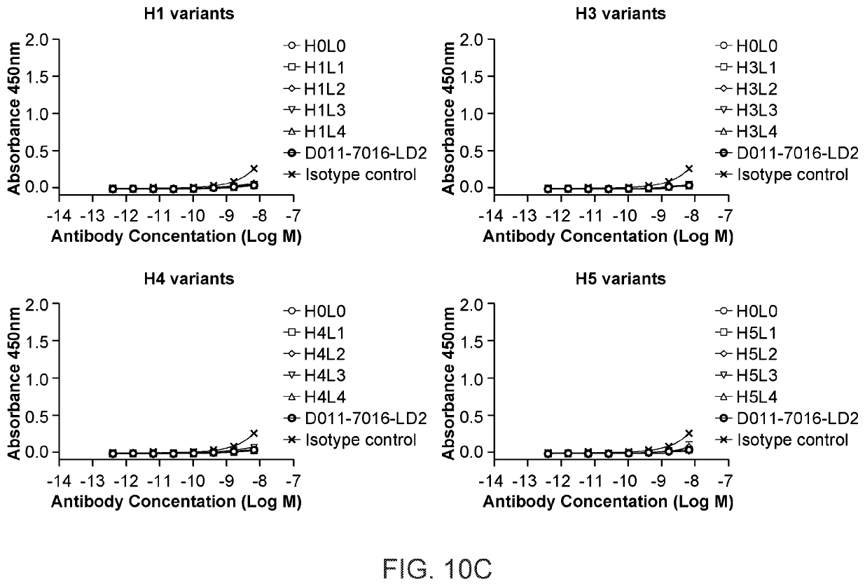

The results are shown in FIG. 10A and tabulated in FIG. 10B. The humanized antibodies were found to bind to 3A6 (FIG. 10A) but did not bind to 3A6-FR1 (VH4-34 FR1 mutant: FIG. 10B) or VSTB112 (negative control protein: FIG. 10C).

The binding affinity of each antibody for 3A6 is shown in Table 7A.

TABLE 7

EC₅₀ (Antibody concentration (M))

| Antibody | 3A6 | 3A6-FR1 | VSTB112 |
|---|---|---|---|
| H0L0 | 3.689E−11 | — | — |
| H1L1 | 4.461E−11 | — | — |
| H1L2 | 4.267E−11 | — | — |
| H1L3 | 4.693E−11 | — | — |
| H1L4 | 2.342E−11 | — | — |
| H3L1 | 3.979E−11 | — | — |
| H3L2 | 5.297E−11 | — | — |
| H3L3 | 1.687E−10 | — | — |
| H3L4 | 6.272E−11 | — | — |
| H4L1 | 5.212E−11 | — | — |
| H4L2 | 5.323E−11 | — | — |
| H4L3 | 1.098E−11 | — | — |
| H4L4 | 3.650E−11 | — | — |
| H5L1 | 2.946E−11 | — | — |
| H5L2 | 4.319E−11 | — | — |
| H5L3 | 2.82E−10 | — | — |
| H5L4 | 4.953E−11 | — | — |
| D011-7016-LD2 | 2.541E−11 | — | — |
| Isotype control | — | — | — |

Taba, Zano, and 3A6 have the same Frl-4 and CDR1 and CDR2 in the HC region but a different CDR3. Taba, Zano, and 3A6 have different LC. The specificity of humanized antibodies was independent of HC CDR3 and the LC as indicated by experiments determining the EC50 geomean MFI (M) on VH4-34 overexpressing CHO cells (Table 7B) The LD2 antibody was also tested in these experiments.

TABLE 7B
EC₅₀ geomean MFI (M) on CHO overexpressing cells

| | Taba | Zano | 3A6 |
|---|---|---|---|
| 7016-LD2 | 2.8E−10 | 6.92E−10 | 5.19E−10 |
| 7016-H0L0 | 3.76E−10 | 4.53E−10 | 6.05E−10 |
| 7016-H5L1 | 3.13E−10 | 6.45E−10 | 5.90E−10 |
| 7016-H5L2 | 2.84E−10 | 6.50E−10 | 5.90E−10 |

Humanized antibodies H0L0, H1L4, H3L1, H4L2, H5L1, H5L2, and H5L4 were then tested for their ability to bind to cell line OCI-Ly3 that endogenously expresses antibodies containing VH4-34. Binding affinity was compared to parental clone D011-7016-LD2, a negative control Pfeiffer cell line, and an isotype control.

Binding was assessed by FACS. Briefly, cells were assayed in PBS with 0.5% BSA and 2 mM EDTA. Live dead staining was performed with a live dead cell dye for 15 minutes at 4° C. 100 k cells per well were incubated with the VH4-34-binding antibody or isotype control for 60 mins at 4° C. Following this, cells were incubated with a secondary PE goat anti-mouse IgG (1:300) for 30 mins at 4° C. and PE readout was measured.

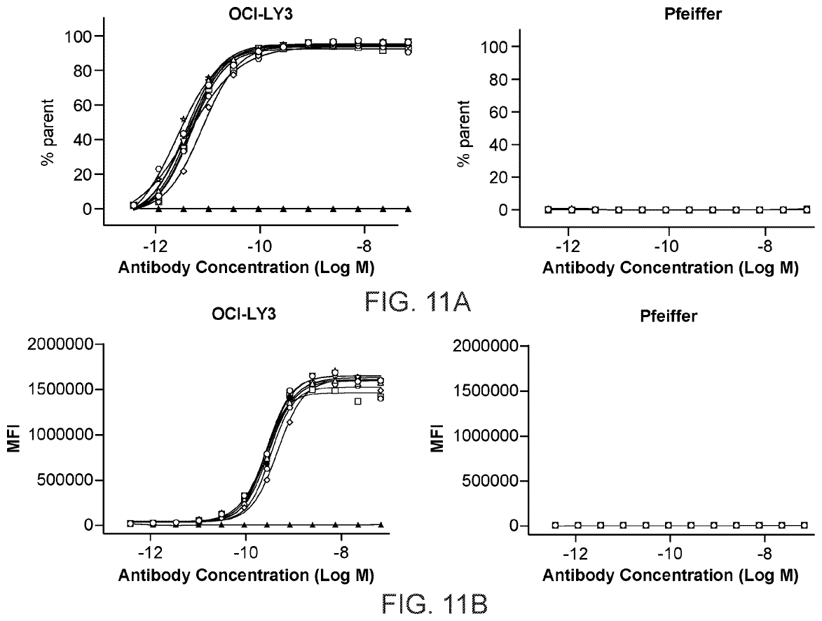

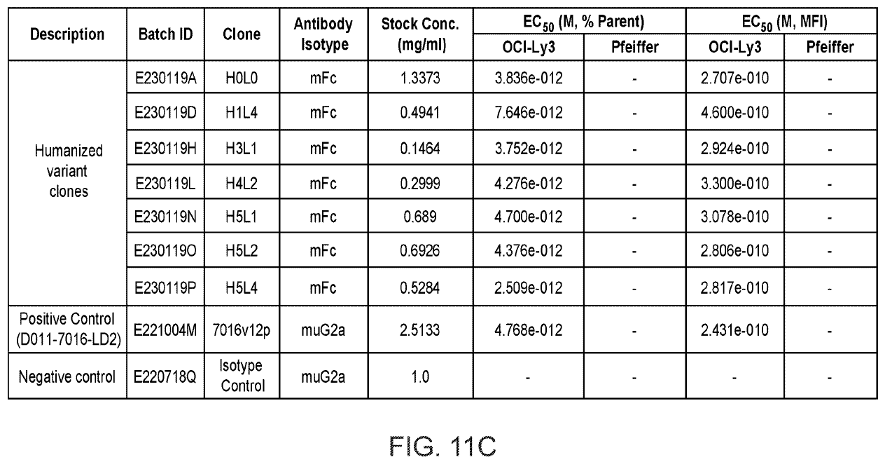

The results are shown in FIGS. 11A and 11B and tabulated in FIG. 11C. All tested antibodies bound to VH4-34-expressing OCI-Ly3 cell line with the same potency as parental clone D011-7016-LD2 and did not bind to the negative control cell line.

### Example 5: Humanized VH4-34-Binding Antibodies in Blocking Cold Agglutination

This Example describes the ability of humanized anti-VH4-34 antibodies to reverse cold agglutination of RBCs by displacement of RBC-bound IGM-55.5.

Isolated red blood cells were obtained from a donor. Briefly. 4 mL of whole blood was topped up with PBS and centrifuged at 200×g for 10 min. The platelet fraction in supernatant was removed by aspiration and the cell pellet was washed with 15 mL PBS, mixed well by inverting the tube and centrifuged at 1500 rpm for 5 min. The wash step was repeated thrice. RBCs were resuspended with 10 mL of PBS. 2.5% RBCs was prepared by mixing 250 uL of resuspended RBCs with 9.75 mL of PBS.

100 uL of 2.5% donor RBCs was introduced into each well, with the IGM-55.5 concentration being 3.33 nM (3 g/mL) at 200 uL final volume. Plates were incubated at 4° C. or 10° C. overnight and high resolution images were taken to evaluate agglutination. Subsequently, humanized antibodies H0L0, H5L1, H5L2 and isotype control were serially diluted in PBS introduced into each well at concentrations 100 nM to 0.002 nM. Plates were incubated a second time at 4° C. or 10° C. overnight. High resolution images were taken to observe changes in agglutination state.

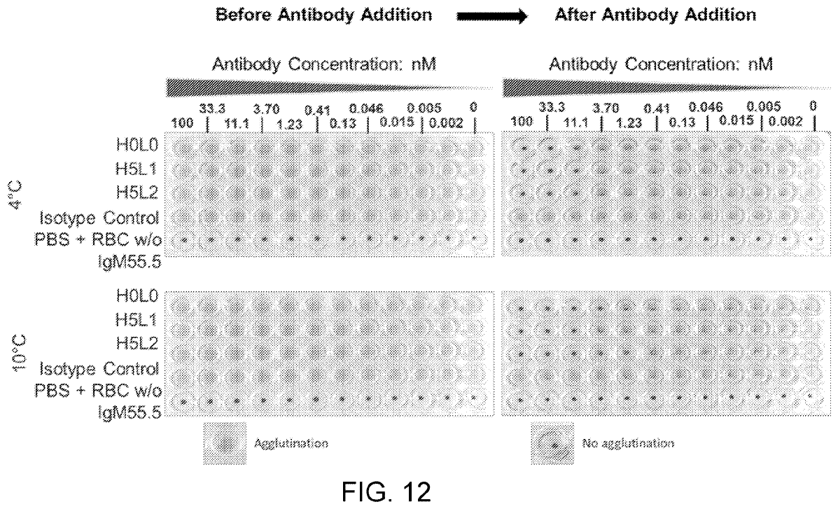

As seen in FIG. 12, at concentrations >3.70-11.1 nM, the VH4-34 humanized antibodies were completely able to reverse cold agglutination induced by 3.33 nM IGM-55.5 at 4 and 10° C.

### Example 6: VH4-34-Binding Antibodies Bind to Autoantibodies Isolated from CAD and SLE Patients and a NHP VH4-73 Antibody

### 6.1 VH4-34-Binding Antibodies Bind to CAD Autoantibodies

Ten autoantibodies isolated from individuals suffering from cold agglutinin disease CAD were isolated and used in Biacore and ELISA binding experiments with an exemplary VH4-34-binding antibody to determine KD and EC₅₀, respectively. The exemplary VH4-34-binding antibody bound to all ten CAD autoantibodies at comparable levels (Table 8).

TABLE 8

| | Biacore KD | ELISA EC50 |
|---|---|---|
| Autoantibody | (nM) | (nM) |
| CAD-2 | 0.0102 | 0.032 |
| CAD-3 | | 0.03 |
| CAD-6 | 0.0136 | 0.018 |
| CAD-7 | 0.0887 | 0.022 |
| CAD-10 | 0.0254 | 0.022 |
| CAD-18 | 0.0365 | 0.021 |
| CAD-19 | 0.0227 | 0.019 |
| CAD20 | 0.0136 | 0.038 |
| CAD-27 | 0.0072 | 0.022 |
| CAD-23 | 0.0165 | 0.015 |

### 5.2 VH4-34-Binding Antibodies Bind to SLE Autoantibodies

Eleven autoantibodies isolated from individuals suffering from systemic lupus erythematosus SLE were isolated and used in Biacore and ELISA binding experiments with an exemplary VH4-34-binding antibody to determine KD and EC₅₀, respectively. The exemplary VH4-34-binding antibody bound to all eleven SLE autoantibodies (Table 9).

TABLE 9

| Autoantibody | Biacore KD (nM) | ELISA IC50 (nM) |
|---|---|---|
| SLE1 | N/A | 0.035 |
| SLE2 | N/A | 0.041 |
| SLE3 | N/A | 0.039 |
| SLE4 | N/A | 0.041 |
| SLE6 | N/A | 0.038 |
| SLE7 | N/A | 0.032 |
| SLE8+ | <0.001 | 0.024 |
| SLE13+ | 0.0133 | 0.028 |
| SLE15+ | 0.0075 | 0.034 |
| SLE16+ | 0.0215 | 0.03 |
| SLE17 | N/A | N/A |

### 6.2 VH4-34-Binding Antibodies Bind NHP VH4-73 but do not Bind to VH4-9 and VH4-4

Other VH4 family members were used in Biacore and ELISA binding experiments with an exemplary VH4-34-binding antibody to determine cross-species binding. The exemplary VH4-34-binding antibody bound to the non-human primate ortholog of VH4-34, NHP VH4-73 Fab as determined by Biacore experiments. The exemplary VH4-34-binding antibody also bound NHP VH4-73 IgG1 as determined by Biacore experiments and IgG as determined by ELISA. The exemplary VH4-34-binding antibody did not bind to any form of h VH4-39 or h VH4-4 (Table 10).

TABLE 10

| | | IgG by ELISA |
|---|---|---|
| | IgG1 (KD, nM) | (EC50, nM) |
| NHP VH4-73 | 0.13 | 0.0037 |
| h VH4-39 | no binding | |
| h VH4-4 | no binding | |

### Example 7: Expression of IGHV4-34 in Various Diseases

This Example describes the analysis ofthe expression ofIGHV4-34 in various diseases.

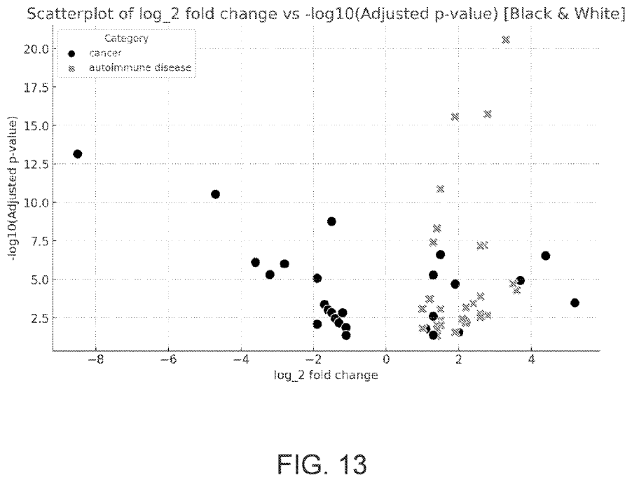

Gene expression analysis was performed by querying the FBI Expression Atlas for studies where human IGHV4-34 is differentially expressed in disease samples compared to normal controls. RNA-sequencing data were analysed by pairwise comparison and log 2 fold changes were determined. 109 studies were identified in which IGHV4-34 was differentially expressed (Benjamini-Hochberg adjusted p-value <0.05). Each study was annotated and grouped as “cancer,” or “autoimmune disease” and plotted in a volcano plot (FIG. 13). IGHV4-34 was found to be upregulated in autoimmune diseases and do9n-regulated in cancer samples, reflecting the dichotomic roles ofB cells. The data are tabulated as seen in Tables 11-l2 below.

TABLE 11

| Category | log2 fold change | −log10 (p-value) |
|---|---|---|
| autoimmune disease | 3.6 | 4.292614 |
| autoimmune disease | 3.5 | 4.709531 |
| autoimmune disease | 3.3 | 20.57466 |
| autoimmune disease | 2.8 | 15.73796 |
| autoimmune disease | 2.8 | 2.670005 |
| autoimmune disease | 2.7 | 7.215124 |
| (rheumatoid arthritis) | | |
| autoimmune disease | 2.6 | 7.163247 |
| autoimmune disease | 2.6 | 3.88266 |
| autoimmune disease | 2.6 | 2.769152 |
| autoimmune disease | 2.6 | 2.557986 |
| autoimmune disease | 2.4 | 3.425932 |
| autoimmune disease | 2.2 | 3.186201 |
| autoimmune disease | 2.2 | 2.18225 |
| autoimmune disease | 2.1 | 2.440945 |
| autoimmune disease | 1.9 | 15.56106 |
| autoimmune disease (lupus) | 1.9 | 1.566161 |
| autoimmune disease | 1.5 | 10.86929 |
| autoimmune disease | 1.5 | 3.059284 |
| autoimmune disease | 1.5 | 2.329917 |
| autoimmune disease | 1.5 | 2.01806 |
| autoimmune disease | 1.4 | 8.304243 |
| autoimmune disease | 1.4 | 2.048904 |
| autoimmune disease | 1.4 | 1.776015 |
| autoimmune disease | 1.4 | 1.316325 |
| (rheumatoid arthritis) | | |
| autoimmune disease | 1.3 | 7.411865 |
| autoimmune disease | 1.2 | 3.726381 |
| autoimmune disease | 1 | 3.071548 |
| autoimmune disease | 1 | 1.81345 |
| cancer (leukemia) | −8.5 | 13.14838 |
| cancer | 5.2 | 3.471052 |
| cancer | −4.7 | 10.5371 |
| cancer | 4.4 | 6.538621 |
| cancer | 3.7 | 4.927568 |
| cancer (acute | −3.6 | 6.109867 |
| lymphoblastic leukemia) | | |
| cancer (acute | −3.2 | 5.319742 |
| lymphoblastic leukemia) | | |
| cancer (acute | −2.8 | 6.007489 |
| lymphoblastic leukemia) | | |
| cancer | 2.2 | 2.247623 |
| cancer | 2 | 1.563864 |
| cancer | −1.9 | 5.065433 |
| cancer | 1.9 | 4.688038 |
| cancer | −1.9 | 2.090055 |
| cancer | −1.7 | 3.377355 |
| cancer | −1.6 | 3.022057 |
| cancer (chronic | −1.5 | 8.761447 |
| lymphocytic leukemia) | | |
| cancer | 1.5 | 6.607219 |
| cancer | −1.5 | 2.843718 |
| cancer | −1.4 | 2.466334 |
| cancer | 1.3 | 5.284269 |
| cancer | 1.3 | 2.606221 |
| cancer | −1.3 | 2.172343 |
| cancer | 1.3 | 1.380578 |
| cancer (acute | −1.2 | 2.842658 |
| lymphoblastic leukemia) | | |
| cancer | −1.1 | 1.863752 |
| cancer | 1.1 | 1.787425 |
| cancer | −1.1 | 1.363211 |
| cancer | −1.1 | 1.363211 |

TABLE 12

| | Log2 | | |
|---|---|---|---|
| | fold | | |
| Disease/phenotype | change | Percentile | p-value |
| chronic | −1.5 | 99 | 1.73 × 10⁻⁹ |
| lymphocytic | | | |
| leukemia | | | |

The data shows that there is increased IGHV4-34 expression in autoimmune disease such as rheumatoid arthritis and lupus decreased expression in cancer such as chronic lymphocytic leukemia.
(ii) a light chain variable (VL) region comprising:
a LC-CDR1 having the amino acid sequence of SEQ ID NO: 62;
a LC-CDR2 having the amino acid sequence YAS;
a LC-CDR3 having the amino acid sequence of SEQ ID NO: 64; or
(e)
(i) a heavy chain variable (VH) region comprising:
a HC-CDR1 having the amino acid sequence of SEQ ID NO: 6:
a HC-CDR2 having the amino acid sequence of SEQ ID NO: 7;
a HC-CDR3 having the amino acid sequence of SEQ ID NO: 8; and
(ii) a light chain variable (VL) region comprising:
a LC-CDR1 having the amino acid sequence of SEQ ID NO: 131;
a LC-CDR2 having the amino acid sequence LVS:
a LC-CDR3 having the amino acid sequence of SEQ ID NO: 16; or
(f)
(i) a heavy chain variable (VH) region comprising:
a HC-CDR1 having the amino acid sequence of SEQ ID NO: 22;
a HC-CDR2 having the amino acid sequence of SEQ ID NO: 23;
a HC-CDR3 having the amino acid sequence of SEQ ID NO: 24; and
(ii) a light chain variable (VL) region comprising:
a LC-CDR1 having the amino acid sequence of SEQ ID NO: 30;
a LC-CDR2 having the amino acid sequence YTS:
a LC-CDR3 having the amino acid sequence of SEQ ID NO: 32; or
(g)
(i) a heavy chain variable (VH) region comprising:
a HC-CDR1 having the amino acid sequence of SEQ ID NO: 6.
a HC-CDR2 having the amino acid sequence of SEQ ID NO: 7;
a HC-CDR3 having the amino acid sequence of SEQ ID NO: 8; and
(ii) a light chain variable (VL) region comprising:
a LC-CDR1 having the amino acid sequence of SEQ ID NO: 162;
a LC-CDR2 having the amino acid sequence LVS:
a LC-CDR3 having the amino acid sequence of SEQ ID NO: 16; or
(h)
(i) a heavy chain variable (VH) region comprising:
a HC-CDR1 having the amino acid sequence of SEQ ID NO: 152:
a HC-CDR2 having the amino acid sequence of SEQ ID NO: 55;
a HC-CDR3 having the amino acid sequence of SEQ ID NO: 56; and
(ii) a light chain variable (VL) region comprising:
a LC-CDR1 having the amino acid sequence of SEQ ID NO: 154:
a LC-CDR2 having the amino acid sequence YAS;
a LC-CDR3 having the amino acid sequence of SEQ ID NO: 64; or
(i)
(i) a heavy chain variable (VH) region comprising:
a HC-CDR1 having the amino acid sequence of SEQ ID NO: 185;
a HC-CDR2 having the amino acid sequence of SEQ ID NO: 55;
a HC-CDR3 having the amino acid sequence of SEQ ID NO: 56; and
(ii) a light chain variable (VL) region comprising:
a LC-CDR1 having the amino acid sequence of SEQ ID NO: 144:
a LC-CDR2 having the amino acid sequence YAS;
a LC-CDR3 having the amino acid sequence of SEQ ID NO: 64; or
(j)
(i) a heavy chain variable (VH) region comprising:
a HC-CDR1 having the amino acid sequence of SEQ ID NO: 6;
a HC-CDR2 having the amino acid sequence of SEQ ID NO: 7:
a HC-CDR3 having the amino acid sequence of SEQ ID NO: 8; and
(ii) a light chain variable (VL) region comprising:
a LC-CDR1 having the amino acid sequence of SEQ ID NO: 14;
a LC-CDR2 having the amino acid sequence LVS;
a LC-CDR3 having the amino acid sequence of SEQ ID NO: 16.
2. The VH4-34 antigen-binding molecule of claim 1, wherein the VH4-34 antigen-binding molecule comprises:
   a VH region comprising an amino acid sequence having at least 80% sequence identity to the amino acid sequence of SEQ ID NO: 5, 21, 37, 53, 125, 141, 151, 156, 167, 171, 176, 181, 182, or 184; and
   a VL region comprising an amino acid sequence having at least 80% sequence identity to the amino acid sequence of SEQ ID NO: 13, 29, 45, 61, 130, 143, 147, 153, 161, 189, 193, 198, 201, 205 or 209.
3. The VH4-34 antigen-binding molecule of any one of claims 1-2, wherein the VH4-34 antigen-binding molecule comprises:
(i) a VH region comprising an amino acid sequence having at least 80% sequence identity to the amino acid sequence of SEQ ID NO: 182; and
   a VL region comprising an amino acid sequence having at least 80% sequence identity to the amino acid sequence of SEQ ID NO: 198;
or
(ii) a VH region comprising an amino acid sequence having at least 80% sequence identity to the amino acid sequence of SEQ ID NO: 182; and
   a VL region comprising an amino acid sequence having at least 80% sequence identity to the amino acid sequence of SEQ ID NO: 193;
or
(iii) a VH region comprising an amino acid sequence having at least 80% sequence identity to the amino acid sequence of SEQ ID NO: 167; and
   a VL region comprising an amino acid sequence having at least 80% sequence identity to the amino acid sequence of SEQ ID NO: 189;
or
(iv) a VH region comprising an amino acid sequence having at least 80% sequence identity to the amino acid sequence of SEQ ID NO: 21; and
   a VL region comprising an amino acid sequence having at least 80% sequence identity to the amino acid sequence of SEQ ID NO: 29;
or
(v) a VH region comprising an amino acid sequence having at least 80% sequence identity to the amino acid sequence of SEQ ID NO: 37; and
   a VL region comprising an amino acid sequence having at least 80% sequence identity to the amino acid sequence of SEQ ID NO: 45;
or
(vi) a VH region comprising an amino acid sequence having at least 80% sequence identity to the amino acid sequence of SEQ ID NO: 53; and
   a VL region comprising an amino acid sequence having at least 80% sequence identity to the amino acid sequence of SEQ ID NO: 61;
or
(vii) a VH region comprising an amino acid sequence having at least 80% sequence identity to the amino acid sequence of SEQ ID NO: 125; and
   a VL region comprising an amino acid sequence having at least 80% sequence identity to the amino acid sequence of SEQ ID NO: 130;
or
(viii) a VH region comprising an amino acid sequence having at least 80% sequence identity to the amino acid sequence of SEQ ID NO: 141; and
   a VL region comprising an amino acid sequence having at least 80% sequence identity to the amino acid sequence of SEQ ID NO: 143;
or
(ix) a VH region comprising an amino acid sequence having at least 80% sequence identity to the amino acid sequence of SEQ ID NO: 141; and
   a VL region comprising an amino acid sequence having at least 80% sequence identity to the amino acid sequence of SEQ ID NO: 147:
or
(x) a VH region comprising an amino acid sequence having at least 80% sequence identity to the amino acid sequence of SEQ ID NO: 156; and
   a VL region comprising an amino acid sequence having at least 80% sequence identity to the amino acid sequence of SEQ ID NO: 161;
or
(xi) a VH region comprising an amino acid sequence having at least 80% sequence identity to the amino acid sequence of SEQ ID NO: 151; and
   a VL region comprising an amino acid sequence having at least 80% sequence identity to the amino acid sequence of SEQ ID NO: 153.
or
(xii) a VH region comprising an amino acid sequence having at least 80% sequence identity to the amino acid sequence of SEQ ID NO: 184; and
   a VL region comprising an amino acid sequence having at least 80% sequence identity to the amino acid sequence of SEQ ID NO: 189, 193, 198, 201, 205 or 209;
or
(xiii) a VH region comprising an amino acid sequence having at least 80% sequence identity to the amino acid sequence of SEQ ID NO: 167; and
   a VL region comprising an amino acid sequence having at least 80% sequence identity to the amino acid sequence of SEQ ID NO: 189, 193, 198, 201, 205 or 209;
or
(xiv) a VH region comprising an amino acid sequence having at least 80% sequence identity to the amino acid sequence of SEQ ID NO: 171; and
   a VL region comprising an amino acid sequence having at least 80% sequence identity to the amino acid sequence of SEQ ID NO: 189, 193, 198, 201, 205 or 209;
or
(xv) a VH region comprising an amino acid sequence having at least 80% sequence identity to the amino acid sequence of SEQ ID NO: 176; and
   a VL region comprising an amino acid sequence having at least 80% sequence identity to the amino acid sequence of SEQ ID NO: 189, 193, 198, 201, 205 or 209;
or
(xvi) a VH region comprising an amino acid sequence having at least 80% sequence identity to the amino acid sequence of SEQ ID NO: 181; and
   a VL region comprising an amino acid sequence having at least 80% sequence identity to the amino acid sequence of SEQ ID NO: 189, 193, 198, 201, 205 or 209;
or
(xvii) a VH region comprising an amino acid sequence having at least 80% sequence identity to the amino acid sequence of SEQ ID NO: 182; and
   a VL region comprising an amino acid sequence having at least 80% sequence identity to the amino acid sequence of SEQ ID NO: 189, 193, 198, 201, 205 or 209;
or
(xviii) a VH region comprising an amino acid sequence having at least 80% sequence identity to the amino acid sequence of SEQ ID NO: 5; and
   a VL region comprising an amino acid sequence having at least 80% sequence identity to the amino acid sequence of SEQ ID NO: 13.
4. The VH4-34 antigen-binding molecule of any one of claims 1-3, wherein the VH4-34 antigen-binding molecule inhibits interaction between VH4-34 and a molecule comprising an N-acetyl lactosamine moiety, optionally wherein the molecule comprising an N-acetyl lactosamine moiety is selected from: an I/I carbohydrate and a CD45 isoform B220.
5. The VH4-34 antigen-binding molecule of any one of claims 1-4, wherein the VH4-34 antigen-binding molecule further comprises an Fc region.
6. The VH4-34 antigen-binding molecule of any one of claims 1-5, wherein the VH4-34 antigen-binding molecule is a multispecific antigen-binding molecule, and wherein the VH4-34 antigen-binding molecule further comprises an antigen-binding domain which binds to an antigen other than VH4-34.
7. A chimeric antigen receptor (CAR) comprising the V1H4-34 antigen-binding molecule of any one of claims 1-6.
8. A nucleic acid, or a plurality of nucleic acids, optionally isolated, encoding the VH4-34 antigen-binding molecule of any one of claims 1-6, or a CAR according to claim 7.
9. An expression vector, or a plurality of expression vectors, comprising a nucleic acid or a plurality of nucleic acids of claim 8.
10. A cell comprising the VH4-34 antigen-binding molecule according of any one of claims 1-6, a CAR according to claim 7, a nucleic acid or a plurality ofnucleic acids according to claim 8, or an expression vector or a plurality of expression vectors according to claim 9.
11. A method comprising culturing a cell of claim 10 under conditions suitable for expression of an antigen-binding molecule or CAR by the cell.
12. A composition comprising the VH4-34 antigen-binding molecule of any one of claims 1-6, a CAR according to claim 7, a nucleic acid or a plurality of nucleic acids according to claim 8, an expression vector or a plurality of expression vectors according to claim 9, or a cell of claim 10, and a pharmaceutically acceptable carrier, diluent, excipient or adjuvant.
13. The VH4-34 antigen-binding molecule of any one of claims 1-6, a CAR according to claim 7, a nucleic acid or a plurality of nucleic acids according to claim 8, an expression vector or a plurality of expression vectors according to claim 9, a cell of claim 10, or a composition of claim 12, for use in a method of medical treatment or prophylaxis.
14. The VH4-34 antigen-binding molecule of any one of claims 1-6, a CAR according to claim 7, a nucleic acid or a plurality of nucleic acids according to claim 8, an expression vector or a plurality of expression vectors according to claim 9, a cell of claim 10, or a composition of claim 12, for use in a method of treatment or prevention of a disease or condition characterised by expression of VH4-34.
15. Use of the VH4-34 antigen-binding molecule of any one of claims 1-6, a CAR according to claim 7, a nucleic acid or a plurality of nucleic acids according to claim 8, an expression vector or a plurality of expression vectors according to claim 9, a cell of claim 10, or a composition of claim 12, in the manufacture of a medicament for treating or preventing a disease or condition characterised by expression of VH4-34.
16. The use of claim 14 or 15, wherein the disease or condition characterised by expression of VH4-34 is an autoimmune disease. 17. The use of claim 16, wherein the autoimmune disease is selected from the group consisting of autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD), and Wiskott-Aldrich syndrome.
18. The use of claim 17, wherein the autoimmune disease is cold agglutinin disease (CAD).
19. The use of claim 17, wherein the autoimmune disease is systemic lupus erythematosus (SLE).
20. The use of claim 14 or 15, wherein the disease or condition characterised by expression of VH4-34 is cancer.
21. The use of claim 20, wherein the cancer is a B cell malignancy.
22. The use of claim 21, wherein the B cell malignancy is selected from the group consisting of a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, and mantle cell lymphoma.
23. The use of claim 22, wherein the B cell malignancy is chronic lymphocytic leukemia (CLL).
24. The use of claim 14 or 15, wherein the disease or condition characterised by expression of VH4-34 is selected from: a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, mantle cell lymphoma, autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD), and Wiskott-Aldrich syndrome.
25. A method of treating or preventing a disease or condition characterised by expression of VH4-34 in a subject, the method comprising administering to the subject the VH4-34 antigen-binding molecule of any one of claims 1-6, a CAR of claim 8, a nucleic acid or a plurality of nucleic acids according to claim 8, an expression vector or a plurality of expression vectors according to claim 9, a cell of claim 10, or a composition of claim 12.
26. The method of claim 25, wherein the disease or condition characterised by expression of VH4-34 is an autoimmune disease.
27. The method of claim 26, wherein the autoimmune disease is selected from the group consisting of autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD), and Wiskott-Aldrich syndrome.
28. The method of claim 27, wherein the autoimmune disease is cold agglutinin disease (CAD).
29. The method of claim 25, wherein the disease or condition characterised by expression of VH4-34 is cancer.
30. The method of claim 29, wherein the cancer is a B cell malignancy.
31. The method of claim 30, wherein the B cell malignancy is selected from the group consisting of a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, and mantle cell lymphoma.
32. The method of claim 31, wherein the B cell malignancy is chronic lymphocytic leukemia (CLL).
33. The method of claim 25, wherein the disease or condition characterised by expression of VH4-34 is selected from: a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, mantle cell lymphoma, autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD), and Wiskott-Aldrich syndrome.
34. Use of the VH4-34 antigen-binding molecule of any one of claims 1-6, to potentiate killing of cells expressing VH4-34.
35. An in vitro complex, optionally isolated, comprising the VH4-34 antigen-binding molecule of any one of claims 1-6 bound to VH4-34.
36. A method for detecting VH4-34 in a sample, comprising contacting a sample containing, or suspected to contain, VH4-34 with the VH4-34 antigen-binding molecule of any one of claims 1-6, and detecting the formation of a complex of the VH4-34 antigen-binding molecule with VH4-34.
37. A method of selecting or stratifying a subject for treatment with a VH4-34-targeted agent, the method comprising contacting, in vitro, a sample from the subject with the VH4-34 antigen-binding molecule of any one of claims 1-6, and detecting the formation of a complex of the VH4-34 antigen-binding molecule with VH4-34.
38. Use of the VH4-34 antigen-binding molecule of any one of claims 1-6 as an in vitro or in vivo diagnostic or prognostic agent.
39. Use of the VH4-34 antigen-binding molecule of any one of claims 1-6 in a method for detecting, localizing or imaging a disease or condition characterised by expression of VH4-34, optionally wherein the disease or condition characterised by expression of VH4-34 is selected from: a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, mantle cell lymphoma, autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD) and Wiskott-Aldrich syndrome.

1. A VH4-34 antigen-binding molecule comprising:
(a)
(i) a heavy chain variable (VH) region comprising:
a HC-CDR1 having the amino acid sequence of SEQ ID NO: 172;
a HC-CDR2 having the amino acid sequence of SEQ ID NO: 55;
a HC-CDR3 having the amino acid sequence of SEQ ID NO: 56; and
(ii) a light chain variable (VL) region comprising:
a LC-CDR1 having the amino acid sequence of SEQ ID NO: 144:
a LC-CDR2 having the amino acid sequence YAS;
a LC-CDR3 having the amino acid sequence of SEQ ID NO: 64; or
(b)
(i) a heavy chain variable (VH) region comprising:
a HC-CDR1 having the amino acid sequence of SEQ ID NO: 142;
a HC-CDR2 having the amino acid sequence of SEQ ID NO: 55;
a HC-CDR3 having the amino acid sequence of SEQ ID NO: 56; and
(ii) a light chain variable (VL) region comprising:
a LC-CDR1 having the amino acid sequence of SEQ ID NO: 144;
a LC-CDR2 having the amino acid sequence YAS;
a LC-CDR3 having the amino acid sequence of SEQ ID NO: 64; or
(c)
(i) a heavy chain variable (VH) region comprising:
a HC-CDR1 having the amino acid sequence of SEQ ID NO: 38;
a HC-CDR2 having the amino acid sequence of SEQ ID NO: 39;
a HC-CDR3 having the amino acid sequence of SEQ ID NO: 40, and
(ii) a light chain variable (VL) region comprising:
a LC-CDR1 having the amino acid sequence of SEQ ID NO: 46;
a LC-CDR2 having the amino acid sequence AAS;
a LC-CDR3 having the amino acid sequence of SEQ ID NO: 48; or
(d)
(i) a heavy chain variable (VH) region comprising:
a HC-CDR1 having the amino acid sequence of SEQ ID NO: 54;
a HC-CDR2 having the amino acid sequence of SEQ ID NO: 55;
a HC-CDR3 having the amino acid sequence of SEQ ID NO: 56; and
(ii) a light chain variable (VL) region comprising:
a LC-CDR1 having the amino acid sequence of SEQ ID NO: 62;
a LC-CDR2 having the amino acid sequence YAS;
a LC-CDR3 having the amino acid sequence of SEQ ID NO: 64; or
(e)
(i) a heavy chain variable (VH) region comprising:
a HC-CDR1 having the amino acid sequence of SEQ ID NO: 6:
a HC-CDR2 having the amino acid sequence of SEQ ID NO: 7;
a HC-CDR3 having the amino acid sequence of SEQ ID NO: 8; and
(ii) a light chain variable (VL) region comprising:
a LC-CDR1 having the amino acid sequence of SEQ ID NO: 131;
a LC-CDR2 having the amino acid sequence LVS:
a LC-CDR3 having the amino acid sequence of SEQ ID NO: 16; or
(f)
(i) a heavy chain variable (VH) region comprising:
a HC-CDR1 having the amino acid sequence of SEQ ID NO: 22;
a HC-CDR2 having the amino acid sequence of SEQ ID NO: 23;
a HC-CDR3 having the amino acid sequence of SEQ ID NO: 24; and
(ii) a light chain variable (VL) region comprising:
a LC-CDR1 having the amino acid sequence of SEQ ID NO: 30;
a LC-CDR2 having the amino acid sequence YTS:
a LC-CDR3 having the amino acid sequence of SEQ ID NO: 32; or
(g)
(i) a heavy chain variable (VH) region comprising:
a HC-CDR1 having the amino acid sequence of SEQ ID NO: 6,
a HC-CDR2 having the amino acid sequence of SEQ ID NO: 7;
a HC-CDR3 having the amino acid sequence of SEQ ID NO: 8; and
(ii) a light chain variable (VL) region comprising:
a LC-CDR1 having the amino acid sequence of SEQ ID NO: 162;
a LC-CDR2 having the amino acid sequence LVS:
a LC-CDR3 having the amino acid sequence of SEQ ID NO: 16; or
(h)
(i) a heavy chain variable (VH) region comprising:
a HC-CDR1 having the amino acid sequence of SEQ ID NO: 152:
a HC-CDR2 having the amino acid sequence of SEQ ID NO: 55;
a HC-CDR3 having the amino acid sequence of SEQ ID NO: 56; and
(ii) a light chain variable (VL) region comprising:
a LC-CDR1 having the amino acid sequence of SEQ ID NO: 154:
a LC-CDR2 having the amino acid sequence YAS;
a LC-CDR3 having the amino acid sequence of SEQ ID NO: 64; or
(i)
(i) a heavy chain variable (VH) region comprising:
a HC-CDR1 having the amino acid sequence of SEQ ID NO: 185;
a HC-CDR2 having the amino acid sequence of SEQ ID NO: 55;
a HC-CDR3 having the amino acid sequence of SEQ ID NO: 56; and
(ii) a light chain variable (VL) region comprising:
a LC-CDR1 having the amino acid sequence of SEQ ID NO: 144:
a LC-CDR2 having the amino acid sequence YAS;
a LC-CDR3 having the amino acid sequence of SEQ ID NO: 64; or
(j)
(i) a heavy chain variable (VH) region comprising:
a HC-CDR1 having the amino acid sequence of SEQ ID NO: 6;
a HC-CDR2 having the amino acid sequence of SEQ ID NO: 7:
a HC-CDR3 having the amino acid sequence of SEQ ID NO: 8; and
(ii) a light chain variable (VL) region comprising:
a LC-CDR1 having the amino acid sequence of SEQ ID NO: 14;
a LC-CDR2 having the amino acid sequence LVS;
a LC-CDR3 having the amino acid sequence of SEQ ID NO: 16.

2. The VH4-34 antigen-binding molecule of claim 1, wherein the VH4-34 antigen-binding molecule comprises:
a VH region comprising an amino acid sequence having at least 80% sequence identity to the amino acid sequence of SEQ ID NO: 5, 21, 37, 53, 125, 141, 151, 156, 167, 171, 176, 181, 182, or 184; and
a VL region comprising an amino acid sequence having at least 80% sequence identity to the amino acid sequence of SEQ ID NO: 13, 29, 45, 61, 130, 143, 147, 153, 161, 189, 193, 198, 201, 205 or 209.

3. The VH4-34 antigen-binding molecule of any one of claims 1-2, wherein the VH4-34 antigen-binding molecule comprises:
(i) a VH region comprising an amino acid sequence having at least 80% sequence identity to the amino acid sequence of SEQ ID NO: 182; and
a VL region comprising an amino acid sequence having at least 80% sequence identity to the amino acid sequence of SEQ ID NO: 198;
or
(ii) a VH region comprising an amino acid sequence having at least 80% sequence identity to the amino acid sequence of SEQ ID NO: 182; and
a VL region comprising an amino acid sequence having at least 80% sequence identity to the amino acid sequence of SEQ ID NO: 193;
or
(iii) a VH region comprising an amino acid sequence having at least 80% sequence identity to the amino acid sequence of SEQ ID NO: 167; and
a VL region comprising an amino acid sequence having at least 80% sequence identity to the amino acid sequence of SEQ ID NO: 189;
or
(iv) a VH region comprising an amino acid sequence having at least 80% sequence identity to the amino acid sequence of SEQ ID NO: 21; and
a VL region comprising an amino acid sequence having at least 80% sequence identity to the amino acid sequence of SEQ ID NO: 29;
or
(v) a VH region comprising an amino acid sequence having at least 80% sequence identity to the amino acid sequence of SEQ ID NO: 37; and
a VL region comprising an amino acid sequence having at least 80% sequence identity to the amino acid sequence of SEQ ID NO: 45;
or
(vi) a VH region comprising an amino acid sequence having at least 80% sequence identity to the amino acid sequence of SEQ ID NO: 53; and
a VL region comprising an amino acid sequence having at least 80% sequence identity to the amino acid sequence of SEQ ID NO: 61;
or
(vii) a VH region comprising an amino acid sequence having at least 80% sequence identity to the amino acid sequence of SEQ ID NO: 125; and
a VL region comprising an amino acid sequence having at least 80% sequence identity to the amino acid sequence of SEQ ID NO: 130;
or
(viii) a VH region comprising an amino acid sequence having at least 80% sequence identity to the amino acid sequence of SEQ ID NO: 141; and
a VL region comprising an amino acid sequence having at least 80% sequence identity to the amino acid sequence of SEQ ID NO: 143;
or
(ix) a VH region comprising an amino acid sequence having at least 80% sequence identity to the amino acid sequence of SEQ ID NO: 141; and
a VL region comprising an amino acid sequence having at least 80% sequence identity to the amino acid sequence of SEQ ID NO: 147:
or
(x) a VH region comprising an amino acid sequence having at least 80% sequence identity to the amino acid sequence of SEQ ID NO: 156; and
a VL region comprising an amino acid sequence having at least 80% sequence identity to the amino acid sequence of SEQ ID NO: 161;
or
(xi) a VH region comprising an amino acid sequence having at least 80% sequence identity to the amino acid sequence of SEQ ID NO: 151; and
a VL region comprising an amino acid sequence having at least 80% sequence identity to the amino acid sequence of SEQ ID NO: 153, or
(xii) a VH region comprising an amino acid sequence having at least 80% sequence identity to the amino acid sequence of SEQ ID NO: 184; and
a VL region comprising an amino acid sequence having at least 80% sequence identity to the amino acid sequence of SEQ ID NO: 189, 193, 198, 201, 205 or 209;
or
(xiii) a VH region comprising an amino acid sequence having at least 80% sequence identity to the amino acid sequence of SEQ ID NO: 167; and
a VL region comprising an amino acid sequence having at least 80% sequence identity to the amino acid sequence of SEQ ID NO: 189, 193, 198, 201, 205 or 209;
or
(xiv) a VH region comprising an amino acid sequence having at least 80% sequence identity to the amino acid sequence of SEQ ID NO: 171; and
a VL region comprising an amino acid sequence having at least 80% sequence identity to the amino acid sequence of SEQ ID NO: 189, 193, 198, 201, 205 or 209;
or
(xv) a VH region comprising an amino acid sequence having at least 80% sequence identity to the amino acid sequence of SEQ ID NO: 176; and
a VL region comprising an amino acid sequence having at least 80% sequence identity to the amino acid sequence of SEQ ID NO: 189, 193, 198, 201, 205 or 209;
or
(xvi) a VH region comprising an amino acid sequence having at least 80% sequence identity to the amino acid sequence of SEQ ID NO: 181; and
a VL region comprising an amino acid sequence having at least 80% sequence identity to the amino acid sequence of SEQ ID NO: 189, 193, 198, 201, 205 or 209;
or
(xvii) a VH region comprising an amino acid sequence having at least 80% sequence identity to the amino acid sequence of SEQ ID NO: 182; and
a VL region comprising an amino acid sequence having at least 80% sequence identity to the amino acid sequence of SEQ ID NO: 189, 193, 198, 201, 205 or 209;
or
(xviii) a VH region comprising an amino acid sequence having at least 80% sequence identity to the amino acid sequence of SEQ ID NO: 5; and
a VL region comprising an amino acid sequence having at least 80% sequence identity to the amino acid sequence of SEQ ID NO: 13.

4. The VH4-34 antigen-binding molecule of any one of claims 1-3, wherein the VH4-34 antigen-binding molecule inhibits interaction between VH4-34 and a molecule comprising an N-acetyl lactosamine moiety, optionally wherein the molecule comprising an N-acetyl lactosamine moiety is selected from: an I/I carbohydrate and a CD45 isoform B220.

5. The VH4-34 antigen-binding molecule of any one of claims 1-4, wherein the VH4-34 antigen-binding molecule further comprises an Fc region.

6. The VH4-34 antigen-binding molecule of any one of claims 1-5, wherein the VH4-34 antigen-binding molecule is a multispecific antigen-binding molecule, and wherein the VH4-34 antigen-binding molecule further comprises an antigen-binding domain which binds to an antigen other than VH4-34.

7. A chimeric antigen receptor (CAR) comprising the V1H4-34 antigen-binding molecule of any one of claims 1-6.

8. A nucleic acid, or a plurality of nucleic acids, optionally isolated, encoding the VH4-34 antigen-binding molecule of any one of claims 1-6, or a CAR according to claim 7.

9. An expression vector, or a plurality of expression vectors, comprising a nucleic acid or a plurality of nucleic acids of claim 8.

10. A cell comprising the VH4-34 antigen-binding molecule according of any one of claims 1-6, a CAR according to claim 7, a nucleic acid or a plurality ofnucleic acids according to claim 8, or an expression vector or a plurality of expression vectors according to claim 9.

11. A method comprising culturing a cell of claim 10 under conditions suitable for expression of an antigen-binding molecule or CAR by the cell.

12. A composition comprising the VH4-34 antigen-binding molecule of any one of claims 1-6, a CAR according to claim 7, a nucleic acid or a plurality of nucleic acids according to claim 8, an expression vector or a plurality of expression vectors according to claim 9, or a cell of claim 10, and a pharmaceutically acceptable carrier, diluent, excipient or adjuvant.

13. The VH4-34 antigen-binding molecule of any one of claims 1-6, a CAR according to claim 7, a nucleic acid or a plurality of nucleic acids according to claim 8, an expression vector or a plurality of expression vectors according to claim 9, a cell of claim 10, or a composition of claim 12, for use in a method of medical treatment or prophylaxis.

14. The VH4-34 antigen-binding molecule of any one of claims 1-6, a CAR according to claim 7, a nucleic acid or a plurality of nucleic acids according to claim 8, an expression vector or a plurality of expression vectors according to claim 9, a cell of claim 10, or a composition of claim 12, for use in a method of treatment or prevention of a disease or condition characterised by expression of VH4-34.

15. Use of the VH4-34 antigen-binding molecule of any one of claims 1-6, a CAR according to claim 7, a nucleic acid or a plurality of nucleic acids according to claim 8, an expression vector or a plurality of expression vectors according to claim 9, a cell of claim 10, or a composition of claim 12, in the manufacture of a medicament for treating or preventing a disease or condition characterised by expression of VH4-34.

16. The use of claim 14 or 15, wherein the disease or condition characterised by expression of VH4-34 is an autoimmune disease.

17. The use of claim 16, wherein the autoimmune disease is selected from the group consisting of autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD), and Wiskott-Aldrich syndrome.

18. The use of claim 17, wherein the autoimmune disease is cold agglutinin disease (CAD).

19. The use of claim 17, wherein the autoimmune disease is systemic lupus erythematosus (SLE).

20. The use of claim 14 or 15, wherein the disease or condition characterised by expression of VH4-34 is cancer.

21. The use of claim 20, wherein the cancer is a B cell malignancy.

22. The use of claim 21, wherein the B cell malignancy is selected from the group consisting of a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, and mantle cell lymphoma.

23. The use of claim 22, wherein the B cell malignancy is chronic lymphocytic leukemia (CLL).

24. The use of claim 14 or 15, wherein the disease or condition characterised by expression of VH4-34 is selected from: a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, mantle cell lymphoma, autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD), and Wiskott-Aldrich syndrome.

25. A method of treating or preventing a disease or condition characterised by expression of VH4-34 in a subject, the method comprising administering to the subject the VH4-34 antigen-binding molecule of any one of claims 1-6, a CAR of claim 8, a nucleic acid or a plurality of nucleic acids according to claim 8, an expression vector or a plurality of expression vectors according to claim 9, a cell of claim 10, or a composition of claim 12.

26. The method of claim 25, wherein the disease or condition characterised by expression of VH4-34 is an autoimmune disease.

27. The method of claim 26, wherein the autoimmune disease is selected from the group consisting of autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD), and Wiskott-Aldrich syndrome.

28. The method of claim 27, wherein the autoimmune disease is cold agglutinin disease (CAD).

29. The method of claim 25, wherein the disease or condition characterised by expression of VH4-34 is cancer.

30. The method of claim 29, wherein the cancer is a B cell malignancy.

31. The method of claim 30, wherein the B cell malignancy is selected from the group consisting of a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, and mantle cell lymphoma.

32. The method of claim 31, wherein the B cell malignancy is chronic lymphocytic leukemia (CLL).

33. The method of claim 25, wherein the disease or condition characterised by expression of VH4-34 is selected from: a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, mantle cell lymphoma, autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD), and Wiskott-Aldrich syndrome.

34. Use of the VH4-34 antigen-binding molecule of any one of claims 1-6, to potentiate killing of cells expressing VH4-34.

35. An in vitro complex, optionally isolated, comprising the VH4-34 antigen-binding molecule of any one of claims 1-6 bound to VH4-34.

36. A method for detecting VH4-34 in a sample, comprising contacting a sample containing, or suspected to contain, VH4-34 with the VH4-34 antigen-binding molecule of any one of claims 1-6, and detecting the formation of a complex of the VH4-34 antigen-binding molecule with VH4-34.

37. A method of selecting or stratifying a subject for treatment with a VH4-34-targeted agent, the method comprising contacting, in vitro, a sample from the subject with the VH4-34 antigen-binding molecule of any one of claims 1-6, and detecting the formation of a complex of the VH4-34 antigen-binding molecule with VH4-34.

38. Use of the VH4-34 antigen-binding molecule of any one of claims 1-6 as an in vitro or in vivo diagnostic or prognostic agent.

39. Use of the VH4-34 antigen-binding molecule of any one of claims 1-6 in a method for detecting, localizing or imaging a disease or condition characterised by expression of VH4-34, optionally wherein the disease or condition characterised by expression of VH4-34 is selected from: a B cell leukemia, hairy cell leukemia (HCL), hairy cell leukemia variant (HCL-V), chronic lymphocytic leukemia (CLL), mutated chronic lymphocytic leukemia (M-CLL), acute lymphoblastic leukemia (ALL), a B cell lymphoma, diffuse large B-cell lymphoma (DLBCL), activated B cell diffuse large B-cell lymphoma (ABC-DLBCL), germinal center B cell diffuse large B-cell lymphoma (GBC-DLBCL), primary central nervous system (CNS) lymphoma, mantle cell lymphoma, autoimmune hemolytic anemia (AIHA), common variable immunodeficiency (CVID), rheumatoid arthritis, systemic lupus erythematosus (SLE), lupus nephritis, central nervous system (CNS) lupus, cold agglutinin disease (CAD) and Wiskott-Aldrich syndrome.
